(19) United States
(12) Patent Application Publication
(10) Pub. No.: US 2020/0224144 A1
(43) Pub. Date: Jul. 16, 2020

(54) SYSTEMS AND METHODS FOR MANUFACTURING BIOLOGICALLY-PRODUCED PRODUCTS

(71) Applicants: Massachusetts Institute of Technology, Cambridge, MA (US); Rensselaer Polytechnic Institute, Troy, NY (US)

(72) Inventors: J. Christopher Love, Somerville, MA (US); Kerry R. Love, Somerville, MA (US); Laura Crowell, Cambridge, MA (US); Alan Stockdale, Providence, RI (US); Richard Dean Braatz, Arlington, MA (US); Amos Enshen Lu, Cambridge, MA (US); Steven Cramer, Troy, NY (US); Steven Timmick, Troy, NY (US); Nicholas Vecchiarello, Troy, NY (US); Chaz Goodwine, Troy, NY (US); Craig A. Mascarenhas, Brookline, MA (US)

(73) Assignees: Massachusetts Institute of Technology, Cambridge, MA (US); Rensselaer Polytechnic Institute, Troy, NY (US)

(21) Appl. No.: 16/499,780

(22) PCT Filed: Mar. 30, 2018
(86) PCT No.: PCT/US2018/025582

§ 371 Date: Sep. 30, 2019

Related U.S. Application Data
(60) Provisional application No. 62/646,236, filed on Mar. 21, 2018.
(60) Provisional application No. 62/644,285, filed on Mar. 16, 2018.
(60) Provisional application No. 62/553,105, filed on Aug. 31, 2017.
(60) Provisional application No. 62/533,765, filed on Jul. 18, 2017.
(60) Provisional application No. 62/480,418, filed on Apr. 1, 2017.
(60) Provisional application No. 62/480,422, filed on Apr. 1, 2017.
(60) Provisional application No. 62/480,436, filed on Apr. 1, 2017.
(60) Provisional application No. 62/480,438, filed on Apr. 1, 2017.
(60) Provisional application No. 62/480,426, filed on Apr. 1, 2017.
(60) Provisional application No. 62/480,427, filed on Apr. 1, 2017.
(60) Provisional application No. 62/480,428, filed on Apr. 1, 2017.
(60) Provisional application No. 62/480,430, filed on Apr. 1, 2017.
(60) Provisional application No. 62/480,432, filed on Apr. 1, 2017.
(60) Provisional application No. 62/480,435, filed on Apr. 1, 2017.

(51) Int. Cl. C12M 1/00; C12M 1/34

(52) U.S. Cl. CPC C12M 29/10; C12M 41/44; C07K 14/56; C12M 47/12; C12M 47/10

(57) ABSTRACT

Aspects of the present disclosure relate to systems and methods for manufacturing biologically-produced pharmaceutical products. Some of the systems described herein comprise an upstream component comprising a bioreactor and at least one filter (e.g., a filter probe) integrated with a downstream component comprising a purification module comprising at least a first partitioning unit and a second partitioning unit. In some embodiments, these integrated biomanufacturing systems may be operated under continuous or conditions and may be capable of efficiently producing pure, high-quality pharmaceutical products.

### RELATED APPLICATIONS

This application claims priority under 35 U.S.C. § 119(e) to U.S. Provisional Patent Application Ser. No. 62/480,418, filed Apr. 1, 2017, and entitled “Using Chromatography Behavior Characteristics of Impurities and Target Products to Design Downstream Processes,” U.S. Provisional Patent Application Ser. No. 62/480,422, filed Apr. 1, 2017, and entitled “Using Chromatography Screens to Characterize Impurities and Target Products,”U.S. Provisional Patent Application Ser. No. 62/480,436, filed Apr. 1, 2017, and entitled “Using Chromatography Screens to Characterize Impurities and Target Products,” U.S. Provisional Patent Application Ser. No. 62/480,438, filed Apr. 1, 2017, and entitled “Using Chromatography Screens to Characterize Impurities and Target Products to Design Downstream Processes,” U.S. Provisional Patent Application Ser. No. 62/480,426, filed Apr. 1, 2017, and entitled “Characterizing Behavior of Impurities and Target Products with Respect to Various Partitioning Techniques,” U.S. Provisional Patent Application Ser. No. 62/480,427, filed Apr. 1, 2017, and entitled “Using Behavior Data of Impurities and Target Proteins to Design Downstream Processes,” U.S. Provisional Patent Application Ser. No. 62/480,428, filed Apr. 1, 2017, and entitled “Process/Equipment for High Concentration/Throughput Rapid Perfusion based Production of Biotherapeutics,” U.S. Provisional Patent Application Ser. No. 62/480,430, filed Apr. 1, 2017, and entitled “Process/Equipment for Producing G-CSF,” U.S. Provisional Patent Application Ser. No. 62/480,432, filed Apr. 1, 2017, and entitled “Process/Equipment for Producing IFN,” U.S. Provisional Patent Application Ser. No. 62/480,435, filed Apr. 1, 2017, and entitled “Process/Equipment for Producing HGH,” U.S. Provisional Patent Application Ser. No. 62/533,765, filed Jul. 18, 2017, and entitled “Systems and Methods for Manufacturing Biologically-Produced Products,” U.S. Provisional Patent Application Ser. No. 62/553,105, filed Aug. 31, 2017, and entitled “Level Sensing Systems for Perfusion-Based Systems and Methods for Manufacturing Biologically-Produced Products,” U.S. Provisional Patent Application Ser. No. 62/644,285, filed Mar. 16, 2018, and entitled “Process/Equipment for Producing IFN,” and U.S. Provisional Patent Application Ser. No. 62/646,236, filed Mar. 21, 2018, and entitled “Process/Equipment for Producing Single-Domain Antibodies,” each of which is incorporated herein by reference in its entirety for all purposes.

### GOVERNMENT FUNDING

This invention was made with Government support under Contract No. N66001-13-C-4025 awarded by the Space and Naval Warfare Systems Center. The Government has certain rights in the invention.

### FIELD

The present invention generally relates to systems and methods for manufacturing biologically-produced products.

### BACKGROUND

Biologically-produced pharmaceutical products, which are therapeutic drugs produced by biological organisms, have revolutionized the pharmaceutical industry. Biological organisms are an attractive source of therapeutic drugs because they are often capable of producing molecules that would be challenging, if not impossible, to synthesize chemically. For example, some biological organisms can be engineered to produce complex proteins, such as antibodies and signaling proteins, which can be used to treat or prevent diseases ranging from cancer to rheumatoid arthritis. There are already hundreds of approved biologically-produced pharmaceutical products on the market and thousands of new products in development, and demand for these products continues to grow. However, conventional systems and methods of manufacturing biologically-produced products, such as proteins, are not systematic, continuous, integrated, or modular, resulting in production systems and methods that are often slow, cumbersome, expensive, and/or not amenable to miniaturization. Accordingly, improved systems and methods that are systematic, more integrated, more continuous, and/or modular are needed to allow for more efficient protein production and purification with machines that are portable, amenable to miniaturization, and/or easier to use by operators with a variety of skill levels. Such improved systems and methods would be of significant interest to the biopharmaceutical industry, but would also have applications in bio-energy, medical research, pollution remediation, manufacturing, agriculture, and other fields.

### SUMMARY

The present invention generally relates to systems and methods for manufacturing biologically-produced products. The subject matter of the present invention involves, in some cases, interrelated products, alternative solutions to a particular problem, and/or a plurality of different uses of one or more systems and/or articles.

Certain aspects relate to a biomanufacturing system. In one embodiment, the system comprises a perfusion bioreactor. In certain embodiments, the perfusion bioreactor comprises a reaction chamber configured to receive at least one feed stream comprising at least one cell culture medium. In certain embodiments, the perfusion bioreactor comprises a suspension comprising the at least one cell culture medium and at least a first type of biological cells configured to express at least one biologically-produced product. In some embodiments, the system comprises at least one filter probe at least partially submerged in the suspension. In certain embodiments, the at least one filter probe is configured to produce at least one filtrate stream lean in the first type of biological cells relative to the suspension, wherein the at least one filtrate stream comprises the at least one biologically-produced product. In some embodiments, the system comprises an adjustment module fluidically connected to the perfusion bioreactor, wherein the adjustment module is configured to adjust one or more properties of the at least one filtrate stream to produce an adjusted filtrate stream. In some embodiments, the system comprises a purification module fluidically connected to the adjustment module, wherein the purification module is configured to remove at least a first type of impurity and a second type of impurity from the adjusted filtrate stream to produce a purified filtrate stream. In certain embodiments, the purification module comprises a first partitioning unit configured to remove at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product. In certain embodiments, the purification module comprises a second partitioning unit configured to remove at least a second type of impurity from the first partitioned filtrate stream to produce a second partitioned filtrate stream lean in the second type of impurity relative to the first partitioned filtrate stream, wherein the second partitioned filtrate stream comprises the at least one biologically-produced product. In some embodiments, the system is configured to be continuously operated.

Certain aspects relate to a method of producing at least one biologically-produced product. In one embodiment, the method comprises supplying at least one feed stream comprising at least one cell culture medium to a perfusion bioreactor at a first flow rate. In some embodiments, the method comprises producing, within the perfusion bioreactor, a suspension comprising the at least one cell culture medium and at least a first type of biological cells expressing the at least one biologically-produced product. In some embodiments, the method comprises causing at least a portion of the suspension to flow through at least one filter probe to produce at least one filtrate stream lean in the first type of biological cells, wherein the at least one filtrate stream comprises the at least one biologically-produced product, wherein the at least one filter probe is at least partially submerged in the suspension. In some embodiments, the method comprises adjusting one or more properties of the at least one filtrate stream to produce an adjusted filtrate stream. In some embodiments, the method comprises removing, within a purification module, at least a first type of impurity and a second type of impurity from the adjusted filtrate stream to produce a purified filtrate stream flowing at a second flow rate, wherein the purified filtrate stream comprises the at least one biologically-produced product and is lean in the first type of impurity and the second type of impurity relative to the adjusted filtrate stream. In certain embodiments, producing the purified filtrate stream comprises removing, within a first partitioning unit, at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product. In certain embodiments, producing the purified filtrate stream comprises removing, within a second partitioning unit, at least the second type of impurity from the first partitioned filtrate stream to produce a second partitioned filtrate stream lean in the second type of impurity relative to the first partitioned filtrate stream, wherein the second partitioned filtrate stream comprises the at least one biologically-produced product.

Another embodiment relates to a biomanufacturing system. The system comprises a perfusion bioreactor. In certain embodiments, the perfusion bioreactor comprises a reaction chamber configured to receive at least one feed stream comprising at least one cell culture medium. In certain embodiments, the perfusion bioreactor comprises a suspension comprising the at least one cell culture medium and at least a first type of biological cells configured to express at least one biologically-produced product. In some embodiments, the system comprises a level sensing system configured to measure a level of the suspension in the reactor chamber of the perfusion bioreactor. In some embodiments, the system comprises at least one filter probe at least partially submerged in the suspension. In certain embodiments, the at least one filter probe is configured to produce at least one filtrate stream lean in the first type of biological cells relative to the suspension, wherein the at least one filtrate stream comprises the at least one biologically-produced product. In some embodiments, the system comprises an adjustment module fluidically connected to the perfusion bioreactor, wherein the adjustment module is configured to adjust one or more properties of the at least one filtrate stream to produce an adjusted filtrate stream. In some embodiments, the system comprises a purification module fluidically connected to the adjustment module, wherein the purification module is configured to remove at least a first type of impurity and a second type of impurity from the adjusted filtrate stream to produce a purified filtrate stream. In certain embodiments, the purification module comprises a first partitioning unit configured to remove at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product. In certain embodiments, the purification module comprises a second partitioning unit configured to remove at least a second type of impurity from the first partitioned filtrate stream to produce a second partitioned filtrate stream lean in the second type of impurity relative to the first partitioned filtrate stream, wherein the second partitioned filtrate stream comprises the at least one biologically-produced product. In some embodiments, the system is configured to be continuously operated.

One embodiment relates to a method of producing at least one biologically-produced product. In some embodiments, the method comprises supplying at least one feed stream comprising at least one cell culture medium to a perfusion bioreactor at a first flow rate. In some embodiments, the method comprises producing, within the perfusion bioreactor, a suspension comprising the at least one cell culture medium and at least a first type of biological cells expressing the at least one biologically-produced product. In some embodiments, the method comprises measuring a level of the suspension in a reaction chamber of the perfusion bioreactor using a level sensing system. In some embodiments, the method comprises causing at least a portion of the suspension to flow through at least one filter probe to produce at least one filtrate stream lean in the first type of biological cells, wherein the at least one filtrate stream comprises the at least one biologically-produced product, wherein the at least one filter probe is at least partially submerged in the suspension. In some embodiments, the method comprises adjusting one or more properties of the at least one filtrate stream to produce an adjusted filtrate stream. In some embodiments, the method comprises removing, within a purification module, at least a first type of impurity and a second type of impurity from the adjusted filtrate stream to produce a purified filtrate stream flowing at a second flow rate, wherein the purified filtrate stream comprises the at least one biologically-produced product and is lean in the first type of impurity and the second type of impurity relative to the adjusted filtrate stream. In certain embodiments, producing the purified filtrate stream comprises removing, within a first partitioning unit, at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product. In certain embodiments, producing the purified filtrate stream comprises removing, within a second partitioning unit, at least the second type of impurity from the first partitioned filtrate stream to produce a second partitioned filtrate stream lean in the second type of impurity relative to the first partitioned filtrate stream, wherein the second partitioned filtrate stream comprises the at least one biologically-produced product.

One embodiment relates to a system for producing G-CSF. In some embodiments, the system comprises a bioreactor, wherein the bioreactor comprises a reaction chamber containing a suspension comprising at least one cell culture medium and at least a first type of biological cells configured to express G-CSF. In some embodiments, the system comprises at least one filter, wherein the at least one filter is configured to receive an output of the bioreactor and produce at least one filtrate lean in the first type of biological cells relative to the suspension, wherein the at least one filtrate comprises G-CSF. In some embodiments, the system comprises a purification module, wherein the purification module is configured to remove at least a first type of impurity, a second type of impurity, and a third type of impurity from the first filtrate to produce a purified filtrate. In certain embodiments, the purification module comprises a first column comprising a multimodal cation exchange resin; a second column comprising an anion exchange resin; and a third column comprising an HCIC resin.

Another embodiment relates to a method of producing G-CSF. In some embodiments, the method comprises supplying at least one cell culture medium to a bioreactor. In some embodiments, the method comprises producing, within the bioreactor, a suspension comprising the at least one cell culture medium and at least a first type of biological cells expressing G-CSF. In some embodiments, the method comprises causing at least a portion of the suspension to flow through at least one filter to produce at least one filtrate lean in the first type of biological cells, wherein the at least one filtrate comprises G-CSF. In some embodiments, the method comprises flowing the at least one filtrate through a purification module to produce a purified filtrate. In certain embodiments, producing the purified filtrate comprises flowing the at least one filtrate through a first column comprising a multimodal cation exchange resin; and collecting one or more first fractions comprising G-CSF from an outflow of the first column. In certain embodiments, producing the purified filtrate further comprises flowing the one or more first fractions through a second column comprising an anion exchange resin; and collecting one or more second fractions comprising G-CSF from an outflow of the second column. In certain embodiments, producing the purified filtrate further comprises flowing the one or more second fractions through a third column comprising an HCIC resin; and collecting one or more third fractions comprising G-CSF from an outflow of the third column.

One embodiment relates to a system for producing interferon-α2b (IFN). In some embodiments, the system comprises a bioreactor, wherein the bioreactor comprises a reaction chamber containing a suspension comprising at least one cell culture medium and at least a first type of biological cells configured to express interferon-α2b. In some embodiments, the system comprises at least one filter, wherein the at least one filter is configured to receive an output of the bioreactor and produce at least one filtrate lean in the first type of biological cells relative to the suspension, wherein the at least one filtrate comprises interferon-α2b. In some embodiments, the system comprises a purification module, wherein the purification module is configured to remove at least a first type of impurity, a second type of impurity, and a third type of impurity from the first filtrate to produce a purified filtrate. In certain embodiments, the purification module comprises a first column comprising a multimodal cation exchange resin; a second column comprising an HCIC resin; and a third column comprising a cation exchange resin.

Another embodiment relates to a method of producing interferon-α2b (IFN). In some embodiments, the method comprises supplying at least one cell culture medium to a bioreactor. In some embodiments, the method comprises producing, within the bioreactor, a suspension comprising the at least one cell culture medium and at least a first type of biological cells expressing interferon-α2b. In some embodiments, the method comprises causing at least a portion of the suspension to flow through at least one filter to produce at least one filtrate lean in the first type of biological cells, wherein the at least one filtrate comprises interferon-α2b; and flowing the at least one filtrate through a purification module to produce a purified filtrate. In some embodiments, producing the purified filtrate comprises flowing the at least one filtrate through a first column comprising a multimodal cation exchange resin; and collecting one or more first fractions comprising interferon-α2b from an outflow of the first column. In some embodiments, producing the purified filtrate further comprises flowing the one or more first fractions through a second column comprising an HCIC resin; and collecting one or more second fractions comprising interferon-α2b from an outflow of the second column. In some embodiments, producing the purified filtrate further comprises flowing the one or more second fractions through a third column comprising a cation exchange resin; and collecting one or more third fractions comprising interferon-α2b from an outflow of the third column.

One embodiment relates to a system for producing interferon-α2b (IFN). In some embodiments, the system comprises a bioreactor, wherein the bioreactor comprises a reaction chamber containing a suspension comprising at least one cell culture medium and at least a first type of biological cells configured to express interferon-α2b. In some embodiments, the system comprises at least one filter, wherein the at least one filter is configured to receive an output of the bioreactor and produce at least one filtrate lean in the first type of biological cells relative to the suspension, wherein the at least one filtrate comprises interferon-α2b. In some embodiments, the system comprises a purification module, wherein the purification module is configured to remove at least a first type of impurity, a second type of impurity, and a third type of impurity from the first filtrate to produce a purified filtrate. In certain embodiments, the purification module comprises a first column comprising a multimodal cation exchange resin; a second column comprising a flow-through resin; and a third column comprising an anion exchange resin.

Another embodiment relates to a method of producing interferon-α2b (IFN). In some embodiments, the method comprises supplying at least one cell culture medium to a bioreactor. In some embodiments, the method comprises producing, within the bioreactor, a suspension comprising the at least one cell culture medium and at least a first type of biological cells expressing interferon-α2b. In some embodiments, the method comprises causing at least a portion of the suspension to flow through at least one filter to produce at least one filtrate lean in the first type of biological cells, wherein the at least one filtrate comprises interferon-α2b; and flowing the at least one filtrate through a purification module to produce a purified filtrate. In some embodiments, producing the purified filtrate comprises flowing the at least one filtrate through a first column comprising a multimodal cation exchange resin; and collecting one or more first fractions comprising interferon-α2b from an outflow of the first column. In some embodiments, producing the purified filtrate further comprises flowing the one or more first fractions through a second column comprising a flow-through resin; and collecting one or more second fractions comprising interferon-α2b from an outflow of the second column. In some embodiments, producing the purified filtrate further comprises flowing the one or more second fractions through a third column comprising an anion exchange resin; and collecting one or more third fractions comprising interferon-α2b from an outflow of the third column.

One embodiment relates to a system for producing human growth hormone. In some embodiments, the system comprises a bioreactor, wherein the bioreactor comprises a reaction chamber containing a suspension comprising at least one cell culture medium and at least a first type of biological cells configured to express human growth hormone. In some embodiments, the system comprises at least one filter, wherein the at least one filter is configured to receive an output of the bioreactor and produce at least one filtrate lean in the first type of biological cells relative to the suspension, wherein the at least one filtrate comprises human growth hormone. In some embodiments, the system comprises a purification module, wherein the purification module is configured to remove at least a first type of impurity and a second type of impurity from the at least one filtrate to produce a purified filtrate. In certain embodiments, the purification module comprises a first column comprising a multimodal cation exchange resin; and a second column comprising an anion exchange resin.

Another embodiment relates to a method of producing human growth hormone. In some embodiments, the method comprises supplying at least one cell culture medium to a bioreactor. In some embodiments, the method comprises producing, within the bioreactor, a suspension comprising the at least one cell culture medium and at least a first type of biological cells expressing human growth hormone. In some embodiments, the method comprises causing at least a portion of the suspension to flow through at least one filter to produce at least one filtrate lean in the first type of biological cells, wherein the at least one filtrate comprises human growth hormone. In some embodiments, the method comprises flowing the at least one filtrate through a purification module to produce a purified filtrate. In certain embodiments, producing the purified filtrate comprises flowing the at least one filtrate through a first column comprising a multimodal cation exchange resin; and collecting one or more first fractions comprising human growth hormone from an outflow of the first column. In certain embodiments, producing the purified filtrate further comprises flowing the one or more first fractions through a second column comprising an anion exchange resin; and collecting one or more second fractions comprising human growth hormone from an outflow of the second column.

Another embodiment relates to a system for producing a single-domain antibody. In some embodiments, the system comprises a bioreactor, wherein the bioreactor comprises a reaction chamber containing a suspension comprising at least one cell culture medium and at least a first type of biological cells configured to express the single-domain antibody. In some embodiments, the system comprises at least one filter, wherein the at least one filter is configured to receive an output of the bioreactor and produce at least one filtrate lean in the first type of biological cells relative to the suspension, wherein the at least one filtrate comprises the single-domain antibody. In some embodiments, the system comprises a purification module, wherein the purification module is configured to remove at least a first type of impurity and a second type of impurity from the at least one filtrate to produce a purified filtrate. In certain embodiments, the purification module comprises a first column comprising a multimodal cation exchange resin; and a second column comprising an anion exchange resin.

One embodiment relates to a method of producing a single-domain antibody. In some embodiments, the method comprises supplying at least one cell culture medium to a bioreactor. In some embodiments, the method comprises producing, within the bioreactor, a suspension comprising the at least one cell culture medium and at least a first type of biological cells expressing a single-domain antibody. In some embodiments, the method comprises causing at least a portion of the suspension to flow through at least one filter to produce at least one filtrate lean in the first type of biological cells, wherein the at least one filtrate comprises the single-domain antibody. In some embodiments, the method comprises flowing the at least one filtrate through a purification module to produce a purified filtrate. In certain embodiments, producing the purified filtrate comprises flowing the at least one filtrate through a first column comprising a multimodal cation exchange resin; and collecting one or more first fractions comprising the single-domain antibody from an outflow of the first column. In certain embodiments, producing the purified filtrate further comprises flowing the one or more first fractions through a second column comprising an anion exchange resin; and collecting one or more second fractions comprising the single-domain antibody from an outflow of the second column.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram of an exemplary downstream purification process of an exemplary biological manufacturing system, the downstream purification process comprising a plurality of partitioning steps, according to some embodiments;

FIG. 2 is a schematic diagram of an exemplary set of experiments comprising separately subjecting a target product and impurities to a partitioning step, and analyzing the outputs of the partitioning step to obtain partitioning data for the target product and partitioning data for the impurities, according to some embodiments;

FIG. 3 is a schematic diagram of an exemplary partitioning step using a chromatography technique, which includes introducing an input into an inflow comprising a mobile phase material that is caused to flow through a structure comprising a stationary phase material from which fractions are collected as output, according to some embodiments;

FIG. 4 is a schematic diagram of a plurality of exemplary chromatograms that may result from analyzing the exemplary fractions shown in FIG. 3, according to some embodiments;

FIG. 5 is a schematic diagram of an exemplary data arrangement that may include data collected from a plurality of experiments conducted using different partitioning techniques and/or different parameters, according to some embodiments;

FIG. 6 is a schematic diagram of an exemplary process that may be used to generate and evaluate candidate processes, according to some embodiments;

FIG. 7A is a schematic diagram of an exemplary clusters of partitioning steps, and a list of exemplary candidate processes generated, according to some embodiments;

FIG. 7B is a schematic diagram of an exemplary data arrangement that may include data collected from a plurality of experiments conducted using different partitioning techniques and/or different parameters, according to some embodiments;

FIG. 7C shows an illustrative process 715 for predicting behaviors of host cell proteins, according to some embodiments;

FIG. 7D shows an illustrative chromatogram 720, according to some embodiments;

FIG. 8 shows, schematically, an illustrative computer 8000 on which any aspect of the present disclosure may be implemented;

FIG. 9A is a schematic diagram of an exemplary biomanufacturing system comprising a bioreactor, a filter, and a purification module, according to some embodiments;

FIG. 9B is a schematic diagram of an exemplary biomanufacturing system comprising a bioreactor, a filter, an adjustment module, and a purification module, according to some embodiments;

FIG. 9C is a schematic diagram of an exemplary biomanufacturing system comprising a bioreactor, a filter, a purification module, and a formulation module, according to some embodiments;

FIG. 9D is a schematic diagram of an exemplary biomanufacturing system comprising a bioreactor, a filter, an adjustment module, a purification module, and a formulation module, according to some embodiments;

FIG. 10A is, according to some embodiments, a purification module comprising a first partitioning unit and a second partitioning unit; and

FIG. 10B is, according to some embodiments, a purification module comprising a first partitioning unit, a second partitioning unit, and a third partitioning unit.

FIG. 11 is a three-dimensional plot showing two-dimensional chromatographic data for each fraction eluting from a column using a gradient method;

FIG. 12 is a schematic diagram illustrating an initial purification process for granulocyte-colony stimulating factor (G-CSF) determined by a downstream process generation tool, and the final purification process closely derived from the process generation tool;

FIG. 13 is a representative AKTA chromatogram for a pharmaceutical product human growth hormone (hGH) eluting from a column while NaCl concentration in the mobile phase mixture is continuously varied;

FIG. 14A is a superposition of chromatograms for three orthogonal partitioning steps in a downstream purification process;

FIG. 14B is a superposition of chromatograms for three non-orthogonal partitioning steps in a downstream purification process;

FIG. 15 is a schematic diagram illustrating an initial purification process for human growth hormone (hGH) determined by a downstream process generation tool, and the final purification process closely derived from the process generation tool;

FIG. 16A shows a reverse phase ultra high pressure liquid chromatography (RP-UPLC) chromatogram of partially purified interferon α-2β (IFN);

FIG. 16B shows chromatograms for ten fractions collected from the RP-UPLC fractionation of IFN;

FIG. 17 shows a representative mass spectrum from electrospray ionization mass spectrometry (ESI-MS) carried out on a fraction of IFN from RP-UPLC;

FIG. 18 shows a chromatogram for analyzing the purity of IFN after downstream process purification using a selected process from a process selection software tool;

FIG. 19 shows a composition profile using data from reversed phase-high performance liquid chromatography (RP-HPLC);

FIG. 20 shows chromatograms analyzing a representative purified IFN sample before and after deglycosylation;

FIG. 21 shows sodium dodecyl sulfate polyacrylamide gel electrophoresis (SDS-PAGE) results and other product quality data, where the product was granulocyte-colony stimulating factor (G-CSF) and the purification process was determined by a process development method according to certain embodiments of the present disclosure;

FIG. 22 shows the absorption spectra associated with the outflow from a downstream purification process selected by a process development method according to certain embodiments of the present disclosure, across multiple cycles and multiple trials;

FIG. 23 shows measurements of human growth hormone (hGH) concentration before purification (top) and the number of purified dose equivalents of hGH after purification (bottom);

FIG. 24 shows an SDS-PAGE gel result for purification of hGH;

FIG. 25 shows bioactivity of experimentally purified hGH (experimental) as compared with the WHO International Standard;

FIG. 26 shows sodium dodecyl sulfate polyacrylamide gel electrophoresis (SDS-PAGE) results and other product quality data, where the product was IFN and the purification process was determined by a process development method according to certain embodiments of the present disclosure;

FIG. 27 shows, according to certain embodiments, a schematic illustration of an exemplary system comprising a bioreactor and a magnetic level sensing system;

FIG. 28A shows a schematic illustration of an exemplary system comprising a bioreactor and an optical level sensing system comprising a probe comprising a plurality of colored bands, according to some embodiments;

FIG. 28B shows a schematic illustration of an exemplary system comprising a bioreactor and an optical level sensing system comprising a colored float, according to some embodiments;

FIG. 28C shows a schematic illustration of an exemplary system comprising a bioreactor and an optical level sensing system comprising a colored agitator shaft, according to some embodiments;

FIG. 29 is a schematic diagram illustrating an initial purification process for IFN determined by a downstream process generation tool, and the final purification process closely derived from the process generation tool;

FIG. 30 shows reverse phase ultra-high pressure liquid chromatography (RP-UPLC) chromatograms of a cell culture fluid containing IFN prior to purification and after each purification step;

FIG. 31 is a schematic diagram illustrating an initial purification process for a single-domain antibody determined by a downstream process generation tool, and the final purification process closely derived from the process generation tool;

FIG. 32A shows SDS-PAGE results for unpurified single-domain camelid antibody 3B2 and and the single-domain camelid antibody 3B2 after undergoing the final purification process in FIG. 31;

FIG. 32B shows SDS-PAGE results for unpurified single-domain camelid antibody 2KD1 and and the single-domain camelid antibody 2KD1 after undergoing the final purification process in FIG. 31;

FIG. 33A shows bioactivity of experimentally purified G-CSF as compared with the WHO International Standard;

FIG. 33B shows circular dichroism of experimentally purified G-CSF as compared with a standard;

FIG. 34A shows plasma concentrations over time in rats treated with different doses of experimentally purified G-CSF over time as compared to a standard;

FIG. 34B shows relative neutrophil counts in rats treated with different doses of experimentally purified G-CSF as compared to a standard;

FIG. 35A shows a schematic illustration of an exemplary system comprising a bioreactor and a process monitoring and control system, according to some embodiments;

FIG. 35B shows a schematic illustration of an exemplary system comprising a process monitoring and control system, according to some embodiments;

FIG. 35C shows a schematic illustration of an exemplary system comprising a process monitoring and control system, according to some embodiments;

FIG. 35D shows a schematic illustration of an exemplary system comprising a process monitoring and control system, according to some embodiments;

FIG. 35E shows a schematic illustration of an exemplary system comprising a process monitoring and control system, according to some embodiments;

FIG. 35F shows a schematic illustration of an exemplary facility comprising a process monitoring and control system, according to some embodiments; and

FIG. 36 is a flow diagram of an exemplary process for generating and evaluating candidate processes based on process and product-related impurity rankings, according to some embodiments;

FIG. 37 is, according to some embodiments, an exemplary chromatogram of a sample comprising IFN and product-related impurities;

FIG. 38 is a representation of candidate sequences plotted according to host-related impurity removal rank and product variant removal rank, according to some embodiments;

FIG. 39 is, according to some embodiments, an exemplary list of highly-ranked candidate sequences for removing host-related and product-related impurities during IFN purification;

FIG. 40 is a schematic representation of a proposed IFN purification process and a refined IFN purification process, according to some embodiments;

FIG. 41 is, according to some embodiments, a representation of product concentration, product recovery, HCP concentration, DNA concentration, and product variant content for each partitioning step of an IFN purification process;

FIG. 42 is an exemplary sequence of partitioning steps for IFN purification, according to some embodiments; and

FIG. 43 is, according to some embodiments, an exemplary sequence of partitioning steps for IFN purification.

### DETAILED DESCRIPTION

Aspects of the present disclosure relate to systems and methods for manufacturing biologically-produced products, which may include pharmaceutical and/or protein products. Some of the systems described herein comprise an upstream component comprising a bioreactor and at least one filter (e.g., a filter probe) integrated with a downstream component comprising a purification module comprising at least a first partitioning unit and a second partitioning unit. In some embodiments, these integrated biological manufacturing systems may be operated under continuous conditions and may be capable of efficiently producing pure, high-quality pharmaceutical and/or protein products. In some embodiments, these integrated biological systems may be operated as semi-continuous processes. A semi-continuous process is a process characterized by periods of continuous operation intentionally interrupted by periods of non-operation.

In some cases, the downstream component of the integrated system can be designed through a framework that involves generating a plurality of data sets and using the plurality of data sets to evaluate candidate sequences of partitioning units and/or partitioning conditions. This framework may, in some cases, allow for the efficient design of systems to capture and purify various biologically produced products, such as pharmaceutical and/or protein products. Accordingly, the systems and methods described herein may provide a flexible platform for efficiently manufacturing a wide array of biologically produced products, which may include pharmaceutical and/or protein products.

Typically, in conventional approaches, downstream processing systems are designed based on engineers' experience and intuition. For instance, a chromatography engineer may select a bind-elute step based on a target product to be recovered, identify a fraction in which the target product is eluted, and analyze the fraction to determine which impurities are present. The engineer may then select and experiment with one or more subsequent chromatography steps that the engineer believes will be effective in partitioning the target product from the identified impurities. The engineer may repeat this design process with different chromatography techniques (e.g., ion exchange chromatography, size exclusion chromatography, hydrophobic charge induction chromatography, etc.) and/or different sets of parameters (e.g., different chromatographic resins, pH gradient vs. salt gradient, etc.), and may select a sequence of chromatography steps that achieve one or more objectives (e.g., speed, cost, purity, yield, etc.).

The inventors have recognized and appreciated that such a highly subjective and sequential design process is typically costly and time consuming, and may result in suboptimal downstream processes. For instance, choosing chromatography techniques and/or parameters in a step-by-step fashion may be akin to a greedy algorithm that chooses a local optimum at each step. Such an approach may not always lead to a global optimum. For example, for a list of two candidate processes and a starting mixture of a product and 100 impurities, a first candidate process may remove impurities 1-90 and a second candidate process may remove impurities 1-100, so the second candidate process may be more desirable overall. However, the first candidate process may comprise a partitioning step F that is effective in removing impurities 1-80 followed by a partitioning step G that is effective in removing impurities 70-90, while the second candidate process may comprise a partitioning step H that is effective in removing impurities 40-100 followed by partitioning step J that is effective in removing impurities 1-50. A greedy algorithm may select the first candidate process because the partitioning step F is more effective than the partitioning step H, even though the second candidate process is more effective overall. In some embodiments, an improved framework is provided for downstream process (e.g. purification) design. The framework may include generating a plurality of data sets that may be used to generate and evaluate candidate processes, where each candidate process may include a sequence of partitioning steps. In some embodiments, a redundant sequence may be selected for assuring clearance (e.g., 1-80 and 60-100).

In some embodiments, a partitioning step may be represented based on a partitioning technique and/or a set of one or more parameters for the partitioning technique. Examples of partitioning techniques include, but are not limited to, chromatography, filtration, crystallization, density differential separations, extraction, applied force-based separations, or any of a wide variety of other separation or purification techniques known in the field of biologically-based production of pharmaceutical products. Non-limiting illustrative suitable categories of partitioning techniques, non-limiting examples of partitioning techniques falling under those categories, and parameters influencing the outcome of the partitioning techniques are described in Table 1.

TABLE 1

| Category of | Examples of | Parameters Influencing |
|---|---|---|
| Partitioning | Partitioning | Outcomes of Partitioning |
| Techniques | Techniques | Techniques |
| chromatography | ion exchange | mobile phase material |
| | chromatography (IEC) | properties (e.g. solvent(s), |
| | size exclusion | pH, salt concentration, etc.) |
| | chromatography (SEC) | stationary phase material |
| | hydrophobic charge | properties (e.g. charge |
| | induction chromatography | density, polarity, binding |
| | (HCIC) | specificity, binding affinity, |
| | high performance liquid | etc.) |
| | chromatography (HPLC) | other operating conditions |
| | reversed phase-ultra high | (e.g. flow rate, temperature, |
| | pressure liquid | etc.). |
| | chromatography (RP-UPLC) | |
| | multimodal chromatography | |
| filtration | tangential flow filtration | membrane molecular |
| | (TFF) | weight cutoff |
| | filtration by monolith | membrane material |
| | filtration by membrane | salt concentration |
| | filtration by sieve | sample volume |
| | filtration by mesh | pressure drop across |
| | microfiltration | filtration unit |
| | nanofiltration | |
| | ultrafiltration | |
| | reverse osmosis | |
| | forward osmosis | |
| | dialysis | |
| crystallization | fractional crystallization | concentration of materials |
| | zone refining | to be crystallized |
| | recrystallization. | temperature |
| | | polarity |
| | | ionic strength of the |
| | | solution out of which |
| | | crystals are formed |
| density | flotation | temperature |
| differential | flocculation | relative density of product |
| separations | precipitation | and impurities |
| | sedimentation | for centrifugation: angular |
| | centrifugation | velocity |
| extraction | leaching | relative polarity of the two |
| | liquid-liquid extraction | phases into which different |
| | solid phase extraction | components will partition |
| | | the relative polarity of |
| | | product and impurities |
| | | temperature |
| applied force-based | centrifugation | for centrifugation: |
| separations | field flow fractionation | temperature, relative |
| | electrophoresis | density of product and |
| | magnetic separation | impurities, and angular |
| | | velocity |
| | | for field flow fractionation: |
| | | parabolic flow-velocity, |
| | | field strength, and fluidic |
| | | channel length |
| | | for electrophoresis: field |
| | | strength and the relative |
| | | mass and surface charge of |
| | | product and impurities in |
| | | the field |
| | | for magnetic separation: |
| | | field strength and the |
| | | relative mass and magnetic |
| | | susceptibility of product |
| | | and impurities in the field |

In some embodiments, candidate processes may be generated by first grouping available partitioning steps into multiple clusters so that partitioning steps within each cluster are functionally similar. Two partitioning steps may be grouped into a same cluster based on functional similarity, even if the partitioning steps are based on different partitioning techniques. An ordering of clusters may then be selected (where a cluster may, although need not, appear multiple times), and a candidate process may be generated by selecting a partitioning step from each cluster while maintaining the ordering of the clusters. In some embodiments, different orderings of clusters may be selected. In this manner, more candidate processes may be considered, compared to where candidate processes are designed in a step-by-step fashion. As a result, a better process may be discovered. In some embodiments, a candidate process may be evaluated based on one or more criteria, such as a number of steps in the sequence, need for an adjustment of one or more conditions between two consecutive steps (e.g., pH, salt concentration, etc.), cost, product purity, product yield, product concentration, product activity, etc. For instance, a numerical score may be generated, which may, although need not, reflect a penalty for one or more undesirable aspects of a candidate sequence. As an example, a penalty may be imposed on any adjustment required between two consecutive steps, and an even higher penalty may be imposed if the adjustment is costly and/or difficult to implement (e.g., an adjustment of salt concentration). One or more best scoring processes may then be selected.

In some embodiments, one or more best scoring processes may be evaluated using one or more known experimental methods to refine one or more conditions for one or more partitioning step. For instance, for a chromatography step, column load conditions, wash conditions, elution conditions, etc. may be refined.

In some embodiments, a criterion may relate to a degree to which partitioning steps in a candidate process are orthogonal to each other. Orthogonality between two or more partitioning steps, as is understood by the skilled practitioner, is achieved when the partitioning steps have selectivity for different impurities or sets of impurities. For instance, a partitioning step A and a subsequent partitioning step B may be considered orthogonal if the partitioning step B is effective in removing impurities that co-elute from the partitioning step A with a target product. Thus, a higher degree of orthogonality may be desirable. For example, for a set of three candidate partitioning steps—partitioning step C, partitioning step D, and partitioning step E—and a starting mixture of a product and 100 impurities, the step C may be effective in removing impurities 1-80, the step D may be effective in removing impurities 40-90, and step E may be effective in removing impurities 81-100. A two-step process starting with the step C followed by the step D may recover the product with impurities 90-100 still present. A two-step process starting with the step C followed by the step E, by contrast, may recover the product with zero impurities. Although the step D on its own may be a better performing partitioning step than the step E, the step D may not be sufficiently orthogonal to the step C to result in a high performing process. On the other hand, the step E on its own may be a poor partitioning step, but together with the step C may provide a high performing process because all impurities are removed from the product by the end of the process.

In some embodiments, a criterion may relate to a degree to which partitioning steps in a candidate process are complementary to each other. Complementarity between two or more partitioning steps, as is understood by the skilled practitioner, is achieved when an outcome of a process incorporating the partitioning steps results in overall high impurity removal, even in the absence of orthogonality. For instance, a partitioning step A and a subsequent partitioning step B may be considered complementary if the partitioning step A is effective in increasing a surface charge on impurity M, and the partitioning step B is effective in binding the impurity M only when the impurity M′s surface charge is above a certain threshold. Thus, passing the impurity M through the partitioning step A may improve effectiveness of the partitioning step B in removing the impurity M from the target product. Thus, a higher degree of complementarity may be desirable.

In some embodiments, a plurality of weights may be assigned, respectively, to a plurality of criteria. Such weights may reflect relative importance and abundance among the plurality of criteria. For instance, a numerical score may be generated for each criterion, and the plurality of weights may be used to combine the plurality of scores (e.g., via a weighted sum) into an overall score.

The inventors have recognized and appreciated that downstream process design may be informed by an understanding of how certain impurities behave with respect to a partitioning technique, relative to how a target product behaves with respect to the partitioning technique.

Such an understanding may be used to facilitate in silico evaluation of the partitioning technique, even where physical properties of the impurities are not precisely characterized. For instance, an understanding of how much of certain impurities is co-eluted with the target product in a bind-elute chromatography step, and/or how much of the impurities is eluted after the target product in a subsequent flow-through chromatography step, may be sufficient for evaluating orthogonality of the chromatography steps, without having to precisely identify the impurities or their physical properties.

Accordingly, in some embodiments, a downstream process design framework may include, for each partitioning step of a plurality of partitioning steps, data indicative of how the target product behaves with respect to the partitioning step, as well as data indicative of how one or more impurities behave with respect to the partitioning step. For example, the target product may be a biologically produced product (which may be a pharmaceutical and/or protein product), and the one or more impurities may include one or more upstream process related impurities such as host cell proteins, host cell DNA, media components, etc., and/or one or more downstream process related impurities such as leachants, extractables, and residual proteins such as Protein A used in resins for chromatography. Additionally, or alternatively, the one or more impurities may include one or more product-related impurities such as product variants, product aggregates, etc. As illustrative, non-limiting examples, product-related impurities may comprise N-terminal additions, substitutions, and/or deletions; C-terminal additions, substitutions, and/or deletions; one or more misincorporated amino acids; acidic or basic species; one or more post-translational modifications, including but not limited to glycosylation, glycation, trisulfide bonds, oxidation, and deamidation; proteolytically-cleaved variants; charged variants; and/or product aggregates.

The inventors have recognized and appreciated that, by allowing different partitioning techniques to be used within a single process, more candidate processes may be considered, and as a result a better process may be found. For instance, a process may begin with centrifugation to remove some impurities by a density differential, followed by a filtration step and then a chromatography step.

Accordingly, in some embodiments, a downstream process design framework may include a plurality of data sets for each of a plurality of partitioning techniques. For each partitioning technique, the plurality of data sets may include target product data and impurity data (e.g., as described above) for each partitioning step of a plurality partitioning steps that use the partitioning technique. For example, the partitioning technique may be IEC, and the plurality partitioning steps may be a plurality of IEC steps, each with a different parameter set (e.g., different resins, buffers, etc.) The plurality of data sets for IEC may include target product data and impurity data for each parameter set.

In some embodiments, data sets may be stored in a manner that allows retrieval based on partitioning technique and/or one or more parameters.

As discussed above, the inventors have recognized and appreciated that precise characterizations of physical properties of impurities may not be necessary in evaluating candidate processes. In some embodiments, a data set for a partitioning step may be generated by conducting one or more experiments using the partitioning step. For instance, one or more experiments may be conducted with each parameter set of a plurality of parameter sets. Raw and/or processed data from the one or more experiments may be stored in the data set.

In some embodiments, one or more experiments may be designed to study how a target product, and/or one or more impurities, behave with respect to the partitioning technique and/or the parameter set. As an example, two IEC experiments may be conducted using the same resin, pH gradient, and operating conditions. The first experiment may be conducted on a cell culture fluid where a target protein is not expressed, while the second experiment may be conducted on a pure solution of the target protein. Thus, data from the first experiment may be indicative of how one or more process related impurities behave with respect to the partitioning technique and the parameter set, whereas data from the second experiment may be indicative of how the target protein behaves with respect to the partitioning technique and the parameter set.

As another example, two IEC experiments may be conducted using the same resin, pH gradient, and operating conditions, and the first experiment may be conducted on a cell culture fluid where a target protein is not expressed, but the second experiment may be conducted on a cell culture fluid where the target protein is expressed. The same organism may be used to produce both cell culture fluids. Thus, data from the first experiment may be indicative of how one or more process related impurities behave with respect to the partitioning technique and the parameter set, whereas data from the second experiment may be indicative of how the target protein and one or more product-related impurities (e.g., one or more variants and/or aggregates of the target protein) behave with respect to the partitioning technique and the parameter set.

As yet another example, two IEC experiments may be conducted using the same resin, pH gradient, and operating conditions, but the first experiment may be conducted on a cell culture fluid where a first target protein is expressed, while the second experiment may be conducted on a cell culture fluid where a second target protein is expressed. The same organism may be used to produce both cell culture fluids, and the two target proteins may differ in one or more properties (e.g., different molecular weights). The inventors have recognized and appreciated that the two cell culture fluids may have similar impurities and therefore similar impurity behavior. Thus, impurity behavior may be determined by comparing results from the two experiments.

Any suitable raw and/or processed data may be stored in a data set for a partitioning technique. For instance, any one or more suitable techniques may be used to analyze an output of a partitioning step (e.g., one or more fractions collected from a chromatography step), and an outcome of the analysis may be stored. Non-limiting examples of analysis techniques are listed in Table 2 below.

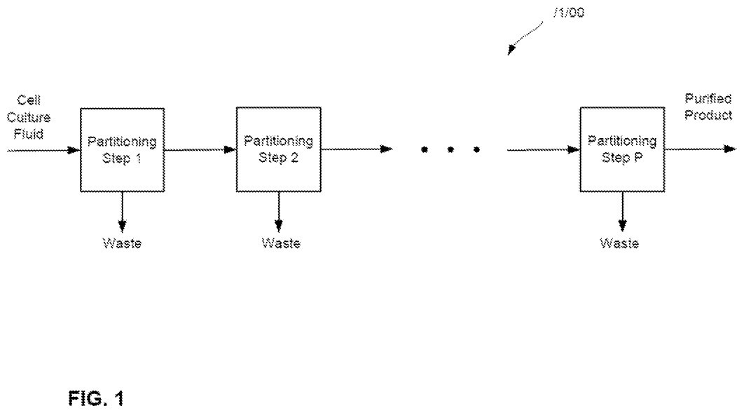

FIG. 1 shows an illustrative downstream process 100, in accordance with some embodiments. The process 100 may be a continuous flow process having a sequence of P partitioning steps, where each partitioning step may be represented based on a partitioning technique (e.g., IEC, SEC, HCIC, TFF, etc.) and a set of one or more parameters (e.g., materials, input conditions, operating conditions, output conditions, etc.).

In some embodiments, an input of partitioning step 1 may include a cell culture fluid, which may be an output of an upstream process (e.g., a bioreactor process) using any suitable organism, such as yeast, Chinese hamster ovary (CHO), E. coli, etc. This cell culture fluid may include a target protein, as well as upstream process related impurities such as host cell proteins, host cell DNA, etc. The process 100 may be designed to remove these impurities and output a purified product at partitioning step P. Additionally, or alternatively, the process 100 may be designed to remove downstream process related impurities (e.g., leachants) and/or product-related impurities (e.g., product variants, product aggregates, etc.).

Although not shown in FIG. 1, one or more adjustment units may be included between any pair of adjacent partitioning steps. For example, an adjustment (e.g., pH, salt concentration, etc.) may be made to an outflow of a partitioning step to match an input condition of an immediate following partitioning step.

In some embodiments, the process 100 may be selected from a set of candidate sequences of partitioning steps using one or more optimization techniques. For instance, a set of candidate sequences may be reduced by eliminating candidate sequences that do not satisfy one or more constraints. The remaining candidate sequences may be evaluated based on one or more criteria, and a selection may be made accordingly.

The number of partitioning steps P may be selected using a cost/benefit analysis. Typically, the greater the number of partitioning steps, the higher the purity of the product, which may stand as a benefit. However, a greater number of partitioning steps, typically, results in lower overall yields and higher overall costs. For pharmaceutical products, a minimum of two partitioning steps are generally required/recommended by the FDA. Generally, a maximum of ten partitioning steps are used in industrial separations, above which costs are prohibitive and/or product yields are too low. The design of the downstream process may involve minimizing the number of partitioning steps in the process while maintaining recovery of the target product, product purity, and/or product activity. However, aspects of the present disclosure are not limited to the use of ten or fewer, or any particular number of, partitioning steps. For instance, in some embodiments, four or more steps may be used, six or more steps may be used, ten or more steps may be used, etc.

As discussed above, the inventors have recognized and appreciated that downstream process design may be informed by an understanding of how certain impurities behave with respect to a partitioning technique, relative to how a target product behaves with respect to the partitioning technique. Accordingly, in some embodiments, one or more experiments may be designed to study how a target product and one or more impurities behave with respect to a partitioning technique and a parameter set.

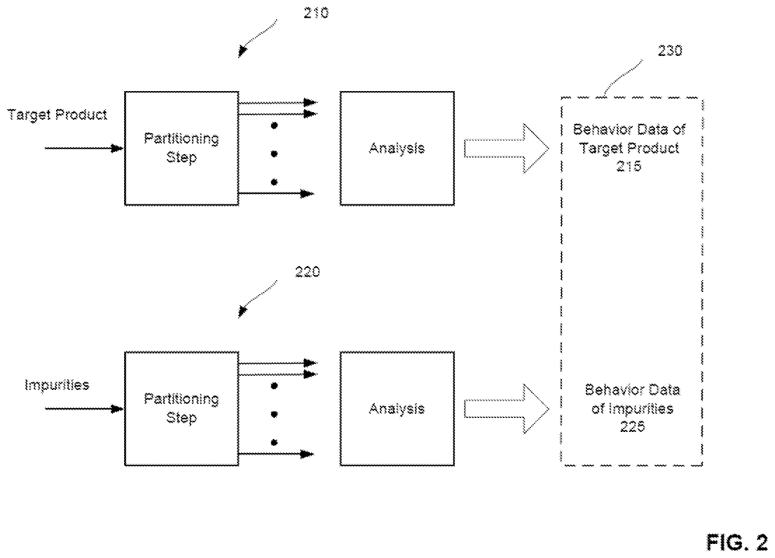

FIG. 2 shows illustrative experiments 210 and 220, in accordance with some embodiments. In this example, the experiments 210 and 220 may be designed to determine how one or more impurities of interest behave with respect to a partitioning step, relative to how a target product behaves with respect to the partitioning step. For instance, the experiments 210 and 220 may use the same partitioning step, but an input of the experiment 210 may include the target product but not the one or more impurities of interest, while an input of the experiment 220 may include the one or more impurities of interest but not the target product.

In some embodiments, the target product may be a protein to be produced using a cell culture. An input of the experiment 210 may include a pure sample of the target product dissolved in a solution (e.g., an aqueous solution), while an input of the experiment 220 may include a cell culture fluid in which the target product is not expressed. However, this arrangement is not required, as in some embodiments, an input of the experiment 210 may include a cell culture fluid in which the target product is expressed, where the cell culture fluid is produced using the same organism as for the input of the experiment 220.

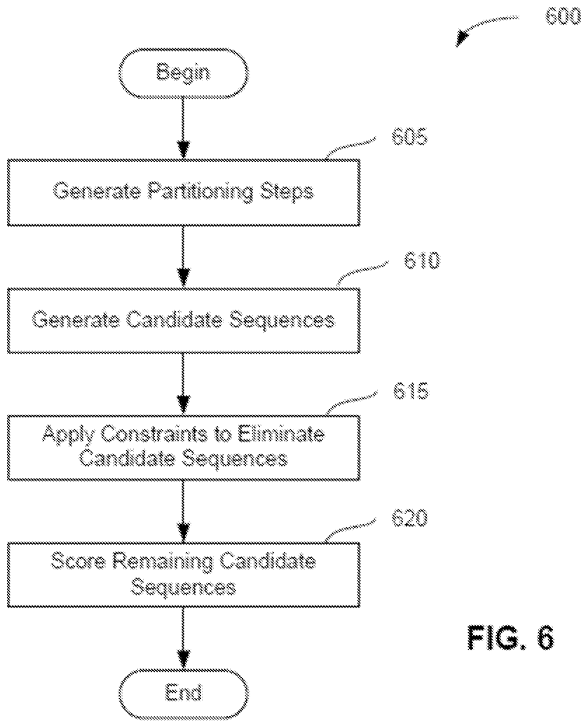

In some embodiments, one or more fractions collected from the experiment 210 may be analyzed using one or more analysis techniques, and resulting data 215 may be stored (and may subsequently be used to design downstream processes, e.g., as discussed below in connection with FIG. 6). This data may be indicative of how the target product behaves with respect to the partitioning step. Similarly, one or more fractions collected from the experiment 220 may be analyzed using the same one or more analysis techniques, and resulting data 225 may be stored. This data may be indicative of how the one or more impurities of interest behave with respect to the partitioning step.

The inventors have recognized and appreciated that, in some instances, the data 225 (behavior of one or more impurities) may be generated once for a host organism, and may be re-used when a different pharmaceutical and/or protein product is to be made using the same host organism. In this manner, only the data 215 (behavior of target product) may be re-generated when a different product is to be produced using the same host. Likewise, the data 215 (behavior of target product) may be generated once for a target product, and may be re-used when the target product is to be made using a different host organism. In this manner, only the data 225 (behavior of one or more impurities) may be re-generated when the same target product is to be produced using a different host. This may allow efficient design of downstream processes for different combinations of products and hosts.

In some embodiments, the data 215 and the data 225 may be stored in a data set 230, which may be associated with one or more tags indicating the partitioning step used in the experiments 210 and 220 and/or the one or more analysis techniques used to analyze the collected fractions. For instance, there may be a tag indicating a partitioning technique used in the partitioning step (e.g., IEC, SEC, HCIC, TFF, etc.), one or more tags indicating one or more parameters for the partitioning technique (e.g., resin, gradient, input pH, flow rate, etc. for a chromatography step), and a tag indicating an analysis technique (e.g., LC-MS, ELISA, MALDI, UV, SDS-PAGE, IEF, etc.). In this manner, the data set 230 may be retrieved by querying based on one or more tags.

Although various details of implementation are described herein, it should be appreciated that such details are illustrative of more general systems and methods. As one example, in some embodiments, the data 215 (behavior of target product) and the data 225 (behavior of one or more impurities) may be compared and one or more differences may be stored instead of, or in addition to, the data 215 and the data 225. As another example, aspects of the present disclosure are not limited to the use of tags, as other ways to organize and/or search for data may also be used.

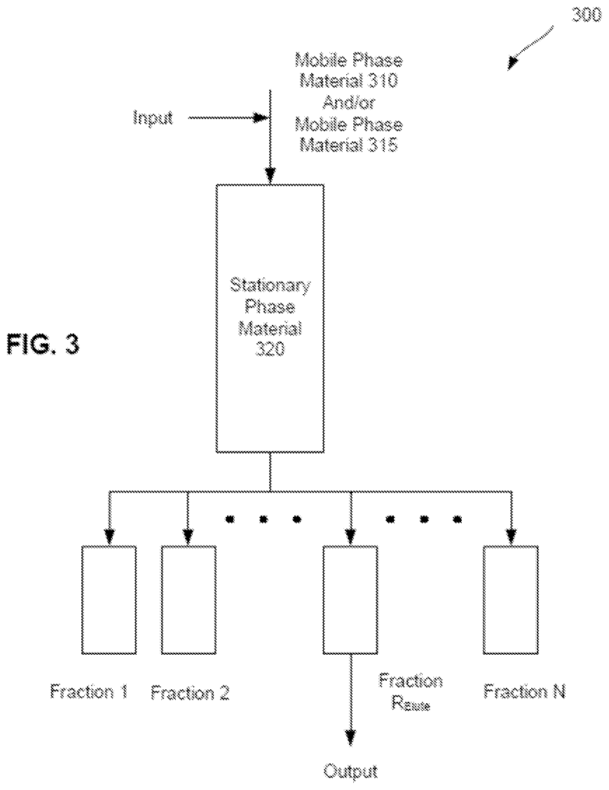

FIG. 3 shows an illustrative partitioning step 300, in accordance with some embodiments. The partitioning step 300 may be an example of a partitioning step used in the illustrative process 100 shown in FIG. 1 and/or the illustrative experiments 210 and 220 shown in FIG. 2.

In the example shown in FIG. 3, the partitioning step 300 uses a chromatography technique (e.g., IEC, SEC, HCIC, etc.), which includes introducing an input into an inflow comprising a mobile phase material 310 and/or a mobile phase material 315. The inflow, with the input introduced, may be caused to flow through a structure (e.g., column, membrane, etc.) comprising a stationary phase material 320 (e.g., resin). In some embodiments, a ratio between the mobile phase material 310 and the mobile phase material 315 may change over time. Such a gradient may cause different materials in the input (e.g., target protein vs. one or more impurities) to be retained in the structure for different amounts of time, which may allow partitioning of the different materials.

In some embodiments, a pH gradient may be used, in which case the two mobile phase materials 310 and 315 may have different pH values. The inflow may begin with a first ratio (e.g., 100% mobile phase material 310 and 0% mobile phase material 315) at a first time, and end with a second ratio (e.g., 0% mobile phase material 310 and 100% mobile phase material 315) at a second time. The ratio may vary in any suitable manner between the first and second times. For example, the ratio may vary continuously (e.g., linearly or according to some other continuous function), and/or there may be one or more discrete changes.

In some embodiments, a salt gradient may be used, instead of, or in addition to, a pH gradient. In this case, the two mobile phase materials 310 and 315 may have different concentrations of one or more salts, and a ratio of these materials may change over time in any suitable manner as described above.

In some embodiments, one or more fractions may be collected from an outflow of the structure. For instance, fraction 1 may be collected during a first time interval, fraction 2 may be collected during a second time interval, etc. These time intervals may, although need not, be non-overlapping consecutive intervals.

In the example shown in FIG. 3, the partitioning step 300 may be a bind-elute step, where a target product may be retained by the stationary phase material 320 for a longer period of time compared to one or more impurities of interest. In a bind-elute step, the target product and impurities are first loaded onto a stationary phase material 320 using a first mobile phase material 310. The target product is then eluted from the stationary phase material using one or more mobile phase compositions different from that of the loading condition, for example a time-varying volume ratio of mobile phase material 310 and mobile phase material 315, leaving one or more impurities of interest bound to the column. The bound impurities are then eluted from the column during a cleaning or regeneration step.

In some embodiments, the partitioning step 300 may be a flow-through step. In a flow-through step, the target product flows through the column containing the stationary phase material, leaving one or more impurities of interest bound to the stationary phase material. However, aspects of the present disclosure are not limited to the use of a bind-elute step or a flow-through step, or any chromatography step at all.

In some embodiments, the partitioning step 300 may be used to study a behavior of a target product and/or one or more impurities of interest, and each fraction of the plurality of fractions may be analyzed to obtain data relating to chemical composition of the fraction. In some embodiments, the partitioning step 300 may be used to partition the target product from the one or more impurities of interest, and one or more fractions in which the target product is eluted (e.g., fraction R_{Elute} shown in FIG. 3) may be considered an output of the partitioning step 300.

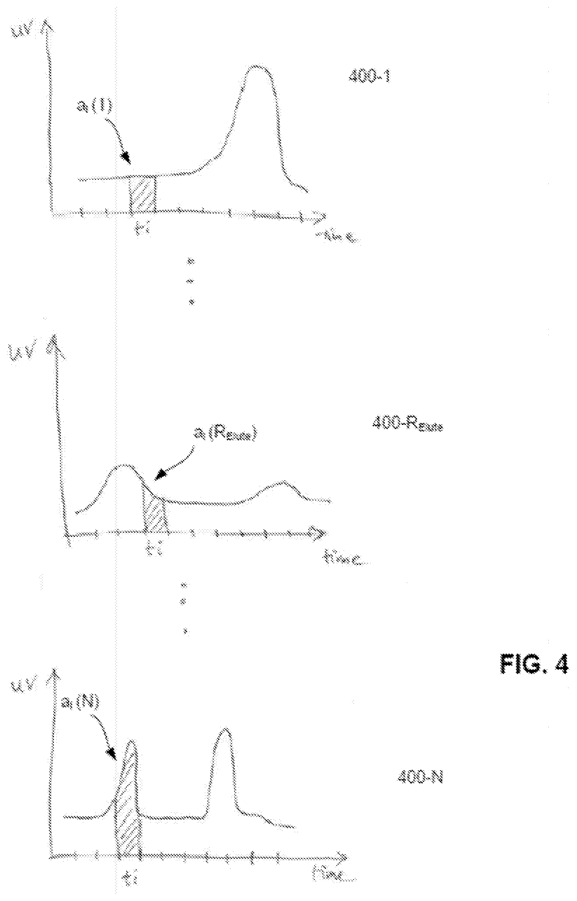

FIG. 4 shows a plurality of illustrative chromatograms 400-1, . . . , 400-N, in accordance with some embodiments. The chromatograms 400-1, . . . , 400-N may result, respectively, from analyzing the illustrative fractions 1, . . . , N shown in FIG. 3. As one example, an input to the illustrative partitioning step 300 may include a cell culture fluid in which a target protein is not expressed, so that the chromatograms 400-1, . . . , 400-N may represent behavior of one or more impurities of interest. Thus, one or more of the chromatograms 400-1, . . . , 400-N may be stored as the illustrative data 225 shown in FIG. 2.

As another example, an input to the illustrative partitioning step 300 may include a pure sample of a target protein dissolved in an aqueous solution, so that the chromatograms 400-1, . . . , 400-N may represent behavior of the target protein. Thus, one or more of the chromatograms 400-1, . . . , 400-N may be stored as the illustrative data 215 shown in FIG. 2.

In the example shown in FIG. 4, each of the chromatograms 400-1, . . . , 400-N may be generated by analyzing the corresponding fraction using a chromatography technique. As such, these chromatograms may sometimes be referred to as “analytical chromatograms.” In an embodiment in which a chromatography technique is used in the illustrative partitioning step 300 to obtain the illustrative fractions 1, . . . , N, a different chromatography technique (e.g., a higher resolution chromatography technique) may be used to analyze each fraction. This may sometimes be referred to as “two-dimensional” chromatography. For instance, in some embodiments, each fraction may be analyzed by reversed phase liquid chromatography (RPLC), reversed phase-ultra high pressure liquid chromatography (RP-UPLC), or size exclusion chromatography (SEC).

The inventors have recognized and appreciated that it may be efficient to use a rapid chromatography technique to analyze one or more fractions. A chromatography technique may be considered “rapid” if a fraction may be analyzed in no more than 15 minutes. However, aspects of the present disclosure are not limited to the use of a rapid chromatography technique.

It should also be appreciated that aspects of the present disclosure are not limited to the use of chromatography to analyze fractions collected from a partitioning step. For example, in some instances, product-related impurities (e.g., product variants, product aggregates, etc.) may not be expected to be present in significant quantities, and a collected fraction may be analyzed directly using any suitable analysis technique, without being further partitioned by a higher resolution chromatography technique.

In some embodiments, each of the chromatograms 400-1, . . . , 400-N may be generated by measuring UV absorbance at an outflow of an analytical chromatography step and plotting the measurement against time. UV absorbance may be measured at one or more wavelengths, and the chromatograms 400-1, . . . , 400-N shown in FIG. 4 may correspond to a particular wavelength. For example, suitable wavelengths may include, but are not limited to: 210 nm, which may allow detection of a large number of different molecules; 260 nm, which corresponds to absorption maximum of DNA; and 280 nm, which corresponds to an absorbance peak in protein spectra. Thus, in some embodiments, multiple sets of chromatograms may be stored, each being similar to the chromatograms 400-1, . . . , 400-N and corresponding to a different wavelength. However, it should be appreciated that aspects of the present disclosure are not limited to the use of any particular wavelength. For instance, in some embodiments, a complete optical spectrum may be used.

It should be appreciated that aspects of the present disclosure are not limited to the use of chromatography or UV absorbance analysis to analyze fractions collected from a partitioning step. Other examples of analytical techniques include, but are not limited to, LC-MS, ELISA, MALDI, SDS-PAGE, IEF, etc. Any one or more of these and/or other analytical techniques may be used to generate behavior data for a target product and/or one or more impurities of interest.

In some embodiments, one or more of the chromatograms 400-1, . . . , 400-N may be stored in discretized form, for example, to reduce storage and/or to speed up one or more optimization techniques that use the chromatograms 400-1, . . . , 400-N to evaluate candidate downstream processes. For instance, a plurality of time intervals t₁, . . . , t_{K} may be selected. These intervals may, although need not, be non-overlapping consecutive intervals. For each i=1, . . . , K, and n=1, . . . , N, an integral aᵢ (n) of the chromatogram 400-n may be calculated over the interval tᵢ, as shown in FIG. 4. In some embodiments, these integrals may be stored as the illustrative data 215 or the illustrative data 225 shown in FIG. 2.

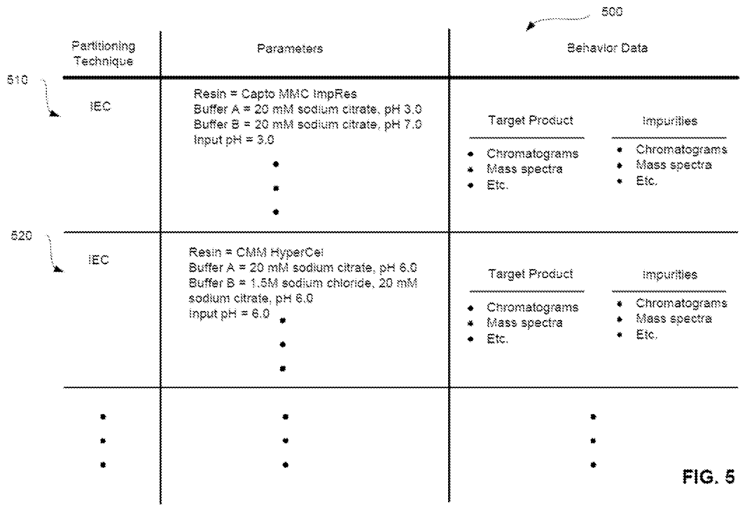

FIG. 5 shows an illustrative data arrangement 500, in accordance with some embodiments. The data arrangement 500 may include data collected from a plurality of experiments conducted using different partitioning techniques and/or different parameters, for example, as described above in connection with FIGS. 1-4.

In the example shown in FIG. 5, the data arrangement 500 includes a plurality of data sets. Each data set may correspond to a partitioning step, which may be represented based on a partitioning technique and/or a set of one or more parameters for the partitioning technique. As one example, there may be a data set 510 for an IEC step using a Capto MMC ImpRes resin and a pH gradient. As another example, there may be a data set 520 for an IEC step using a CMM HyperCel resin and a salt gradient.

Examples of partitioning techniques and associated parameters include, but are not limited to, those listed in Table 1 above. However, it should be appreciated that aspects of the present disclosure are not limited to the use of any particular partitioning technique or parameter.

In some embodiments, one or more output conditions may be stored in a data set for a partitioning step. Examples of output conditions include, but are not limited to, pH, temperature, flow rate, etc. In some embodiments, such an output condition may be used to determine whether an adjustment is needed to match an input condition of an immediately following partitioning step.

In some embodiments, each data set may include data indicative of how a target protein and/or one or more impurities of interest behave with respect to the partitioning step associated with the data set. Such data may be obtained using any one or more suitable analytical techniques. For instance, chromatography in combination with UV absorbance analysis may be used to generate a plurality of chromatograms (e.g., as shown in FIG. 4 and discussed above). Alternatively, or additionally, LC-MS may be used to generate a plurality of mass spectra.

Some examples of analytical techniques are provided below. However, it should be appreciated that aspects of the present disclosure are not limited to the use of any particular analytical technique. Non-limiting illustrative suitable categories of analytical techniques and non-limiting examples of analytical techniques falling under those categories, are described in Table 2.

TABLE 2

| Category of Analytical Technique | Examples of Analytical Techniques |
|---|---|
| involving detection of | double-stranded DNA (dsDNA) quantitation |
| a biomolecule or class | assay (e.g., PicoGreen ®) |
| of biomolecules (e.g., | enzyme-linked immunosorbent assay |
| based on affinity of an | (ELISA) |
| analyte to a detecting | real-time polymerase chain reaction (qPCR) |
| molecule) | western blot |
| involving use of at least | liquid chromatography-mass spectrometry |
| one mass differential | (e.g., LC-MS or LC-MS/MS) |
| between species in output | matrix-assisted laser desorption/ionization |
| of partitioning step | (MALDI) |
| | gel electrophoresis (e.g., sodium dodecyl |
| | sulfate polyacrylamide gel electrophoresis, |
| | also referred to asSDS-PAGE) |
| | size exclusion chromatography (SEC) |
| involving differences | reversed phase liquid chromatography (e.g., |
| between the way each | RPLC or reversed phase-ultra high pressure |
| species in output of | liquid chromatography, also referred to as |
| partitioning step interacts | RP-UPLC) |
| with stationary phase | ion exchange chromatography (IEX) |
| | gel electrophoresis (e.g., SDS-PAGE) |
| | isoelectric focusing (IEF) |
| involving differences | Raman spectroscopy |
| between the way each | ultraviolet (UV) absorbance analysis (e.g., |
| species in output of | UV or variable length UV) |
| partitioning step interacts | Fourier transform infrared spectroscopy |
| with electromagnetic radiation | (FTIR) |
| | Differential refractometry (DRI) |
| | Fluorescence detection |
| | Multiangle light scattering (MALS) |

As discussed above, the inventors have recognized and appreciated that data sets such as the illustrative data sets shown in FIG. 5 may be used to improve downstream process design, for example, by broadening a pool of candidate processes from which one or more optimal processes may be selected, and/or by allowing in silico evaluation of candidate processes.

In some embodiments, the illustrative data sets shown in FIG. 5 may be stored in such a way to allow queries for all partitioning steps that meet one or more requirements. For example, the data sets shown in FIG. 5 may be queried to determine all partitioning steps capable of removing at least a certain percentage of impurities. The percentage of impurities removed may be calculated from data collected, for example, using chromatography. As another example, the data sets shown in FIG. 5 may be queried to determine all partitioning steps that result in at least a certain produce yield. The yield may be calculated from data collected, for example, using chromatography. As another example, the data sets shown in FIG. 5 may be queried to determine all partitioning steps that result in product having at least a required activity of the target product, where activity may be calculated from data collected, for example, using ELISA.

FIG. 6 shows an illustrative process 600 that may be used to generate and evaluate candidate processes, in accordance with some embodiments. The process 600 may use data sets such as the illustrative data sets shown in FIG. 5 to generate and select candidate processes such as the illustrative process 100 shown in FIG. 1.

At act 605, a plurality of available partitioning steps may be generated. As discussed above, a partitioning step may be represented based on a partitioning technique (e.g., IEC, SEC, HCIC, TFF, etc.) and/or one or more parameters for the partitioning technique. Accordingly, in some embodiments, a plurality of parameter sets may be generated for each available partitioning technique. As an example, for chromatography, different combinations of available resin, gradient, and/or one or more conditions (e.g., temperature, flow rate, etc.) may be generated. Each such parameter set may represent a different partitioning step.

In some embodiments, one or more partitioning techniques and/or one or more parameters may be determined based on user input. For instance, a user interface may be provided via which a user may indicate which one or more partitioning techniques and/or materials (e.g., chromatography resins and/or buffers) are available. Additionally, or alternatively, the user interface may allow a user to specify one or more conditions (e.g., based on available equipment, product to be made, amount of product desired, amount of time available, etc.).

At act 610, a plurality of candidate sequences may be generated. The inventors have recognized and appreciated that, in many instances, at least two partitioning steps may be needed to effectively partition a target product from one or more impurities, and/or at least two partitioning steps may be desired to meet expectations of regulatory approval agencies. The inventors have further recognized and appreciated that, in many instances, three partitioning steps may be sufficient to effectively partition a target product from one or more impurities. Accordingly, in some embodiments, only two-step sequences and three-step sequences are included. In some other embodiments, only two-step sequences or only three-step sequences are included. However, neither is required, as in some embodiments, sequences with more than three steps (e.g., more than four steps, more than five steps, more than six steps, more than seven steps, more than eight steps, more than nine steps, more than ten steps, etc.), and/or single partitioning steps (i.e., sequences of length 1), may be included. For instance, the inventors have recognized and appreciated that one or more of the techniques described herein may be used to speed up evaluation of candidate sequences, so that a larger length (and hence a broader pool of candidate sequences) may be efficiently evaluated.

In some embodiments, every possible sequence of a desired length may be included. However, the inventors have recognized and appreciated that such an inclusive approach may result in too many candidate sequences, because it may take too much time and/or computing resource to evaluate all of the candidate sequences. Accordingly, in some embodiments, one or more constraints may be used during generation of candidate sequences.

In some embodiments, a sequence may be included only if the sequence recovers a target product. The sequence may be considered to recover the target product if, for each partitioning step in the sequence, no target product is retained, and/or all target product is available for a next step in the sequence. For example, in a case where a partitioning step in a candidate sequence is a chromatography step in a column format, whether or not the partitioning step recovers the product may be determined by performing a regeneration process for the chromatography column and analyzing an eluting solution by spectrophotometry to determine a presence or absence of the target product.

In some embodiments, one or more constraints may relate to an initial step in a sequence. As one example, resins may be categorized as multimodal cation (MMC), multimodal anion (MMA), HCIC, etc., and a constraint may be imposed based on resin category. For instance, a constraint may prohibit using a partitioning step with an HCIC resin as a capture step.

As another example, partitioning steps may be categorized as bind-elute or flow-through, and a constraint may be imposed accordingly. In some embodiments, categorization of partitioning steps may be performed based on behavior data of a target product. For instance, target product behavior data for a partitioning step may be accessed (e.g., from the illustrative data arrangement 500 shown in FIG. 5). With reference to the example shown in FIG. 3, target product behavior data for the partitioning step 300 may indicate the target product was initially retained by the stationary phase material but ultimately eluted at fraction R_{Elute}. As such, the partitioning step 300 may be categorized as a bind-elute step.

In some embodiments, a constraint may be imposed according to how a partitioning step is categorized. For instance, only those partitioning steps categorized as bind-elute steps may be used for capture (e.g., to concentrate the product early in the process). In some embodiments, one or more constraints may relate to a degree of orthogonality between steps. As an example, a same resin may not be used in two different steps in a sequence, as using a resin a second time may provide minimal benefit.

In some embodiments, one or more constraints may relate to connectivity between steps. As an example, an output condition (e.g., pH, salt concentration, etc.) of a partitioning step must sufficiently match an input condition of an immediately following partitioning step in a sequence of partitioning steps. Otherwise, the sequence may not be included as a candidate sequence. In this manner, no adjustment may be needed between steps, which may facilitate integrated manufacturing.

Any suitable combination of these and/or other constraints may be used during generation of candidate sequences, as aspects of the present disclosure are not limited to the use of any particular constraint, or any constraint at all.

Returning to FIG. 6, one or more additional constraints may be applied, at act 615, to eliminate one or more candidate sequences generated at act 610. The inventors have recognized and appreciated that some constraints may be dynamic. For instance, whether such a constraint is satisfied by a partitioning step in a sequence may depend on which one or more partitioning steps precede the partitioning step at issue. As an example, again with reference to the example shown in FIG. 3, target product behavior data for the partitioning step 300 may indicate the target product was initially retained by the stationary phase material but ultimately eluted at fraction R_{Elute} during a screening experiment. The partitioning step 300 may be categorized as a bind-elute step when the partitioning step 300 is considered in isolation. However, if one or more preceding partitioning steps in a candidate sequence are such that a load condition (e.g., pH or salt concentration) of the partitioning step 300, when used within the candidate sequence, are similar to a load condition at a point after R_{Elute} in the gradient of the screening experiment, the partitioning step 300 may be categorized as implicit flow-through in the context of that particular candidate sequence. Otherwise, the partitioning step 300 may be categorized as bind-elute in the context of that particular candidate sequence. (A partitioning step may be categorized as explicit flow-through in the context of a candidate sequence if the partitioning step is categorized as flow-through in isolation, for instance, because the target product is not retained initially.)

Accordingly, in some embodiments, one or more dynamic constraints may be applied to eliminated one or more candidate sequences constructed at act 610. As an example, candidate sequences having multiple flow-through steps, including both implicit and explicit flow-through steps, may be eliminated.

At act 620, remaining candidate sequences may be scored, and one or more best scoring sequences may be output (e.g., stored in a computer-readable file, shown to a user on a computer screen, communicated to a computer program for automating a biological manufacturing plant, etc.). In some embodiments, all candidate sequences may be output (e.g., in ranked order).

The scoring may be done in any suitable manner. In some embodiments, a candidate sequence may be scored based on one or more criteria, such as a number of steps in the sequence, lack of need for an adjustment between two consecutive steps (e.g., of pH, conductivity, etc.), cost, product purity, product yield, product concentration, product activity, etc. For instance, a numerical score may be generated, which may, although need not, reflect a penalty for one or more undesirable aspects of a candidate sequence. As an example, a penalty may be imposed on any adjustment required between two consecutive steps, and an even higher penalty may be imposed if the adjustment is costly and/or difficult to implement (e.g., a salt concentration adjustment). However, it should be appreciated that aspects of the present disclosure are not limited to the use of a penalty score. In some embodiments, a score may be generated that reflects a reward, in addition to, or instead of, a penalty score.

In some embodiments, a score may be calculated that is indicative of a degree to which partitioning steps in a candidate sequence are orthogonal to each other. For instance, a partitioning step A and a subsequent partitioning step B may be considered orthogonal if the partitioning step B is effective in removing impurities that co-elute with a target product in the partitioning step A.

As an example, for an embodiment involving a sequence of chromatographic partitioning steps, a penalty score may be calculated as follows using behavior data of one or more impurities of interest.

$$score = {\sum }_{i = 1}^{K} ( {\prod }_{j = 1}^{P} {A}_{i , j} )$$

where,

$$\{ \begin{matrix}j ∈ F , & {A}_{i , j} = {( {\sum }_{n = {R}_{Elute} ( j ) + 1}^{N} {a}_{i , j} ( n ) )}^{- 1} \\ j ∈ B , & {A}_{i , j} = {a}_{i , j} ( {R}_{Elute} ( j ) )\end{matrix}$$

In this example, there are P partitioning steps in each candidate sequence. For each i=1, . . . , K, j=1, . . . , P, and n=1, . . . , N, a_{i,j} (n) is an integral, over the i^{th} time interval, of a chromatogram obtained at a given wavelength from the n^{th} fraction (e.g., as shown in FIG. 4 and discussed above), where the n^{th} fraction is obtained using the j^{th} partitioning step in the candidate sequence. In some embodiments, these integrals may be accessed (e.g., from the illustrative data arrangement 500 shown in FIG. 5) by specifying a partitioning technique (e.g., IEC) and/or one or more parameters (e.g., resin and gradient) of the j^{th} partitioning step.

In the illustrative formula above, F denotes a set of flow-through steps, B denotes a set of bind-elute steps, and fraction R_{Elute}(j) is a fraction in which the target product is eluted during the j^{th} partitioning step. The value R_{Elute}(j) may be determined based on target product behavior data for the j^{th} partitioning step, which may be accessed (e.g., from the illustrative data arrangement 500 shown in FIG. 5) by specifying a partitioning technique (e.g., IEC) and/or one or more parameters (e.g., resin and gradient) of the j^{th} partitioning step.

Thus, for a bind-elute step (j in the set B), the above formula penalizes impurities co-eluted with the target product (as reflected by the value a_{i,j} (R_{Elute}(j))). By contrast, for a flow-through step (j in the set F), the above formula rewards impurities eluted after the target product (as reflected by the reciprocal of the sum of the values a_{i,j} (n) for n greater than R_{Elute}(j)).

The inventors have recognized and appreciated that the above formula may functionally captures orthogonality. For example, if the term Ai,j of a flow-throw step is large, the product Πⱼ₌₁^{p}A_{i,j} may be small even if the term the term A_{i,j} of a bind-elute step in the same sequence is large. However, it should be appreciated that aspects of the present disclosure are not limited to the use of the above formula, or any formula designed based on orthogonality. For instance, in some embodiments, one or more scores may be calculated to capture complementarity, instead of, or in addition to, orthogonality.

In some embodiments, one or more best scoring processes may be evaluated using one or more known experimental methods to refine one or more conditions for one or more partitioning step. For instance, for a chromatography step, column load conditions, wash conditions, elution conditions, etc. may be refined.

As discussed above, the inventors have recognized and appreciated that some partitioning steps may be functionally similar to each other, even if the partitioning steps use different partitioning techniques and/or different parameters (e.g., different resins, gradients, etc.). For example, two partitioning steps may produce similar results when used to partition a target product from one or more impurities.

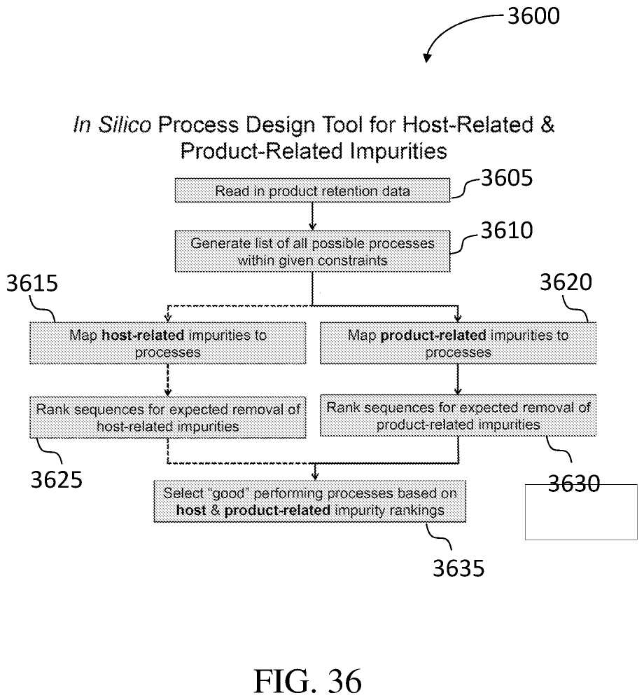

FIG. 36 shows an illustrative process 3600 that may be used to generate and evaluate candidate processes for removal of process-related impurities (e.g., host-related impurities) and/or product-related impurities, in accordance with some embodiments. As discussed above, product-related impurities include, but are not limited to, N-terminal additions, substitutions, and/or deletions; C-terminal additions, substitutions, and/or deletions; one or more misincorporated amino acids; acidic or basic species; one or more post-translational modifications, including but not limited to glycosylation, glycation, trisulfide bonds, oxidation, and deamidation; proteolytically-cleaved variants; charged variants; and product aggregates.

Process 3600 may use data sets such as the illustrative data sets shown in FIG. 5 to generate and select candidate processes such as the illustrative process 100 shown in FIG.

At act 3605, a plurality of available partitioning steps may be generated, and product retention data may be read for each available partitioning step. As discussed above, a partitioning step may be represented based on a partitioning technique (e.g., IEC, SEC, HCIC, TFF, etc.) and/or one or more parameters for the partitioning technique. Accordingly, in some embodiments, a plurality of parameter sets may be generated for each available partitioning technique. As an example, for chromatography, different combinations of available resin, gradient, and/or one or more conditions (e.g., temperature, flow rate, etc.) may be generated. Each such parameter set may represent a different partitioning step.

In some embodiments, one or more partitioning techniques for removal of process-related impurities (e.g., host-related impurities) and/or product-related impurities and/or one or more parameters may be determined based on user input. For instance, a user interface may be provided via which a user may indicate which one or more partitioning techniques and/or materials (e.g., chromatography resins and/or buffers) are available. Additionally, or alternatively, the user interface may allow a user to specify one or more conditions (e.g., based on available equipment, product to be made, amount of product desired, amount of time available, etc.).

At act 3610, a plurality of candidate sequences for removal of process-related impurities (e.g., host-related impurities) and/or product-related impurities may be generated. As described above, the inventors have recognized and appreciated that, in many instances, sequences with two and/or three partitioning steps may be desirable. However, in some embodiments, sequences with more than three steps (e.g., more than four steps, more than five steps, more than six steps, more than seven steps, more than eight steps, more than nine steps, more than ten steps, etc.), and/or single partitioning steps (i.e., sequences of length 1), may be included. For instance, the inventors have recognized and appreciated that one or more of the techniques described herein may be used to speed up evaluation of candidate sequences, so that a larger length (and hence a broader pool of candidate sequences) may be efficiently evaluated.

In some embodiments, every possible sequence of a desired length may be included. However, the inventors have recognized and appreciated that such an inclusive approach may result in too many candidate sequences for removal of process-related impurities (e.g., host-related impurities) and/or product-related impurities, because it may take too much time and/or computing resource to evaluate all of the candidate sequences. Accordingly, in some embodiments, one or more constraints may be used during generation of candidate sequences.

In some embodiments, a sequence for removal of process-related impurities (e.g., host-related impurities) and/or product-related impurities may be included only if the sequence recovers a target product. The sequence may be considered to recover the target product if, for each partitioning step in the sequence, no target product is retained, and/or all target product is available for a next step in the sequence. For example, in a case where a partitioning step in a candidate sequence is a chromatography step in a column format, whether or not the partitioning step recovers the product may be determined by performing a regeneration process for the chromatography column and analyzing an eluting solution by spectrophotometry to determine a presence or absence of the target product.

In some embodiments, one or more constraints may relate to an initial step in a sequence. As one example, resins may be categorized as multimodal cation (MMC), multimodal anion (MMA), HCIC, etc., and a constraint may be imposed based on resin category. For instance, a constraint may prohibit using a partitioning step with an HCIC resin as a capture step.

As another example, partitioning steps may be categorized as bind-elute or flow-through, and a constraint may be imposed accordingly. In some embodiments, categorization of partitioning steps may be performed based on behavior data of a target product. For instance, target product behavior data for a partitioning step may be accessed (e.g., from the illustrative data arrangement 500 shown in FIG. 5). With reference to the example shown in FIG. 3, target product behavior data for the partitioning step 300 may indicate the target product was initially retained by the stationary phase material but ultimately eluted at fraction RElute. As such, the partitioning step 300 may be categorized as a bind-elute step.

In some embodiments, a constraint may be imposed according to how a partitioning step is categorized. For instance, only those partitioning steps categorized as bind-elute steps may be used for capture (e.g., to concentrate the product early in the process). In some embodiments, one or more constraints may relate to a degree of orthogonality between steps. As an example, a same resin may not be used in two different steps in a sequence, as using a resin a second time may provide minimal benefit.

In some embodiments, one or more constraints may relate to connectivity between steps. As an example, an output condition (e.g., pH, salt concentration, etc.) of a partitioning step must sufficiently match an input condition of an immediately following partitioning step in a sequence of partitioning steps. Otherwise, the sequence may not be included as a candidate sequence. In this manner, no adjustment may be needed between steps, which may facilitate integrated manufacturing, or improve volumetric productivity or efficiency of operation of a process by reducing total numbers of steps required.

Any suitable combination of these and/or other constraints may be used during generation of candidate sequences, as aspects of the present disclosure are not limited to the use of any particular constraint, or any constraint at all.

Returning to FIG. 36, one or more additional constraints may be applied, at act 3610, to eliminate one or more candidate sequences generated at act 3610. The inventors have recognized and appreciated that some constraints may be dynamic. For instance, whether such a constraint is satisfied by a partitioning step in a sequence may depend on which one or more partitioning steps precede the partitioning step at issue. As an example, candidate sequences having multiple flow-through steps, including both implicit and explicit flow-through steps, may be eliminated.

At act 3615, the data collected for each partitioning step in a sequence on process-related impurities (e.g., host-related impurities) may be mapped to the remaining candidate sequences (e.g., processes). Following this, at act 3625, the remaining candidate sequences may be scored or ranked for expected removal of process-related impurities (e.g., host-related impurities), and one or more best scoring sequences may be output (e.g., stored in a computer-readable file, shown to a user on a computer screen, communicated to a computer program for automating a biological manufacturing plant, etc.). In some embodiments, all candidate sequences for removal of process-related impurities (e.g., host-related impurities) may be output (e.g., in ranked order).

The scoring may be done in any suitable manner. In some embodiments, a candidate sequence for removal of process-related impurities (e.g., host-related impurities) may be scored based on one or more criteria, such as a number of steps in the sequence, lack of need for an adjustment between two consecutive steps (e.g., of pH, conductivity, etc.), cost, product purity, product yield, product concentration, product activity, etc. For instance, a numerical score may be generated, which may, although need not, reflect a penalty for one or more undesirable aspects of a candidate sequence. As an example, a penalty may be imposed on any adjustment required between two consecutive steps, and an even higher penalty may be imposed if the adjustment is costly and/or difficult to implement (e.g., a salt concentration adjustment). However, it should be appreciated that aspects of the present disclosure are not limited to the use of a penalty score. In some embodiments, a score may be generated that reflects a reward, in addition to, or instead of, a penalty score. As described above, in some embodiments, a score may be calculated that is indicative of a degree to which partitioning steps in a candidate sequence are orthogonal to each other. For instance, a partitioning step A and a subsequent partitioning step B may be considered orthogonal if the partitioning step B is effective in removing impurities that co-elute with a target product in the partitioning step A. However, it should be appreciated that aspects of the present disclosure are not limited to the use of the above formula, or any formula designed based on orthogonality. For instance, in some embodiments, one or more scores may be calculated to capture complementarity, instead of, or in addition to, orthogonality.

At act 3620, the data collected for each partitioning step in a sequence on product-related impurities may be mapped to the candidate sequences (e.g., processes) remaining from act 3610. Following this, at act 3630, the remaining candidate sequences may be scored or ranked for expected removal of product-related impurities, and one or more best scoring sequences may be output (e.g., stored in a computer-readable file, shown to a user on a computer screen, communicated to a computer program for automating a biological manufacturing plant, etc.). In some embodiments, all candidate sequences for removal of product-related impurities may be output (e.g., in ranked order).

The scoring may be done in any suitable manner. In some embodiments, a candidate sequence for removal of product-related impurities may be scored in a manner described herein as being used for the scoring of candidate sequences for removal of process-related impurities (e.g., host-related impurities). In some embodiments, a candidate sequence for removal of product-related impurities may be scored based on a metric of selectivity. In a non-limiting illustrative embodiment, for each partitioning step, all chromatographic curves of the product and the product-related impurities may be normalized based on area under the curve. From there, in the same non-limiting illustrative embodiment, the percent overlap between the product and each product-related impurity may be considered for a given partitioning step in a given candidate sequence generated from act 3610. This selectivity metric may be used to score processes for removal of product-related impurities.

Following the performance of acts 3625 and 3630, a selection step may be performed at act 3635, wherein sequences (e.g., processes) are selected that perform well based on the performance rankings resulting from acts 3625 and 3630. Act 3635 selects sequences that perform well in removal of both process-related impurities (e.g., host-related impurities) and product-related impurities. As an illustrative example, each candidate sequence may be assigned a combined score comprising a combination (e.g., a weighted or non-weighted sum) of a first score based on removal of process-related impurities and a second score based on removal of product-related impurities. The candidate sequences may be ranked based on the combined scores, and act 3635 may select one or more top-ranking sequences.

In some embodiments, one or more top-ranking sequences may be evaluated using one or more known experimental methods to refine one or more conditions for one or more partitioning step. For instance, for a chromatography step, column load conditions, wash conditions, elution conditions, etc. may be refined.

As discussed above, the inventors have recognized and appreciated that some partitioning steps may be functionally similar to each other, even if the partitioning steps use different partitioning techniques and/or different parameters (e.g., different resins, gradients, etc.). For example, two partitioning steps may produce similar results when used to partition a target product from one or more impurities.

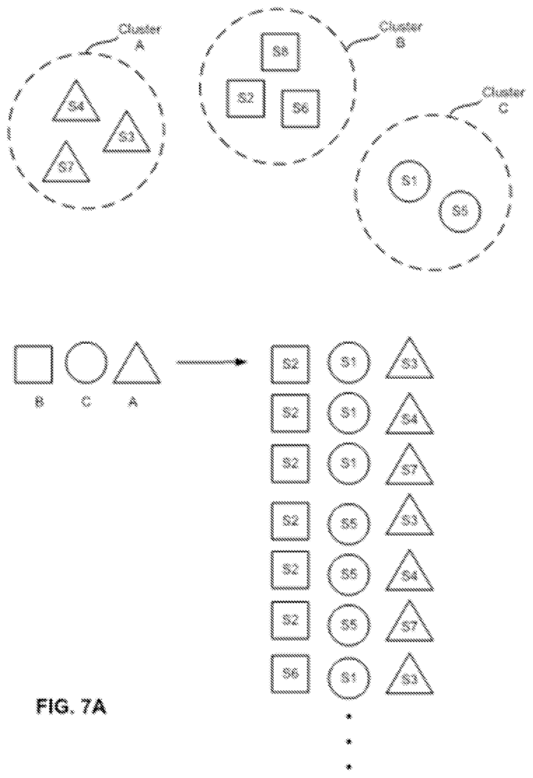

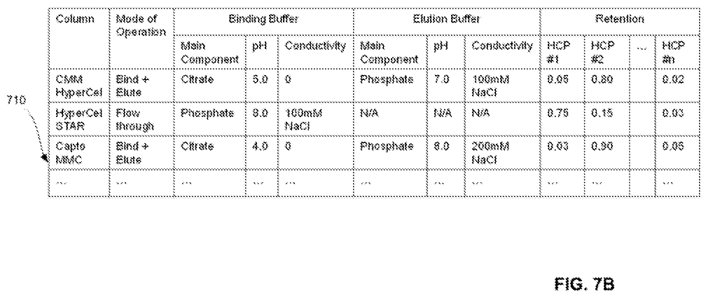

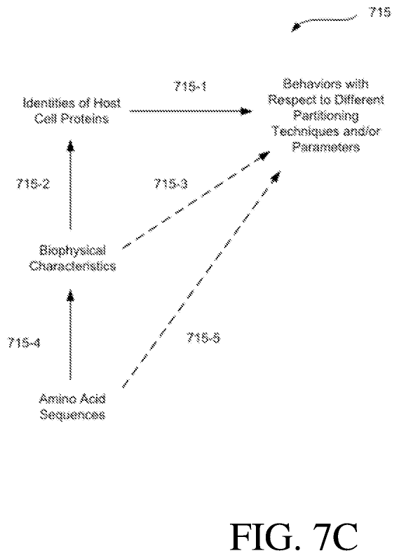

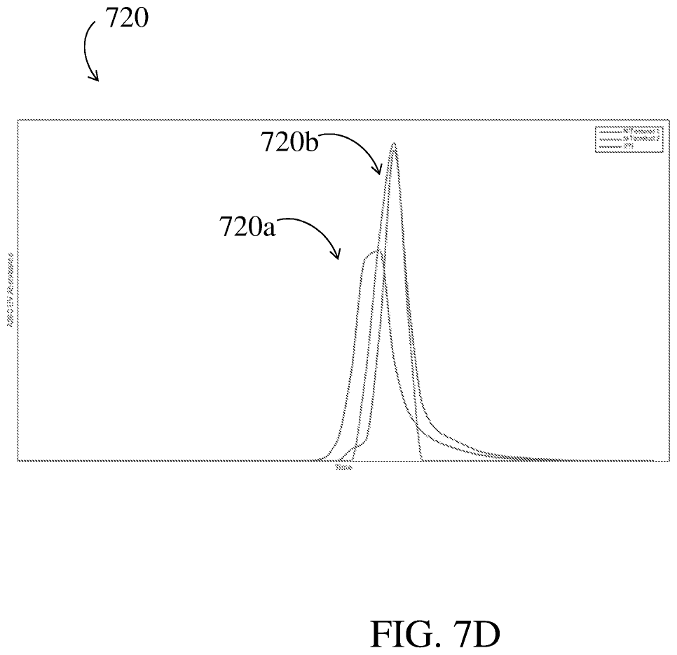

The inventors have recognized and appreciated that generation of candidate processes (e.g., at act 610 of the illustrative process 600 shown in FIG. 6) may be simplified by first grouping available partitioning steps into clusters based on functional similarity. FIG. 7A shows illustrative clusters A, B, and C of partitioning steps, in accordance with some embodiments. In this example, cluster A includes partitioning steps S3, S4, and S7, partitioning steps S2, S6, and S8, and cluster C includes partitioning steps S1 and S5. Partitioning steps within each cluster may be functionally similar.

In some embodiments, an ordering of clusters may be selected (where a cluster may, although need not, appear multiple times), and a candidate process may be generated by selecting a partitioning step from each cluster while maintaining the ordering of the clusters. For instance, with reference to FIG. 7A, it may be determined that B, C, A may be a desirable ordering, and candidate processes may be generated to include all possible sequences in which a first step is in cluster B, a second step is in cluster C, and a third step is in cluster A. Thus, as shown in FIG. 7A, multiple candidate sequences may be generated, such as S2.S1.S3, S2.S1.S4, etc. In some embodiments, multiple desirable orderings of clusters may be used, which may lead to an even broader set of candidate sequences.

In some embodiments, functional similarity may be defined explicitly based on existing knowledge. For instance, as discussed above in connection with FIG. 6, a chromatography step may be categorized as bind-elute or flow-through based on target product behavior data. Additionally, or alternatively, functional similarity may be learned automatically, for example, using one or more machine learning techniques (e.g., clustering algorithms). The inventors have recognized and appreciated that machine learning techniques may provide improved clustering as more data is collected. For instance, in some embodiments, biological manufacturing production runs may be monitored. Data collected therefrom may be used to obtain an improved set of clusters.

The inventors have further recognized and appreciated that functional similarity may depend on an analysis technique used to analyze fractions obtained using the partitioning steps. For instance, a first analytical technique (e.g., RPLC) may provide information regarding purity, while a second analytical technique (e.g., ELISA) may provide information regarding product concentration. As a result, two partitioning steps may be functionally similar with respect to the first analytical technique (e.g., similar purities) but not with respective to the second analytical technique (e.g., different concentrations), or vice versa.

In some embodiments, clustering may be performed based on multiple analytical techniques. For instance, two partitioning steps may be considered functionally similar if the steps are functionally similar with respect to each of the analytical techniques (e.g., similar concentration and similar purity). However, it should be appreciated that aspects of the present disclosure are not limited to the use of multiple analytical techniques to perform clustering, or any clustering at all. Thus, the inventors have recognized and appreciated that fractions obtained from partitioning experiments (e.g., chromatography screening experiments) may be analyzed, and data sets may be stored based on results of analyzing the fractions, and that such data sets may be used to efficiently develop downstream processes. For example, such data sets may be used to select downstream processes, where orthogonality is used as a criterion.

FIG. 7B shows an illustrative data arrangement 705, in accordance with some embodiments. The data arrangement 705 may be generated by conducting different partitioning experiments on a same cell culture fluid. For instance, data may be collected from a plurality of experiments conducted using different partitioning techniques and/or different parameters (e.g., as described above in connection with FIGS. 1-4). Although FIG. 7B only shows data from chromatographic experiments, it should be appreciated that aspects of the present disclosure are not so limited.

In some embodiments, one or more automated techniques may be used to perform the partitioning experiments rapidly. Examples of automated techniques include, but are not limited to, a robotic fluid handling workstation, pre-packed chromatography columns and/or filter plates, etc.). Such techniques may allow a large number of experiments to be conducted, and hence a large amount of data to be collected, in a small amount of time.

In some embodiments, the cell culture fluid may include multiple host cell proteins. Samples collected from the experiments may be analyzed using one or more suitable techniques. In a non-limiting example, a liquid chromatography mass spectrometry (LC/MS) technique may be used, and results may be compared to LC/MS data of known host cell proteins (e.g., those routinely detected in pichia cell culture supernatant) to identify which host cell proteins are retained. Additionally, or alternatively, a retention percentage may be determined for each host cell protein. Thus, the data arrangement 705 may indicate which partitioning techniques and/or parameters are effective in removing which host cell proteins.

In the example shown in FIG. 7B, the data arrangement 705 includes a plurality of data sets. Each data set may correspond to a partitioning step, which may be represented based on a partitioning technique and/or a set of one or more parameters for the partitioning technique. As one example, there may be a data set 710 for an IEC step using a column with Capto MMC resin, operating in bind-elute mode with some suitable binding buffer and some suitable elution buffer.

In some embodiments, each data set may include data indicative of how one or more host cell proteins (e.g., a target protein and/or one or more impurities of interest) behave with respect to the partitioning step associated with the data set. For instance, each data set may include a retention percentage for each host cell protein. As discussed herein, the inventors have recognized and appreciated that data sets such as the illustrative data sets shown in FIG. 7B may be used to improve downstream process design. For instance, in some embodiments, candidate processes of three or fewer steps may be generated and scored using the illustrative data sets shown in FIG. 7B, and a process may be selected that minimizes overall retention of host cell proteins other than a target protein while retaining the target protein, and/or maximizes recovery of the target protein while removing other host cell proteins.

In some embodiments, the illustrative data sets shown in FIG. 7B may be stored in such a way to allow queries for all partitioning steps that meet one or more requirements. For example, the data sets shown in FIG. 7B may be queried to determine all partitioning steps capable of removing at least a first percentage of a first host cell protein while retaining at least a second percentage of a second host cell protein. In this manner, the data sets shown in FIG. 7B may be used to cluster partitioning techniques and/or parameters based on functional similarity. Effective clustering may improve downstream process design, for example, by allowing adjustments to solve connectivity issues, reduce usage of materials, replace costly materials with cheaper alternatives, etc.

FIG. 7C shows an illustrative process 715 for predicting behaviors of host cell proteins, in accordance with some embodiments. For instance, the process 715 may be used to predict how a host cell protein may behave with respect to different partitioning techniques and/or different parameters for such techniques.

At act 715-1, data may be collected and/or analyzed to map identities of host cell proteins to behaviors with respect to different partitioning techniques and/or parameters. For instance, data sets such as those shown in the illustrative data arrangement 705 of FIG. 7B may be used to map a given host cell protein to retention percentages recorded for that host cell protein under different partitioning techniques and/or parameters.

At act 715-2, one or more biophysical characteristics of proteins may be considered. Examples of biophysical characteristics include, but are not limited to, isoelectric point (pI), secondary structure, tertiary structure, hydrophobicity, size, number of certain amino acids (e.g., number of lysines), etc. For each value (or range of values) for a biophysical characteristic, one or more host cell proteins having that value (or falling into that range of values) may be identified. At act 715-3, behaviors of such host cell proteins with respect to different partitioning techniques and/or parameters (e.g., as determined at act 715-1) may be analyzed to identify potential correlations. For instance, host cell proteins may be grouped based on a biophysical characteristic (e.g., pI), and one or more partitioning techniques and/or parameters suitable for separating host cell proteins with different values of that biophysical characteristic may be identified. FIG. 7D shows an illustrative chromatogram 720, in accordance with some embodiments. The chromatogram 720 demonstrates that a certain resin (e.g., SP Sepharose) at a certain pH (e.g., 4.0) may be suitable for separating IFN (e.g., peak 720a) and two N-terminal product variants (e.g., peaks 720b). This may be used to infer that the resin and the pH value may be suitable for separating proteins with pI values similar to IFN and the two N-terminal product variants.

At act 715-4, one or more amino acid sequences may be mapped to one or more corresponding biophysical characteristics. For instance, given an amino acid sequence, a lookup may be performed in one or more databases to determine one or more biophysical characteristics of that protein.

At act 715-5, a predictive model may be built to predict, based on amino acid sequence, how different proteins may behave with respect to different partitioning techniques and/or parameters. For instance, the one or more biophysical characteristics identified at act 715-4 for each amino acid sequence may be used to retrieve behavior data with respect to different partitioning techniques and/or parameters, and the retrieved behavior data may be used to train a machine learning model.

In some embodiments, partitioning experiments may be performed to obtain data relating to how host-related impurities behave with respect to a partitioning technique (e.g., a chromatography technique).

In some embodiments, partitioning experiments may be performed to obtain data relating to how a target product and/or one or more product-related impurities behave with respect to a partitioning technique (e.g., a chromatography technique).

In some embodiments, chromatography experiments may be performed using various chromatography materials, including multimodal, ion exchange, hydrophobic charge induction, and salt tolerant chromatographic materials.

In some embodiments, chromatography experiments may be performed using various linear salt and pH gradients in a column format.

In some embodiments, chromatography experiments may be performed using batch adsorption at various salt and pH conditions.

In some embodiments, fractions obtained from partitioning experiments (e.g., chromatography screening experiments) may be analyzed using any suitable analytical technique, and analysis results may be used to quantify orthogonality.

In some embodiments, fractions obtained from partitioning experiments (e.g., chromatography screening experiments) may be analyzed using a high resolution chromatographic technique.

In some embodiments, fractions obtained from partitioning experiments (e.g., chromatography screening experiments) may be analyzed using a reversed phase based high resolution chromatographic technique.

In some embodiments, fractions obtained from partitioning experiments (e.g., chromatography screening experiments) may be analyzed using a size exclusion phase based high resolution chromatographic technique.

In some embodiments, fractions obtained from partitioning experiments (e.g., chromatography screening experiments) may be analyzed using a mass spectrometry technique.

In some embodiments, fractions obtained from partitioning experiments (e.g., chromatography screening experiments) may be analyzed using a chip based (e.g., microfluidic chip, assay chip) technique.

In some embodiments, results from analyzing fractions obtained from partitioning experiments (e.g., chromatography screening experiments) may be organized to facilitate retrieval and/or querying. For instance, a data set may be stored in association with one or more identifiers indicating partitioning technique used (e.g., IEC, SEC, HCIC, etc.), materials used (e.g., resin, gradient, etc.), fraction number, one or more detection parameters (e.g. wavelength of UV absorbance), etc.

In some embodiments, data from analyzing fractions obtained from partitioning experiments (e.g., chromatography screening experiments) may be discretized. For instance, a chromatogram resulting from analyzing the fractions using high resolution chromatography may be integrated over discrete time intervals to obtain discrete values, which may be stored as a vector.

In some embodiments, data relating to how a target product and/or one or more product-related impurities behave with respect to a partitioning technique (e.g., a chromatography technique) may be interrogated to identify sequences of partitioning steps which will recover the product while satisfying one or more constraints.

In some embodiments, one or more constraints may relate to a number of partitioning steps in a sequence, connectivity between steps, a mode of operation of a step, an ordering of steps, identities of steps, a material used (e.g., prohibition against use of a single resin in more than one step), etc.

In some embodiments, modes of operation may include flow-through, weak partitioning, bind-elute, isocratic, and/or gradient.

In some embodiments, data relating to how a target product and/or one or more product-related impurities behave with respect to various partitioning techniques, and/or data relating to how one or more host-related impurities behave with respect to various partitioning techniques, may be used to rank candidate sequences of portioning steps based on likelihoods of removing host-related impurities and product-related impurities, with appropriate weightings.

In some embodiments, one or more top ranking processes may be selected and process refinement may be performed to accommodate for effects of column load, column washes, and elution conditions, for example, to improve product purity and product recovery. For instance, multiple high ranking processes may be refined, and results may be compared.

In some embodiments, process refinement may include using column modeling to facilitate identification of process conditions which satisfy product specific purity and recovery requirements.

In some embodiments, one or more of the above described techniques may be used to design a downstream process for hGH from yeast (e.g., Pichia pastoris) cell culture supernatant.

In some embodiments, one or more of the above described techniques may be used to design a downstream process for GCSF from yeast (e.g., Pichia pastoris) cell culture supernatant.

In some embodiments, one or more of the above described techniques may be used to design a downstream process for interferon-alpha 2B from yeast (e.g., Pichia pastoris) cell culture supernatant.

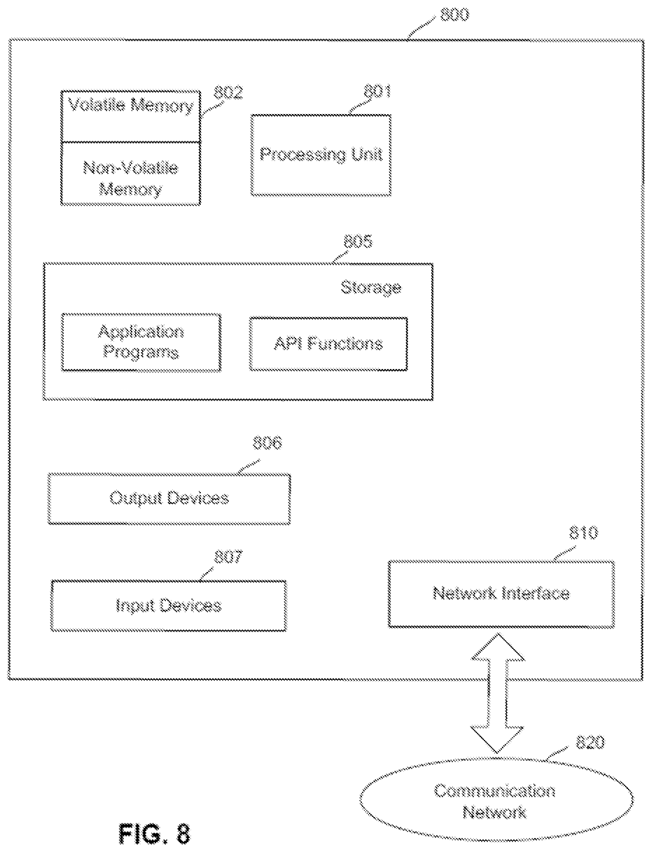

FIG. 8 shows, schematically, an illustrative computer 800 on which any aspect of the present disclosure may be implemented. In the embodiment shown in FIG. 8, the computer 800 includes a processing unit 801 having one or more processors and a non-transitory computer-readable storage medium 802 that may include, for example, volatile and/or non-volatile memory. The memory 802 may store one or more instructions to program the processing unit 801 to perform any of the functions described herein. The computer 800 may also include other types of non-transitory computer-readable medium, such as storage 805 (e.g., one or more disk drives) in addition to the system memory 802. The storage 805 may also store one or more application programs and/or external components used by application programs (e.g., software libraries), which may be loaded into the memory 802.

The computer 800 may have one or more input devices and/or output devices, such as devices 806 and 807 illustrated in FIG. 8. These devices can be used, among other things, to present a user interface. Examples of output devices that can be used to provide a user interface include printers or display screens for visual presentation of output and speakers or other sound generating devices for audible presentation of output. Examples of input devices that may be used for a user interface include keyboards and pointing devices, such as mice, touch pads, and digitizing tablets. As another example, the input devices 807 may include a microphone for capturing audio signals, and the output devices 806 may include a display screen for visually rendering, and/or a speaker for audibly rendering, recognized text.

As shown in FIG. 8, the computer 800 may also comprise one or more network interfaces (e.g., the network interface 810) to enable communication via various networks (e.g., the network 820). Examples of networks include a local area network or a wide area network, such as an enterprise network or the Internet. Such networks may be based on any suitable technology and may operate according to any suitable protocol and may include wireless networks, wired networks, and/or fiber optic networks.

In some embodiments, one or more aspects of the present disclosure may be implemented on a server. As an example, one or more data sets such as the illustrative data sets 510 and 520 shown in FIG. 5 may be stored on a first server. As another example, one or more of the acts of the illustrative process 600 shown in FIG. 6 may be performed by a second server, which may be the same as, or different from, the first server. In the latter case, the first and second servers may be connected via one or more networks. For instance, the second server may be programmed to request data from the first server via the one or more networks. Additionally, or alternatively, the second server may be programmed to receive raw data relating to one or more production runs (e.g., from one or more sensors, directly or indirectly), process the raw data, and/or transmit the raw and/or processed data to the first server. The first server may use such data in any suitable manner, for example, to update one or more data sets (e.g., the illustrative data sets 510 and 520 shown in FIG. 5), and/or to perform clustering (e.g., as described in connection with FIG. 7A).

Any suitable computer may be used as a server. In some embodiments, a server may include a single computer, or multiple computers configured to perform parallel processing. Any suitable types of computers may be used, such as a desktops, laptops, tablets, smartphones, etc. Additionally, or alternatively, a server may include computing resources from one or more public and/or private clouds.

In some embodiments, one or more computers may be used to present a user interface (e.g., to receive user input and/or display results as discussed in connection with the illustrative process 600 shown in FIG. 6). The user interface may be presented in any suitable manner, for example, via a web browser or some other application running on a computer.

### Overall System and Method

Certain systems and methods described herein are systems and methods of manufacturing biologically-produced products, which may include pharmaceutical and/or protein products. In some cases, these systems and methods incorporate innovations in the upstream and/or downstream processes. In certain embodiments, the above-described framework involving generating a plurality of data sets and using the plurality of data sets to evaluate candidate sequences of partitioning units and/or partitioning conditions is used to design downstream processes for purifying at least one biologically-produced product. Some embodiments described herein are inventive systems and methods of manufacturing exemplary biologically-produced products, including G-CSF, a single-domain antibody (e.g., nanobody), hGH, and IFN-α2b. In certain of these embodiments, at least one step was designed using the above-described design framework.

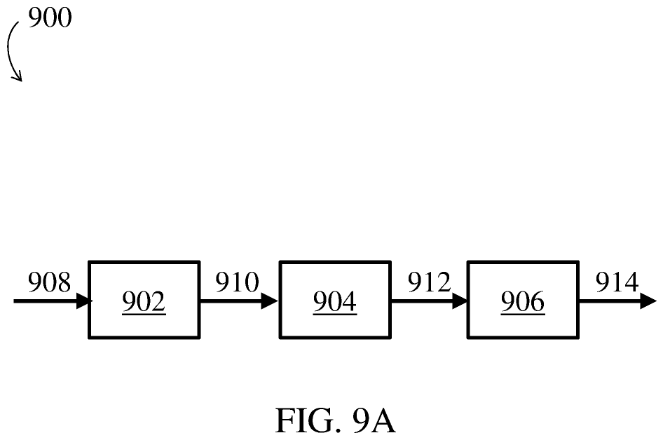

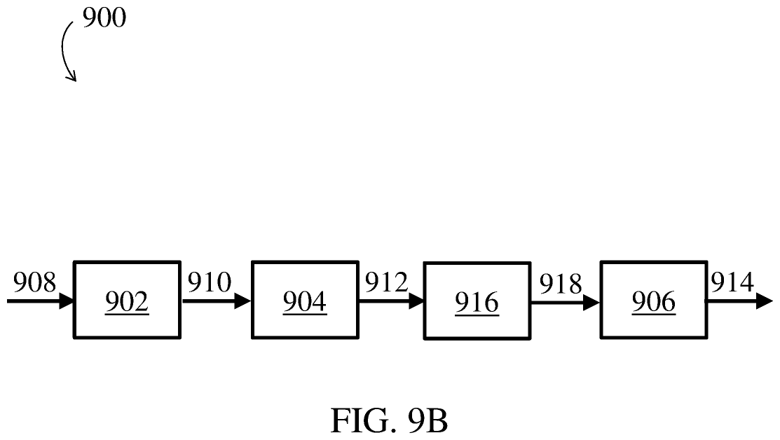

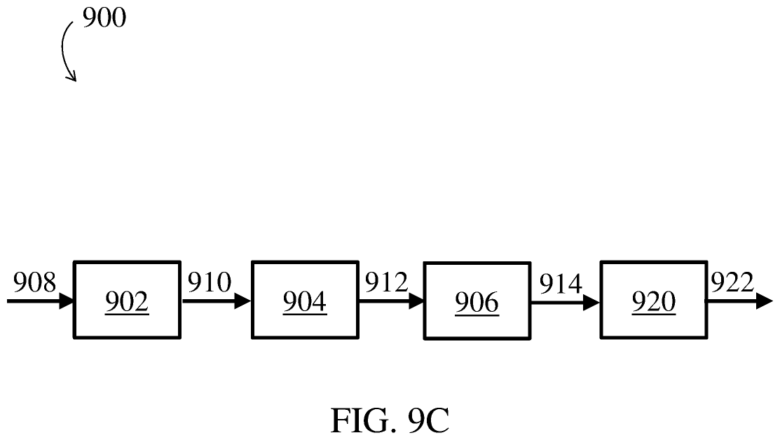

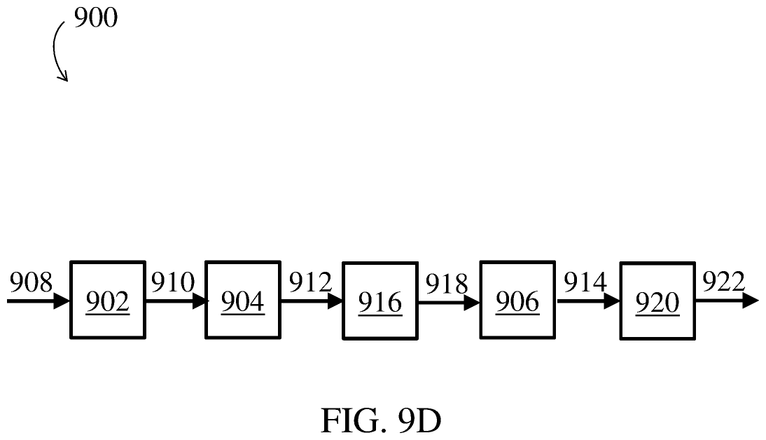

FIG. 9A is a schematic diagram of exemplary biomanufacturing system 900, according to some embodiments. In FIG. 9A, biomanufacturing system 900 comprises bioreactor 902. In some embodiments, bioreactor 902 is configured to promote the growth and maintenance of at least a first type of biological cells configured to express at least one biologically-produced product. In certain embodiments, for example, bioreactor 902 comprises a reaction chamber. According to some embodiments, the reaction chamber of bioreactor 902 contains a suspension comprising a cell culture medium (e.g., a growth cell culture medium configured to promote growth of the first type of biological cells, a production cell culture medium configured to promote expression of the at least one biologically-produced product) and the first type of biological cells.

As shown in FIG. 9A, biomanufacturing system 900 further comprises filter 904, according to some embodiments. In certain embodiments, filter 904 is fluidically connected (e.g., directly fluidically connected) to bioreactor 902. In some cases, for example, filter 904 is at least partially submerged in the suspension contained in the reaction chamber of bioreactor 902. In some embodiments, filter 904 is configured to allow at least a portion of the cell culture medium to flow through filter 904 as a filtrate while causing the biological cells to be retained within the reactor chamber as a retentate.

In some embodiments, biomanufacturing system 900 further comprises purification module 906. According to some embodiments, purification module 906 is fluidically connected (e.g., directly fluidically connected) to filter 904 and/or bioreactor 902. Purification module 906 may, in some embodiments, be configured to remove at least a first type of impurity and a second type of impurity from an output of filter 904 and/or bioreactor 902. In certain cases, purification module 906 comprises a first partitioning unit configured to remove at least the first type of impurity and a second partitioning unit configured to remove at least the second type of impurity. In certain embodiments, the first partitioning unit is fluidically connected (e.g., directly fluidically connected) to the second partitioning unit. In some embodiments, the sequence of partitioning units and/or partitioning conditions may be designed using the above-described framework.

It should be understood that direct fluidic connection between the first partitioning unit and the second partitioning unit may allow for compact design and efficient space utilization in the biomanufacturing system. In some conventional systems, direct fluidic connection between the first partitioning unit and the second partitioning unit would not be possible due to the need for additional processing steps (e.g., hold steps, modifications step) between the first partitioning unit and the second partitioning unit.

In operation, bioreactor 902 receives a first type of biological cells configured to express at least one biologically-produced product, according to some embodiments. In some embodiments, bioreactor 902 further receives feed stream 908 comprising a cell culture medium. The cell culture medium may, for example, be a growth cell culture medium configured to promote growth of the first type of biological cells and/or a production cell culture medium configured to promote expression of the at least one biologically-produced product. In some embodiments, the first type of biological cells are suspended in the cell culture medium, such that the reactor chamber of bioreactor 902 contains a suspension comprising the first type of biological cells and the cell culture medium. According to some embodiments, the first type of biological cells in the suspension proliferate and/or express the at least one biologically-produced product. In certain embodiments, the first type of biological cells secrete the at least one biologically-produced product into the cell culture medium of the suspension.

In some embodiments, at least a portion of the suspension is directed to flow through filter 904 as cell suspension stream 910 to produce first filtrate 912. According to some embodiments, first filtrate 912 comprises the at least one biologically-produced product and is lean in the first type of biological cells relative to cell suspension stream 910.

In some embodiments, first filtrate 912 is directed to flow to purification module 906 to produce purified filtrate 914. In some cases, purified filtrate 914 comprises the at least one biologically-produced product and is lean in at least a first type of impurity and a second type of impurity relative to first filtrate 912. According to certain embodiments, first filtrate 912 is directed to flow to a first partitioning unit of purification module 906 to remove at least the first type of impurity to produce a first partitioned filtrate that comprises the at least one biologically-produced product and is lean in the first type of impurity relative to first filtrate 912. In certain embodiments, the first partitioned filtrate is subsequently directed to flow to a second partitioning unit of purification module 906 to remove at least the second type of impurity to produce a second partitioned filtrate that comprises the at least one biologically-produced product and is lean in the second type of impurity relative to the first partitioned filtrate. In certain embodiments, the second partitioned filtrate is directed to flow to additional partitioning units within purification module 906. In certain embodiments, the second partitioned filtrate is collected as purified filtrate 914. As discussed in further detail below, in some embodiments, purified filtrate 914 is directed to flow to additional modules of biomanufacturing system 900 for further processing. In some embodiments, purified filtrate 914 is discharged from biomanufacturing system 900 as a biologically-produced product stream.

As shown in FIG. 9B, in some embodiments, biomanufacturing system 900 further comprises optional adjustment module 916. In some embodiments, optional adjustment module 916 is fluidically connected (e.g., directly fluidically connected) to filter 904, bioreactor 902, and/or purification module 906. Adjustment module 916 may be configured to adjust one or more properties of an outflow of bioreactor 902 and/or filter 904. In operation, for example, adjustment module 916 may receive first filtrate 912 and/or cell suspension stream 910 and may adjust (e.g., increase, decrease) one or more properties (e.g., pH, conductivity, stability, flow rate, pressure) of first filtrate 912 and/or cell suspension stream 910 to produce adjusted filtrate 918. In certain embodiments, adjustment module 916 may increase or decrease the pH of first filtrate 912 and/or cell suspension stream 910, and adjusted filtrate 918 may be a pH-adjusted filtrate. In some embodiments, adjustment module 916 may increase or decrease the flow rate and/or pressure of first filtrate 912. For instance, adjustment module 916 may increase or decrease the flow rate and/or pressure of first filtrate 912, e.g., during a process step and/or prior to arrival at a subsequent module (e.g., purification module 906). In some such embodiments, adjustment of one or more properties (e.g., pH, conductivity, stability, flow rate, pressure) of the filtrate during a process step and/or prior to the arrival at a subsequent module may place the filtrate in suitable condition for a subsequent step. In some embodiments, adjusted filtrate 918 is directed to flow to purification module 906. According to certain embodiments, one or more properties of adjusted filtrate 918 may be compatible with a partitioning technique and associated conditions applied by a first partitioning unit of purification module 906.

In some embodiments, biomanufacturing system 900 further comprises optional formulation module 920. For example, FIG. 9C illustrates exemplary biomanufacturing system 900 comprising bioreactor 902, filter 904, purification module 906, and formulation module 920. In some embodiments, as shown in FIG. 9D, biomanufacturing system 900 comprises both optional adjustment module 916 and optional formulation module 920 in addition to bioreactor 902, filter 904, and purification module 906. As shown in FIGS. 9C-9D, optional formulation module 920 may be fluidically connected (e.g., directly fluidically connected) to purification module 906. In some embodiments, optional formulation module 920 is configured to further process an output of purification module 906 (e.g., purified filtrate 914) to produce a formulated product. According to certain embodiments, for example, optional formulation module 920 comprises a filtration unit (e.g., a tangential flow filtration (TFF) device). In some cases, the filtration unit concentrates and/or further purifies the at least one biologically-produced product. In certain embodiments, optional formulation module 920 comprises a viral filtration unit configured to remove and/or inactivate one or more viruses that may be present in purified filtrate 914. In certain embodiments, optional formulation module 920 comprises a product packaging unit configured to deposit portions of purified filtrate 914 into one or more aseptic and/or sterile containers (e.g., bags, vials) configured to store a biologically-produced product. In operation, optional formulation module 920 may receive purified filtrate 914 and produce formulated product stream 922, according to some embodiments. In some embodiments, formulation unit 920 comprises a dilution adjustment unit. In some embodiments, the dilution adjustment unit is configured to add a diluent to an output of the purification module (e.g., the purified filtrate). Non-limiting examples of suitable diluents include polar protic solvents (e.g., water, aqueous solutions, buffers, methanol, ethanol, acetic acid), polar aprotic solvents (e.g. dimethylsulfoxide, acetonitrile, dimethylformamide, acetone), and nonpolar solvents (e.g., pentane, hexane, cyclohexane, benzene). In some embodiments, the diluent may include agents to stabilize the formulated purified filtrate to improve stability. Non-limiting examaples include antioxidants (e.g., sodium bisulfite, sodium metabisulfite, ascorbate, sodium sulfite, thioglycerol), bulking agents (e.g., mannitol, dextran, glycine), viscosity enhancers/reducers or surfactants (e.g., polysorbate, 20, polysorbate 80), chelating agents (e.g., EDTA), preservatives (e.g., thimersol, sorbic acid), cryoprotectants (e.g., sucrose, trehalose, sorbitol), lyoprotectants, and adjuvants.(e.g., TLR agonists, CpG DNA, alum).

In some embodiments, biomanufacturing system 900 (as illustrated in any one of FIGS. 9A-9D) is an integrated system. An integrated system generally refers to a system in which each system component is directly fluidically connected to at least one other system component such that a fluidic path (e.g., a closed fluidic path) exists from a first component to a last component of the system. According to some embodiments, for example, each component of biomanufacturing system 900 is directly fluidically connected to at least one other component of biomanufacturing system 900. In certain embodiments, bioreactor 902 is directly fluidically connected to filter 904, and filter 904 is directly fluidically connected to purification module 906. In certain other embodiments, bioreactor 902 is directly fluidically connected to filter 902, filter 902 is directly fluidically connected to optional adjustment module 916, optional adjustment module is directly fluidically connected to purification module 906, and purification module 906 is directly connected to optional formulation module 920. In some embodiments, biomanufacturing system 900 comprises a fluidic path from a first module (e.g., bioreactor 902) to an end module of system 900 (e.g., purification module 906, optional formulation module 920).

In some embodiments, biomanufacturing system 900 (as illustrated in any one of FIGS. 9A-9D) comprises one or more isolators. An isolator generally refers to an air-tight enclosure providing a barrier to the surrounding ambient environment (e.g., a HEPA-filtered enclosure). In some cases, an isolator may provide a reduced particulate environment (e.g., by means of positive pressure). An isolator may use laminar air flow to achieve a reduced particulate environment (e.g., a biosafety cabinet). In some cases, an isolator may advantageously limit microbiological contamination and achieve aseptic conditions, of importance to the manufacture of biologically-produced products. An isolator may have a design compliant with Current Good Manufacturing Practices (CGMPs).

In some embodiments, biomanufacturing system 900 comprises at least two modules that are housed under a single isolator. For example, biomanufacturing system 900 may further comprise at least one isolator housing any two of the following modules: bioreactor 902, filter 904, purification module 906, adjustment module 916, and formulation module 920. In certain embodiments, at least one isolator houses one or more of the following combinations of modules: bioreactor 902, filter 904, and purification module 906; bioreactor 902, filter 904, and adjustment module 916; bioreactor 902, filter 904, and formulation module 920; adjustment module 916 and purification module 906; adjustment module 916 and formulation module 920; or purification module 906 and adjustment module 916. In certain embodiments, a single isolator houses all modules of biomanufacturing system 900 and aseptically isolates all components from the surrounding environment. For example, a single isolator may house bioreactor 902, filter 904, adjustment module 916, and purification module 906. As another example, a single isolator may house bioreactor 902, filter 904, adjustment module 916, purification module 906, and formulation module 920. In some embodiments, biomanufacturing system 900 comprises two or more isolators. In certain embodiments, each of the two or more isolators house at least two modules of biomanufacturing system 900.

As used herein, a direct fluid connection exists between a first component and a second component (and the two components are said to be “directly fluidically connected” to each other) when they are fluidically connected to each other such that the composition of a connecting fluid stream does not substantially change (i.e., no phase change occurs and no fluid component changes in relative abundance by more than 5%) as it flows from the first component to the second component. As an illustrative example, a first component and a second component are “directly fluidically connected” if a connecting fluid stream undergoes changes in pressure and/or temperature during passage from the first component to the second component, but not if the connecting fluid stream undergoes a separation step or a chemical reaction that substantially alters the chemical composition of the connecting fluid stream during passage from the first component to the second component. In some embodiments, one or more fluidic connections (e.g., direct fluidic connections) between one or more modules are “functionally closed” (e.g., assembled so as to maintain aseptic conditions within the one or more modules).

In some embodiments, biomanufacturing system 900 (as illustrated in any one of FIGS. 9A-9D) is a perfusion system. In certain embodiments, biomanufacturing system 900 may be operated under continuous and/or semi-continuous conditions. A system is generally considered to be operated under continuous conditions if at least an input stream and an output stream of the system have a non-zero flow rate over a specified period of time. According to some embodiments, at least one component of biomanufacturing system 900 (e.g., bioreactor 902, filter 904, adjustment module 916, purification module 906, formulation module 920) is operated under continuous and/or semi-continuous conditions. In some embodiments, each component of biomanufacturing system 900 is operated under continuous conditions. In certain embodiments, biomanufacturing system 900 as a whole is operated under continuous and/or semi-continuous conditions. According to some embodiments, each component of biomanufacturing system 900 is directly fluidically connected to at least one other component such that a fluid stream flows from one component to the other. For example, in some embodiments, first filtrate 912 is a first filtrate stream. In some embodiments, adjusted filtrate 918 is an adjusted filtrate stream. In some embodiments, purified filtrate 914 is a purified filtrate stream.

According to certain embodiments, a biomanufacturing system (e.g., system 900) comprises a bioreactor (e.g., bioreactor 902) comprising a reactor chamber having an internal volume in the range of about 50 mL to about 1 L. In some cases, a feed stream (e.g., stream 908) and at least one of a purified filtrate stream (e.g., stream 914) and a formulated product stream (e.g., stream 922) of the biomanufacturing system each have a flow rate of at least about 0.01 mL/min, at least about 0.05 mL/min, at least about 0.1 mL/min, at least about 0.15 mL/min, at least about 0.2 mL/min, at least about 0.3 mL/min, at least about 0.4 mL/min, at least about 0.5 mL/min, at least about 0.6 mL/min, at least about 0.7 mL/min, at least about 0.8 mL/min, at least about 0.9 mL/min, at least about 1 mL/min, at least about 1.5 mL/min, or at least about 2 mL/min over a specified time period. In some embodiments, the feed stream (e.g., stream 908) and at least one of the purified filtrate stream (e.g., stream 914) and the formulated product stream (e.g., stream 922) of the biomanufacturing system each have a flow rate in the range of about 0.01 mL/min to about 0.1 mL/min, 0.01 mL/min to about 0.5 mL/min, about 0.01 mL/min to about 1 mL/min, about 0.01 mL/min to about 2 mL/min, about 0.03 mL/min to about 0.1 mL/min, 0.05 mL/min to about 0.1 mL/min, about 0.05 mL/min to about 0.5 mL/min, about 0.05 mL/min to about 1 mL/min, about 0.05 mL/min to about 2 mL/min, about 0.07 mL/min to about 0.2 mL/min, about 0.1 mL/min to about 0.4 mL/min, about 0.1 mL/min to about 1 mL/min, about 0.3 mL/min to about 1 mL/min, about 0.5 mL/min to about 1 mL/min, about 0.5 mL/min to about 2 mL/min, or about 1 mL/min to about 2 mL/min over a specified time period. The flow rate of any fluid stream within a biomanufacturing system may be measured using any suitable flow rate measurement device known in the art. Non-limiting examples of suitable flow rate measurement devices include ultrasonic flow meters, paddle wheel flow meters, rotameters, vortex flow meters, magnetic flow meters, turbine flow meters, and optical flow sensors (e.g., microparticle or bubble detection devices).

In some cases, each fluid stream of a biomanufacturing system comprising a reactor chamber having an internal volume in the range of about 50 mL to about 1 L (e.g., feed stream 908, cell suspension stream 910, first filtrate stream 912, adjusted filtrate stream 918, purified filtrate stream 914, formulated product stream 922) has a flow rate of at least about 0.01 mL/min, at least about 0.05 mL/min, at least about 0.1 mL/min, at least about 0.15 mL/min, at least about 0.2 mL/min, at least about 0.3 mL/min, at least about 0.4 mL/min, at least about 0.5 mL/min, at least about 0.6 mL/min, at least about 0.7 mL/min, at least about 0.8 mL/min, at least about 0.9 mL/min, at least about 1 mL/min, at least about 1.5 mL/min, or at least about 2 mL/min over a specified time period. In some embodiments, each fluid stream of this biomanufacturing system has a flow rate in the range of about 0.01 mL/min to about 0.1 mL/min, 0.01 mL/min to about 0.5 mL/min, about 0.01 mL/min to about 1 mL/min, about 0.01 mL/min to about 2 mL/min, about 0.03 mL/min to about 0.1 mL/min, 0.05 mL/min to about 0.1 mL/min, about 0.05 mL/min to about 0.5 mL/min, about 0.05 mL/min to about 1 mL/min, about 0.05 mL/min to about 2 mL/min, about 0.07 mL/min to about 0.2 mL/min, about 0.1 mL/min to about 0.4 mL/min, about 0.1 mL/min to about 1 mL/min, about 0.3 mL/min to about 1 mL/min, about 0.5 mL/min to about 1 mL/min, about 0.5 mL/min to about 2 mL/min, or about 1 mL/min to about 2 mL/min over a specified time period.

In some embodiments, the specified time period over which the flow rate is measured is at least about 1 hour, at least about 2 hours, at least about 5 hours, at least about 10 hours, at least about 1 day, at least about 2 days, at least about 3 days, at least about 4 days, at least about 5 days, at least about 6 days, at least about 7 days, at least about 2 weeks, at least about 5 weeks, or at least about 10 weeks.

In some embodiments, a biomanufacturing system comprising a reactor chamber having an internal volume in the range of about 50 mL to about 1 L produces at least about 10 μg, at least about 50 μg, at least about 100 μg, at least about 500 μg, at least about 1 mg, at least about 5 mg, at least about 10 mg, at least about 20 mg, at least about 50 mg, at least about 100 mg, at least about 200 mg, at least about 500 mg, at least about 1 g, at least about 2 g, at least about 5 g, at least about 10 g, at least about 20 g, or at least about 50 g of the at least one biologically-produced product per day. In some embodiments, the system is configured to produce an amount of the at least one biologically-produced product in the range of about 10 μg to about 1 mg, about 10 μg to about 10 mg, about 10 μg to about 50 mg, about 10 μg to about 100 mg, about 10 μg to about 500 mg, about 10 μg to about 1 g, about 10 p.g to about 5 g, about 10 μg to about 10 g, about 10 μg to about 50 g, about 100 μg to about 1 mg, about 100 μg to about 10 mg, about 100 μg to about 50 mg, about 100 μg to about 100 mg, about 100 μg to about 500 mg, about 100 μg to about 1 g, about 100 μg to about 5 g, about 100 μg to about 10 g, about 100 μg to about 50 g, about 1 mg to about 10 mg, about 1 mg to about 50 mg, about 1 mg to about 100 mg, about 1 mg to about 500 mg, about 1 mg to about 1 g, about 1 mg to about 5 g, about 1 mg to about 10 g, about 1 g to about 50 g, about 10 mg to about 50 mg, about 10 mg to about 100 mg, about 10 mg to about 500 mg, about 10 mg to about 1 g, about 10 mg to about 5 g, about 10 mg to about 10 g, about 10 mg to about 50 g, about 50 mg to about 100 mg, about 50 mg to about 200 mg, about 50 mg to about 500 mg, about 50 mg to about 1 g, about 50 mg to about 2 g, about 50 mg to about 5 g, about 50 mg to about 10 g, about 50 mg to about 20 g, about 50 mg to about 50 g, about 100 mg to about 500 mg, about 100 mg to about 1 g, about 100 mg to about 2 g, about 100 mg to about 5 g, about 100 mg to about 10 g, about 100 mg to about 20 g, about 100 mg to about 50 g, about 500 mg to about 1 g, about 500 mg to about 2 g, about 500 mg to about 5 g, about 500 mg to about 10 g, about 500 mg to about 50 g, about 1 g to about 5 g, about 1 g to about 10 g, or about 1 g to about 50 g per day.

According to certain embodiments, a biomanufacturing system (e.g., system 900) comprises a bioreactor (e.g., bioreactor 902) comprising a reactor chamber having an internal volume in the range of about 1 L to about 10 L. In some cases, a feed stream (e.g., stream 908) and at least one of a purified filtrate stream (e.g., stream 914) and a formulated product stream (e.g., stream 922) of the biomanufacturing system each have a flow rate of at least about 0.5 mL/min, at least about 1 mL/min, at least about 1.5 mL/min, at least about 2 mL/min, at least about 5 mL/min, at least about 10 mL/min, at least about 15 mL/min, or at least about 20 mL/min over a specified time period. In some embodiments, the feed stream (e.g., stream 908) and at least one of the purified filtrate stream (e.g., stream 914) and the formulated product stream (e.g., stream 922) of the biomanufacturing system each have a flow rate in the range of about 0.5 mL/min to about 2 mL/min, about 0.5 mL/min to about 5 mL/min, about 0.5 mL/min to about 10 mL/min, about 0.5 mL/min to about 15 mL/min, about 0.5 mL/min to about 20 mL/min, about 3 mL/min to about 10 mL/min, about 5 mL/min to about 10 mL/min, about 5 mL/min to about 15 mL/min, about 5 mL/min to about 20 mL/min, about 7 mL/min to about 20 mL/min, about 10 mL/min to about 20 mL/min, or about 15 mL/min to about 20 mL/min over a specified time period.

In some cases, each fluid stream of a biomanufacturing system comprising a reactor chamber having an internal volume in the range of about 1 L to about 10 L (e.g., feed stream 908, cell suspension stream 910, first filtrate stream 912, adjusted filtrate stream 918, purified filtrate stream 914, formulated product stream 922) has a flow rate of at least about 0.5 mL/min, at least about 1 mL/min, at least about 1.5 mL/min, at least about 2 mL/min, at least about 5 mL/min, at least about 10 mL/min, at least about 15 mL/min, or at least about 20 mL/min over a specified time period. In some embodiments, each fluid stream of this biomanufacturing system has a flow rate in the range of about 0.5 mL/min to about 2 mL/min, about 0.5 mL/min to about 5 mL/min, about 0.5 mL/min to about 10 mL/min, about 0.5 mL/min to about 15 mL/min, about 0.5 mL/min to about 20 mL/min, about 3 mL/min to about 10 mL/min, about 5 mL/min to about 10 mL/min, about 5 mL/min to about 15 mL/min, about 5 mL/min to about 20 mL/min, about 7 mL/min to about 20 mL/min, about 10 mL/min to about 20 mL/min, or about 15 mL/min to about 20 mL/min over a specified time period.

In some embodiments, the specified time period over which the flow rate is measured is at least about 1 hour, at least about 2 hours, at least about 5 hours, at least about 10 hours, at least about 1 day, at least about 2 days, at least about 3 days, at least about 4 days, at least about 5 days, at least about 6 days, at least about 7 days, at least about 2 weeks, at least about 5 weeks, or at least about 10 weeks.

In some embodiments, a biomanufacturing system comprising a reactor chamber having an internal volume in the range of about 1 L to about 10 L produces at least about 100 μg, at least about 1 mg, at least about 5 mg, at least about 10 mg, at least about 50 mg, at least about 100 mg, at least about 500 mg, at least about 1 g, at least about 5 g, at least about 10 g, at least about 50 g, at least about 100 g, at least about 200 g, or at least about 500 g of the at least one biologically-produced product per day. In some embodiments, the system is configured to produce an amount of the at least one biologically-produced product in the range of about 100 μg to about 1 mg, about 100 μg to about 10 mg, about 100 μg to about 50 mg, about 100 μg to about 100 mg, about 100 μg to about 500 mg, about 100 μg to about 1 g, about 100 μg to about 5 g, about 100 μg to about 10 g, about 100 μg to about 50 g, about 100 μg to about 100 g, about 100 μg to about 500 g, about 1 mg to about 10 mg, about 1 mg to about 50 mg, about 1 mg to about 100 mg, about 1 mg to about 500 mg, about 1 mg to about 1 g, about 1 mg to about 5 g, about 1 mg to about 10 g, about 1 mg to about 50 g, about 1 mg to about 100 g, about 1 mg to about 500 g, about 100 mg to about 500 mg, about 100 mg to about 1 g, about 100 mg to about 5 g, about 100 mg to about 10 g, about 100 mg to about 50 g, about 100 mg to about 100 g, about 100 mg to about 500 g, about 1 g to about 5 g, about 1 g to about 10 g, about 1 g to about 50 g, about 1 g to about 100 g, about 1 g to about 500 g, about 10 g to about 50 g, about 10 g to about 100 g, about 10 g to about 500 g, or about 100 g to about 500 g per day. According to certain embodiments, a biomanufacturing system (e.g., system 900) comprises a bioreactor (e.g., bioreactor 902) comprising a reactor chamber having an internal volume in the range of about 10 L to about 100 L. In some cases, a feed stream (e.g., stream 908) and at least one of a purified filtrate stream (e.g., stream 914) and a formulated product stream (e.g., stream 922) of the biomanufacturing system each have a flow rate of at least about 5 mL/min, at least about 10 mL/min, at least about 15 mL/min, at least about 20 mL/min, at least about 50 mL/min, at least about 100 mL/min, at least about 150 mL/min, or at least about 200 mL/min over a specified time period. In some embodiments, the feed stream (e.g., stream 908) and at least one of the purified filtrate stream (e.g., stream 914) and the formulated product stream (e.g., stream 922) of the biomanufacturing system each have a flow rate in the range of about 5 mL/min to about 20 mL/min, about 5 mL/min to about 100 mL/min, about 5 mL/min to about 150 mL/min, about 5 mL/min to about 200 mL/min, about 10 mL/min to about 50 mL/min, about 10 mL/min to about 100 mL/min, about 10 mL/min to about 150 mL/min, about 10 mL/min to about 200 mL/min, about 35 mL/min to about 100 mL/min, about 35 mL/min to about 150 mL/min, about 35 mL/min to about 200 mL/min, about 50 mL/min to about 100 mL/min, about 50 mL/min to about 150 mL/min, about 50 mL/min to about 200 mL/min, or about 100 mL/min to about 200 mL/min over a specified time period.

In some cases, each fluid stream of a biomanufacturing system comprising a reactor chamber having an internal volume in the range of about 10 L to about 100 L (e.g., feed stream 908, cell suspension stream 910, first filtrate stream 912, adjusted filtrate stream 918, purified filtrate stream 914, formulated product stream 922) has a flow rate of at least about 5 mL/min, at least about 10 mL/min, at least about 15 mL/min, at least about 20 mL/min, at least about 50 mL/min, at least about 100 mL/min, at least about 150 mL/min, or at least about 200 mL/min over a specified time period. In some embodiments, each fluid stream of this biomanufacturing system has a flow rate in the range of about 5 mL/min to about 20 mL/min, about 5 mL/min to about 100 mL/min, about 5 mL/min to about 150 mL/min, about 5 mL/min to about 200 mL/min, about 10 mL/min to about 50 mL/min, about 10 mL/min to about 100 mL/min, about 10 mL/min to about 150 mL/min, about 10 mL/min to about 200 mL/min, about 35 mL/min to about 100 mL/min, about 35 mL/min to about 150 mL/min, about 35 mL/min to about 200 mL/min, about 50 mL/min to about 100 mL/min, about 50 mL/min to about 150 mL/min, about 50 mL/min to about 200 mL/min, or about 100 mL/min to about 200 mL/min over a specified time period.

In some embodiments, the specified time period over which the flow rate is measured is at least about 1 hour, at least about 2 hours, at least about 5 hours, at least about 10 hours, at least about 1 day, at least about 2 days, at least about 3 days, at least about 4 days, at least about 5 days, at least about 6 days, at least about 7 days, at least about 2 weeks, at least about 5 weeks, or at least about 10 weeks.

In some embodiments, a biomanufacturing system comprising a reactor chamber having an internal volume in the range of about 10 L to about 100 L produces at least about 1 mg, at least about 5 mg, at least about 10 mg, at least about 50 mg, at least about 100 mg, at least about 200 mg, at least about 500 mg, at least about 1 g, at least about 2 g, at least about 5 g, at least about 10 g, at least about 50 g, at least about 100 g, at least about 200 g, at least about 500 g, at least about 1 kg, or at least about 5 kg of the at least one biologically-produced product per day. In some embodiments, the system is configured to produce an amount of the at least one biologically-produced product in the range of about 1 mg to about 10 mg, about 1 mg to about 50 mg, about 1 mg to about 100 mg, about 1 mg to about 500 mg, about 1 mg to about 1 g, about 1 mg to about 5 g, about 1 mg to about 10 g, about 1 mg to about 50 g, about 1 mg to about 100 g, about 1 mg to about 500 g, about 1 mg to about 1 kg, about 1 mg to about 5 kg, about 10 mg to about 50 mg, about 10 mg to about 100 mg, about 10 mg to about 500 mg, about 10 mg to about 1 g, about 10 mg to about 5 g, about 10 mg to about 10 g, about 10 mg to about 50 g, about 10 mg to about 100 g, about 10 mg to about 500 g, about 10 mg to about 1 kg, about 10 mg to about 5 kg, about 100 mg to about 500 mg, about 100 mg to about 1 g, about 100 mg to about 5 g, about 100 mg to about 10 g, about 100 mg to about 50 g, about 100 mg to about 100 g, about 100 mg to about 500 g, about 100 mg to about 1 kg, about 100 mg to about 5 kg, about 500 mg to about 1 g, about 1 g to about 5 g, about 1 g to about 10 g, about 1 g to about 50 g, about 1 g to about 100 g, about 1 g to about 500 g, about 1 g to about 1 kg, about 1 g to about 5 kg, about 10 g to about 50 g, about 10 g to about 100 g, about 10 g to about 500 g, about 10 g to about 1 kg, about 10 g to about 5 kg, about 100 g to about 500 g, about 100 g to about 1 kg, about 100 g to about 5 kg, about 500 g to about 1 kg, about 500 g to about 5 kg, or about 1 kg to about 5 kg per day.

Bioreactor

In some embodiments, the bioreactor (e.g., bioreactor 902 in FIG. 9) is a perfusion bioreactor. A perfusion bioreactor generally refers to a bioreactor that is continuously operated (e.g., an input stream and an output stream have a non-zero flow rate over a specified period of time) such that cells are retained within a reactor chamber of the bioreactor but at least a portion of the cell culture medium is continuously removed (and replenished). In some instances, a perfusion bioreactor may be associated with certain advantages compared to a fed-batch bioreactor (e.g., a bioreactor in which cells, media, and products remain in the bioreactor until the end of a run), such as higher cell concentrations and product yields, lower levels of accumulated waste, immediate availability and reduced degradation (e.g., oxidation, aggregation, deamidation, proteolysis) of target biologically-produced products, and more consistent expression profiles. In some cases, a perfusion bioreactor may permit rescue of a product in the event of contamination. In addition, due to the higher cell concentrations that can be achieved in a perfusion bioreactor, a perfusion bioreactor having a certain level of productivity may have a smaller physical size than a comparable fed-batch bioreactor having the same level of productivity. The smaller physical size may make perfusion bioreactors particularly attractive for disposable biomanufacturing systems and maximizing volumetric productivity of a manufacturing process or facility.

In some embodiments, the bioreactor is a chemostat. A chemostat generally refers to a bioreactor that is continuously operated such that an input stream comprising a cell culture medium is continuously supplied and an output stream comprising at least a portion of the cell culture medium and the biological cells is continuously removed such that the wet cell weight of the biological cells is maintained at a substantially constant value. In some embodiments, the bioreactor is a continuous stirred tank reactor (CSTR).

In some embodiments, the bioreactor comprises a reactor chamber. In certain embodiments, the reactor chamber has an internal volume (i.e., a volume capable of containing a fluid such as a cell suspension) of at least about 50 mL, at least about 100 mL, at least about 200 mL, at least about 500 mL, at least about 1 L, at least about 2 L, at least about 5 L, at least about 10 L, at least about 50 L, or at least about 100 L. In some embodiments, the reactor chamber has an internal volume of about 100 L or less, about 50 L or less, about 10 L or less, about 5 L or less, about 2 L or less, about 1 L or less, about 500 mL or less, about 200 mL or less, about 100 mL or less, or about 50 mL or less. In some embodiments, the reactor chamber has an internal volume in the range of about 50 mL to about 100 mL, about 50 mL to about 500 mL, about 50 mL to about 1 L, about 50 mL to about 5 L, about 50 mL to about 10 L, about 50 mL to about 50 L, about 50 mL to about 100 L, about 100 mL to about 500 mL, about 100 mL to about 1 L, about 100 mL to about 5 L, about 100 mL to about 10 L, about 100 mL to about 50 L, about 100 mL to about 100 L, about 500 mL to about 1 L, about 500 mL to about 5 L, about 500 mL to about 10 L, about 500 mL to about 50 L, about 500 mL to about 100 L, about 1 L to about 10 L, about 1 L to about 50 L, about 1 L to about 100 L, about 10 L to about 50 mL, about 10 L to about 100 L, or about 50 L to about 100 L.

The reactor chamber of the bioreactor may have any suitable shape. According to certain embodiments, for example, the reactor chamber may be substantially cylindrical. The reactor chamber also may be formed of any suitable material. Non-limiting examples of a suitable material include stainless steel, glass, and plastic. In some embodiments, the reactor chamber comprises one or more internal components, such as an agitator and/or impeller. An agitator and/or impeller may, for example, promote suspension of the cells within the cell culture medium and/or may maintain a sufficiently high level of dissolved oxygen within a cell culture. The impeller may be any suitable type of impeller. Non-limiting examples of suitable types of impellers include Rushton impellers, Marine impellers, and angled-Rushton impellers. In certain embodiments, the reaction chamber of a bioreactor comprises 1, 2, 3, or more impellers. In some instances, 2 or more impellers may be arranged on a single shaft. In those instances, the 2 or more impellers may be Rushton impellers, Marine impellers, and/or angled-Rushton impellers. In some embodiments, the bioreactor is fluidically connected (e.g., directly fluidically connected) to a gas concentration device or a gas source (e.g., gas tank). In certain cases, the gas concentration device is an oxygen concentrator.

In some embodiments, the bioreactor is operated in at least two phases: a cell growth phase and a biologically-produced product production phase. According to certain embodiments, in the cell growth phase, the bioreactor receives a first type of biological cells configured to express at least one biologically-produced product (i.e., the bioreactor is “inoculated” with the first type of biological cells) and receives a feed stream comprising a growth cell culture medium configured to promote the growth of the first type of biological cells. In some embodiments, the first type of biological cells are incubated in the growth cell culture medium for a period of at least about 1 hour, at least about 6 hours, at least about 12 hours, at least about 24 hours, at least about 32 hours, at least about 36 hours, at least about 2 days, at least about 3 days, at least about 4 days, at least about 5 days, at least about 6 days, at least about 7 days, or at least about 14 days. In some embodiments, the first type of biological cells are incubated in the growth cell culture medium for a period in the range of about 1 hour to about 12 hours, about 1 hour to about 24 hours, about 1 hour to about 32 hours, about 1 hour to about 36 hours, about 1 hour to about 48 hours, about 1 hour to about 72 hours, about 1 hour to about 4 days, about 1 hour to about 5 days, about 1 hour to about 6 days, about 1 hour to about 7 days, about 1 hour to about 14 days, about 12 hours to about 24 hours, about 12 hours to about 36 hours, about 12 hours to about 48 hours, about 12 hours to about 72 hours, about 12 hours to about 4 days, about 12 hours to about 5 days, about 12 hours to about 6 days, about 12 hours to about 7 days, about 12 hours to about 14 days, about 24 hours to about 36 hours, about 24 hours to about 48 hours, about 24 hours to about 72 hours, about 24 hours to about 4 days, about 24 hours to about 5 days, about 24 hours to about 6 days, about 24 hours to about 7 days, about 24 hours to about 14 days, about 36 hours to about 72 hours, about 3 days to about 4 days, about 3 days to about 5 days, about 3 days to about 6 days, about 3 days to about 7 days, about 3 days to about 14 days, about 4 days to about 7 days, about 4 days to about 14 days, or about 5 days to about 7 days, about 5 days to about 14 days, about 7 days to about 14 days, or about 10 days to about 14 days.

In some embodiments, the first type of biological cells are incubated in the growth cell culture medium until they reach a wet cell weight of at least about 150 g/L, at least about 200 g/L, at least about 250 g/L, at least about 300 g/L, at least about 350 g/L, at least about 400 g/L, at least about 450 g/L, or at least about 500 g/L. In some embodiments, the first type of biological cells are incubated in the growth cell culture medium until they reach a wet cell weight in the range of about 150 g/L to about 200 g/L, about 150 g/L to about 300 g/L, about 150 g/L to about 400 g/L, about 150 g/L to about 500 g/L, about 200 g/L to about 300 g/L, about 200 g/L to about 400 g/L, about 200 g/L to about 500 g/L, about 300 g/L to about 500 g/L, or about 400 g/L to about 500 g/L. The wet cell weight may be measured by a mass balance.

According to some embodiments, the cell growth phase is ended by removing the growth cell culture medium from the reaction chamber of the bioreactor. In some embodiments, the biologically-produced product production phase is initiated by introducing a production cell culture medium configured to promote expression of the at least one biologically-produced product into the reaction chamber. According to some embodiments, in the biologically-produced product production phase, the bioreactor receives a feed stream comprising the production cell culture medium.

In some embodiments, the first type of biological cells suspended in the production cell culture medium produce at least one biologically-produced product for a period of at least about 1 day, at least about 2 days, at least about 3 days, at least about 4 days, at least about 5 days, at least about 6 days, at least about 7 days, at least about 2 weeks, at least about 4 weeks, at least about 6 weeks, or at least about 10 weeks. In some embodiments, the first type of biological cells suspended in the production cell culture medium produce at least one biologically-produced product for a period in the range of about 1 day to about 7 days, about 1 day to about 2 weeks, about 1 day to about 4 weeks, about 1 day to about 6 weeks, about 1 day to about 10 weeks, about 7 days to about 2 weeks, about 7 days to about 4 weeks, about 7 days to about 6 weeks, about 7 days to about 10 weeks, about 4 weeks to about 6 weeks, or about 4 weeks to about 10 weeks.

In some embodiments, the first type of biological cells suspended in the production cell culture medium have a wet cell weight of at least about 150 g/L, at least about 200 g/L, at least about 250 g/L, at least about 300 g/L, at least about 350 g/L, at least about 400 g/L, at least about 450 g/L, or at least about 500 g/L. In some embodiments, the first type of biological cells suspended in the production cell culture medium have a wet cell weight in the range of about 150 g/L to about 200 g/L, about 150 g/L to about 300 g/L, about 150 g/L to about 400 g/L, about 150 g/L to about 500 g/L, about 200 g/L to about 300 g/L, about 200 g/L to about 400 g/L, about 200 g/L to about 500 g/L, about 300 g/L to about 500 g/L, or about 400 g/L to about 500 g/L.

In some embodiments, the first type of biological cells are configured to express at least one biologically-produced product. For example, the first type of biological cells may be genetically engineered to express at least one biologically-produced product (e.g., via site-directed mutagenesis, gene insertion, viral vectors, microinjection, plasmids, recombinant DNA, metal nanoparticles, electroporation, chemical poration). In some embodiments, the biologically-produced product is a protein product and/or a pharmaceutical product. Non-limiting examples of a suitable biologically-produced product include a cytokine, an antibody, an antibody fragment, a single-domain antibody (e.g., a nanobody), a hormone, an enzyme, a growth factor, a blood factor, a recombinant immunogen, a recombinant vaccine or subunit, and a fusion protein. In some embodiments, the antibody is a single-chain antibody, a bispecific antibody, and/or a monoclonal antibody. In some embodiments, the cytokine is an interferon. According to certain embodiments, the at least one biologically-produced product comprises human growth hormone (hGH), granulocyte-colony stimulating factor (G-CSF), a single-domain antibody (e.g., nanobody), and/or interferon-αlb (IFN-α2b).

In some embodiments, the first type of biological cells is a prokaryotic cell. Non-limiting examples of prokaryotic cells include cyanobacteria algae and bacteria. The bacterium may be a gram-negative bacterium, including, but not limited to, including Escherichia, Salmonella, Shigella, Pseudomonas, Neisseria, Chlamydia, Yersinia, Moraxella, Haemophilus, Helicobacter, Acinetobacter, Stenotrophomonas, Bdellovibrio, Legionella, and acetic acid bacteria. In other embodiments, the bacterium may be a gram-positive bacterium, including, but not limited to, Streptococcus, Staphylococcus, Corynebacterium, Listeria, Bacillus, Clostridium, Lactobacillus, and Mycobacterium.

In some embodiments, the first type of biological cells is a lower eukaryote. Lower eukaryotes include yeast, fungi, collar-flagellates, microsporidia, alveolates (e.g., dinoflagellates), stramenopiles (e.g, brown algae, protozoa), rhodophyta (e.g., red algae), plants (e.g., green algae, plant cells, moss) and other protists. In some embodiments, the first type of biological cells are microalgae cells. A non-limiting example of microalgae cells is Chlamydomonas reinhardtii cells. In some embodiments, the first type of biological cells are diatom cells. A non-limiting example of diatom cells is Phaeodactylum tricornutum cells.

In some embodiments, the first type of biological cells is a yeast cell. Examples of yeast cells include, but are not limited to, Arxula adeninivorans, Aureobasidium pullulans, Aureobasidium melanogenum, Aureobasidium namibiae, Aureobasidium subglaciale, Brettanomyces bruxellensis, Brettanomyces claussenii, Candida albicans, Candida auris, Candida bracarensis, Candida bromeliacearum, Candida dubliniensis, Candida glabrata, Candida humilis, Candida keroseneae, Candida krusei, Candida lusitaniae, Candida oleophila, Candida parapsilosis, Candida rhizophoriensis, Candida sharkiensis, Candida stellate, Candida theae, Candida tolerans, Candida tropicalis, Candida ubatubensis, Candida viswanathii, Candida zemplinina, Cryptococcus gattii, Cryptococcus neoformans, Debaryomyces hansenii, Hansenula polymorpha, Hanseniaspora guilliermondii, Kluyveromyces lactis and like kinds, Kluyveromyces marxianus, Leucosporidium frigidum, Macrorhabdus ornithogaster, Malassezia caprae, Malassezia dermatis, Malassezia equine, Malassezia japonica, Malassezia nana, Malassezia sympodialis, Ogataea methanolica, Ogataea polymorpha, Pachysolen tannophilus, Pichia anomala, Pichia guilliermondii, Pichia pastoris, Pichia stipites, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Rhodotorula cladiensis, Rhodotorula evergladiensis, Saccharomyces bayanus, Saccharomyces boulardii, Saccharomyces cerevisiae, Saccharomyces paradoxus, Schizosaccharomyces pombe, Yarrowia lipolytica, and Zygosaccharomyces bailii. In one embodiment, the yeast is Pichia pastoris.

In some embodiments, the first type of biological cells is a filamentous fungi. Non-limiting examples of filamentous fungi include Trichoderma, for example from Trichoderma reesei; Neurospora, for example from Neurospora crassa; Sordaria, for example from Sordaria macrospora; Aspergillus, for example from Aspergillus niger, Aspergillus nidulans, Aspergillus oryzae, or from Aspergillus sojae; Fonsecaea, for example from Fonsecaea pedrosoi; Cladosporium, for example from Cladosporium carrionii; Chrysosporium luchiowense; Fusarium sp. (for example, Fusarium gramineum, Fusarium venenatum); Physcomitrella patens; or Phialophora, for example from Phialophora verrucosa.

The smaller secretome of organisms such as Pichia pastoris and similar cells are surprisingly advantageous in the context of designing and operating the integrated systems of certain embodiments. For example, Pichia pastoris, which is a methylotrophic yeast, contains the necessary cellular machinery for protein folding, glycosylation, and secretion, so it can be used to produce complex heterologous proteins used as therapeutics. However, its smaller secretome (e.g., the smaller number of proteins expected to contain a secretion signal peptide for entry into the secretory system and eventual secretion into the cell culture medium) allows for more streamlined downstream (e.g., purification) processes than higher eukaryotic cells.

In some embodiments, the reaction chamber contains a growth cell culture medium configured to promote growth of the first type of biological cells. Non-limiting examples of a suitable growth cell culture medium include chemically defined media comprising a carbon source, buffered glycerol-complex medium (BMGY), basal salt media, FM22, and d'Anjou media. Non-limiting examples of suitable additives include methanol, glycerol, sorbitol, glucose, arabinose, corn syrup, corn steep liquor, mannose, galactose, lactose trehaolse, maltitol, xylose, ribose, melibiose, maltose, raffinose, inulin, inositol, sorbose, arabitol, xylitol, ribitol, myo-inositol, glucono-1,5-lactone, lactate, quinic acid, gluconate, and trehalose. In some embodiments, the pH of the growth cell culture medium is at least about 4.0, at least about 5.0, at least about 5.5, at least about 6.0, at least about 6.5, at least about 7.0, at least about 7.5, at least about 8.0, or at least about 8.5. In some embodiments, the pH of the growth cell culture medium is about 8.5 or less, about 8.0 or less, about 7.5 or less, about 7.0 or less, about 6.5 or less, about 6.0 or less, about 5.5 or less, about 5.0 or less, or about 4.0 or less. In some embodiments, the pH of the growth cell culture medium is in the range of about 4.0 to about 6.0, about 4.0 to about 7.0, about 4.0 to about 8.0, about 4.0 to about 8.5, about 5.0 to about 7.0, about 5.0 to about 8.0, about 5.0 to about 8.5, about 6.0 to about 7.0, about 6.0 to about 8.0, about 6.0 to about 8.5, about 7.0 to about 8.0, or about 7.0 to about 8.5. The pH of the growth cell culture medium may be measured according to any method known in the art. For example, the pH may be measured using a digital pH meter.

In some embodiments, the reaction chamber contains a production cell culture medium configured to promote expression of at least one biologically-produced product by the first type of biological cells. Non-limiting examples of a suitable production cell culture medium include chemically defined media comprising a carbon source and/or other additive for induction of protein expression, buffered methanol-complex medium (BMMY), basal salt media with methanol, FM22 with methanol, and d′Anjou media with methanol. Non-limiting examples of suitable additives include methanol, glycerol, sorbitol, glucose, arabinose, corn syrup, corn steep liquor, mannose, galactose, lactose trehaolse, maltitol, xylose, ribose, melibiose, maltose, raffinose, inulin, inositol, sorbose, arabitol, xylitol, ribitol, myo-inositol, glucono-1,5-lactone, lactate, quinic acid, gluconate, and trehalose. In some embodiments, the pH of the production cell culture medium is at least about 4.0, at least about 5.0, at least about 5.5, at least about 6.0, at least about 6.5, at least about 7.0, at least about 7.5, at least about 8.0, or at least about 8.5. In some embodiments, the pH of the production cell culture medium is about 8.5 or less, about 8.0 or less, about 7.5 or less, about 7.0 or less, about 6.5 or less, about 6.0 or less, about 5.5 or less, about 5.0 or less, or about 4.0 or less. In some embodiments, the pH of the production cell culture medium is in the range of about 4.0 to about 6.0, about 4.0 to about 7.0, about 4.0 to about 8.0, about 4.0 to about 8.5, about 5.0 to about 7.0, about 5.0 to about 8.0, about 5.0 to about 8.5, about 6.0 to about 7.0, about 6.0 to about 8.0, about 6.0 to about 8.5, about 7.0 to about 8.0, or about 7.0 to about 8.5.

### Level Sensing System

In some embodiments, the biomanufacturing system (e.g., system 900 in FIG. 9) comprises at least one level sensing system configured to sense a level (e.g., height) of a liquid (e.g., a suspension comprising the first type of biological cells and a cell culture medium) contained within a fluid-containing vessel (e.g., a reaction chamber of a bioreactor). In some cases, the level sensing system may provide input (e.g., a liquid level value) to a process control system, and a rate of perfusion and/or feeding (e.g., nutrient feeding) may be modified based on the liquid level value. The level sensing system may be used for point-level process control or continuous process control. In some instances, the level sensing system may provide real-time, online monitoring of liquid level within the bioreactor. In certain cases, a process control system may verify a signal from the level sensing system for a designated period of time (e.g., at least about 10 seconds) before processing the signal.

According to some embodiments, the level sensing system is a non-invasive level sensing system. In some cases, non-invasive level sensing systems are associated with certain advantages, such as reduced risk of contamination, reduced geometric complexity, and cost savings. In single-use bioreactors, non-invasive level sensing systems may be particular advantageous because they may allow for reuse of the level sensing system without requiring sterilization. However, conventional non-invasive level sensing systems that have been used in other industries are generally unsuitable for use in bioreactors. For example, external capacitive level sensors, which measure change in dielectric constant through a vessel wall, are generally unsuitable for measuring liquid levels in a bioreactor because the ionic conductivity of the bioreactor liquid generally changes during the course of a run and interferes with dielectric constant measurements. Methods employing load cells and pressure transducers are similarly unsuitable for measuring liquid levels in a bioreactor since vibrational noise and variations in the density of the bioreactor liquid may interfere with measurements. Time-of-flight methods, such as ultrasonic or laser-based methods, are also generally unsuitable for measuring liquid levels in a bioreactor because such methods often require very high resolution and are susceptible to change due to a wide range of parameters.

In some embodiments, the level sensing system may be a magnetic level sensing system and/or an optical level sensing system. In some cases, a magnetic and/or optical level sensing system may advantageously provide accurate readings of a liquid level in a bioreactor since the magnetic and/or optical level sensing system may not rely upon properties (e.g., dielectric constant, density) of the bioreactor liquid to measure the liquid level. In addition, a magnetic and/or optical level sensing system may be associated with other advantages compared to other types of level sensing system, including reduced vessel and head plate complexity, reduced risk of contamination, reduced in-vessel wiring requirements, and increased cost savings. In some cases, the magnetic and/or optical level sensing system may allow continuous real-time monitoring.

According to some embodiments, the level sensing system is an in-vessel level sensing system. In certain embodiments, the level sensing system comprises a capacitance-based probe (e.g., an in-vessel capacitance probe).

### Magnetic Level Sensing System

In certain embodiments, the level sensing system is a magnetic level sensing system. In some embodiments, the magnetic level sensing system comprises a magnetic float, a non-magnetic shaft, and one or more magnetically-activated switches (e.g., reed switches). A schematic illustration of an exemplary magnetic level sensing system is shown in FIG. 27. In particular, FIG. 27 shows bioreactor 2710, which comprises reaction chamber 2720 containing a bioreactor liquid (e.g., a cell suspension) 2730. Magnetic float 2740 and non-magnetic shaft 2750 are positioned inside reaction chamber 2720, and array 2760 of magnetically-activated switches is positioned outside reaction chamber 2720. As shown in FIG. 27, magnetic float 2740 encircles non-magnetic shaft 2750 and can move in a vertical direction along non-magnetic shaft 2750. As an illustrative, non-limiting example, array 2760 comprises five magnetically-activated switches. Each switch of array 2760 corresponds to a different colored LED.

In operation, magnetic float 2740 may float on the surface of bioreactor liquid 2730. As the height of bioreactor liquid 2730 increases or decreases, magnetic float 2740 may move vertically along non-magnetic shaft 2750. As magnetic float 2740 moves vertically along non-magnetic shaft 2750, magnetic float 2740 may become sufficiently close to a magnetically-activated switch of array 2760 to activate the switch, and an LED may be illuminated. Based on the color of the LED, an observer may be able to determine the level of bioreactor liquid 2730 within reaction chamber 2720.

In some embodiments, the magnetic float of the magnetic level sensing system comprises a flotation ring. The flotation ring may comprise any material suitable for use in a bioreactor. In certain embodiments, the material of the flotation ring is biocompatible.

In some instances, exposure of cells (e.g., the first type of biological cells) to a biocompatible material (e.g., the material of the flotation ring) results in the death of a relatively low percentage of cells (or substantially no cells). In certain embodiments, exposure of cells to a biocompatible material results in a cell death percentage of about 20% or less, about 15% or less, about 10% or less, about 5% or less, about 2% or less, about 1% or less, about 0.5% or less, about 0.2% or less, or about 0.1% or less. In some cases, exposure of cells to a biocompatible material results in the death of substantially no cells. The cell death percentage may be calculated as the number of dead cells divided by the number of live cells, multiplied by 100. The numbers of dead and live cells may be measured according to any method known in the art, such as flow cytometry or a Trypan blue exclusion assay. In some cases, a biocompatible material (e.g., the material of the flotation ring) may be inserted or injected into a living subject (e.g., a human or non-human animal) without inducing an adverse response (e.g., inflammation or other immune response). In some embodiments, a biocompatible material meets USP Class VI requirements, complies with the ISO 10993 standard, and/or has been designated as Generally Recognized as Safe. The biocompatible material may be a plastic, metal, ceramic, or other suitable material. Non-limiting examples of suitable biocompatible materials include titanium, titanium alloys, stainless steel, cobalt-chromium alloys, glass, alumina, polyethylene, polypropylene, polyethylene terephthalate, polymethyl methacrylate, polyvinyl alcohol, polyvinyl chloride, polystyrenes, polyamides, polyesters, polyurethanes, and silicones.

In some embodiments, the material of the flotation ring can withstand sterilization. A material that can withstand sterilization generally refers to a material that can withstand exposure to radiation (e.g., gamma radiation), steam, dry heat, sterilizing chemicals (e.g., ethylene oxide, nitrogen dioxide, ozone, hydrogen peroxide).

In certain embodiments, the material of the flotation ring has a specific gravity less than the liquid within the bioreactor. A non-limiting example of a material having a suitable specific gravity is polystyrene foam. In some embodiments, the flotation ring comprises a material (e.g., a biocompatible material) encompassing a hollow region comprising a gas (e.g., air). The material encompassing the hollow region may be any suitable material.

In some embodiments, the magnetic float of the magnetic level sensing system comprises one or more magnets. According to some embodiments, the one or more magnets are substantially equidistantly spaced around the flotation ring. In some such embodiments, magnetic material may be substantially uniformly distributed around the flotation ring, which may advantageously result in a rotation-independent magnetic field. In some embodiments, the one or more magnets comprise at least 1 magnet, at least 2 magnets, at least 3 magnets, at least 4 magnets, at least 5 magnets, at least 6 magnets, at least 7 magnets, at least 8 magnets, at least 9 magnets, or at least 10 magnets. In certain cases, the one or more magnets comprise between about 1 and 2 magnets, between 1 and 5 magnets, between 1 and 10 magnets, between 2 and 5 magnets, between about 2 and 10 magnets, or between about 5 and 10 magnets.

In some embodiments, the one or more magnets of the magnetic float are sufficiently strong to generate a magnetic field that can penetrate a wall of a bioreactor reaction chamber. In some embodiments, at least one of the one or more magnets has a pull force of at least about 1 pound, at least about 2 pounds, at least about 3 pounds, at least about 4 pounds, at least about 5 pounds, at least about 5.5 pounds, at least about 6 pounds, at least about 6.5 pounds, at least about 7 pounds, at least about 8 pounds, at least about 9 pounds, at least about 10 pounds, at least about 20 pounds, at least about 30 pounds, at least about 40 pounds, or at least about 50 pounds. In certain embodiments, at least one of the one or more magnets has a pull force between about 1 pound and about 5 pounds, between about 1 pound and about 10 pounds, between about 1 pound and about 20 pounds, between about 1 pound and about 30 pounds, between about 1 pound and about 40 pounds, between about 1 pound and about 50 pounds, between about 5 pounds and about 10 pounds, between about 5 pounds and about 20 pounds, between about 5 pounds and about 30 pounds, between about 5 pounds and about 40 pounds, between about 5 pounds and about 50 pounds, between about 10 pounds and about 20 pounds, between about 10 pounds and about 30 pounds, between about 10 pounds and about 40 pounds, between about 10 pounds and about 50 pounds, between about 20 pounds and about 50 pounds, between 30 pounds and about 50 pounds, or between about 40 pounds and about 50 pounds.

The one or more magnets of the magnetic float may comprise any suitable magnetic material. Non-limiting examples of suitable magnetic materials include neodymium iron boron (NdFeB), samarium cobalt (SmCo), and aluminum nickel cobalt (Alnico). In certain embodiments, the magnetic material is coated (e.g., to promote biocompatibility). Examples of suitable coating materials include, but are not limited to, parylene, silicone, nickel, titanium, and titanium nitride.

The one or more magnets may have any suitable size. In some embodiments, at least one of the one or more magnets has a diameter and/or thickness of at least about 0.05 inches (in), at least about 0.1 in, at least about 0.2 in, at least about 0.5 in, at least about 1 in, at least about 1.5 in, at least about 2 in, at least about 3 in, at least about 4 in, or at least about 5 in. In some embodiments, at least one of the one or more magnets has a diameter and/or thickness of about 5 in or less, about 4 in or less, about 3 in or less, about 2 in or less, about 1.5 in or less, about 1 in or less, about 0.5 in or less, about 0.2 in or less, about 0.1 in or less, or about 0.05 in or less. In some embodiments, at least one of the one or more magnets has a diameter and/or thickness between about 0.05 in and about 0.1 in, between about 0.05 in and about 0.5 in, between about 0.05 in and about 1 in, between about 0.05 in and about 2 in, between about 0.05 in and about 5 in, between about 0.1 in and about 0.5 in, between about 0.1 in and about 1 in, between about 0.1 in and about 2 in, between about 0.1 in and about 5 in, between about 0.5 in and about 1 in, between about 0.5 in and about 2 in, between about 0.5 in and about 5 in, between about 1 in and 5 in, or between about 2 in and about 5 in.

In some embodiments, the magnetic level sensing system comprises a non-magnetic shaft configured such that a magnetic float can move vertically along the non-magnetic shaft. In certain instances, the non-magnetic shaft comprises a probe (e.g., a probe used for process control) or a baffle. In some cases, the non-magnetic shaft is positioned sufficiently closely to a wall of a reactor chamber of a bioreactor such that at least a portion of the magnetic field of the magnetic float can penetrate the reactor chamber wall. The non-magnetic shaft may be formed of any non-magnetic material suitable for use in a bioreactor. In some embodiments, the material of the non-magnetic shaft is biocompatible. In some instances, the material of the non-magnetic shaft can withstand sterilization. Non-limiting examples of suitable materials for the non-magnetic shaft include metals (e.g., titanium, titanium alloys, stainless steel, cobalt-chromium alloys), glass, plastics (e.g., polyethylene, polypropylene, polyethylene terephthalate, polymethyl methacrylate, polyvinyl alcohol, polyvinyl chloride, polystyrenes, polyamides, polyesters, polyurethanes, silicones), and ceramics (e.g., alumina).

In some embodiments, the magnetic level sensing system further comprises one or more magnetically-activated switches. In certain instances, the one or more magnetically-activated switches comprise one or more reed switches. A reed switch generally refers to an electric switch that turns on in the presence of a magnetic field. In certain instances, the one or more magnetically-activated switches comprise one or more Hall Effect sensors. A Hall Effect sensor generally refers to a transducer that varies output in response to a magnetic field. In certain instances, a Hall Effect sensor acts as a substantially continuous sensor due to its ability to linearly increase output voltage (until saturation) with increasing magnetic field density.

The magnetic level sensing system may comprise any number of magnetically-activated switches. In certain embodiments, the magnetic level sensing system comprises at least 1 switch, at least 2 switches, at least 5 switches, at least 10 switches, at least 20 switches, at least 50 switches, or at least 100 switches. In certain embodiments, the magnetic level sensing system comprises 100 switches or less, 50 switches or less, 20 switches or less, 10 switches or less, 5 switches or less, 2 switches or less, or 1 switch. In some embodiments, the magnetic level sensing system comprises between 1 and 5 switches, between 1 and 10 switches, between 1 and 20 switches, between 1 and 50 switches, between 1 and 100 switches, between 5 and 10 switches, between 5 and 20 switches, between 5 and 50 switches, between 5 and 100 switches, between 10 and 20 switches, between 10 and 50 switches, between 10 and 100 switches, between 20 and 100 switches, or between 50 and 100 switches. The switches may be any suitable type of switch. Examples of suitable types of switches include, but are not limited to, single pole-single throw switches, single pole-double throw switches, normal-open switches, and normal-close switches.

In some embodiments, the one or more magnetically-activated switches are positioned external to a bioreactor. In some cases, the one or more magnetically-activated switches are positioned equidistant to each other. The one or more magnetically-activated switches may, in certain instances, be attached to an adhesive strip that can be externally mounted to a bioreactor. In some embodiments, at least one switch of the one or more magnetically-activated switches is placed in series with a light-emitting diode (LED), a current sensor, a switch state detector, a processor, or another electronic component. In certain embodiments, each switch of the one or more magnetically-activated switches is placed in series with an LED, a current sensor, a switch state detector, a processor, or another electronic component. In some instances, each switch of the one or more magnetically-activated switches is placed in series with an LED of a different color. In some such instances, the LEDs provide visual information regarding a liquid level within a bioreactor.

Certain embodiments are directed to kits comprising one or more components of the magnetic level sensing system. In some instances, the kit comprises a magnetic float and one or more magnetically-activated switches. In some embodiments, the kit comprises a magnetic float and a strip (e.g., an adhesive strip) comprising one or more magnetically-activated switches. In certain cases, the kit may further comprise a non-magnetic shaft. In certain instances, the kit further comprises one or more containers.

Some embodiments are directed to bioreactors (e.g., single-use bioreactors) comprising one or more components of the magnetic level sensing system. In certain instances, for example, a magnetic float and/or a non-magnetic shaft may be manufactured as part of the bioreactor.

### Optical Level Sensing System

In certain embodiments, the level sensing system is an optical level sensing system (e.g., a system in which a liquid level is measured based on one or more visual cues). According to some embodiments, the optical level sensing system may be a discrete optical level sensing system, a continuous coordinate-based optical level sensing system, and/or a continuous area-based optical level sensing system. The optical level sensing system may function at varying cultivation densities. In some cases, the optical level sensing system may accurately measure bioreactor liquid level at a cultivation density corresponding to an optical density of at least about 0.01 OD₆₀₀, at least about 0.02 OD₆₀₀, at least about 0.05 OD₆₀₀, at least about 0.1 OD₆₀₀, at least about 0.15 OD₆₀₀, at least about 0.2 OD₆₀₀, at least about 0.25 OD₆₀₀, at least about 0.3 OD₆₀₀, at least about 0.4 OD₆₀₀, at least about 0.5 OD₆₀₀, at least about 1 OD₆₀₀, or at least about 5 OD₆₀₀. In some embodiments, the optical level sensing system may accurate measure bioreactor liquid level at a cultivation density corresponding to an optical density of about 5 OD600 or less, about 1 OD600 or less, about 0.5 OD600 or less, about 0.4 OD600 or less, about 0.3 OD₆₀₀ or less, about 0.25 OD₆₀₀ or less, about 0.2 OD₆₀₀ or less, about 0.15 OD₆₀₀ or less, about 0.1 OD₆₀₀ or less, about 0.05 OD₆₀₀ or less, about 0.02 OD₆₀₀ or less, or about 0.01 OD₆₀₀ or less, or less. In some embodiments, the optical level sensing system may accurate measure bioreactor liquid level at a cultivation density corresponding to an optical density between about 0.01 OD₆₀₀ and about 0.05 OD₆₀₀, between about 0.01 OD₆₀₀ and about 0.1 OD₆₀₀, between about 0.01 OD₆₀₀ and about 0.15 OD₆₀₀, between about 0.01 OD₆₀₀ and about 0.2 OD₆₀₀, between about 0.01 OD₆₀₀ and about 0.25 OD₆₀₀, between about 0.01 OD₆₀₀ and about 0.3 OD₆₀₀, between about 0.01 OD₆₀₀ and about 0.5 OD₆₀₀, between about 0.01 OD₆₀₀ and about 1 OD₆₀₀, between about 0.01 OD₆₀₀ and about 5 OD₆₀₀, between about 0.05 OD₆₀₀ and about 0.1 OD₆₀₀, between about 0.05 OD₆₀₀ and about 0.15 OD₆₀₀, between about 0.05 OD₆₀₀ and about 0.2 OD₆₀₀, between about 0.05 OD₆₀₀ and about 0.25 OD₆₀₀, between about 0.05 OD₆₀₀ and about 0.3 OD₆₀₀, between about 0.05 OD₆₀₀ and about 0.5 OD₆₀₀, between about 0.05 OD₆₀₀ and about 1 OD₆₀₀, between about 0.05 OD₆₀₀ and about 5 OD₆₀₀, between about 0.1 OD₆₀₀ and about 0.15 OD₆₀₀, between about 0.1 OD₆₀₀ and about 0.2 OD₆₀₀, between about 0.1 OD₆₀₀ and about 0.25 OD₆₀₀, between about 0.1 OD₆₀₀ and about 0.3 OD₆₀₀, between about 0.1 OD₆₀₀ and about 0.5 OD₆₀₀, between about 0.1 OD₆₀₀ and about 1 OD₆₀₀, between about 0.1 OD₆₀₀ and about 5 OD₆₀₀, between about 0.15 OD₆₀₀ and about 0.2 OD₆₀₀, between about 0.15 OD₆₀₀ and about 0.25 OD₆₀₀, between about 0.15 OD₆₀₀ and about 0.3 OD₆₀₀, between about 0.15 OD₆₀₀ and about 0.5 OD₆₀₀, between about 0.15 OD₆₀₀ and about 1 OD₆₀₀, between about 0.15 OD₆₀₀ and about 5 OD₆₀₀, between about 0.2 OD₆₀₀ and about 0.25 OD₆₀₀, between about 0.2 OD₆₀₀ and about 0.3 OD₆₀₀, between about 0.2 OD₆₀₀ and about 0.5 OD₆₀₀, between about 0.2 OD₆₀₀ and about 1 OD₆₀₀, between about 0.2 OD₆₀₀ and about 5 OD₆₀₀, between about 0.25 OD₆₀₀ and about 0.3 OD₆₀₀, between about 0.25 OD₆₀₀ and about 0.5 OD₆₀₀, between about 0.25 OD₆₀₀ and about 1 OD₆₀₀, between about 0.25 OD₆₀₀ and about 5 OD₆₀₀, between about 0.3 OD₆₀₀ and about 0.5 OD₆₀₀, between about 0.3 OD₆₀₀ and about 1 OD₆₀₀, between about 0.3 OD₆₀₀ and about 5 OD₆₀₀, between about 0.5 OD₆₀₀ and about 1 OD₆₀₀, between about 0.5 OD₆₀₀ and about 5 OD₆₀₀, or between about 1 OD₆₀₀ and about 5 OD₆₀₀. In some cases, the optical level sensing system may accurately measure the level of a bioreactor liquid that is substantially opaque corresponding to an optical density of 100 OD600 units or more.

In some embodiments, an optical level sensing system may permit monitoring of additional aspects of a bioreactor with a single system. As a non-limiting, illustrative example, image processing may detect foaming of a bioreactor liquid, which can result in fluids entering regions or sensors at the top of the reactor (e.g., off-gassing). In certain instances, an image that is used for level sensing may also be used to monitor foam levels, which may advantageously allow closed-loop control of anti-foam additives. As another illustrative example, an optical level sensing system may permit estimation of the optical opacity of a bioreactor liquid (e.g., relative to the reference color of an optical float or a probe), which may provide a method of determining cell culture density.

### Discrete Optical Level Sensing System

In some embodiments, the optical level sensing system is a discrete optical level sensing system (e.g., an optical level sensing system comprising a plurality of discrete visual markings). In some embodiments, the discrete optical level sensing system comprises a probe having two or more discrete visual markings. In some embodiments, at least one discrete visual marking comprises a band having a color distinct from the color of a bioreactor fluid (e.g., a cell suspension). In certain instances, a probe comprises at least 2 colored bands, at least 3 colored bands, at least 4 colored bands, at least 5 colored bands, at least 10 colored bands, at least 20 colored bands, or at least 50 colored bands. In some embodiments, each colored band of a probe has a distinct color (e.g., the colors are sufficiently different that they can be visually distinguished from each other and from the bioreactor liquid). In some instances, each colored band of the probe is associated with a pre-defined liquid level. It should be understood that though certain examples and embodiments of optical level sensing are described with respect to color, any suitable visual marking may be used. In some embodiments, visual markings may comprise a high-contrast region with differential light scattering properties, a different material of distinct color or contrast, a geometric form or pattern, or any other fiduciary marking to indicate a region of contrast.

The probe of the discrete optical level sensing system may or may not be an existing component of a bioreactor. In certain instances, for example, the probe comprises a shaft of an agitator or impeller of a bioreactor. In certain other instances, the probe comprises a separate component (e.g., an otherwise non-functional baffle). The probe may comprise any suitable material. In some embodiments, the material of the probe is biocompatible. In some embodiments, the material of the probe can withstand sterilization. Non-limiting examples of suitable materials for the probe include metals (e.g., titanium, titanium alloys, stainless steel, cobalt-chromium alloys), glass, plastics (e.g., polyethylene, polypropylene, polyethylene terephthalate, polymethyl methacrylate, polyvinyl alcohol, polyvinyl chloride, polystyrenes, polyamides, polyesters, polyurethanes, silicones), and ceramics (e.g., alumina).

In some embodiments, the probe is coated or otherwise surrounded by a biocompatible material. In certain instances, the probe is positioned inside an optically transparent sleeve within a bioreactor. In embodiments in which the probe is coated or surrounded by a biocompatible material, or otherwise not in direct contact with biological cells (e.g., the first type of biological cells), the probe itself may or may not comprise a biocompatible material.

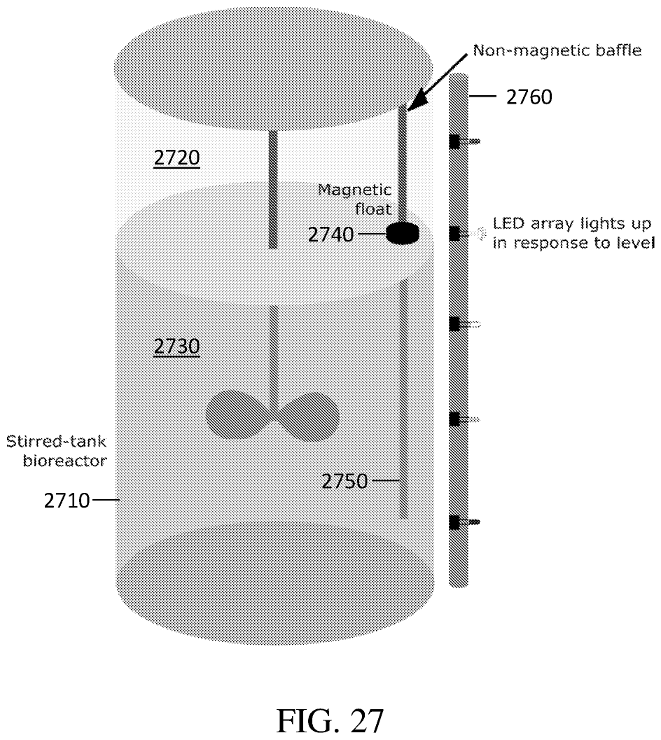

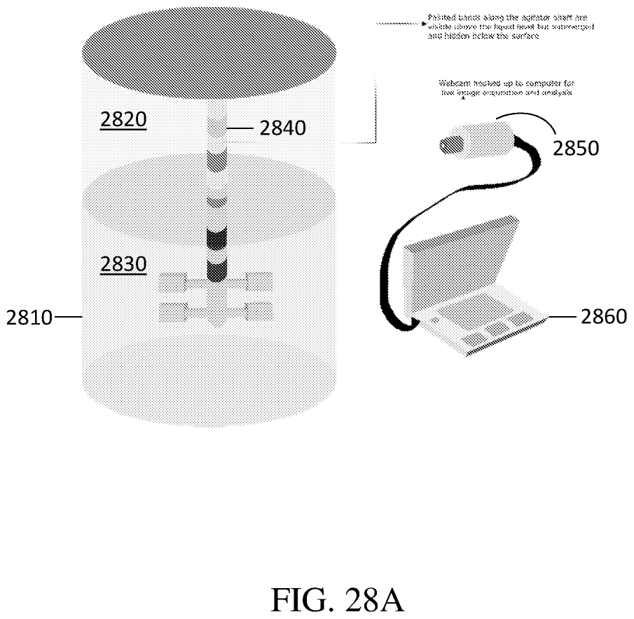

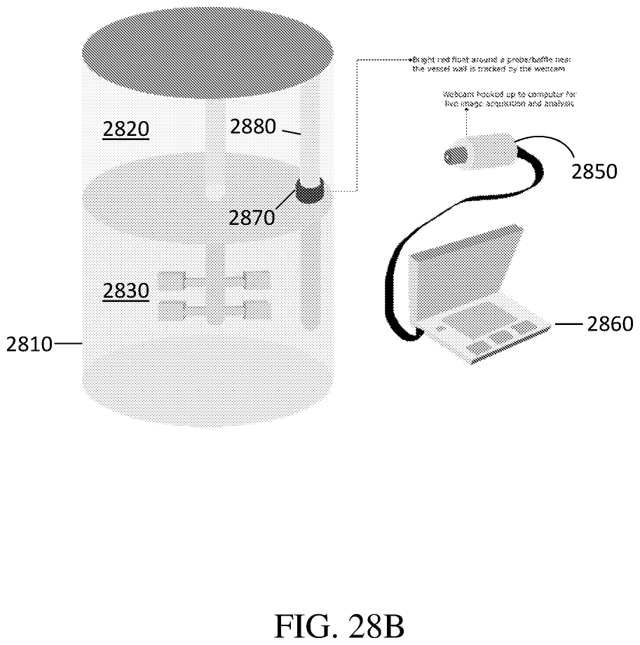

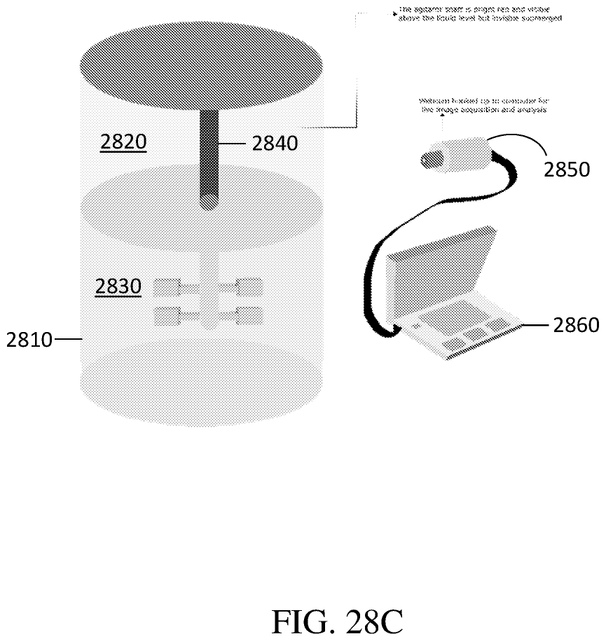

A schematic illustration of an exemplary discrete optical level sensor is shown in FIG. 28A. FIG. 28A shows bioreactor 2810, which comprises reaction chamber 2820 containing a bioreactor liquid (e.g., a cell suspension) 2830. FIG. 28A also shows agitator shaft 2840, which comprises five colored bands, within bioreactor 2810. Camera 2850 and computer 2860 are positioned outside bioreactor 2810.

In operation, the level of bioreactor liquid 2830 may rise and/or fall, rendering different colored bands visible to camera 2850. Camera 2850 may be used to acquire images of agitator shaft 2840, and the images may be processed by one or more algorithms.

In some embodiments, a color image acquired by an image acquisition device (e.g., camera 2850) may be transmitted (e.g., electronically transmitted) to a computer (e.g., computer 2860) configured to run one or more image processing algorithms. In some embodiments, the one or more image processing algorithms comprise a chrominance-based binarization (CBB) algorithm. In some instances, the CBB algorithm may convert a color image to a binary (e.g., black and white) image by selecting for colors of interest. In certain embodiments, the acquired image may be converted from the RGB (red, green, blue) space to the HSV (hue, saturation, value) space. The HSV image may then be filtered using thresholding of the different hue, saturation, and value data against the known ranges of the colors of interest. To remove optical and physical noise, the holes in the binary image (e.g., a couple black pixels among many white ones) may be filled (e.g., by using the “imfill” function in Matlab) in order to ensure objects remain together and are not compromised.

In some embodiments, a colored object detection (COD) algorithm may be used to identify discrete colored objects in a binary image generated by the CBB algorithm. In some cases, the COD algorithm may apply a Gaussian blur and filter to the binary image. In certain instances, this may smooth erroneous pixels, physical and optical imperfections, and may reduce vibrational noise. In some cases, the COD algorithm is performed by clustering binary data and creating “blobs” that represent objects of a specific color in the original image. To avoid flecks of the specific color in the image, and other minor objects of the same color, an area-based filter may be applied to retain objects within a certain pixel area range. The pixel area range may vary based on the colored object being searched for. As an illustrative example, the COD algorithm may filter out objects smaller than 40 pixels when searching for a colored float or painted bands, and may filter out objects smaller than 500 pixels when searching for a colored shaft. The COD algorithm may then count the retained objects of the specific color. Only one object should remain for each color of interest.

In some embodiments, a Painted Bands (PB) algorithm may use information from the COD algorithm to determine a level of liquid within the bioreactor. In some instances, the number of objects for each band color may be counted in the COD algorithm and fed to the PB algorithm. The PB algorithm may use those counts to detect the presence or absence of a specific painted band in the image and correlate that to a point level being above or below certain values associated with the bands at those levels.

### Continuous Coordinate-Based Optical Level Sensing System

In some embodiments, the optical level sensing system is a continuous coordinate-based optical level sensing system. In certain embodiments, the continuous coordinate-based optical level sensing system comprises a probe and an optical float configured to move vertically along the probe. In some instances, a level of liquid within a bioreactor may be determined from coordinates of the optical float. In certain embodiments, the optical float has a color distinct from the color of a liquid within the bioreactor (e.g., a cell suspension). Examples of suitable colors for the optical float include, but are not limited to, red, green, blue, orange, and purple.

In some embodiments, the continuous coordinate-based optical level sensing system comprises an optical float comprising a flotation ring. The flotation ring may comprise any material suitable for use in a bioreactor. In certain embodiments, the material of the flotation ring is biocompatible. In certain embodiments, the material of the flotation ring can withstand sterilization. Non-limiting examples of suitable materials for the flotation ring include metals (e.g., titanium, titanium alloys, stainless steel, cobalt-chromium alloys), glass, plastics (e.g., polyethylene, polypropylene, polyethylene terephthalate, polymethyl methacrylate, polyvinyl alcohol, polyvinyl chloride, polystyrenes, polyamides, polyesters, polyurethanes, silicones), and ceramics (e.g., alumina).

The flotation ring may comprise any suitable material having a specific gravity less than the liquid within the bioreactor. Examples of materials having a suitable specific gravity include, but are not limited to, polypropylene and polystyrene foam. In some embodiments, the flotation ring comprises a material (e.g., a biocompatible material) encompassing a hollow region comprising a gas (e.g., air). The material encompassing the hollow region may be any suitable material.

In certain embodiments, the flotation ring may comprise a colored material (e.g., a colored plastic). In some cases, one or more suitable colors may be imparted to the flotation ring according to any method known in the art. In certain instances, the flotation ring may be painted a color and/or wrapped in a colored tape.

In some embodiments, the continuous coordinate-based optical level sensing system comprises a probe. The probe may or may not be an existing component of a bioreactor. In certain instances, for example, the probe comprises a shaft of an agitator or impeller of a bioreactor. In certain other instances, the probe comprises a separate component (e.g., an otherwise non-functional baffle). The probe may be formed of any material suitable for use in a bioreactor. In some embodiments, the material of the probe is biocompatible. In some embodiments, the material of the probe can withstand sterilization. Non-limiting examples of suitable materials for the probe include metals (e.g., titanium, titanium alloys, stainless steel, cobalt-chromium alloys), glass, plastics (e.g., polyethylene, polypropylene, polyethylene terephthalate, polymethyl methacrylate, polyvinyl alcohol, polyvinyl chloride, polystyrenes, polyamides, polyesters, polyurethanes, silicones), and ceramics (e.g., alumina).

A schematic illustration of an exemplary continuous coordinate-based optical level sensing system is shown in FIG. 28B. In FIG. 28B, optical float 2870 and probe 2880 are positioned within reaction chamber 2820 of bioreactor 2810, which also contains bioreactor liquid 2830. As shown in FIG. 28B, probe 2880 may be at least partially submerged in bioreactor liquid 2830, and optical float 2870 may float on the surface of bioreactor liquid 2830. Camera 2850 and computer 2860 may be positioned outside bioreactor 2810.

In operation, optical float 2870 may move vertically along probe 2880 as the level of bioreactor liquid 2830 rises and falls. Camera 2850 may obtain images of optical float 2870, and the acquired images may be transmitted to computer 2860, which may run one or more image processing algorithms.

In some embodiments, a chrominance-based binarization (CBB) algorithm (e.g., the CBB algorithm described above) may be employed to convert a color image acquired by a camera (e.g., camera 2850) to a binary image. In certain embodiments, a colored object detection (COD) algorithm (e.g., the COD algorithm described above) may be used to identify discrete colored objects in a binary image generated by the CBB algorithm.

In some embodiments, a colored float (CF) algorithm may use information from the COD algorithm to determine a level of liquid within the bioreactor. In certain embodiments, the CF algorithm may detect the location of the colored float (e.g., a red float) and determine its centroid. In some instances, by pre-determined geometric calculations and knowledge of camera-acquired image specifications, the centroid of the colored float that was detected may be used to correlate to liquid level.

It should be understood that though certain examples and embodiments of optical level sensing are described with respect to color, any suitable visual marking may be used. In some embodiments, visual markings may comprise a high-contrast region with differential light scattering properties, a different material of distinct color or contrast, a geometric form or pattern, or any other fiduciary marking to indicate a region of contrast.

### Continuous Area-Based Optical Level Sensing System

In some embodiments, the optical level sensing system is a continuous area-based optical level sensing system. In certain embodiments, the continuous area-based optical level sensing system comprises a probe having a color distinct from the color of a liquid within the bioreactor. In some instances, a level of liquid within a bioreactor may be determined from the amount (e.g., area) of the colored probe that is visible (e.g., the portion of the probe that is not submerged in the bioreactor liquid).

The probe may or may not be an existing component of a bioreactor. In certain instances, for example, the probe comprises a shaft of an agitator or impeller of a bioreactor. In certain other instances, the probe comprises a separate component (e.g., an otherwise non-functional baffle). The probe may be formed of any material suitable for use in a bioreactor. In some embodiments, the material of the probe is biocompatible. In some embodiments, the material of the probe can withstand sterilization. Non-limiting examples of suitable materials for the probe include metals (e.g., titanium, titanium alloys, stainless steel, cobalt-chromium alloys), glass, plastics (e.g., polyethylene, polypropylene, polyethylene terephthalate, polymethyl methacrylate, polyvinyl alcohol, polyvinyl chloride, polystyrenes, polyamides, polyesters, polyurethanes, silicones), and ceramics (e.g., alumina).

The probe may have any suitable color. Examples of suitable colors for the probe include, but are not limited to, red, green, blue, orange, and purple. In certain embodiments, the probe may comprise a colored material (e.g., a colored plastic). In some cases, the color may be imparted to the probe according to any method known in the art. In certain embodiments, at least a portion of the probe may be painted and/or wrapped in colored tape.

A schematic illustration of an exemplary continuous area-based optical level sensing system is shown in FIG. 28C. In FIG. 28C, agitator shaft 2840 of bioreactor 2810 has been colored red (e.g., by wrapping bright red tape around the shaft). Camera 2850 and computer 2860 are positioned outside bioreactor 2810.

In operation, the level of bioreactor liquid 2830 may rise and/or fall, which may change the amount of colored agitator shaft 2840 that is visible to camera 2850. Camera 2850 may be used to acquire images of agitator shaft 2840, and the images may be processed by one or more algorithms.

In some embodiments, a chrominance-based binarization (CBB) algorithm (e.g., the CBB algorithm described above) may be employed to convert a color image acquired by a camera (e.g., camera 2850) to a binary image. In certain embodiments, a colored object detection (COD) algorithm (e.g., the COD algorithm described above) may be used to identify discrete colored objects in a binary image generated by the CBB algorithm.

In some embodiments, a colored shaft (CS) algorithm may use information from the COD algorithm to determine a level of liquid within the bioreactor. In certain embodiments, the

CS algorithm may detect the residual size of the colored probe. By pre-determined geometrical calculations and knowledge of camera-acquired image specifications, the area of the colored probe may then be used to determine the liquid level.

Certain embodiments are directed to kits comprising one or more components of the optical level sensing system. In some instances, the kit comprises an optical float, a colored probe, and/or a probe having two or more discrete visual markings. In some embodiments, the kit further comprises a camera. In some embodiments, the kit further comprises one or more containers.

Some embodiments are directed to bioreactors (e.g., single-use bioreactors) comprising one or more components of the optical level sensing system. In certain instances, for example, a colored probe, a probe having two or more discrete visual markings, and/or an optical float may be manufactured as part of the bioreactor.

It should be understood that though certain examples and embodiments of optical level sensing are described with respect to color, any suitable visual marking may be used. In some embodiments, visual markings may comprise a high-contrast region with differential light scattering properties, a different material of distinct color or contrast, a geometric form or pattern, or any other fiduciary marking to indicate a region of contrast.

### Filter

In some embodiments, the biomanufacturing system (e.g., system 900 in FIG. 9) comprises at least one filter (e.g., filter 104). According to certain embodiments, the at least one filter is directly fluidically connected to the bioreactor. For example, in certain cases, the filter is at least partially submerged in the cell suspension (i.e., the suspension comprising the first type of biological cells and the cell culture medium) contained in the reaction chamber of the bioreactor. In some embodiments, the filter is configured to at least partially separate the biological cells from the cell culture media. According to certain embodiments, for example, the filter is configured to allow a first filtrate comprising the cell culture medium and at least one biologically-produced product to exit the reactor chamber of the bioreactor while retaining the biological cells within the reactor chamber. The filter may be configured for dead-end filtration or tangential flow filtration.

In some embodiments, the filter is a filter probe. Suitable filter probes include those described in a to U.S. Provisional Patent Application Ser. No. 62/553,104, filed Aug. 31, 2017, and entitled “Filtration Systems and Methods for Manufacturing Biologically-Produced Products, which is incorporated herein by reference in its entirety for all purposes.

The filter probe may have any suitable size or shape. In certain embodiments, for example, the filter probe is substantially cylindrical. In certain embodiments, the filter probe comprises a plurality of fibers. In some embodiments, the filter probe is constructed of materials that are chemically stable upon exposure to the cell culture medium (e.g., growth cell culture medium, production cell culture medium). In some embodiments, the filter probe is constructed of materials that are chemically stable upon exposure to methanol and/or glycerol. In some embodiments, the filter comprises a ceramic filter and/or a filtration membrane. In some embodiments, the at least one filter has a pore size that is sufficiently large to allow the at least one biologically-produced product to pass through the filter but sufficiently small to prevent the passage of the first type of biological cells. In certain embodiments, the filter has a pore size of at least about 0.01 microns (μm), at least about 0.02 μm, at least about 0.05 μm, at least about 0.08 μm, at least about 0.1 μm, at least about 0.2 μm, at least about 0.3 μm, at least about 0.4 μm, at least about 0.5 μm, at least about 0.8 μm, at least about 1 μm, at least about 2 μm, or at least about 3 μm. In some embodiments, the filter has a pore size of about 4 μm or less, about 3 μm or less, about 2 μm or less, about 1 μm or less, about 0.8 μm or less, about 0.5 μm or less, about 0.4 μm or less, about 0.3 μm or less, about 0.2 μm or less, about 0.1 μm or less, about 0.08 μm or less, about 0.05 μm or less, about 0.02 μm or less, or about 0.01 μm or less. In some embodiments, the filter has a pore size in the range of about 0.01 μm to about 0.05 μm, about 0.01 μm to about 0.1 μm, about 0.01 μm to about 0.5 μm, about 0.01 μm to about 1 μm, about 0.01 μm to about 2 μm, about 0.01 μm to about 3 μm, about 0.01 μm to about 4 μm, about 0.05 μm to about 0.1 μm, about 0.05 μm to about 0.5 μm, about 0.05 μm to about 1 μm, about 0.05 μm to about 2 μm, about 0.05 μm to about 3 μm, about 0.05 μm to about 4 μm, about 0.1 μm to about 0.5 μm, about 0.1 μm to about 1 μm, about 0.1 μm to about 2 μm, about 0.1 μm to about 3 μm, about 0.1 μm to about 4 μm, about 0.2 μm to about 0.5 μm, about 0.2 μm to about 0.8 μm, about 0.2 μm to about 1 μm, about 0.2 μm to about 2 μm, about 0.2 μm to about 3 μm, about 0.2 μm to about 4 μm, or about 0.5 μm to about 1 μm, about 0.5 μm to about 2 μm, about 0.5 μm to about 3 μm, about 0.5 μm to about 4 μm. In some embodiments, the filter has a pore size in the range of about about 0.2 μm to about 0.8 μm.

In some embodiments, the at least one filter has a sufficiently large surface area exposed to the cell suspension in the reactor chamber of the bioreactor that at least a portion of the cell suspension (e.g., the first filtrate stream) flows through the at least one filter at a relatively high flow rate.

In certain embodiments in which the bioreactor comprises a reactor chamber having an internal volume of about 50 mL to about 1 L, the first filtrate stream has a flow rate of at least about 0.01 mL/min, at least about 0.05 mL/min, at least about 0.1 mL/min, at least about 0.15 mL/min, at least about 0.2 mL/min, at least about 0.3 mL/min, at least about 0.4 mL/min, at least about 0.5 mL/min, at least about 0.6 mL/min, at least about 0.7 mL/min, at least about 0.8 mL/min, at least about 0.9 mL/min, at least about 1 mL/min, at least about 1.5 mL/min, or at least about 2 mL/min over a specified time period. In some embodiments, the first filtrate stream has a flow rate in the range of about 0.01 mL/min to about 0.1 mL/min, 0.01 mL/min to about 0.5 mL/min, about 0.01 mL/min to about 1 mL/min, about 0.01 mL/min to about 2 mL/min, about 0.03 mL/min to about 0.1 mL/min, 0.05 mL/min to about 0.1 mL/min, about 0.05 mL/min to about 0.5 mL/min, about 0.05 mL/min to about 1 mL/min, about 0.05 mL/min to about 2 mL/min, about 0.07 mL/min to about 0.2 mL/min, about 0.1 mL/min to about 0.4 mL/min, about 0.1 mL/min to about 1 mL/min, about 0.3 mL/min to about 1 mL/min, about 0.5 mL/min to about 1 mL/min, about 0.5 mL/min to about 2 mL/min, or about 1 mL/min to about 2 mL/min over a specified time period.

In certain embodiments in which the bioreactor comprises a reactor chamber having an internal volume of about 1 L to about 10 L, the first filtrate stream has a flow rate of at least about 0.5 mL/min, at least about 1 mL/min, at least about 1.5 mL/min, at least about 2 mL/min, at least about 5 mL/min, at least about 10 mL/min, at least about 15 mL/min, or at least about 20 mL/min over a specified time period. In some embodiments, the first filtrate stream has a flow rate in the range of about 0.5 mL/min to about 2 mL/min, about 0.5 mL/min to about 5 mL/min, about 0.5 mL/min to about 10 mL/min, about 0.5 mL/min to about 15 mL/min, about 0.5 mL/min to about 20 mL/min, about 3 mL/min to about 10 mL/min, about 5 mL/min to about 10 mL/min, about 5 mL/min to about 15 mL/min, about 5 mL/min to about 20 mL/min, about 7 mL/min to about 20 mL/min, about 10 mL/min to about 20 mL/min, or about 15 mL/min to about 20 mL/min over a specified time period.

In certain embodiments in which the bioreactor comprises a reactor chamber having an internal volume of about 10 L to about 100 L, the first filtrate stream has a flow rate of at least about 5 mL/min, at least about 10 mL/min, at least about 15 mL/min, at least about 20 mL/min, at least about 50 mL/min, at least about 100 mL/min, at least about 150 mL/min, or at least about 200 mL/min over a specified time period. In some embodiments, the first filtrate stream has a flow rate in the range of about 5 mL/min to about 20 mL/min, about 5 mL/min to about 100 mL/min, about 5 mL/min to about 150 mL/min, about 5 mL/min to about 200 mL/min, about 10 mL/min to about 50 mL/min, about 10 mL/min to about 100 mL/min, about 10 mL/min to about 150 mL/min, about 10 mL/min to about 200 mL/min, about 35 mL/min to about 100 mL/min, about 35 mL/min to about 150 mL/min, about 35 mL/min to about 200 mL/min, about 50 mL/min to about 100 mL/min, about 50 mL/min to about 150 mL/min, about 50 mL/min to about 200 mL/min, or about 100 mL/min to about 200 mL/min over a specified time period.

In some embodiments, the specified time period is at least about 1 hour, at least about 2 hours, at least about 5 hours, at least about 10 hours, at least about 1 day, at least about 2 days, at least about 3 days, at least about 4 days, at least about 5 days, at least about 6 days, at least about 7 days, at least about 2 weeks, at least about 5 weeks, or at least about 10 weeks.

In some embodiments, the at least one filter comprises a plurality of filters. In some embodiments, the at least one filter comprises at least 2 filters, at least 3 filters, at least 4 filters, at least 5 filters, at least 10 filters, or at least 11 filters. In some embodiments, the at least one filter comprises 1 to 2 filters, 1 to 5 filters, 1 to 10 filters, 1 to 11 filters, 2 to 5 filters, 2 to 10 filters, 2 to 11 filters, 5 to 10 filters, 5 to 11 filters, or 10 to 11 filters.

In some embodiments, the first filtrate (e.g., stream 912 in FIGS. 9A-9D) flowing through the filter is lean in the first type of biological cells relative to the cell suspension contained in the bioreactor. In certain embodiments, for example, the wet cell weight of the first type of biological cells in the first filtrate is about 1 μg/L or less. The ratio of wet cell weight of the first type of biological cells in the growth medium in the bioreactor to the wet cell weight of the first type of biological cells in the first filtrate should be at least 1×10⁶, at least 1×10⁷, at least 1×10⁸, or at least 1×10⁹.

### Adjustment Module

In some embodiments, the biomanufacturing system (e.g., system 900 in FIG. 9) comprises an optional adjustment module (e.g., adjustment module 916). In some embodiments, the adjustment module is configured to adjust (e.g., increase, decrease) one or more properties (e.g., pH, conductivity, biologically-produced product stability, flow rate, pressure) of a fluid stream (e.g., a first filtrate from the filter, a cell suspension stream from the bioreactor). According to some embodiments, the one or more properties comprise pH, and the adjustment module is configured to increase or decrease the pH of a fluid stream. As an illustrative example, the adjustment module may receive the first filtrate from the filter and adjust the pH of the first filtrate to produce an adjusted filtrate. In some embodiments, the adjusted filtrate has a pH that is compatible with a first partitioning unit of the purification module. For example, according to certain embodiments, the first partitioning unit of the purification module may comprise a chromatographic combination comprising a first stationary phase material and a first mobile phase material, where the first mobile phase material has a pH. In some embodiments, the difference between the pH of the adjusted filtrate and the pH of the first mobile phase material of the first partitioning unit of the purification module is about 4 or less, about 3 or less, about 2 or less, about 1 or less, about 0.5 or less, about 0.4 or less, about 0.3 or less, about 0.2 or less, about 0.1 or less, about 0.05 or less, or about 0.0. In some embodiments, the difference between the pH of the adjusted filtrate and the pH of the first mobile phase material of the first partitioning unit of the purification module is in the range of about 0.0 to about 0.1, about 0.0 to about 0.2, about 0.0 to about 0.3, about 0.0 to about 0.4, or about 0.0 to about 0.5. In some embodiments, the difference between the pH of the adjusted filtrate and the pH of the first mobile phase material of the first partitioning unit of the purification module is in the range of about 0.1 to about 1, about 0.1 to about 2, about 0.1 to about 3, or about 0.1 to about 4.

In some embodiments, the pH of a fluid stream may be adjusted by adding a pH-adjusting composition (e.g., an acid, a base) to the fluid stream. In certain embodiments, for example, an acid may be added to the fluid stream to decrease the pH of the stream. Non-limiting examples of suitable acids include citric acid, acetic acid, 4-(2-hydroxyethyl)-1-piperazineethanesulfonic acid (HEPES), hydrochloric acid, sulfuric acid, phosphoric acid, and nitric acid. In certain embodiments, a base may be added to the fluid stream to increase the pH of the stream. Examples of suitable bases include, but are not limited to, ammonium hydroxide, sodium hydroxide, potassium hydroxide, calcium hydroxide, phosphate monobasic, phosphate dibasic, and tris(hydroxymethyl)aminomethane (Tris).

According to some embodiments, the one or more properties comprise conductivity, and the adjustment module is configured to increase or decrease the conductivity of a fluid stream. As an illustrative example, the adjustment module may receive the first filtrate from the filter and adjust the conductivity of the first filtrate to produce an adjusted filtrate. In some embodiments, the adjusted filtrate has a conductivity that is compatible with a first partitioning unit of the purification module. For example, according to certain embodiments, the first partitioning unit of the purification module comprises a chromatographic combination comprising a first stationary phase material and a first mobile phase material, where the first mobile phase material has a conductivity. In some embodiments, the difference between the conductivity of the adjusted filtrate and the conductivity of the first mobile phase material of the first partitioning unit of the purification module is 50 mS/cm or less, about 40 mS/cm or less, about 30 mS/cm or less, about 20 mS/cm or less, about 10 mS/cm or less, about 5 mS/cm or less, about 4 mS/cm or less, about 3 mS/cm or less, about 2 mS/cm or less, about 1 mS/cm or less, about 0.5 mS/cm or less, about 0.4 mS/cm or less, about 0.3 mS/cm or less, about 0.2 mS/cm or less, about 0.1 mS/cm or less, about 0.05 mS/cm or less, or about 0.0 mS/cm. In some embodiments, the difference between the conductivity of the adjusted filtrate and the first mobile phase material of the first partitioning unit of the purification module is in the range of about 0.0 mS/cm to about 0.1 mS/cm, about 0.0 mS/cm to about 0.2 mS/cm, about 0.0 mS/cm to about 0.3 mS/cm, about 0.0 mS/cm to about 0.4 mS/cm, about 0.0 mS/cm to about 0.5 mS/cm, about 0.0 mS/cm to about 1 mS/cm, about 0.0 mS/cm to about 5 mS/cm, about 0.0 mS/cm to about 10 mS/cm about 0.0 mS/cm to about 20 mS/cm, or about 0.0 mS/cm to about 50 mS/cm.

In some embodiments, the conductivity of a fluid stream may be adjusted by adding a conductivity-adjusting composition (e.g., a salt, a diluent) to the fluid stream. In some embodiments, for example, one or more salts may be added to the fluid stream to increase the conductivity of the stream. A non-limiting example of a suitable salt is sodium chloride. In some embodiments, a diluent may be added to the fluid stream to decrease the conductivity of the stream. A non-limiting example of a suitable diluent is water.

According to some embodiments, the one or more properties comprise biologically-produced product stability, and the adjustment module is configured to increase the stability of the biologically-produced product in a fluid stream. As an illustrative example, the adjustment module may receive the first filtrate from the filter and adjust the stability of the biologically-produced product in the first filtrate to produce an adjusted filtrate. In some embodiments, the stability of the biologically-produced product in a fluid stream may be adjusted by adding a stability-adjusting composition to the fluid stream. For example, the biologically-produced product present in the fluid stream may have one or more hydrophobic portions, while the remainder of the fluid stream (e.g., a cell culture medium) may be substantially hydrophilic. In certain embodiments, addition of one or more stability-adjusting compositions may enhance the stability of the biologically-produced product within the fluid stream. In some cases, the one or more stability-adjusting compositions comprise a surfactant, a lyoprotectant, a shear protectant, and/or an organic solvent. Non-limiting examples of suitable surfactants, lyoprotectants, shear protectants, and/or organic solvents include polysorbate 80, polysorbate 20, tween 20, triton-X 100, CHAPS, Breox, trehalose, sucrose, sorbitol, maltitol, and hexylene glycol.

According to some embodiments, the one or more properties comprise flow rate and/or pressure. As an illustrative example, the adjustment module may allow matching of desired flow rates between two or more components (e.g., units, modules). In some such cases, the adjustment module may serve as a flow rate and/or pressure converter between a first component (e.g., unit, module, bioreactor) and a second component (e.g., unit, module, purification module)

In some embodiments, the adjustment module is configured to minimize hold time after adjustment. In some cases, minimizing hold time after adjustment may advantageously maximize product quality. In some embodiments, the hold time of the adjusted filtrate is about 24 hours or less, about 18 hours or less, about 12 hours or less, about 6 hours or less, about 1 hour or less, about 30 minutes or less, or about 10 minutes or less. In some embodiments, the hold time of the adjusted filtrate is in the range of about 10 minutes to about 30 minutes, about 10 minutes to about 1 hour, about 10 minutes to about 6 hours, about 10 minutes to about 12 hours, about 10 minutes to about 18 hours, about 10 minutes to about 24 hours, about 30 minutes to about 1 hour, about 30 minutes to about 6 hours, about 30 minutes to about 12 hours, about 30 minutes to about 18 hours, about 30 minutes to about 24 hours, about 1 hour to about 6 hours, about 1 hour to about 12 hours, about 1 hour to about 18 hours, about 1 hour to about 24 hours, or about 6 hour to about 12 hours, about 6 hour to about 18 hours, about 6 hours to about 24 hours. In some embodiments, the adjustment module comprises a surge tank. In certain cases, the surge tank has a volume of about 50 mL to about 2 L, about 2 L to about 10 L, or about 10 L to about 100 L. In some cases, the surge tank may advantageously facilitate connection of an upstream process associated with a first flow rate and a downstream process associated with a second, different flow rate. For instance, in some embodiments, a surge tank may help to release pressure from the filter.

In some embodiments, the adjustment module comprises a level sensing system. In certain instances, the level sensing system is configured to sense a level of a fluid in a fluid-containing vessel (e.g., a surge tank) of the adjustment module. In some embodiments, the level sensing system of the adjustment module comprises a capacitance-based probe (e.g., an in-vessel capacitance-based probe). In some embodiments, the level sensing system of the adjustment module comprises a magnetic level sensing system and/or an optical level sensing system.

### Purification Module

In some embodiments, the biomanufacturing system (e.g., system 100) comprises a purification module (e.g., purification module 106) configured to remove at least a first type of impurity and a second type of impurity from a fluid (e.g., a cell suspension stream from the bioreactor, a first filtrate from the at least one filter, an adjusted filtrate from the adjustment module) to produce a purified filtrate. The purification module may comprise any number of partitioning units configured to remove at least one type of impurity from a fluid stream. In certain embodiments, the purification module comprises at least 2 partitioning units, at least 3 partitioning units, at least 4 partitioning units, at least 5 partitioning units, or at least 10 partitioning units. In some embodiments, the purification module comprises 2 to 5 partitioning units, 2 to 10 partitioning units, or 5 to 10 partitioning units.

According to some embodiments, the purification module comprises a first partitioning unit configured to remove at least the first type of impurity from the fluid stream to produce a first partitioned filtrate. In some embodiments, the purification module further comprises a second partitioning unit configured to remove at least the second type of impurity from the first partitioned filtrate to produce a second partitioned filtrate. In certain cases, the second partitioning unit is fluidically connected (e.g., directly fluidically connected) to the first partitioning unit.

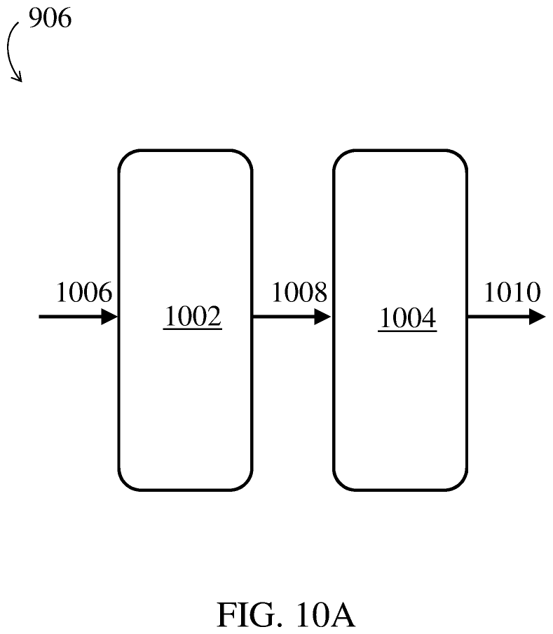

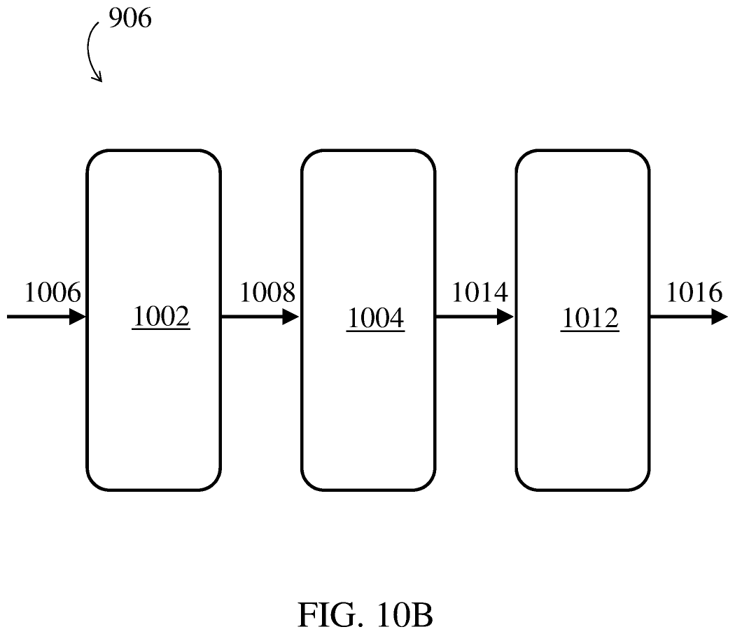

FIG. 10A illustrates an exemplary purification module 906 comprising first partitioning unit 1002 and second partitioning unit 1004. In operation, first partitioning unit 1002 receives input stream 1006, according to some embodiments. In certain embodiments, input stream 1006 comprises an adjusted filtrate stream from an adjustment module (e.g., adjusted filtrate stream 918 from adjustment module 916). In certain embodiments, input stream 1006 comprises a first filtrate stream from a filter (e.g., first filtrate stream 912 from filter 904). In certain embodiments, input stream 1006 comprises a cell suspension stream from a bioreactor (e.g., cell suspension stream 910 from bioreactor 902). In some embodiments, first partitioning unit 1002 applies a first partitioning technique to input stream 1006 to remove at least a first type of impurity from input stream 1006 to produce first partitioned filtrate 1008. In certain embodiments, first partitioned filtrate 1008 is directed to flow to second partitioning unit 1004. According to some embodiments, second partitioning unit 1004 applies a second partitioning technique to first partitioned filtrate 1008 to remove at least a second type of impurity from first partitioned filtrate 1008 to produce second partitioned filtrate 1010. The second partitioning technique may be the same or different from the first partitioning technique. In some embodiments, second partitioned filtrate 1010 is directed to flow to additional partitioning units of purification module 906. In some embodiments, second partitioned filtrate 1010 is collected as purified filtrate 914.

In some embodiments, the purification module further comprises a third partitioning unit configured to remove at least a third type of impurity from the second partitioned filtrate to produce a third partitioned filtrate. In certain cases, the third partitioning unit is fluidically connected (e.g., directly fluidically connected) to the second partitioning unit. For example, FIG. 10B illustrates exemplary purification module 906 comprising first partitioning unit 1002, second partitioning unit 1004, and third partitioning unit 1012. In operation, second partitioned filtrate 1010 from second partitioning unit 1004 is directed to flow to third partitioning unit 1012, according to some embodiments. In certain embodiments, third partitioning unit 1012 applies a third partitioning technique to second partitioned filtrate 1014 to produce third partitioned filtrate 1016. The third partitioning technique may be the same or different from the first partitioning technique and/or the second partitioning technique. In some embodiments, third partitioned filtrate 1016 is directed to flow to additional partitioning units of purification module 906. In some embodiments, third partitioned filtrate 1016 is collected as purified filtrate 914.

Any of the partitioning units (e.g., first partitioning unit, second partitioning unit, third partitioning unit) of the purification module may independently apply any partitioning technique. In some embodiments, the partitioning technique comprises chromatography, filtration, precipitation, crystallization, and/or extraction. The partitioning technique applied by one partitioning unit of the purification module may be the same or different from the partitioning technique applied by any other partitioning unit of the purification module.

In some embodiments, the partitioning technique applied by at least one partitioning unit of the purification module comprises chromatography. In certain embodiments, for example, the at least one partitioning unit comprises a column comprising a first stationary phase material. In some embodiments, the first stationary phase material is a cation exchange resin, a multimodal cation exchange resin, an anion exchange resin (e.g., a salt-tolerant anion exchange resin), a multimodal anion exchange resin, a hydrophobic charge induction chromatography (HCIC) resin, or an affinity chromatography resin. Non-limiting examples of suitable cation exchange resins include SP Sepharose HP. Non-limiting examples of suitable multi-modal cation exchange resins include Capto MMC, Capto MMC ImpRes, Nuvia cPrime, Toyopearl MX-Trp-650M, CMM HyperCel, and Eshmuno HCX. Non-limiting examples of anion exchange resins (e.g., salt-tolerant anion exchange resins) include HyperCel STAR AX, Toyopearl NH2-750F, and Q Sepharose HP. Non-limiting examples of suitable multi-modal anion exchange resins include Capto Adhere, PPA HyperCel, and HEA HyperCel. Non-limiting examples of suitable HCIC resins include MEP HyperCel, PPA HyperCel, and HEA HyperCel. Non-limiting examples of suitable affinity chromatography resins include MabSelect SuRe, KappaSelect, Eshmuno A, ProSep A, and immobilized antibody resins.

According to some embodiments, the column is associated with one or more mobile phase materials (i.e., one or more fluids that flow through the stationary phase material of the column). Non-limiting examples of suitable mobile phase materials include sodium citrate, sodium phosphate, sodium chloride, sodium acetate, Tris-HCl, glycine, and histidine. In some embodiments, the mobile phase material has a pH of at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, or at least about 9.0. In some embodiments, the mobile phase material has a pH of about 9.0 or less, about 8.5 or less, about 8.0 or less, about 7.5 or less, about 7.0 or less, about 6.5 or less, about 6.0 or less, about 5.5 or less, about 5.0 or less, about 4.5 or less, about 4.0 or less, about 3.5 or less, or about 3.0 or less. In some embodiments, the mobile phase material has a pH in the range of about 3.0 to about 5.0, about 3.0 to about 6.0, about 3.0 to about 7.0, about 3.0 to about 8.0, about 3.0 to about 9.0, about 4.0 to about 6.0, about 4.0 to about 7.0, about 4.0 to about 8.0, about 4.0 to about 9.0, about 5.0 to about 7.0, about 5.0 to about 8.0, about 5.0 to about 9.0, about 6.0 to about 7.0, about 6.0 to about 8.0, about 6.0 to about 9.0, about 7.0 to about 8.0, or about 7.0 to about 9.0.

In some embodiments, the mobile phase material comprises a salt (e.g., sodium chloride). In some embodiments, the mobile phase material has a salt (e.g., sodium chloride) concentration of at least about 10 mM, at least about 20 mM, at least about 50 mM, at least about 100 mM, at least about 150 mM, at least about 200 mM, at least about 250 mM, at least about 300 mM, at least about 350 mM, at least about 400 mM, at least about 450 mM, at least about 500 mM, at least about 1 M, at least about 1.5 M, or at least about 2 M. In some embodiments, the mobile phase material has a salt concentration of about 2 M or less, about 1.5 M or less, about 1 M or less, about 500 mM or less, about 450 mM or less, about 400 mM or less, about 350 mM or less, about 300 mM or less, about 250 mM or less, about 200 mM or less, about 150 mM or less, about 100 mM or less, about 50 mM or less, about 20 mM or less, or about 10 mM or less. In some embodiments, the mobile phase material has a salt concentration in the range of about 10 mM to about 150 mM, about 10 mM to about 250 mM, about 10 mM to about 500 mM, about 10 mM to about 1 M, about 10 mM to about 1.5 M, about 10 mM to about 2 M, about 50 mM to about 150 mM, about 50 mM to about 250 mM, about 50 mM to about 500 mM, about 50 mM to about 1 M, about 50 mM to about 1.5 M, about 50 mM to about 2 M, about 100 mM to about 250 mM, about 100 mM to about 500 mM, about 100 mM to about 1 M, about 100 mM to about 1.5 M, about 100 mM to about 2 M, about 250 mM to about 500 mM, about 250 mM to about 1 M, about 250 mM to about 1.5 M, about 250 mM to about 2 M, about 500 mM to about 1 M, about 500 mM to about 1.5 M, about 500 mM to about 2 M, or about 1 M to about 2 M.

The column may be operated in bind-elute mode, flow-through mode, or any other suitable mode. In bind-elute mode, two or more mobile phase materials may be directed to flow through the first stationary phase material of the column. In some embodiments, a first mobile phase material that is directed to flow through the column is configured to promote the binding of the at least one biologically-produced product to the first stationary phase material. Non-limiting examples of a suitable first mobile phase material (e.g., a bind buffer) include phosphate buffer, citrate buffer, formate buffer, acetate buffer, and tris buffer. In some embodiments, a second mobile phase material that is directed to flow through the column is configured to wash one or more materials other than the at least one biologically-produced product from the first stationary phase material. Non-limiting examples of a suitable second mobile phase material (e.g., a wash buffer) include, but are not limited to, phosphate buffer, citrate buffer, formate buffer, acetate buffer, and tris buffer. In some embodiments, the second mobile phase material is substantially similar to the first mobile phase material in terms of the types of components in the mobile phase (e.g. first phosphate buffer and second phosphate buffer), but substantially differs in at least one property (e.g. pH, ionic strength, etc.). In some embodiments, a third mobile phase material that is directed to flow through the column is configured to elute the at least one biologically-produced product from the first stationary phase material. Non-limiting examples of a suitable third mobile phase material (e.g., an elute buffer) include, but are not limited to, phosphate buffer, citrate buffer, formate buffer, acetate buffer, and tris buffer. In some embodiments, one or more fractions comprising the at least one biologically-produced product may be collected after the third mobile phase material is directed to flow through the column.

In flow-through mode, one or more mobile phase materials may be directed to flow through the first stationary phase material of the column. In some embodiments, a first mobile phase material that is directed to flow through the column is configured to promote the binding of one or more types of impurities to the stationary phase material. In some embodiments, the at least one biologically-produced product may “flow through” the stationary phase. In some embodiments, one or more fractions comprising the at least one biologically-produced product may be collected after the first mobile phase material is directed to flow through the column. Non-limiting examples of a suitable first mobile phase material include phosphate buffer, citrate buffer, formate buffer, acetate buffer, and tris buffer.

If a purification module comprises more than one partitioning unit applying chromatography as a partitioning technique, the first stationary phase material of the column of each partitioning unit may be the same or different. The one or more mobile phase materials associated with the column each partitioning unit may similarly be the same or different. In some embodiments, the partitioning technique applied by at least one partitioning unit of the purification module comprises filtration. According to certain embodiments, the filtration technique comprises tangential flow filtration (also referred to as cross-flow filtration). A person of ordinary skill in the art would understand tangential flow filtration to refer to a type of filtration in which a fluid stream travels tangentially across the surface of a filter (e.g., a filtration membrane, a monolith). According to certain embodiments, the filtration technique comprises dead-end filtration. A person of ordinary skill in the art would understand dead-end filtration to refer to a type of filtration in which a fluid stream travels perpendicularly across the surface of a filter.

In some embodiments, a filter of the at least one partitioning unit (e.g., a filter in either a tangential flow filtration device or a dead-end filtration device) is a filtration membrane. In some cases, the filtration membrane comprises a plurality of pores having a pore size. In some embodiments, components of the fluid stream having a size smaller than the pore size of the filtration membrane may travel through the filtration membrane as part of a filtrate. In some embodiments, components of the fluid stream having a size larger than the pore size of the filtration membrane may be prevented from traveling through the filtration membrane and may be retained as part of a retentate. In certain embodiments, the filtration membrane has a pore size that permits passage of the at least one biologically-produced product and prohibits passage of one or more types of impurity in a fluid stream. In certain embodiments, the filtration membrane has a pore size that permits passage of one or more types of impurity and prohibits passage of the at least one biologically-produced product. In some embodiments, the filtration membrane has a pore size of at least about 0.01 μm, at least about 0.02 μm, at least about 0.05 μm, at least about 0.08 μm, at least about 0.1 μm, at least about 0.2 μm, at least about 0.3 μm, at least about 0.4 μm, at least about 0.5 μm, or at least about 1 μm. In some embodiments, the filtration membrane has a pore size of about 1 μm or less, about 0.5 μm or less, about 0.4 μm or less, about 0.3 μm or less, about 0.2 μm or less, about 0.1 μm or less, about 0.08 μm or less, about 0.05 μm or less, about 0.02 μm or less, or about 0.01 μm or less. In some embodiments, the filtration membrane has a pore size in the range of about 0.01 μm to about 0.05 μm, about 0.01 μm to about 0.1 μm, about 0.01 μm to about 0.5 μm, about 0.01 μm to about 1 μm, about 0.1 μm to about 0.5 μm, about 0.1 μm to about 1 μm, about 0.2 μm to about 0.5 μm, about 0.2 μm to about 1 μm, or about 0.5 μm to about 1 μm.

In some embodiments, the filter of the at least one partitioning unit is a monolith. A monolith generally refers to a filter formed from a porous solid material (e.g., a ceramic material). In some embodiments, components of the fluid stream having a size smaller than the average pore size of the monolith may travel through the monolith as part of a filtrate. In some embodiments, components of the fluid stream having a size larger than the pore size of the monolith may be prevented from traveling through the filtration membrane and may be retained as part of a retentate. In certain embodiments, the monolith has an average pore size that permits passage of the at least one biologically-produced product and prohibits passage of one or more types of impurity in a fluid stream. In certain embodiments, the monolith has an average pore size that permits passage of one or more types of impurity and prohibits passage of the at least one biologically-produced product. In some embodiments, the monolith has an average pore size of at least about 0.1 microns (μm), at least about 0.2 μm, at least about 0.3 μm, at least about 0.4 μm, at least about 0.5 μm, or at least about 1 μm. In some embodiments, the monolith has an average pore size of about 1 μm or less, about 0.5 μm or less, about 0.4 μm or less, about 0.3 μm or less, about 0.2 μm or less, or about 0.1 μm or less. In some embodiments, the monolith has an average pore size in the range of about 0.1 μm to about 0.5 μm, about 0.1 μm to about 1 μm, about 0.2 μm to about 0.5 μm, about 0.2 μm to about 1 μm, or about 0.5 μm to about 1 μm.

In some embodiments, the partitioning technique applied by at least one partitioning unit of the purification module comprises precipitation. In certain embodiments, the at least one partitioning unit comprises a precipitation apparatus. In some embodiments, the precipitation apparatus comprises a static mixer and/or a T-mixer. The precipitation apparatus may comprise a vessel (e.g., a settling tank), according to some embodiments. In some cases, the vessel may be sized to provide a fluid stream with sufficient residence time within the vessel for one or more types of impurity to precipitate from the fluid stream. In some embodiments, the residence time of a fluid stream flowing through the precipitation apparatus is at least about 5 minutes, at least about 10 minutes, at least about 15 minutes, at least about 20 minutes, at least about 30 minutes, at least about 60 minutes, at least about 2 hours, at least about 5 hours, or at least about 10 hours. In some embodiments, the residence time is in the range of about 5 minutes to about 10 minutes, about 5 minutes to about 30 minutes, about 5 minutes to about 60 minutes, about 5 minutes to about 5 hours, about 5 minutes to about 10 hours, about 30 minutes to about 60 minutes, about 30 minutes to about 5 hours, about 30 minutes to about 10 hours, about 60 minutes to about 5 hours, about 60 minutes to about 10 hours, or about 5 hours to about 10 hours. The residence time may be calculated by dividing the volume of the vessel by the volumetric flow rate of the fluid stream flowing through the vessel.

In some embodiments, the partitioning technique applied by at least one partitioning unit of the purification module comprises crystallization. In certain embodiments, the at least one partitioning unit comprises a crystallization apparatus. The crystallization apparatus may comprise a vessel (e.g., a crystallization tank), according to some embodiments. In some cases, the vessel may be sized to provide a fluid stream with sufficient residence time within the vessel for one or more types of impurity to crystallize. In some embodiments, the residence time of a fluid stream flowing through the crystallization apparatus is at least about 5 minutes, at least about 10 minutes, at least about 15 minutes, at least about 20 minutes, at least about 30 minutes, at least about 60 minutes, at least about 2 hours, at least about 5 hours, or at least about 10 hours. In some embodiments, the residence time is in the range of about 5 minutes to about 10 minutes, about 5 minutes to about 30 minutes, about 5 minutes to about 60 minutes, about 5 minutes to about 5 hours, about 5 minutes to about 10 hours, about 30 minutes to about 60 minutes, about 30 minutes to about 5 hours, about 30 minutes to about 10 hours, about 60 minutes to about 5 hours, about 60 minutes to about 10 hours, or about 5 hours to about 10 hours.

In some embodiments, the partitioning technique applied by at least one partitioning unit of the purification module comprises extraction. In certain embodiments, for example, the at least one partitioning unit comprises an extraction apparatus (e.g., a liquid-liquid extraction apparatus). In some embodiments, an extraction apparatus may be configured to receive a first solvent and a second solvent immiscible in the first solvent. In some embodiments, the extraction apparatus may be configured to further receive a fluid stream. In some embodiments, at least one component of the fluid stream (e.g., the at least one biologically-produced product) is miscible in a first solvent and at least one type of impurity in the fluid stream is miscible in a second, different solvent. In some embodiments, the solvent comprising the at least one biologically-produced product may be collected.

In some embodiments, the partitioning module further comprises at least one buffer delivery module configured to deliver at least one buffer to at least one partitioning unit. The at least one buffer delivery module may comprise one or more reservoirs containing one or more buffers. In some embodiments, the at least one buffer delivery module is in fluidic communication (e.g., direct fluidic communication) with at least one partitioning unit, at least two partitioning units, or at least three partitioning units. In some embodiments, the partitioning module contains at least one, at least two, or at least three buffer delivery modules.

In some embodiments, the purification module is configured to remove a relatively large percentage of at least a first type of impurity and at least a second type of impurity. In some embodiments, the purification module is configured to remove at least about 50%, at least about 75%, at least about 90%, at least about 95%, or at least about 99% of the first type of impurity and/or the second type of impurity from an input stream received by the purification module.

Formulation module

In some embodiments, the biomanufacturing system further comprises a formulation module. In some embodiments, the optional formulation module is configured to further process an output of the purification module to produce a formulated product. According to certain embodiments, for example, the formulation module may comprise a filtration unit configured to concentrate and/or further purify the at least one biologically-produced product, a viral filtration unit configured to remove and/or inactivate one or more viruses, and/or a product packaging unit configured to package doses of the at least one biologically-produced product into one or more sterile containers. In operation, the formulation module may receive a fluid stream (e.g., a purified filtrate stream) from the purification module and produce a formulated product stream.

In some embodiments, the formulation module comprises a filtration unit. According to some embodiments, the filtration unit is configured to increase the concentration of the at least one biologically-produced product in a fluid stream and/or further remove one or more types of impurity from the fluid stream. In some cases, the filtration unit comprises a tangential flow filtration (TFF) device. In certain embodiments, the TFF device comprises an ultrafiltration membrane. In some embodiments, the ultrafiltration membrane has a pore size of at least about 0.005 μm, at least about 0.01 μm, at least about 0.02 μm, at least about 0.03 μm, at least about 0.04 μm, at least about 0.05 μm, at least about 0.06 μm, at least about 0.07 μm, at least about 0.08 μm, at least about 0.09 μm, at least about 0.1 μm, at least about 0.2 μm, at least about 0.3 μm, about least about 0.4 μm, or at least about 0.5 μtn. In some embodiments, the ultrafiltration membrane has a pore size of about 0.5 μm or less, about 0.4 μm or less, about 0.3 μm or less, about 0.2 μm or less, about 0.1 μm or less, about 0.09 μm or less, about 0.08 μm or less, about 0.07 μm or less, about 0.06 μm or less, about 0.05 μm or less, about 0.04 μm or less, about 0.03 μm or less, about 0.02 μm or less, about 0.01 μm or less, or about 0.005 μm or less. In some embodiments, the ultrafiltration membrane has a pore size in the range of about 0.005 μm to about 0.01 μm, about 0.005 μm to about 0.05 μm, about 0.005 μm to about 0.1 μm, about 0.005 μm to about 0.2 μm, about 0.005 μm to about 0.3 μm, about 0.005 μm to about 0.4 μm, about 0.005 μm to about 0.5 μm, about 0.01 μm to about 0.05 μm, about 0.01 μm to about 0.1 μm, about 0.01 μm to about 0.2 μm , about 0.01 μm to about 0.3 μm, about 0.01 μm to about 0.4 μm, about 0.01 μm to about 0.5 μm, about 0.05 μm to about 0.1 μm, about 0.05 μm to about 0.2 μm, about 0.05 μm to about 0.3 μm, about 0.05 μm to about 0.4 μm, about 0.05 μm to about 0.5 μm, or about 0.1 μm to about 0.5.

In some embodiments, the formulation module comprises a viral filtration unit. The viral filtration unit may be configured to receive a fluid stream and remove and/or inactivate one or more viruses from the fluid stream. In some embodiments, the viral filtration unit comprises a nanofiltration membrane. In some embodiments, the nanofiltration membrane has a pore size of about 500 nm or less, about 200 nm or less, about 100 nm or less, about 50 nm or less, about 20 nm or less, or about 10 nm or less. In some embodiments, the nanofiltration membrane has a pore size in the range of about 10 nm to about 20 nm, about 10 nm to about 50 nm, about 10 nm to about 100 nm, about 10 nm to about 200 nm, about 10 nm to about 500 nm, about 50 nm to about 100 nm, about 50 nm to about 200 nm, about 50 nm to about 500 nm, or about 100 nm to about 500 nm.

According to certain embodiments, the formulation module comprises a dilution adjustment unit. In some embodiments, the dilution adjustment unit is configured to add a diluent to an output of the purification module (e.g., the purified filtrate). Non-limiting examples of suitable diluents include polar protic solvents (e.g., water, aqueous solutions, buffers, methanol, ethanol, acetic acid), polar aprotic solvents (e.g. dimethylsulfoxide, acetonitrile, dimethylformamide, acetone), and nonpolar solvents (e.g., pentane, hexane, cyclohexane, benzene). In some embodiments, the diluent may include agents to stabilize the formulated purified filtrate to improve stability. Non-limiting examaples include antioxidants (e.g., sodium bisulfite, sodium metabisulfite, ascorbate, sodium sulfite, thioglycerol), bulking agents (e.g., mannitol, dextran, glycine), viscosity enhancers/reducers or surfactants (e.g., polysorbate, 20, polysorbate 80), chelating agents (e.g., EDTA), preservatives (e.g., thimersol, sorbic acid), cryoprotectants (e.g., sucrose, trehalose, sorbitol), lyoprotectants, and adjuv ants.(e.g., TLR agonists, CpG DNA, alum).

According to certain embodiments, the formulation module comprises a product packaging unit. In some embodiments, the product packaging unit is configured to package one or more doses of the at least one biologically-produced product into one or more containers. Non-limiting examples of a suitable containers include bags (e.g., bags configured to store intravenous liquid), vials, syringes, and bottles. The one or more containers may have any shape and may be formed of any suitable material (e.g., plastic, glass). In some embodiments, the one or more containers are airtight containers. In some embodiments, the one or more containers are sterile containers. In operation, the product packaging unit may receive a fluid stream comprising at least one biologically-produced product (e.g., a purified filtrate from a purification module) and package one or more portions of the fluid stream in one or more containers.

In other embodiments, the formulation module may not comprise a product packaging unit. In some embodiments, the product packaging unit may be a separate module that is distinct from the formulation module. In general, a product packaging module has the features described above with respect to the product packaging unit.

### Process Monitoring and Control Systems

In some embodiments, the biomanufacturing system (e.g., system 900 in FIG. 9) comprises at least one process monitoring and control system. The process monitoring and control system may be configured to monitor and/or control the biomanufacturing system. In some embodiments, the process monitoring and control system may be configured to monitor and/or control the biomanufacturing system as a plurality of separate (e.g., spatially separate) systems for monitoring and/or control that communicate with each other, as a single system, or as any number of overlapping and/or non-overlapping systems. In certain embodiments, the process monitoring and control system may be configured to transmit information derived fom the monitoring to another component, module, system, and/or a user, identify a problem occurring in the biomanufacturing system, translate the information derived from monitoring into corrective action, alert a user about a problem and/or a corrective action, and/or implement the corrective action based at least in part on information derived from monitoring. In some embodiments, the process monitoring and control system may comprise one or more sensors (e.g., camera). The sensor(s) may measure or otherwise monitor one or more characteristics (e.g., fluid level, presence of bubbles, cell density, cell growth, agitator or impeller speed, valve position, rmp of pumps, fluid flow rates) of the system (e.g., module or component thereof) and produce one or more signals (e.g., images) indicative of the characteristic(s). The signal(s) (e.g., image) may be transmitted to a unit or units, which is/are in electrical communication with the sensor(s). In some embodiments, the unit may be a controller that is configured to control one or more parameters of the system. In such embodiments, the controller may be operatively associated with one or more components (e.g., module, pump, valves) of the system and/or with one or more processors for controlling component(s) of the system. For example, the controller may be operatively associated with one or more processors for controlling flow rate, valves, pumps, fluid levels, agitator or impeller speed, bubble vents, selection of reagent type, selection of reagent concentration, incubation time, selection of the ratio of reagents, the addition of an additive, or combinations thereof. Optionally, the controller may also be operatively associated with other components such as a user interface and an external communication unit (e.g., a USB, flash drive), and/or other components, as described in more detail below. The user interface may be used to display the signal(s), alert the user of a problem with the system or a certain module or component thereof, and/or receive operation instructions from the user.

As used herein, a unit that is “operatively associated with” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected (e.g., via channels) so as to cause or enable the components so associated to perform their intended functionality. For instance, in some embodiments, the controller may be electronically coupled to a component via a wireless or wired electronic connection. For example, the controller may be electronically coupled via a wireless or wired electronic connection to one or more processors for controlling flow rate, temperature, selection of reagent type, selection of reagent concentration, reaction time, selection of the ratio of reagents, the addition of an additive, or combinations thereof. In certain embodiments, the controller may be electronically coupled via a wireless or wired electronic connection to a temperature regulator for one or more fluid streams and/or the reactor. In some embodiments, the controller may be electronically coupled via a wireless or wired electronic connection to a fluid flow source (e.g., pump) for one or more fluid streams and/or the reactor. In certain embodiments, the controller may be electronically coupled via a wireless or wired electronic connection to one or more processors for controlling one or more selection of reagents (e.g., type, concentration, ratio).

In general, a unit (e.g., controller) may be used to conduct process monitoring and/or control by the use of feedback from one or more processes taking place in the biomanufacturing system. In some embodiments, a unit (e.g., controller) may be used to partially or fully automate the system. For instance, a controller may be configured to receive signal(s) (e.g., images) from the one or more sensors, to quantitatively analyze one or more signals or a pattern of signals (e.g., images), to compare one or more signals or a pattern of signals with other signals (e.g., reference signal) or values pre-programmed into the controller, and/or to modulate one or more parameters to control operation of the biomanufacturing system. For example, based at least in part on information derived from the signal, one or more parameters of the system may be modulated during a process and/or prior to and/or during a subsequent process in the system. In certain embodiments, the process operations are partially automated and may require minimal human intervention. This may allow a user or computer to partially operate the system to manufacture biologically-produced products without having any expertise in the biomanufacturing processes, equipment, or their operations. In some embodiments, the process operations are fully automated without any human intervention. This may allow a user or computer to operate the system to manufacture biologically-produced products without having any expertise in the biomanufacturing processes, equipment, or their operations.

In some embodiments, a user analyze the signal(s) from the sensor and identify the presence, absence, and/or source of a problem. In some such embodiments, a unit within the process monitoring and control system and or a user may determine appropriate corrective action to be implemented during the process and/or prior to and/or during a subsequent process. In certain embodiments, the unit may automatically identify a problem, determine the source of the problem, and/or implement appropriate corrective action. In some cases, a user may be involved in identifying a problem, determining the source of the problem, and/or implementing appropriate corrective action.

In some embodiments, implementation of the corrective action may be performed via a controller. For instance, a signal or pattern of signals (e.g., images) produced by the sensor(s) (e.g., cameras) can be transmitted to a controller. In some cases, the controller compares the signal or pattern of signals to a second set of signal(s). The second signal or pattern of signals may be, for example, signal(s) determined previously in the biomanufacturing system, or reference signal(s). In some cases, a reference signal or pattern of signals includes one or more threshold values or a range of threshold values. The controller may compare a first signal or pattern of signals with a second signal or pattern of signals (e.g., reference signals), and determine whether to modulate one or more parameters in the system. For instance, the controller can determine problems that have occurred or are occurring in the biomanufacturing system, and the controller may send one or more signal(s) to one or more components to cause modulation of a parameter in all or portions of the system. That is, the measured signal or pattern of signals (e.g., image or pattern of images) can be used by the controller to generate a drive signal and provide feedback control to the system. For example, based (at least in part) on the signal(s) received by the controller, this feedback can be used to modulate a parameter of the system by controlling, e.g., one or more of a pump, vacuum, valve, etc. The modulation may be performed, in certain embodiments, by the controller sending one or more drive signals to an appropriate component of the biomanufacturing system to actuate that or another component. Any suitable valve drive electronics circuit may be used to receive a drive signal and convert the drive signal to a voltage, current, or other signal capable of actuating the component. Alternatively, when corrective action cannot be taken, the controller may send one or more signal(s) to one or more components to cause the system or a component thereof to shut down. In some embodiments, one or more feedback control methods such as proportional control, integral control, proportional-integral control, derivative control, proportional-derivative control, integral-derivative control, nonlinear control, adaptive control, model-based control, and proportional-integral-derivative control can be used by a controller to modulate a parameter or cause the system or a component thereof to shut down.

Regardless of whether corrective action is implemented by a user or a controller, corrective action may be implemented prior to formation of the formulated product stream. In embodiments in which corrective action cannot be taken, the manufacturing of the biologically-produced product may be stopped. In general, the corrective actions may be implemented during the current and/or future biomanufacturing processes.

In certain embodiments, one or more measured signals (e.g., images) is processed or manipulated (e.g., before or after transmission, and/or before being compared to a signal). It should be appreciated, therefore, that when a signal is transmitted (e.g., to a controller, user), compared (e.g., with a reference signal or another signal), or otherwise used in a feedback process, that the raw signal may be used or a processed/manipulated signal based (at least in part) on the raw signal may be used. For example, in some cases, one or more derivative signals of a measured signal can be calculated (e.g., using a differentiator, or any other suitable method) and used to provide feedback. In other cases, signals are normalized (e.g., subtracting a measured signal from a background signal). In one set of embodiments, a signal comprises an optical image.

In some embodiments, the controller may be computer implemented. In general, any suitable calculation methods, steps, simulations, algorithms, systems, and system elements described herein may be implemented and/or controlled using one or more computer implemented controller(s). The methods, steps, controllers, and controller elements described herein are not limited in their implementation to any specific computer system described herein, as many other different machines may be used.

The computer implemented controller(s) can be part of or coupled in operative association with an image analysis system and/or other automated system components, and, in some embodiments, is configured and/or programmed to control and adjust operational parameters, as well as analyze and calculate values. In some embodiments, the computer implemented controller(s) can send and receive reference signals to set and/or control operating parameters of system apparatus. In other embodiments, the computer implemented system(s) can be separate from and/or remotely located with respect to the other system components and may be configured to receive data from one or more remote systems via indirect and/or portable means, such as via portable electronic data storage devices, such as magnetic disks, or via communication over a computer network, such as the Internet or a local intranet.

The computer implemented controller(s) may include several known components and circuitry, including a processing unit (i.e., processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system(s) may be a multi-processor computer system or may include multiple computers connected over a computer network.

In some embodiments, the process monitoring and control system is a non-invasive process monitoring and control system. In some cases, non-invasive process monitoring and control systems are associated with certain advantages, such as reduced risk of contamination, reduced geometric complexity, and cost savings as described herin with respect to the leveling sensor system. In some embodiments, the process monitoring and control system may comprise one or more optical sensor system and/or magnetic sensor system. Regardless of the sensor (e.g., camera) used, in some embodiments, the process monitoring and control system may allow continuous real-time monitoring. In some embodiments, the process monitoring and control system may allow for a relatively high amount of automation. For instance, in some embodiments, at least about 50% (e.g., at least about 60%, at least about 70%, at least about 80%, at least about 90%, 100%) of the modules and/or processes in the biomaufacturing system are automated.

In some embodiments, one or more processes occurring in an upstream component may be monitored by one or more optical sensors (e.g., cameras). For example, optical sensors may be used to monitor fluid volume level, agitator or impeller speed, perfusion probe integrity, and gas sparging. In some embodiments, an upstream process monitoring and control sensor may monitor the level of fluid volume in the bioreactor. In some such cases, the upstream process monitoring and control sensor may operate as described herein with respect to the discrete level sensing system. In some embodiments, the process monitoring and control level sensing sensor may be configured to monitor the accuracy of the in-vessel level control system. In some embodiments, an upstream process monitoring and control sensor may monitor the vortex above the rotating agitator or impeller in the bioreactor. In some such embodiments, the process monitoring and control vortex sensor may be used to monitor the accuracy of the agitator or impeller speed control. In some embodiments, an upstream process monitoring and control sensor may be configured to monitor the filter probe. For example, an upstream process monitoring and control sensor may monitor fouling of the filter probe. In some such embodiments, the process monitoring and control filter probe sensor may be used to monitor indicia of fouling, such as formation of a cellular cake on the filter probe. In certain embodiments, indication of fouling may result in a user or a controller performing corrective action, such as washing the filter probe. As another example, an upstream process monitoring and control sensor may monitor the filtrate stream exiting the filter probe. In some such embodiments, the process monitoring and control filter probe sensor may be used to monitor indicia of filter probe failure (e.g., increased optical opacity of filtrate), such as the presence of cells in the filtrate stream. In certain embodiments, indication of filter probe failure may result in a user or a controller performing corrective action, such as washing the filter probe. In some embodiments, an upstream process monitoring and control sensor may monitor foam levels in the bioreactor. In some such embodiments, the process monitoring and control sensor may be configured to monitor the accuracy of the aeration system. In certain embodiments, over aeration, as indicated by foaming, may result in a user or a controller performing corrective action, such as administering anti-foaming additives or adjusting the aeration rate.

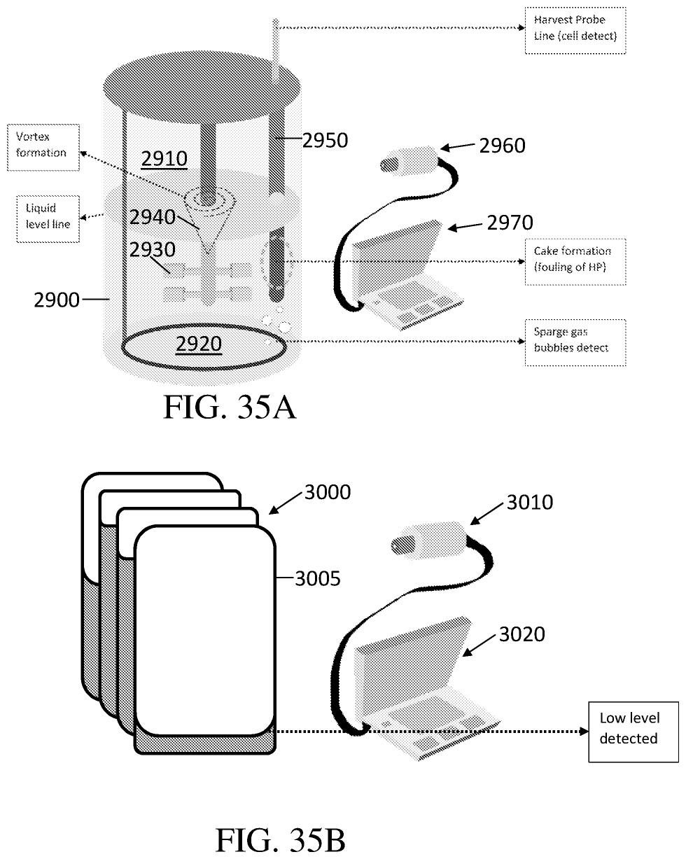

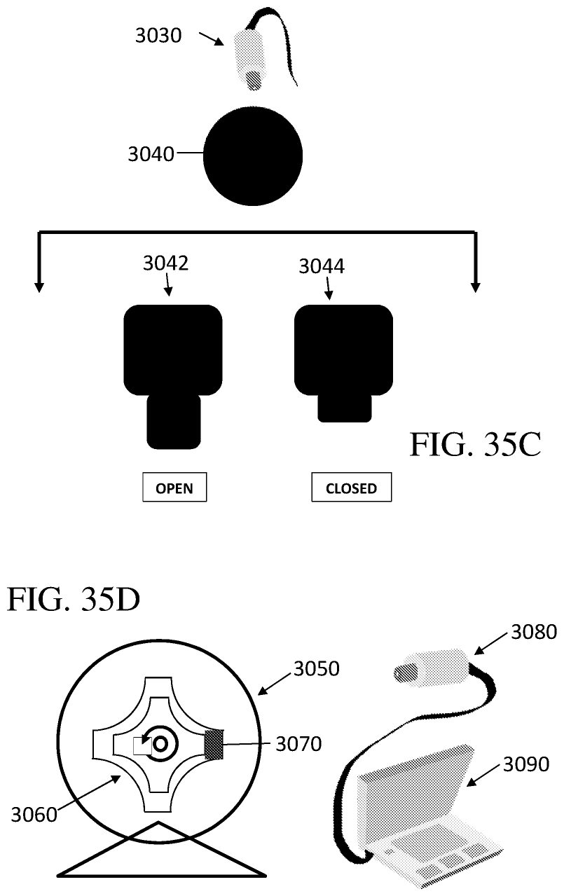

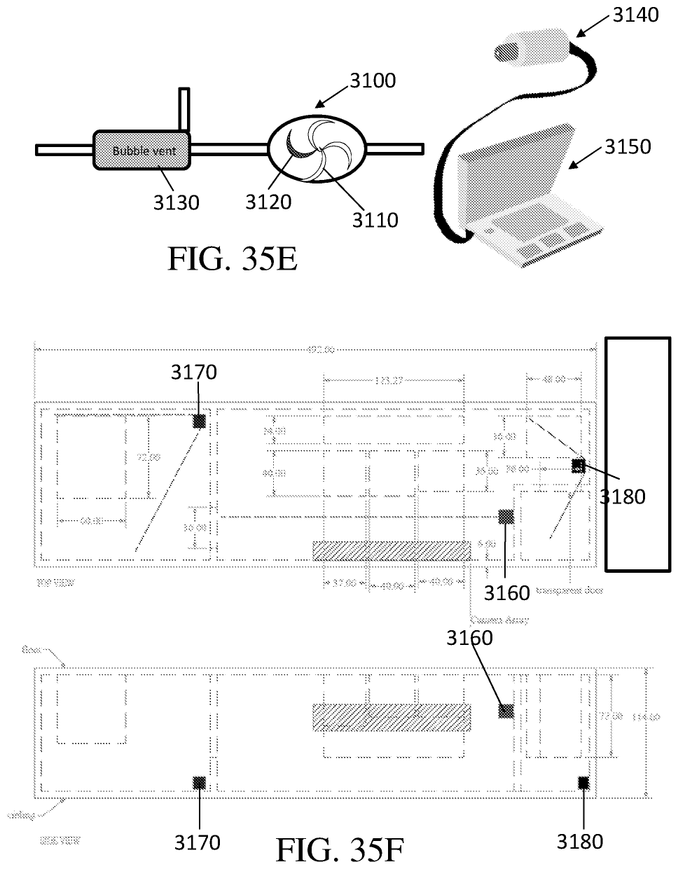

A schematic illustration of an exemplary upstream optical process monitoring and control sensor is shown in FIG. 35A. FIG. 35A shows bioreactor 2900, which comprises reaction chamber 2910 containing a bioreactor liquid (e.g., a cell suspension) 2920. FIG. 35A also shows agitator 2930, which forms a vortex 2940 within bioreactor 2910 and filter probe 2950. Camera 2960 and computer 2970 are positioned outside bioreactor 2910. In operation, camera 2960 may be used to acquire images of the vortex, liquid level, gas bubbles, filter probe, and/or filtrate stream and the images may be processed by one or more algorithms to allow for feedback control as described herein.

As described herein, one or more modules (e.g., bioreactor, purification module, adjustment module) in the biomanufacturing system handle one or more fluids (e.g., liquids). In some embodiments, an optical process monitoring and control sensor may be used to prevent and/or correct problems associated with fluid delivery to a unit and/or a module. For example, the optical sensor may be configured to monitor the level of a fluid. Low levels of fluid may result in a user being alerted and/or a controller implementing a corrective action, such as connection to a new liquid supply source. As another example, an optical process monitoring and control sensor may be configured to validate that the correct fluid is entering the biomanufacturing system and/or component thereof (e.g., a module, a unit) by reading an identification element (e.g., visible registration mark, barcode) associated with (e.g., on the surface of) the container housing the fluid. In some embodiments, an optical process monitoring and control sensor may be configured to validate that a fluid exiting the biomanufacturing system and/or a component thereof (e.g., a module, a unit) is housed within the correct container by reading an identification element (e.g., visible registration mark, barcode) associated with (e.g., on the surface of) the container. In certain embodiments, the optical sensor may be configured to monitor the level of a fluid, which has exited the biomanufacturing system and/or a component thereof (e.g., a module, a unit), in a container. The information derived from the monitoring may be used to determine whether the biomanufacturing system and/or a component thereof (e.g., a module, a unit) produced the expected volume of the fluid.

A schematic illustration of an exemplary optical process monitoring and control sensor configured to monitor fluid handling is shown in FIG. 35B. In FIG. 35B, the level of fluid in containers 3000 is being monitored by camera 3010 connected to computer 3020. Camera 3010 may detect the low level of fluid in container 3005, and computer 3020 may produce a user alert and/or implement a corrective action. Camera 3010 and computer 3020 may be positioned outside container 3000.

In some embodiments, one or more valves in the biomanufacturing system may be monitored by one or more optical sensors. In general, precise control of valve operation allows for optimal delivery of material (e.g., fluids) to the biomanufacturing system and/or component thereof (e.g., module, unit) at the optimal time. In some embodiments, certain processes in the biomanufacturing system may require the opening or closing of a certain sequence of valves, e.g., at certain times. In some embodiments, an optical process monitoring and control sensor may be configured to monitor the position of the valve(s) (e.g., opened, closed, on, off) prior to, during, and/or after a process in the biomanufacturing system and/or component thereof (e.g., module, unit). The optical process monitoring and control sensor may be configured to monitor any suitable valve. For example, an optical process monitoring and control sensor may be configured to monitor the valve position (on or off; opened or closed) of a pinch valve from a position above the pinch valve. As another example, an optical process monitoring and control sensor may be configured to monitor the internal actuator or gate position for a transparent multi-port valve. In one example, an optical process monitoring and control sensor may be configured to monitor the fluid flow before and after valve actuation to determine the direction of fluid flow for an opaque multi-port valve. As another example, an optical process monitoring and control sensor may be configured to monitor the presence or absence of fluid in a fluidic path (e.g., channel) to determine the valve position for valves for fluid input heads. Regardless of the type of valve, the process monitoring and control system may be configured to compare the actual valve position with the programmed valve position to determine if an error has occurred.

A schematic illustration of an exemplary optical process monitoring and control sensor for monitoring valve position is shown in FIG. 35C. In FIG. 35C, camera 3030 is positioned above pinch valve 3040, which may be positioned in an opened 3042 or closed 3044 state, as shown. Camera 3030 may be positioned outside the the valve, the biomanufacturing system, the module, etc. and connected to a computer (not shown).

In some embodiments, one or more pumps in the biomanufacturing system may be monitored by one or more optical sensors. In certain embodiments, pumps may be used to induce fluid flow in the biomanufacturing system and/or components thereof (e.g., module, unit). In some instances, the revolutions per minute (also referred to as rpm) of a pump may be used to control the fluid flow rate. In some embodiments, an optical process monitoring and control sensor may be configured to monitor the rpm of a pump. For example, in a pump with a transparent housing, the optical process monitoring and control sensor may be configured to monitor the rotations of a rotating, colored object inside the pump that is indicative of or otherwise correlates with the revolutions per minute. As another example, for a pump with an opaque housing, a magnetic material may be positioned on or within a rotating portion of the pump. The rotation of the magnetic material may trigger an external reed switch (or hall effect sensor) that counts the number of instances and relates it to pump rpm. Regardless of the type of or the transparency of the pump housing, the process monitoring and control system may be configured to compare the actual rpm with the programmed rpm to determine if an error has occurred.

A schematic illustration of an exemplary optical process monitoring and control sensor for monitoring the rpm of a pump is shown in FIG. 35D. In FIG. 35D, pump 3050 comprises a rotation element 3060 that has a marked (e.g., colored, magnetic) portion 3070. Camera 3080 may be positioned to monitor a signal indicative of the revolutions per minute of marked portion 3070. Camera 3030 may be positioned outside the pump, the biomanufacturing system, the module, etc. and connected to a computer 3090.

In some embodiments, one or more flow rates in the biomanufacturing system may be monitored by one or more optical sensors. In some embodiments, the one or more flow rates may be monitored to confirm the accuracy of one or more flow rate measurement devices. For example, in a flow rate measurement device with a transparent housing, the optical process monitoring and control sensor may be configured to monitor the movement of a colored object inside the flow rate measurement device that is indicative of or otherwise correlates with the flow rate. As another example, for a flow rate measurement device with an opaque housing, a magnetic material may be positioned on or within a portion of the flow rate measurement device that moves due to fluid flow. The movement of the magnetic material may trigger an external reed switch (or hall effect sensor) that counts the number of instances of a certain movement and relates it to flow rate. Regardless of the type of or the transparency of the flow rate measurement device housing, the process monitoring and control system may be configured to compare the actual flow rate with the programmed flow rate and/or the flow rate determined by the flow rate measurement device to determine if an error has occurred.

A schematic illustration of an exemplary optical process monitoring and control sensor for monitoring the flow rate is shown in FIG. 35E. In FIG. 35E, flow rate measurement device 3100 comprises a element 3110, whose movements can be used to derive flow rate, and that has a marked (e.g., colored, magnetic) portion 3120. A bubble vent 3130 may be positioned upstream of flow rate measurement device 3100 to allow for the removal of bubbles from the fluidic path that would skew the flow and/or otherwise result in inaccurate measurements of flow rate. Camera 3140 may be positioned to monitor a signal indicative of the movement of marked portion 3120 (e.g., number of revolutions). Camera 3140 may be positioned outside the biomanufacturing system, the module, flow rate measurement device, etc. and connected to a computer 3150.

In some embodiments, one or more optical sensors may be configured to monitor the facility in which the biomanufacturing system is located. In certain embodiments, the process monitoring and control system may be configured to monitor users and/or the environment (e.g., equipment, doors) around the biomanufacturing system. A schematic illustration of an exemplary optical process monitoring and control system for monitoring users and/or the environment around the biomanufacturing system is shown in FIG. 35F. As shown in FIG. 35F, a sensor (e.g., optical sensor, camera) 3160 may be configured to monitor contamination caused by users by recording the line-of-sight across the system, recording the position of the room separation door to ensure that the door is in the correct position at all times, and/or by monitoring pressure differential to ensure that the door is in the correct position at all times. In event of breach of any of these conditions, an alert may be transmitted to a user. In some embodiments, the process monitoring and control system may comprise a sensor (e.g., optical sensor, camera) 3170 configured to monitor external contamination introduced into the biomanufacturing system during fluid handling (e.g., manipulation of liquid supply sources, waster removal) by a user, monitor the rate of fluid container fill rate, and/or monitor quality metrics, such as lot acceptance rate and invalidated out-of-specification rate, e.g., automatically and/or in real time. In some embodiments, the process monitoring and control system may comprise a sensor (e.g., optical sensor, camera) 3180 configured to monitor media and buffer preparation by a user, monitor the attire of users, and/or monitor other attributes of the user and/or environment to ensure that good manufacturing practice are followed.

In general, the process monitoring and control system may include sensors to monitor any aspect of the biomanufacturing system and/or the facility in which the system is housed that would result in an adverse event (e.g., sub-optimal formulated product being recovered from the system, breach in good manufacturing practice, reduced system efficiency, system damage). For example, sensors may be used to determine leaks, blockages, breach of sterile barriers, user error, etc.

It should be understood that optical sensors as well as the process monitoring and control system are not limited to monitoring and/or deriving information from the biomanufacturing system or a component thereof based on color. It should be understood that though certain examples and embodiments of the process monitoring and control system and associated optical sensors are described with respect to color, any suitable visual marking may be used. In some embodiments, visual markings may comprise a high-contrast region with differential light scattering properties, a different material of distinct color or contrast, a geometric form or pattern, or any other fiduciary marking to indicate a region of contrast.

### Product Characteristics

In some embodiments, a product stream exiting the biomanufacturing system (e.g., a purified filtrate stream, a formulated product stream) has a relatively high concentration of at least one biologically-produced product. In some embodiments, the product stream has a concentration of the at least one biologically-produced product of at least about 1 μg/mL, at least about 2 μg/mL, at least about 5 μg/mL, at least about 10 μg/mL, at least about 20 μg/mL, at least about 50 μg/mL, at least about 100 μg/mL, at least about 200 μg/mL, at least about 500 μg/mL, at least about 1 mg/mL, at least about 2 mg/mL, at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, or at least about 90 mg/mL. In some embodiments, the product stream has a concentration of the at least one biologically-produced product in the range of about 1 μg/mL to about 10 μg/mL, about 1 μg/mL to about 50 μg/mL, about 1 μg/mL to about 100 μg/mL, about 1 μg/mL to about 200 μg/mL, about 1 μg/mL to about 500 μg/mL, about 10 μg/mL to about 50 μg/mL, about 10 μg/mL to about 100 μg/mL, about 10 μg/mL to about 200 μg/mL, about 10 μg/mL to about 500 μg/mL, about 50 μg/mL to about 100 μg/mL, about 50 μg/mL to about 200 μg/mL, about 50 μg/mL to about 500 μg/mL, about 100 μg/mL to about 500 μg/mL, or about 200 μg/mL to about 500 μg/mL. In certain embodiments, the product stream has a concentration of the at least one biologically-produced product in the range of about 1 μg/mL to about 100 mg/mL, about 10 μg/mL to about 100 mg/mL, about 50 μg/mL to about 100 mg/mL, about 100 μg/mL to about 100 mg/mL, about 200 μg/mL to about 100 mg/mL, about 500 μg/mL to about 100 mg/mL, about 1 mg/mL to about 100 mg/mL, about 2 mg/mL to about 100 mg/mL, about 5 mg/mL to about 100 mg/mL, about 10 mg/mL to about 100 mg/mL, or about 20 mg/mL to about 100 mg/mL. One suitable method for measuring the concentration of the at least one biologically-produced product in the product stream is running an enzyme-linked immunosorbent assay (ELISA).

In some embodiments, the product stream (e.g., the purified filtrate stream, the formulated product stream) exiting the biomanufacturing system has a relatively high product purity. Product purity generally refers to the degree to which the product is unmixed with non-product materials. For example, in certain embodiments, the product stream has a purity of at least about 50%, at least about 55%, at least about 60%, at least about 65%, at least about 70%, at least about 75%, at least about 80%, at least about 85%, at least about 90%, at least about 95%, or at least about 99%. In some embodiments, the product stream has a purity in the range of about 50% to about 99%, about 60% to about 99%, about 70% to about 99%, about 75% to about 99%, about 80% to about 99%, about 85% to about 99%, about 90% to about 99%, or about 95% to about 99%. One suitable method of measuring purity is reversed phase liquid chromatography.

In certain embodiments, the product stream exiting the biomanufacturing system has a relatively low concentration of host cell proteins. A host cell generally refers to a biological cell that is engineered to produce a product (e.g., Pichia pastoris), and host cell proteins generally refer to the proteins that are produced by the biological cell that are not the product. In some embodiments, the product stream has a host cell protein concentration of about 1000 ng/(mg product) or less, about 500 ng/(mg product) or less, about 200 ng/(mg product), about 100 ng/(mg product) or less, about 50 ng/(mg product) or less, about 20 ng/(mg product) or less, about 10 ng/(mg product) or less, about 5 ng/(mg product) or less, about 2 ng/(mg product) or less, or about 1 ng/(mg product) or less. In some embodiments, the product stream has a host cell protein concentration in the range of about 1 ng/(mg product) to about 10 ng/(mg product), about 1 ng/(mg product) to about 20 ng/(mg product), about 1 ng/(mg product) to about 50 ng/(mg product), about 1 ng/(mg product) to about 100 ng/(mg product), about 1 ng/(mg product) to about 200 ng/(mg product), about 1 ng/(mg product) to about 500 ng/(mg product), about 1 ng/(mg product) to about 1000 ng/(mg product), about 10 ng/(mg product) to about 50 ng/(mg product), about 10 ng/(mg product) to about 100 ng/(mg product), about 10 ng/(mg product) to about 200 ng/(mg product), about 10 ng/(mg product) to about 500 ng/(mg product), about 10 ng/(mg product) to about 1000 ng/(mg product), about 20 ng/(mg product) to about 100 ng/(mg product), about 20 ng/(mg product) to about 200 ng/(mg product), about 20 ng/(mg product) to about 500 ng/(mg product), about 20 ng/(mg product) to about 1000 ng/(mg product), about 50 ng/(mg product) to about 100 ng/(mg product), about 50 ng/(mg product) to about 200 ng/(mg product), about 50 ng/(mg product) to about 500 ng/(mg product), about 50 ng/(mg product) to about 1000 ng/(mg product), about 100 ng/(mg product) to about 500 ng/(mg product), about 100 ng/(mg product) to about 1000 ng/(mg product), about 200 ng/(mg product) to about 500 ng/(mg product), about 200 ng/(mg product) to about 1000 ng/(mg product), or about 500 ng/(mg product) to about 1000 ng/(mg product). An exemplary method for measuring the host cell protein concentration is ELISA (Cygnus Technologies).

In certain embodiments, the product stream exiting the biomanufacturing system has a concentration of DNA of about 100 ng/(mg product) or less, about 50 ng/(mg product) or less, about 20 ng/(mg product) or less, about 10 ng/(mg product) or less, about 5 ng/(mg product) or less, about 2 ng/(mg product) or less, about 1 ng/(mg product) or less, about 0.5 ng/(mg product) or less, about 0.2 ng/(mg product) or less, about 0.1 ng/(mg product) or less, about 0.05 ng/(mg product) or less, about 0.02 ng/(mg product) or less, about 0.01 ng/(mg product) or less, about 0.005 ng/(mg product) or less, about 0.002 ng/(mg product) or less, or about 0.001 ng/(mg product) or less. In some embodiments, the product stream has a concentration of DNA in the range of about 0.001 ng/(mg product) to about 0.01 ng/(mg product), about 0.001 ng/(mg product) to about 0.05 ng/(mg product), about 0.001 ng/(mg product) to about 0.1 ng/(mg product), about 0.001 ng/(mg product) to about 0.2 ng/(mg product), about 0.001 ng/(mg product) to about 0.5 ng/(mg product), about 0.001 ng/(mg product) to about 1 ng/(mg product), about 0.001 ng/(mg product) to about 10 ng/(mg product), about 0.001 ng/(mg product) to about 20 ng/(mg product), about 0.001 ng/(mg product) to about 50 ng/(mg product), about 0.001 ng/(mg product) to about 100 ng/(mg product), about 0.01 ng/(mg product) to about 0.1 ng/(mg product), about 0.01 ng/(mg product) to about 0.5 ng/(mg product), about 0.01 ng/(mg product) to about 1 ng/(mg product), about 0.01 ng/(mg product) to about 10 ng/(mg product), about 0.01 ng/(mg product) to about 20 ng/(mg product), about 0.01 ng/(mg product) to about 50 ng/(mg product), about 0.01 ng/(mg product) to about 100 ng/(mg product), about 0.1 ng/(mg product) to about 0.5 ng/(mg product), about 0.1 ng/(mg product) to about 1 ng/(mg product), about 0.1 ng/(mg product) to about 10 ng/(mg product), about 0.1 ng/(mg product) to about 50 ng/(mg product), about 0.1 ng/(mg product) to about 100 ng/(mg product),about 1 ng/(mg product) to about 10 ng/(mg product), about 1 ng/(mg product) to about 20 ng/(mg product), about 1 ng/(mg product) to about 50 ng/(mg product), about 1 ng/(mg product) to about 100 ng/(mg product), about 10 ng/(mg product) to about 50 ng/(mg product), about 10 ng/(mg product) to about 100 ng/(mg product), about 20 ng/(mg product) to about 100 ng/(mg product), or about 50 ng/(mg product) to about 100 ng/(mg product). An exemplary method for measuring DNA concentration is the Quant-iT™ PicoGreen® dsDNA Assay Kit (ThermoFisher).

In some embodiments, the product stream has a relatively low concentration of aggregates. In some embodiments, the percentage of aggregates present in the product stream is about 5.00% or less, about 2.00% or less, about 1.50% or less, about 1.00% or less, about 0.50% or less, about 0.20% or less, or about 0.10% or less. In some embodiments, the percentage of aggregates present in the product stream is in the range of about 0.10% to about 0.50%, about 0.10% to about 1.00%, about 0.10% to about 1.50%, about 0.10% to about 2.00%, about 0.10% to about 5.00%, about 0.50% to about 1.00%, about 0.50% to about 1.50%, about 0.50% to about 2.00%, about 0.50% to about 5.00%, about 1.00% to about 2.00%, about 1.00% to about 5.00%, or about 2.00% to about 5.00%. An exemplary method for measuring the percentage of aggregates is size exclusion chromatography.

In some embodiments, the product stream has a relatively high potency. In some embodiments, the product stream has a potency that is at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, or at least about 200% of standard potency as evaluated against a reference product such as WHO standard material. In some embodiments, the product stream has a potency that is about 50% to about 100%, about 50% to about 125%, about 50% to about 150%, about 50% to about 175%, about 50% to about 200%, about 60% to about 100%, about 60% to about 125%, about 60% to about 150%, about 60% to about 175%, about 60% to about 200%, about 70% to about 100%, about 70% to about 125%, about 70% to about 150%, about 70% to about 175%, about 70% to about 200%, about 75% to about 100%, about 75% to about 125%, about 75% to about 150%, about 75% to about 175%, about 75% to about 200%, about 80% to about 99%, about 80% to about 125%, about 80% to about 150%, about 80% to about 175%, about 80% to about 200%, about 85% to about 100%, about 85% to about 125%, about 85% to about 150%, about 85% to about 175%, about 85% to about 200%, about 90% to about 100%, about 90% to about 125%, about 90% to about 150%, about 90% to about 175%, about 90% to about 200%, about 100% to about 125%, about 100% to about 150%, about 100% to about 175%, about 100% to about 200%, or about 150% to about 200% of standard potency. An exemplary method for measuring potency is running a cell-based proliferation assay.

### System Characteristics

Certain biomanufacturing systems described herein have a relatively small footprint. As used herein, the footprint of a system generally refers to the sum of the surface areas of the bottom surfaces of each surface component (e.g., the surfaces in contact with the floor). In some cases, a relatively small footprint may advantageously facilitate transport of the system (e.g., via a motor vehicle). In certain cases, the biomanufacturing system has a footprint of about 20 m² or less, about 10 m² or less, about 5 m² or less, about 1 m² or less, or about 0.5 m² or less. In some embodiments, the biomanufacturing system has a footprint of about 0.5 m² to about 5 m², about 0.5 m² to about 10 m², about 0.5 m² to about 20 m², about 1 m² to about 5 m², about 1 m² to about 10 m², about 1 m² to about 20 m², about 5 m² to about 10 m², about 5 m² to about 20 m², or about 10 m² to about 20 m².

In some embodiments, the biomanufacturing system has a relatively low maximum height. The maximum height of the system may refer to the maximum vertical distance between a bottom surface of the system and a top surface of the system. In some cases, a relatively low maximum height may advantageously facilitate transport of the system (e.g., via a motor vehicle). In some embodiments, the biomanufacturing system has a maximum height of about 3 m or less, about 2 m or less, about 1 m or less, about 0.5 m or less, or about 0.1 m or less. In some embodiments, the biomanufacturing system has a maximum height in the range of about 0.1 m to about 0.5 m, about 0.1 m to about 1 m, about 0.1 m to about 2 m, or about 0.1 m to about 3 m.

In some embodiments, one or more modules of the biomanufacturing system (e.g., bioreactor 902, filter 904, adjustment module 916, purification module 906, formulation module 920) are disposable. In some embodiments, one or more modules is configured for single use. In some embodiments, each module of the biomanufacturing system is disposable. In some embodiments, each module of the biomanufacturing system is configured for single use. In some cases, a single use (e.g., disposable) bioreactor may be associated with certain advantages, such as a lower cross-contamination risk, simplified handling, high flexibility, and time and cost savings (e.g., due to pre-sterilized components). In some embodiments, the volumetric productivity of the biomanufacturign systems described herein may be relatively high per unit volume of the system.

In some embodiments, one or more modules of the biomanufacturing system (e.g., bioreactor 902, filter 904, adjustment module 916, purification module 906, formulation module 920) are configured to be cleaned in place. In some embodiments, a cleaning solution may be directed to flow through one or more fluidic paths within one or more modules of the biomanufacturing system. In some embodiments, the cleaning solution comprises hot water, steam, sodium hydroxide, ozone, hydrogen peroxide, bleach, alcohols (e.g., methanol, ethanol), and/or surfactant solutions. In some embodiments, the cleaning solution may sanitize and/or sterilize the one or more fluidic paths. In some embodiments, the cleaning may facilitate reuse of one or more modules of the biomanufacturing system. In some embodiments, the cleaning may facilitate reuse of all modules of the biomanufacturing system.

In general, the biomanufacturing system described herein does not suffer from one or more limitations of conventional systems. For instance, in conventional approaches, additional process steps to adjust the pH, conductivity, composition, and concentration of eluted fluids, or temporary storage steps, are often inserted between at least some (e.g., each) step of the complete sequence of operations in a unit and/or module. These additional steps add costs, time, or other inefficiencies when seeking to maximize the productivity of the process. The biomanufacturing system described herein have been designed to minimize and/or substantial reduce the total number of steps, units, and/or modules necessary to manufacture a biologically-produced product.

In general, the biomanufacturing system may be configured to manufacture biologically-produced products (e.g., pharmaceutically acceptable formulations comprising biologically-produced products, purified biologically-produced products) using a relatively small number of modules (e.g., between about 2 and about 10, between about 2 and about 8, between about 2 and about 6, between about 2 and about 5).

In general, the biomanufacturing system may be configured to perform one or more process steps (e.g., adjustment, purification, formulation, all process steps in a module) in an automated fashion. One feature of the system, according to certain embodiments, may be ease of configurability so that different biologically-produced products can be obtained on the same system.

In some embodiments, one or more modules of the biomanufacturing system (e.g., bioreactor 902, filter 904, adjustment module 916, purification module 906, formulation module 920) are configured to be modular and/or portable. In some such embodiments, the modularity and/or portability of a module(s) may allow for the replacement and/or removal of a module(s). In certain embodiments, a biomanufacturing system comprising a modular design may have a standardization of sizes so that modules may be easily exchanged and/or replaced.

As described above, the biomanufacturing system may include a process and monitoring control system associated with the overall system and/or a component thereof (e.g., a module). In some embodiments, one or more portions of the process and monitoring control system may be in physical contact with the biomanufacturing system and/or a component thereof (e.g., a module) or otherwise integrated with the biomanufacturing system and/or a component thereof. In some embodiments, at least a portion (e.g., all) of the process and monitoring control system is not in physical contact with the biomanufacturing system and/or a component thereof (e.g., a module) or otherwise spatially distinct and/or physically separate from the biomanufacturing system. In general, the process and monitoring control system can be used to operate (e.g., autonomously) various components of the biomanufacturing system. In general, any calculation methods, steps, simulations, algorithms, systems, and system elements described herein may be implemented and/or controlled using the process and monitoring control system. In some embodiments, an automated biomanufacturing system may comprise software capable of performing different optimization algorithms such as simplex, conjugate gradient, and/or interior point methods. In certain embodiments, an automated biomanufacturing system may allow the user to treat the system as a black box. Automation of a biomanufacturing system may be accomplished by a variety of suitable automation systems.

Certain embodiments are directed to kits comprising one or more modules or other components of the biomanufacturing system. In some instances, the kit comprises bioreactor 902, filter 904, adjustment module 916, purification module 906, and/or formulation module 920. Any of the kits described here may further comprise one or more modules, components, and/or systems for manufacturing the biologically-produced product described herein. For example, a kit may comprise a level sensing system. Further, the kit may also comprise an instruction manual providing guidance for using the kit to manufacture one or more biologically-produced product.

### G-CSF-Specific Systems and Methods

Some embodiments described herein relate to systems for producing granulocyte colony-stimulating factor (G-CSF). According to some embodiments, the system comprises a bioreactor (e.g., a perfusion bioreactor), at least one filter, and a purification module. The bioreactor may comprise a reaction chamber, and, as described above, may have any suitable shape and be formed of any suitable material. In some embodiments, the reaction chamber contains a suspension comprising at least one cell culture medium and at least a first type of biological cells configured to express G-CSF. In some embodiments, the biological cells are yeast cells. In certain cases, the yeast cells are Pichia pastoris cells. In some embodiments, for example during a cell growth phase, the at least one cell culture medium comprises chemically defined media comprising a carbon source or buffered glycerol-complex medium (BMGY). In some embodiments, for example during a G-CSF production phase, the at least one cell culture medium comprises chemically defined media comprising a carbon source and/or other additive for induction of protein expression or buffered methanol-complex media (BMMY).

In some embodiments, the at least one filter of the system comprises a filter probe, a filtration membrane, and/or a ceramic filter. The at least one filter may, in some embodiments, be fluidically connected (e.g., directly fluidically connected) to the bioreactor. For example, in certain embodiments, the at least one filter comprises at least one filter probe at least partially submerged in the suspension in the bioreactor. In some embodiments, the at least one filter is configured to receive an output of the bioreactor and produce at least one filtrate lean in the first type of biological cells relative to the suspension. In some embodiments, the at least one filtrate comprises G-CSF.

In some embodiments, the system further comprises an adjustment module configured to adjust one or more properties (e.g., pH, conductivity, product stability) of an input stream received by the adjustment module (e.g., the at least one filtrate) to produce an adjusted output stream (e.g., an adjusted filtrate). According to some embodiments, the adjustment module is fluidically connected (e.g., directly fluidically connected) to the bioreactor, the at least one filter, and/or the purification module. In some embodiments, the adjustment module comprises a surge tank. In certain cases, the surge tank has a volume of about 50 mL to about 2 L, about 2 L to about 10 L, or about 10 L to about 100 L. In some cases, the surge tank may advantageously facilitate connection of an upstream process associated with a first flow rate and/or pressure and a downstream process associated with a second, different flow rate and/or pressure. For instance, in some embodiments, a surge tank may help to release pressure from the filter.

In certain illustrative embodiments, the adjustment module is configured to increase or decrease the pH of the at least one filtrate to produce a pH-adjusted filtrate. In some embodiments, the pH of the adjusted filtrate is compatible with a first column of the purification module. In certain cases, adjusting the pH of the at least one filtrate may facilitate effective capture of G-CSF on the first column of the purification module. In addition, adjusting the pH of the at least one filtrate may advantageously reduce the amount of product aggregates, increase product yield, increase G-CSF stability, and/or maximize product quality. In some embodiments, the pH-adjusted filtrate has a pH of about 4.0, about 5.0, about 5.5, about 6.0, about 6.5, about 7.0, about 7.5, about 8.0, or about 9.0.

In some embodiments, the adjustment module is configured to minimize hold time after adjustment. Minimizing hold time after adjustment may, in some cases, advantageously maximize product quality. In certain embodiments, the hold time of the adjusted filtrate is about 24 hours or less, about 18 hours or less, about 12 hours or less, about 6 hours or less, about 1 hour or less, about 30 minutes or less, or about 10 minutes or less. In some embodiments, the hold time of the adjusted filtrate is in the range of about 10 minutes to about 30 minutes, about 10 minutes to about 1 hour, about 10 minutes to about 6 hours, about 10 minutes to about 12 hours, about 10 minutes to about 18 hours, about 10 minutes to about 24 hours, about 30 minutes to about 1 hour, about 30 minutes to about 6 hours, about 30 minutes to about 12 hours, about 30 minutes to about 18 hours, about 30 minutes to about 24 hours, about 1 hour to about 6 hours, about 1 hour to about 12 hours, about 1 hour to about 18 hours, about 1 hour to about 24 hours, about 6 hours to about 12 hours, about 6 hours to about 18 hours, or about 6 hours to about 24 hours.

In some embodiments, the purification module is configured to remove at least a first type of impurity, a second type of impurity, and a third type of impurity from an input stream received by the purification module (e.g., the at least one filtrate, the adjusted filtrate) to produce a purified filtrate. In some embodiments, the purification module is fluidically connected (e.g., directly fluidically connected) to the at least one filter and/or the adjustment module.

In some embodiments, the purification module comprises a first partitioning unit configured to remove at least the first type of impurity, a second partitioning unit configured to remove at least the second type of impurity, and a third partitioning unit configured to remove at least the third type of impurity. In certain embodiments, the design framework described above may be used to generate and evaluate candidate sequences of partitioning steps. Using the design framework, for example, a sequence of chromatography columns and associated conditions suitable for capturing and purifying G-CSF may be identified.

In some embodiments, the purification module comprises a first column comprising a multimodal cation exchange resin. In some embodiments, the first column is configured to remove at least the first type of impurity to produce a first partitioned filtrate that comprises G-CSF and is lean in the first type of impurity relative to the first filtrate. According to certain embodiments, the multimodal cation exchange resin comprises Capto MMC ImpRes, Capto MMC, Nuvia cPrime, Toyopearl MX-Trp-650M, CMM HyperCel, and/or Eshmuno HCX.

In some embodiments, the purification module further comprises a second column comprising an anion exchange resin (e.g., a salt-tolerant anion exchange resin). The second column may be fluidically connected (e.g., directly fluidically connected) to the first column, according to certain embodiments. In some embodiments, the second column is configured to remove at least the second type of impurity to produce a second partitioned filtrate that comprises G-CSF and is lean in the second type of impurity relative to the first partitioned filtrate. In certain cases, the anion exchange resin comprises HyperCel STAR AX and/or Toyopearl NH2-750F.

In some embodiments, the purification module further comprises a third column comprising an HCIC resin. The third column is fluidically connected (e.g., directly fluidically connected) to the second column, according to certain embodiments. In some embodiments, the third column is configured to remove at least the third type of impurity to produce a third partitioned filtrate that comprises G-CSF and is lean in the third type of impurity relative to the second partitioned filtrate. In certain cases, the HCIC resin comprises MEP HyperCel, PPA HyperCel, and/or HEA HyperCel.

In some embodiments, the system further comprises a formulation module configured to produce a formulated product stream. In some embodiments, the formulation module is fluidically connected (e.g., directly fluidically connected) to the purification module. In some embodiments, the formulation module comprises a filtration unit, a viral filtration unit, a dilution adjustment unit, and/or a product packaging unit. According to some embodiments, the filtration unit comprises a tangential flow filtration device.

In some embodiments, the product stream (e.g., the purified filtrate stream from the purification module, the formulated product stream from the formulation module) has a relatively high concentration of G-CSF. In certain embodiments, the product stream has a G-CSF concentration of at least about 0.05 mg/mL, at least about 0.1 mg/mL, at least about 0.2 mg/mL, at least about 0.3 mg/mL, at least about 0.4 mg/mL, at least about 0.5 mg/mL, at least about 1 mg/mL, at least about 5 mg/mL, at least about 10 mg/mL, at least about 25 mg/mL, at least about 50 mg/mL, at least about 75 mg/mL, or at least about at least about 90 mg/mL. In some embodiments, the product stream has a G-CSF concentration in the range of about 0.05 mg/mL to about 100 mg/mL, about 0.5 mg/mL to about 100 mg/mL, or about 1 mg/mL to about 100 mg/mL. In certain embodiments, the product stream has a G-CSF concentration in the range of about 0.05 mg/mL to about 0.2 mg/mL, about 0.05 mg/mL to about 0.3 mg/mL, about 0.05 mg/mL to about 0.4 mg/mL, about 0.05 mg/mL to about 0.5 mg/mL, about 0.1 mg/mL to about 0.3 mg/mL, about 0.1 mg/mL to about 0.4 mg/mL, about 0.1 mg/mL to about 0.5 mg/mL, about 0.2 mg/mL to about 0.3 mg/mL, about 0.2 mg/mL to about 0.4 mg/mL, about 0.2 mg/mL to about 0.5 mg/mL, or about 0.3 mg/mL to about 0.5 mg/mL.

In some embodiments, the product stream has a relatively high product yield. In certain embodiments, the product stream has a product yield of at least about 50%, at least about 60%, at least about 70%, at least about 80%, at least about 90%, at least about 95%, or at least about 99%. In certain embodiments, the product stream has a product yield in the range of about 50% to about 70%, about 50% to about 80%, about 50% to about 90%, about 50% to about 95%, about 50% to about 99%, about 60% to about 80%, about 60% to about 90%, about 60% to about 95%, about 60% to about 99%, about 70% to about 90%, about 70% to about 95%, about 70% to about 99%, about 80% to about 95%, about 80% to about 99%, or about 90% to about 99%. In some embodiments, the product stream has a relatively low concentration of impurities. In certain embodiments, for example, the product stream has a relatively low concentration of host cell proteins. In some embodiments, the product stream has a host cell protein concentration of about 100 ng/(mg G-CSF) or less, about 50 ng/(mg G-CSF) or less, about 20 ng/(mg G-CSF) or less, about 10 ng/(mg G-CSF) or less, about 8 ng/(mg G-CSF) or less, or about 5 ng/(mg G-CSF) or less. In some embodiments, the product stream has a host cell protein concentration in the range of about 0 ng/(mg G-CSF) to about 5 ng/(mg G-CSF), about 0 ng/(mg G-CSF) to about 8 ng/(mg G-CSF), about 0 ng/(mg G-CSF) to about 10 ng/(mg G-CSF), about 0 ng/(mg G-CSF) to about 20 ng/(mg G-CSF), about 0 ng/(mg G-CSF) to about 50 ng/(mg G-CSF), about 0 ng/(mg G-CSF) to about 100 ng/(mg G-CSF), about 5 ng/(mg G-CSF) to about 8 ng/(mg G-CSF), about 5 ng/(mg G-CSF) to about 10 ng/(mg G-CSF), about 5 ng/(mg G-CSF) to about 20 ng/(mg G-CSF), about 5 ng/(mg G-CSF) to about 50 ng/(mg G-CSF), about 5 ng/(mg G-CSF) to about 100 ng/(mg G-CSF), about 10 ng/(mg G-CSF) to about 20 ng/(mg G-CSF), about 10 ng/(mg G-CSF) to about 50 ng/(mg G-CSF), or about 10 ng/(mg G-CSF) to about 100 ng/(mg G-CSF).

In certain embodiments, the product stream has a relatively low concentration of DNA. For example, in some embodiments, the product stream has a DNA concentration of about 100 ng/(mg G-CSF) or less, about 50 ng/(mg G-CSF) or less, about 20 ng/(mg G-CSF) or less, about 10 ng/(mg G-CSF) or less, about 5 ng/(mg G-CSF) or less, about 1 ng/(mg G-CSF) or less, about 0.5 ng/(mg G-CSF) or less, about 0.1 ng/(mg G-CSF) or less, about 0.05 ng/(mg G-CSF) or less, about 0.01 ng/(mg G-CSF) or less, or about 0 ng/(mg G-CSF) (e.g., not detectable). In some embodiments, the product stream has a DNA concentration in the range of about 0 ng/(mg G-CSF) to about 0.01 ng/(mg G-CSF), about 0 ng/(mg G-CSF) to about 0.05 ng/(mg G-CSF), about 0 ng/(mg G-CSF) to about 0.1 ng/(mg G-CSF), about 0 ng/(mg G-CSF) to about 0.5 ng/(mg G-CSF), about 0 ng/(mg G-CSF) to about 1 ng/(mg G-CSF), about 0 ng/(mg G-CSF) to about 5 ng/(mg G-CSF), about 0 ng/(mg G-CSF) to about 10 ng/(mg G-CSF), about 0 ng/(mg G-CSF) to about 20 ng/(mg G-CSF), about 0 ng/(mg G-CSF) to about 50 ng/(mg G-CSF), about 0 ng/(mg

G-CSF) to about 100 ng/(mg G-CSF).

In some embodiments, the product stream contains a relatively low amount of aggregates (e.g., aggregates of the product). In some embodiments, the product stream has an aggregate content of about 2% or less, about 1% or less, about 0.90% or less, about 0.80% or less, about 0.70% or less, about 0.60% or less, or about 0.50% or less, about 0.40% or less, about 0.30% or less, about 0.20% or less, or about 0.10% or less. In some embodiments, the product stream has an aggregate content in the range of about 0.10% to about 0.20%, about 0.10% to about 0.30%, about 0.10% to about 0.40%, about 0.10% to about 0.50%, about 0.10% to about 0.60%, about 0.10% to about 0.70%, about 0.10% to about 0.80%, about 0.10% to about 0.90%, about 0.10% to about 1%, or about 0.10% to about 2%.

In some embodiments, the system is configured to be continuously operated. In certain embodiments, for example, the bioreactor is configured to receive at least one feed stream comprising the at least one cell culture medium. In some embodiments, the at least one filtrate is an at least one filtrate stream. In some embodiments, the purified filtrate is a purified filtrate stream.

In some embodiments, the reactor chamber of the bioreactor has a volume of about 1 L or less. In certain of these embodiments, an input stream to the system (e.g., the at least one feed stream) and an output stream of the system (e.g., the purified filtrate stream, the formulated product stream) each have a flow rate of at least about 0.1 mL/min, at least about 0.5 mL/min, at least about 1.0 mL/min, at least about 1.5 mL/min, or at least about 2 mL/min over a period of at least about 1 day. In some embodiments, the input stream and the output stream each have a flow rate in the range of 0.1 mL/min to about 0.5 mL/min, about 0.1 mL/min to about 1.0 mL/min, about 0.1 mL/min to about 1.5 mL/min, about 0.1 mL/min to about 2 mL/min, about 0.5 mL/min to about 1.0 mL/min, about 0.5 mL/min to about 1.5 mL/min, about 0.5 mL/min to about 2 mL/min, or about 1 mL/min to about 2 mL/min over a period of at least about 1 day. In some embodiments, the system is configured to produce at least about 1 mg, at least about 5 mg, at least about 10 mg, at least about 20 mg, at least about 50 mg, at least about 100 mg, at least about 200 mg, at least about 500 mg, at least about 1 g, at least about 2 g, or at least about 5 g of G-CSF per day. In some embodiments, the system is configured to produce an amount of G-CSF in the range of about 1 mg to about 5 mg, about 1 mg to about 10 mg, about 1 mg to about 20 mg, about 1 mg to about 50 mg, about 1 mg to about 100 mg, about 1 mg to about 500 mg, about 1 mg to about 1 g, about 1 mg to about 2 g, about 1 mg to about 5 g, about 10 mg to about 20 mg, about 10 mg to about 50 mg, about 10 mg to about 100 mg, about 10 mg to about 500 mg, about 10 mg to about 1 g, about 10 mg to about 2 g, about 10 mg to about 5 g, about 50 mg to about 100 mg, about 50 mg to about 500 mg, about 50 mg to about 1 g, about 50 mg to about 2 g, about 50 mg to about 5 g, about 100 mg to about 500 mg, about 100 mg to about 1 g, about 100 mg to about 2 g, about 100 mg to about 5 g, about 500 mg to about 1 g, about 500 mg to about 2 g, about 500 mg to about 5 g, or about 1 g to about 5 g per day.

In some embodiments, the reactor chamber of the bioreactor has a volume of about 1 L to about 10 L. In certain of these embodiments, an input stream to the system (e.g., the at least one feed stream) and an output stream of the system (e.g., the purified filtrate stream, the formulated product stream) each have a flow rate of at least about 0.5 mL/min, at least about 1 mL/min, at least about 5 mL/min, at least about 10 mL/min, or at least about 20 mL/min over a period of at least about 1 day. In certain embodiments, the input stream and the output stream each have a flow rate in the range of about 0.5 mL/min to about 1 mL/min, about 0.5 mL/min to about 5 mL/min, about 0.5 mL/min to about 10 mL/min, about 0.5 mL/min to about 20 mL/min, about 1 mL/min to about 5 mL/min, about 1 mL/min to about 10 mL/min, about 1 mL/min to about 20 mL/min, about 5 mL/min to 10 mL/min, about 5 mL/min to about 20 mL/min, or about 10 mL/min to about 20 mL/min over a period of at least about 1 day. In some of these embodiments, the system is configured to produce at least about 50 mg, at least about 100 mg, at least about 200 mg, at least about 500 mg, at least about 1 g, at least about 5 g, at least about 10 g, or at least about 50 g of G-CSF per day. In some embodiments, the system is configured to produce an amount of G-CSF in the range of about 50 mg to about 100 mg, about 50 mg to about 200 mg, about 50 mg to about 500 mg, about 50 mg to about 1 g, about 50 mg to about 5 g, about 50 mg to about 10 g, about 50 mg to about 50 g, about 100 mg to about 500 mg, about 100 mg to about 1 g, about 100 mg to about 5 g, about 100 mg to about 10 g, about 100 mg to about 50 g, about 500 mg to about 1 g, about 500 mg to about 5 g, about 500 mg to about 10 g, about 500 mg to about 50 g, about 1 g to about 10 g, about 1 g to about 50 g, or about 10 g to about 50 g per day.

In some embodiments, the reactor chamber of the bioreactor has a volume of about 10 L to about 50 L. In certain of these embodiments, an input stream to the system (e.g., the at least one feed stream) and an output stream of the system (e.g., the purified filtrate stream, the formulated product stream) each have a flow rate of at least about 5 mL/min, at least about 10 mL/min, at least about 20 mL/min, at least about 50 mL/min, at least about 100 mL/min, at least about 150 mL/min, or at least about 200 mL/min over a period of at least about 1 day. In some embodiments, the input stream and the output stream each have a flow rate in the range of about 5 mL/min to about 10 mL/min, about 5 mL/min to about 20 mL/min, about 5 mL/min to about 50 mL/min, about 5 mL/min to about 100 mL/min, about 5 mL/min to about 150 mL/min, about 5 mL/min to about 200 mL/min, about 10 mL/min to about 20 mL/min, about 10 mL/min to about 50 mL/min, about 10 mL/min to about 100 mL/min, about 10 mL/min to about 150 mL/min, about 10 mL/min to about 200 mL/min, about 50 mL/min to about 100 mL/min, about 50 mL/min to about 150 mL/min, about 50 mL/min to about 200 mL/min, or about 100 mL/min to about 200 mL/min over a period of at least about 1 day. In some of these embodiments, the system is configured to produce at least about 500 mg, at least about 1 g, at least about 2 g, at least about 5 g, at least about 10 g, at least about 50 g, at least about 100 g, at least about 200 g, or at least about 500 g of G-CSF per day. In some embodiments, the system is configured to produce an amount of G-CSF in the range of about 500 mg to about 1 g, about 500 mg to about 2 g, about 500 mg to about 5 g, about 500 mg to about 10 g, about 500 mg to about 50 g, about 500 mg to about 100 g, about 500 mg to about 200 g, about 500 mg to about 500 g, about 1 g to about 5 g, about 1 g to about 10 g, about 1 g to about 50 g, about 1 g to about 100 g, about 1 g to about 500 g, about 10 g to about 50 g, about 10 g to about 100 g, about 10 g to about 500 g, or about 100 g to about 500 g per day. Some embodiments described herein relate to methods for producing G-CSF. In certain embodiments, the method comprises supplying a growth cell medium to a bioreactor (e.g., a perfusion bioreactor). In some embodiments, the method further comprises incubating a first type of biological cells in the growth cell culture medium for a period of at least one day. In some embodiments, the method further comprises at least partially removing the growth cell culture medium from the bioreactor. In some embodiments, the method further comprises supplying at least one cell culture medium (e.g., a production cell culture medium) to the bioreactor. In some embodiments, the method comprises producing, within the bioreactor, a suspension comprising the at least one cell culture medium and at least a first type of biological cells expressing G-CSF.

In some embodiments, the method further comprises causing at least a portion of the suspension to flow through at least one filter to produce at least one filtrate lean in the first type of biological cells. In some embodiments, the at least one filtrate comprises G-CSF.

In some embodiments, the method comprises flowing the at least one filtrate to an adjustment module (e.g., from the at least one filter to the adjustment module). In some embodiments, the method further comprises adjusting, within the adjustment module, one or more properties (e.g., pH, conductivity, product stability) of the at least one filtrate. In certain embodiments, the pH of the at least one filtrate is increased or decreased (e.g., to be compatible with the first column of the purification module). In some embodiments, increasing the pH of the at least one filtrate comprises adding a base to the at least one filtrate. In some embodiments, decreasing the pH of the at least one filtrate comprises adding an acid to the at least one filtrate. In some embodiments, the method further comprises flowing the at least one filtrate and/or the adjusted filtrate through a first column comprising a multimodal cation exchange resin. In certain embodiments, the multimodal cation exchange resin comprises Capto MMC ImpRes, Capto MMC, Nuvia cPrime, Toyopearl MX-Trp-650M, CMM HyperCel, and/or Eshmuno HCX. In certain non-limiting embodiments, the multimodal cation exchange resin comprises a Capto MMC ImpRes resin.

In certain embodiments, the first column is operated in bind-elute mode. In some embodiments, the method further comprises flowing a first mobile phase material through the first column. In some embodiments, the first mobile phase material is configured to promote binding of G-CSF to the multimodal cation exchange resin. In some embodiments, the first mobile phase material comprises sodium citrate, sodium phosphate, sodium chloride, sodium acetate, Tris-HC1, glycine, and/or histidine. According to certain embodiments, the first mobile phase material comprises 20 mM sodium phosphate and/or 20 mM sodium citrate. In some embodiments, the first mobile phase material has a pH of about 4.0, about 4.5, about 5.0, about 5.5, or about 6.0. In some embodiments, the method further comprises flowing the at least one filtrate and/or the adjusted filtrate through the first column and, subsequently, flowing a second mobile phase material through the first column. In some embodiments, the second mobile phase material is configured to wash one or more impurities from the multimodal cation exchange resin. In some embodiments, the second mobile phase material comprises sodium citrate, sodium phosphate, sodium chloride, sodium acetate, Tris-HCl, glycine, and/or histidine. According to certain embodiments, the second mobile phase material comprises 20 mM sodium phosphate and/or 20 mM sodium citrate. In some embodiments, the second mobile phase material has a pH of about 4.5, about 5.0, about 5.5, about 5.8, about 6.0, about 6.5, or about 7.0. In some embodiments, the second mobile phase material has a salt (e.g., sodium chloride) concentration of about 100 mM, about 125 mM, about 150 mM, about 175 mM, or about 200 mM. In some embodiments, the method further comprises subsequently flowing a third mobile phase material through the first column. In some embodiments, the third mobile phase material is configured to elute G-CSF from the multimodal cation exchange resin. In some embodiments, the third mobile phase material comprises sodium citrate, sodium phosphate, sodium chloride, sodium acetate, Tris-HC1, glycine, and/or histidine. According to certain embodiments, the third mobile phase material comprises 20 mM sodium phosphate and/or 20 mM sodium citrate. In some embodiments, the third mobile phase material has a pH of about 6.0, 6.5, 7.0, 7.5, or 8.0. In some embodiments, the third mobile phase material has a salt (e.g., sodium chloride) concentration of about 100 mM, about 125 mM, about 150 mM, about 175 mM, or about 200 mM. In some embodiments, the method further comprises collecting one or more first fractions comprising G-CSF from an outflow of the first column. In some embodiments, the one or more first fractions are lean in the first type of impurity relative to the at least one filtrate or the adjusted filtrate. In some embodiments, the one or more first fractions have a concentration of the first type of impurity that is at least about 50%, at least about 75%, at least about 90%, at least about 95%, or at least about 99% less than the concentration of the first type of impurity in the at least one filtrate or the adjusted filtrate.

In some embodiments, the method further comprises flowing the one or more first fractions through a second column comprising an anion exchange resin. In some embodiments, the anion exchange resin comprises a HyperCel STAR AX resin and/or a Toyopearl NH2-750F resin. In certain embodiments, the second column is operated in flow-through mode. In some embodiments, the method comprises flowing through a first mobile phase material through the second column. In some embodiments, the first mobile phase material comprises sodium citrate, sodium phosphate, sodium chloride, sodium acetate, Tris-HCl, glycine, and/or histidine. According to certain embodiments, the first mobile phase material comprises 20 mM sodium phosphate and/or 20 mM sodium citrate. In some embodiments, the first mobile phase material has a pH of about 6.0, about 6.5, about 7.0, about 7.5, or about 8.0. In some embodiments, the first mobile phase material has a salt (e.g., sodium chloride) concentration of about 100 mM, about 125 mM, about 150 mM, about 175 mM, or about 200 mM. In some embodiments, the method further comprises collecting one or more second fractions comprising G-CSF from an outflow of the second column. In some embodiments, the one or more second fractions are lean in the second type of impurity relative to the first fractions. In some embodiments, the one or more second fractions have a concentration of the second type of impurity that is at least about 50%, at least about 75%, at least about 90%, at least about 95%, or at least about 99% less than the concentration of the second type of impurity in the first fractions.

In some embodiments, the method further comprises flowing the one or more second fractions through a third column comprising an HCIC resin. In some embodiments, the HCIC resin comprises an MEP HyperCel resin, a PPA HyperCel resin, and/or an HEA HyperCel resin. In some embodiments, the third column is operated in bind-elute mode. In some embodiments, the method further comprises flowing a first mobile phase material through the third column prior to flowing the second fractions through the third column. In some embodiments, the first mobile phase material is configured to promote binding of G-CSF to the HCIC resin. In some embodiments, the first mobile phase material has a pH of about 6.0, about 6.5, about 7.0, about 7.5, or about 8.0. In some embodiments, the first mobile phase material has a salt concentration of about 100 mM, about 125 mM, about 150 mM, about 175 mM, or about 200 mM. In some embodiments, the method further comprises flowing a second mobile phase material through the third column after flowing the second fractions through the third column. In some embodiments, the second mobile phase material has a pH of about 4.5, about 5.0, about 5.5, about 6.0, or about 6.5. In some embodiments, the second mobile phase has a salt concentration less than about 200 mM, about 175 mM, about 150 mM, about 125 mM, or about 100 mM. In some embodiments, the method further comprises flowing a third mobile phase material through the third column. In some embodiments, the third mobile phase material is configured to elute G-CSF from the third column. In some embodiments, the third mobile phase material has a pH of about 2.0, about 2.5, about 3.0, about 3.5, or about 4.0. In some embodiments, the third mobile phase material has a salt concentration less than about 200 mM, about 175 mM, about 150 mM, about 125 mM, or about 100 mM. In some embodiments, the method further comprises collecting one or more third fractions comprising G-CSF from an outflow of the third column. In some embodiments, the one or more third fractions are lean in a third type of impurity relative to the second fractions. In some embodiments, the one or more third fractions have a concentration of the third type of impurity that is at least about 50%, at least about 75%, at least about 90%, at least about 95%, or at least about 99% less than the concentration of the third type of impurity in the second fractions. In some embodiments, the third fractions are collected as a biologically-produced product stream.

In some embodiments, the method further comprises flowing the third fractions to a formulation module to produce a formulated product stream. In some embodiments, flowing the purified filtrate through the formulation module comprises flowing the purified filtrate through a tangential flow filtration device. In some embodiments, flowing the purified filtrate through the formulation module comprises flowing the purified filtrate through a viral filtration unit. In some embodiments, the formulated product stream is lean in one or more viruses relative to the purified filtrate stream. In some embodiments, flowing the purified filtrate through the formulation module comprises flowing the purified filtrate through a dilution adjustment unit. In some embodiments, flowing the purified filtrate through the dilution adjustment unit comprises adding a diluent to the purified filtrate. In some embodiments, flowing the purified filtrate through the formulation module comprises depositing one or more portions of the purified filtrate stream into one or more containers (e.g., bags, vials, syringes, bottles). In some embodiments, the one or more containers are aseptic and/or sterile containers.

hGH-specific systems and methods

Some embodiments described herein relate to systems for producing human growth hormone (hGH). According to some embodiments, the system comprises a bioreactor (e.g., a perfusion bioreactor). The bioreactor may comprise a reaction chamber, and, as described above, may have any suitable shape and be formed of any suitable material. In some embodiments, the reaction chamber contains a suspension comprising at least one cell culture medium and at least a first type of biological cells configured to express hGH. In some embodiments, the biological cells are yeast cells. In some embodiments, the yeast cells are Pichia pastoris cells. In some embodiments, for example during a cell growth phase, the at least one cell culture medium comprises chemically defined media comprising a carbon source or buffered glycerol-complex medium (BMGY). In some embodiments, for example during an hGH production phase, the at least one cell culture medium comprises chemically defined media comprising a carbon source and/or other additive for induction of protein expression or buffered methanol-complex media (BMMY).

In some embodiments, the system further comprises at least one filter. In certain embodiments, the at least one filter comprises a filter probe, a filtration membrane, and/or a ceramic filter. The at least one filter may, in some embodiments, be fluidically connected (e.g., directly fluidically connected) to the bioreactor. For example, the at least one filter may comprise at least one filter probe at least partially submerged in the suspension in the bioreactor. In some embodiments, the at least one filter is configured to receive an output of the bioreactor and produce at least one filtrate lean in the first type of biological cells relative to the suspension. In some embodiments, the at least one filtrate comprises hGH.

In some embodiments, the system further comprises an adjustment module configured to adjust one or more properties (e.g., pH, conductivity, product stability) of an input stream received by the adjustment module (e.g., the at least one filtrate) to produce an adjusted output stream (e.g., an adjusted filtrate). In some embodiments, the adjustment module is fluidically connected (e.g., directly fluidically connected) to the bioreactor, the at least one filter, and/or the purification module. In some embodiments, the adjustment module comprises a surge tank. In certain cases, the surge tank has a volume of about 50 mL to about 2 L, about 2 L to about 10 L, or about 10 L to about 100 L. In some cases, the surge tank may advantageously facilitate connection of an upstream process associated with a first flow rate and/or pressure and a downstream process associated with a second, different flow rate and/or pressure. For instance, in some embodiments, a surge tank may help to release pressure from the filter.

In certain illustrative embodiments, the adjustment module is configured to increase or decrease the pH of the at least one filtrate to produce a pH-adjusted filtrate. In some embodiments, the pH of the adjusted filtrate is compatible with a first column of the purification module. In certain cases, adjusting the pH of the at least one filtrate may facilitate effective capture of hGH on the first column of the purification module. In addition, adjusting the pH of the at least one filtrate may advantageously reduce the amount of product aggregates, increase product yield, increase hGH stability, and/or increase product quality. In some embodiments, the pH-adjusted filtrate has a pH of about 4.0, about 5.0, about 5.5, about 6.0, about 6.5, about 7.0, about 7.5, about 8.0, or about 9.0.

In some embodiments, the adjustment module is configured to minimize hold time after adjustment. Minimizing hold time after adjustment may, in some cases, advantageously maximize product quality. In certain embodiments, the hold time of the adjusted filtrate is about 12 hours or less, about 6 hours or less, about 1 hour or less, about 30 minutes or less, or about 10 minutes or less. In some embodiments, the hold time of the adjusted filtrate is in the range of about 10 minutes to about 30 minutes, about 10 minutes to about 1 hour, about 10 minutes to about 6 hours, about 10 minutes to about 12 hours, about 10 minutes to about 18 hours, about 10 minutes to about 24 hours, about 30 minutes to about 1 hour, about 30 minutes to about 6 hours, about 30 minutes to about 12 hours, about 30 minutes to about 18 hour, about 30 minutes to about 24 hours, about 1 hour to about 6 hours, about 1 hour to about 12 hours, about 1 hour to about 18 hours, about 1 hour to about 24 hours, about 6 hour to about 12 hours, about 6 hour to about 18 hours, or about 6 hours to about 24 hours.

In some embodiments, the system further comprises a purification module configured to remove at least a first type of impurity, a second type of impurity, and a third type of impurity from an input stream received by the purification module (e.g., the at least one filtrate, the adjusted filtrate) to produce a purified filtrate. In some embodiments, the purification module is fluidically connected (e.g., directly fluidically connected) to the at least one filter and/or the adjustment module.

In some embodiments, the purification module comprises a first partitioning unit configured to remove at least the first type of impurity, a second partitioning unit configured to remove at least the second type of impurity, and a third partitioning unit configured to remove at least the third type of impurity. In certain embodiments, the design framework described above may be used to generate and evaluate candidate sequences of partitioning steps. Using the design framework, for example, a sequence of chromatography columns and associated conditions suitable for capturing and purifying hGH may be identified.

In some embodiments, the purification module comprises a first column comprising a multimodal cation exchange resin. In some embodiments, the first column is configured to remove at least the first type of impurity to produce a first partitioned filtrate lean in the first type of impurity relative to the first filtrate. In some embodiments, the first partitioned filtrate comprises hGH. In some embodiments, the multimodal cation exchange resin comprises Capto MMC, Capto MMC ImpRes, Nuvia cPrime, Toyopearl MX-Trp-650M, CMM HyperCel, and/or Eshmuno HCX.

In some embodiments, the purification module further comprises a second column comprising an anion exchange resin. In some embodiments, the second column is fluidically connected (e.g., directly fluidically connected) to the first column. In some embodiments, the second column is configured to remove at least the second type of impurity to produce a second partitioned filtrate lean in the second type of impurity relative to the first partitioned filtrate. In some embodiments, the second partitioned filtrate comprises hGH. In some embodiments, the anion exchange resin comprises HyperCel STAR AX and/or Toyopearl NH2-750F.

In some embodiments, the purification module optionally comprises a third column comprising an HCIC resin. In some embodiments, the third column is fluidically connected (e.g., directly fluidically connected) to the second column. In some embodiments, the third column is configured to remove at least the third type of impurity to produce a third partitioned filtrate lean in the third type of impurity relative to the second partitioned filtrate. In some embodiments, the third partitioned filtrate comprises hGH. In some embodiments, the HCIC resin comprises MEP HyperCel, PPA HyperCel, and/or HEA HyperCel.

In some embodiments, the system further comprises a formulation module configured to produce a formulated product stream. In some embodiments, the formulation module is fluidically connected (e.g., directly fluidically connected) to the purification module. In some embodiments, the formulation module comprises a filtration unit, a viral filtration unit, and/or a product packaging unit. In some embodiments, the filtration unit comprises a tangential flow filtration device.

In some embodiments, the product stream (e.g., the purified filtrate stream from the purification module, the formulated product stream from the formulation module) has a relatively high concentration of hGH. In certain embodiments, the product stream has an hGH concentration of at least about 0.05 mg/mL, at least about 0.1 mg/mL, at least about 0.2 mg/mL, at least about 0.3 mg/mL, at least about 0.4 mg/mL, at least about 0.5 mg/mL, at least about 0.6 mg/mL, at least about 0.7 mg/mL, at least about 0.8 mg/mL, at least about 0.9 mg/mL, at least about 1 mg/mL, at least about 5 mg/mL, at least about 10 mg/mL, at least about 25 mg/mL, at least about 50 mg/mL, at least about 75 mg/mL, or at least about at least about 90 mg/mL. In some embodiments, the product stream has a hGH concentration in the range of about 0.05 mg/mL to about 100 mg/mL, about 0.5 mg/mL to about 100 mg/mL, or about 1 mg/mL to about 100 mg/mL. In certain embodiments, the product stream has an hGH concentration in the range of about 0.05 mg/mL to about 0.5 mg/mL, about 0.05 mg/mL to about 0.6 mg/mL, about 0.05 mg/mL to about 0.7 mg/mL, about 0.05 mg/mL to about 0.8 mg/mL, about 0.05 mg/mL to about 0.9 mg/mL, about 0.05 mg/mL to about 1.0 mg/mL, about 0.1 mg/mL to about 0.5 mg/mL, about 0.1 mg/mL to about 0.6 mg/mL, about 0.1 mg/mL to about 0.7 mg/mL, about 0.1 mg/mL to about 0.8 mg/mL, about 0.1 mg/mL to about 0.9 mg/mL, about 0.1 mg/mL to about 1 mg/mL, about 0.5 mg/mL to about 0.7 mg/mL, about 0.5 mg/mL to about 0.8 mg/mL, about 0.5 mg/mL to about 0.9 mg/mL, about 0.5 mg/mL to about 1 mg/mL, about 0.6 mg/mL to about 0.8 mg/mL, about 0.6 mg/mL to about 0.9 mg/mL, about 0.6 mg/mL to about 1 mg/mL, or about 0.8 mg/mL to about 1 mg/mL.

In some embodiments, the product stream has a relatively high product yield. In certain embodiments, the product stream has a product yield of at least about 50%, at least about 60%, at least about 70%, at least about 80%, at least about 90%, at least about 95%, or at least about 99%. In certain embodiments, the product stream has a product yield in the range of about 50% to about 70%, about 50% to about 80%, about 50% to about 90%, about 50% to about 95%, about 50% to about 99%, about 60% to about 80%, about 60% to about 90%, about 60% to about 95%, about 60% to about 99%, about 70% to about 90%, about 70% to about 95%, about 70% to about 99%, about 80% to about 95%, about 80% to about 99%, or about 90% to about 99%.

In some embodiments, the product stream (e.g., the purified filtrate stream from the purification module, the formulated pharmaceutical product stream from the formulation module) has a relatively low concentration of impurities. In certain embodiments, for example, the product stream has a relatively low concentration of host cell proteins. In some embodiments, the product stream has a host cell protein concentration of about 50 ng/(mg hGH) or less, about 20 ng/(mg hGH) or less, about 15 ng/(mg hGH) or less, about 13.72 ng/(mg hGH) or less, about 10 ng/(mg hGH) or less, or about 5 ng/(mg hGH) or less. In some embodiments, the product stream has a host cell protein concentration in the range of about 0 ng/(mg hGH) to about 5 ng/(mg hGH), about 0 ng/(mg hGH) to about 10 ng/(mg hGH), about 0 ng/(mg hGH) to about 13.72 ng/(mg hGH), about 0 ng/(mg hGH) to about 15 ng/(mg hGH), about 0 ng/(mg hGH) to about 20 ng/(mg hGH), about 0 ng/(mg hGH) to about 50 ng/(mg hGH), about 5 ng/(mg hGH) to about 10 ng/(mg hGH), about 5 ng/(mg hGH) to about 13.72 ng/(mg hGH), about 5 ng/(mg hGH) to about 15 ng/(mg hGH), about 5 ng/(mg hGH) to about 20 ng/(mg hGH), or about 5 ng/(mg hGH) to about 50 ng/(mg hGH).

In certain embodiments, the product stream has a relatively low concentration of DNA. For example, in some embodiments, the product stream has a DNA concentration of about 500 ng/(mg hGH) or less, about 200 ng/(mg hGH) or less, about 100 ng/(mg hGH) or less, about 77 ng/(mg hGH) or less, about 50 ng/(mg hGH) or less, about 20 ng/(mg hGH) or less, or about 10 ng/(mg hGH) or less. In some embodiments, the product stream has a DNA concentration in the range of about 10 ng/(mg hGH) to about 50 ng/(mg hGH), about 10 ng/(mg hGH) to about 100 ng/(mg hGH), about 10 ng/(mg hGH) to about 200 ng/(mg hGH), about 10 ng/(mg hGH) to about 500 ng/(mg hGH), about 50 ng/(mg hGH) to about 100 ng/(mg hGH), about 50 ng/(mg hGH) to about 200 ng/(mg hGH), or about 50 ng/(mg hGH) to about 500 ng/(mg hGH).

In some embodiments, the product stream contains a relatively low amount of aggregates (e.g., aggregates of the product). In some embodiments, the product stream has an aggregate content of about 2% or less, about 1% or less, about 0.5% or less, about 0.48% or less, about 0.4% or less, about 0.3% or less, about 0.2% or less, or about 0.1% or less. In some embodiments, the product stream has an aggregate content in the range of about 0.10% to about 0.20%, about 0.10% to about 0.30%, about 0.10% to about 0.40%, about 0.10% to about 0.50%, about 0.10% to about 0.60%, about 0.10% to about 0.70%, about 0.10% to about 0.80%, about 0.10% to about 0.90%, about 0.10% to about 1%, or about 0.10% to about 2%.

In some embodiments, the system is configured to be continuously operated. In certain embodiments, for example, the bioreactor is configured to receive at least one feed stream comprising the at least one cell culture medium. In some embodiments, the at least one filtrate is an at least one filtrate stream. In some embodiments, the purified filtrate is a purified filtrate stream.

In some embodiments, the reactor chamber of the bioreactor has a volume of about 1 L or less. In certain of these embodiments, an input stream to the system (e.g., the at least one feed stream) and an output stream of the system (e.g., the purified filtrate stream, the formulated product stream) each have a flow rate of at least about 0.1 mL/min, at least about 0.5 mL/min, at least about 1.0 mL/min, at least about 1.5 mL/min, or at least about 2 mL/min over a period of at least about 1 day. In some embodiments, the input stream and the output stream each have a flow rate in the range of 0.1 mL/min to about 0.5 mL/min, about 0.1 mL/min to about 1.0 mL/min, about 0.1 mL/min to about 1.5 mL/min, about 0.1 mL/min to about 2 mL/min, about 0.5 mL/min to about 1.0 mL/min, about 0.5 mL/min to about 1.5 mL/min, about 0.5 mL/min to about 2 mL/min, or about 1 mL/min to about 2 mL/min over a period of at least about 1 day. In some embodiments, the system is configured to produce at least about 1 mg, at least about 5 mg, at least about 10 mg, at least about 20 mg, at least about 50 mg, at least about 100 mg, at least about 500 mg, at least about 1 g, at least about 2 g, at least about 3 g, at least about 4 g, or at least about 50 mg of hGH per day. In some embodiments, the system is configured to produce an amount of hGH in the range of about 1 mg to about 5 mg, about 1 mg to about 10 mg, about 1 mg to about 20 mg, about 1 mg to about 50 mg, about 1 mg to about 100 mg, about 1 mg to about 500 mg, about 1 mg to about 1 g, about 1 mg to about 2 g, about 1 mg to about 3 g, about 1 mg to about 4 g, about 1 mg to about 5 g, about 5 mg to about 10 mg, about 5 mg to about 20 mg, about 5 mg to about 50 mg, about 5 mg to about 100 mg, about 5 mg to about 500 mg, about 5 mg to about 1 g, about 10 mg to about 20 mg, about 10 mg to about 50 mg, about 10 mg to about 100 mg, about 10 mg to about 500 mg, about 10 mg to about 1 g, about 10 mg to about 2 g, about 10 mg to about 3 g, about 10 mg to about 4 g, about 10 mg to about 5 g, about 20 mg to about 50 mg, about 20 mg to about 100 mg, about 20 mg to about 500 mg, about 20 mg to about 1 g, about 20 mg to about 2 g, about 20 mg to about 3 g, about 20 mg to about 4 g, or about 20 mg to about 50 mg per day, about 100 mg to about 500 mg, about 100 mg to about 1 g, about 100 mg to about 2 g, about 100 mg to about 3 g, about 100 mg to about 4 g, about 100 mg to about 5 g, about 500 mg to about 1 g, about 500 mg to about 2 g, about 500 mg to about 3 g, about 500 mg to about 4 g, about 500 mg to about 5 g, about 1 g to about 2 g, about 1 g to about 3 g, about 1 g to about 4 g, or about 1 g to about 5 g.

In some embodiments, the reactor chamber of the bioreactor has a volume of about 1 L to about 10 L. In certain of these embodiments, an input stream to the system (e.g., the at least one feed stream) and an output stream of the system (e.g., the purified filtrate stream, the formulated product stream) each have a flow rate of at least about 0.5 mL/min, at least about 1 mL/min, at least about 5 mL/min, at least about 10 mL/min, or at least about 20 mL/min over a period of at least about 1 day. In certain embodiments, the input stream and the output stream each have a flow rate in the range of about 0.5 mL/min to about 1 mL/min, about 0.5 mL/min to about 5 mL/min, about 0.5 mL/min to about 10 mL/min, about 0.5 mL/min to about 20 mL/min, about 1 mL/min to about 5 mL/min, about 1 mL/min to about 10 mL/min, about 1 mL/min to about 20 mL/min, about 5 mL/min to 10 mL/min, about 5 mL/min to about 20 mL/min, or about 10 mL/min to about 20 mL/min over a period of at least about 1 day. In some of these embodiments, the system is configured to produce at least about 50 mg, at least about 100 mg, at least about 200 mg, at least about 500 mg, at least about 1 g, at least about 5 g, at least about 10 g, or at least about 500 mg of hGH per day. In some embodiments, the system is configured to produce an amount of hGH in the range of about 50 mg to about 100 mg, about 50 mg to about 200 mg, about 50 mg to about 500 mg, 50 mg to about 1 g, about 50 mg to about 5 g, about 50 mg to about 10 g, about 50 mg to about 50 g, about 100 mg to about 200 mg, about 100 mg to about 500 mg, about 100 mg to about 1 g, about 100 mg to about 5 g, about 100 mg to about 10 g, about 100 mg to about 50 g, or about 200 mg to about 500 mg, about 200 mg to about 1 g, about 200 mg to about 5 g, about 200 mg to about 10 g, or about 200 mg to about 500 mg, about 500 mg to about 1 g, about 500 mg to about 5 g, about 500 mg to about 10 g, about 500 mg to about 50 g, about 1 g to about 5 g, about 1 g to about 10 g, about 1 g to about 50 g, about 5 g to about 10 g, about 5 g to about 50 g, or about 10 g to about 50 g per day.

In some embodiments, the reactor chamber of the bioreactor has a volume of about 10 L to about 50 L. In certain of these embodiments, an input stream to the system (e.g., the at least one feed stream) and an output stream of the system (e.g., the purified filtrate stream, the formulated product stream) each have a flow rate of at least about 5 mL/min, at least about 10 mL/min, at least about 20 mL/min, at least about 50 mL/min, at least about 100 mL/min, at least about 150 mL/min, or at least about 200 mL/min over a period of at least about 1 day. In some embodiments, the input stream and the output stream each have a flow rate in the range of about 5 mL/min to about 10 mL/min, about 5 mL/min to about 20 mL/min, about 5 mL/min to about 50 mL/min, about 5 mL/min to about 100 mL/min, about 5 mL/min to about 150 mL/min, about 5 mL/min to about 200 mL/min, about 10 mL/min to about 20 mL/min, about 10 mL/min to about 50 mL/min, about 10 mL/min to about 100 mL/min, about 10 mL/min to about 150 mL/min, about 10 mL/min to about 200 mL/min, about 50 mL/min to about 100 mL/min, about 50 mL/min to about 150 mL/min, about 50 mL/min to about 200 mL/min, or about 100 mL/min to about 200 mL/min over a period of at least about 1 day. In some of these embodiments, the system is configured to produce at least about 500 mg, at least about 1 g, at least about 2 g, at least about 5 g, at least about 10 g, at least about 50 g, at least about 100 g, or at least about 250 g of hGH per day. In some embodiments, the system is configured to produce an amount of hGH in the range of about 500 mg to about 1 g, about 500 mg to about 2 g, about 500 mg to about 5 g, about 500 mg to about 10 g, about 500 mg to about 50 g, about 500 mg to about 100 g, about 500 mg to about 250 g, about 1 g to about 5 g, about 1 g to about 10 g, about 1 g to about 50 g, about 1 g to about 100 g, about 1 g to about 250 g, about 10 g to about 50 g, about 10 g to about 100 g, or about 2 10 g to about 250 g, about 50 g to about 100 g, about 50 g to about 250 g, or about 100 g to about 250 g per day.

Some embodiments described herein relate to methods for producing hGH. In certain embodiments, the method comprises supplying a growth cell medium to a bioreactor (e.g., a perfusion bioreactor). In some embodiments, the method further comprises incubating a first type of biological cells in the growth cell culture medium for a period of at least one day. In some embodiments, the method further comprises at least partially removing the growth cell culture medium from the bioreactor. In some embodiments, the method comprises supplying at least one cell culture medium (e.g., a production cell culture medium) to the bioreactor. In some embodiments, the method comprises producing, within the bioreactor, a suspension comprising the at least one cell culture medium and at least a first type of biological cells comprising hGH.

In some embodiments, the method further comprises causing at least a portion of the suspension to flow through at least one filter to produce at least one filtrate lean in the first type of biological cells. In some embodiments, the at least one filtrate comprises hGH.

In some embodiments, the method comprises flowing the at least one filtrate to an adjustment module (e.g., from the at least one filter to the adjustment module). In some embodiments, the method further comprises adjusting, within the adjustment module, one or more properties (e.g., pH, conductivity, product stability) of the at least one filtrate. In certain embodiments, the pH of the at least one filtrate is increased or decreased (e.g., to be compatible with the first column of the purification module). In some embodiments, increasing the pH of the at least one filtrate comprises adding a base to the at least one filtrate. In some embodiments, decreasing the pH of the at least one filtrate comprises adding an acid to the at least one filtrate.

In some embodiments, the method further comprises flowing the at least one filtrate and/or the adjusted filtrate through a first column comprising a multimodal cation exchange resin. In certain embodiments, the multimodal cation exchange resin comprises Capto MMC ImpRes, Capto MMC, Nuvia cPrime, Toyopearl MX-Trp-650M, CMM HyperCel, and/or Eshmuno HCX. In certain embodiments, the multimodal cation exchange resin is a Capto MMC resin.

In certain embodiments, the first column is operated in bind-elute mode. In some embodiments, the method further comprises flowing a first mobile phase material through the first column. In some embodiments, the first mobile phase material is configured to promote binding of hGH to the multimodal cation exchange resin. In some embodiments, the first mobile phase material comprises sodium citrate, sodium phosphate, sodium chloride, sodium acetate, Tris-HCl, glycine, and/or histidine. According to certain embodiments, the first mobile phase material comprises 20 mM sodium phosphate and/or 20 mM sodium citrate. In some embodiments, the first mobile phase material has a pH of about 4.0, about 4.5, about 5.0. about 5.5, or about 6.0. In some embodiments, the method further comprises flowing the at least one filtrate and/or the adjusted filtrate through the first column and, subsequently, flowing a second mobile phase material through the first column. In some embodiments, the second mobile phase material is configured to wash one or more impurities from the multimodal cation exchange resin. In some embodiments, the second mobile phase material comprises sodium citrate, sodium phosphate, sodium chloride, sodium acetate, Tris-HCl, glycine, and/or histidine. According to certain embodiments, the second mobile phase material comprises 20 mM sodium phosphate and/or 20 mM sodium citrate. In some embodiments, the second mobile phase material has a pH of about 4.0, about 4.5, about 5.0, about 5.5, or about 6.0. In some embodiments, the second mobile phase material has a sodium chloride concentration of about 400 mM, about 450 mM, about 475 mM, about 500 mM, about 525 mM, about 550 mM, or about 600 mM. In some embodiments, the method further comprise subsequently flowing a third mobile phase material through the first column. In some embodiments, the third mobile phase material is configured to elute hGH from the multimodal cation exchange resin. In some embodiments, the third mobile phase material comprises sodium citrate, sodium phosphate, sodium chloride, sodium acetate, Tris-HC1, glycine, and/or histidine. According to certain embodiments, the third mobile phase material comprises 20 mM sodium phosphate and/or 20 mM sodium citrate. In some embodiments, the third mobile phase material has a pH of about 5.0, about 5.5, about 6.0, about 6.5, or about 7.0. In some embodiments, the third mobile phase material has a sodium chloride concentration of about 50 mM, about 75 mM, about 100 mM, about 125 mM, or about 150 mM. In some embodiments, the method further comprises collecting one or more first fractions comprising hGH from an outflow of the first column. In some embodiments, the one or more first fractions are lean in the first type of impurity relative to the at least one filtrate or the adjusted filtrate. In some embodiments, the one or more first fractions have a concentration of the first type of impurity that is at least about 50%, at least about 75%, at least about 90%, at least about 95%, or at least about 99% less than the concentration of the first type of impurity in the at least one filtrate or the adjusted filtrate.

In some embodiments, the method further comprises flowing the one or more first fractions through a second column comprising an anion exchange resin. In some embodiments, the anion exchange resin comprises a HyperCel STAR AX resin and/or a Toyopearl NH2-750F resin. In certain embodiments, the second column is operated in flow-through mode. In some embodiments, the method comprises flowing through a first mobile phase material through the second column. In some embodiments, the first mobile phase material comprises sodium citrate, sodium phosphate, sodium chloride, sodium acetate, Tris-HCl, glycine, and/or histidine. According to certain embodiments, the first mobile phase material comprises 20 mM sodium phosphate and/or 20 mM sodium citrate. In some embodiments, the first mobile phase material has a pH of about 6.0, about 6.5, about 7.0, about 7.5, or about 8.0. In some embodiments, the first mobile phase material has a salt (e.g., sodium chloride) concentration of about 100 mM, about 125 mM, about 150 mM, about 175 mM, or about 200 mM. In some embodiments, the method further comprises collecting one or more second fractions comprising hGH from an outflow of the second column. In some embodiments, the one or more second fractions are lean in the second type of impurity relative to the first fractions. In some embodiments, the one or more second fractions have a concentration of the second type of impurity that is at least about 50%, at least about 75%, at least about 90%, at least about 95%, or at least about 99% less than the concentration of the second type of impurity in the first fractions.

In some embodiments, the method further comprises flowing the one or more second fractions through a third column comprising an HCIC resin. In some embodiments, the HCIC resin comprises an MEP HyperCel resin, a PPA HyperCel resin, and/or an HEA HyperCel resin. In some embodiments, the third column is operated in bind-elute mode. In some embodiments, the method further comprises flowing a first mobile phase material through the third column prior to flowing the second fractions through the third column. In some embodiments, the first mobile phase material is configured to promote binding of hGH to the HCIC resin. In some embodiments, the first mobile phase material has a pH of about 5.0, about 5.5, about 6.0, about 6.5, or about 7.0. In some embodiments, the first mobile phase material has a sodium chloride concentration of about 50 mM, about 75 mM, about 100 mM, about 125 mM, or about 150 mM. In some embodiments, the method further comprises flowing a second mobile phase material through the third column after flowing the second fractions through the third column. In some embodiments, the second mobile phase material has a pH of about 4.0, about 4.5, about 5.1, about 5.5, or about 6.0. In some embodiments, the second mobile phase material has a sodium chloride concentration less than about 200 nm, about 150 nm, about 100 nm, or about 50 nm. In some embodiments, the method further comprises flowing a third mobile phase material through the third column. In some embodiments, the third mobile phase material is configured to elute hGH from the third column. In some embodiments, the third mobile phase material has a pH of about 2.0, about 2.5, about 3.0, about 3.5, or about 4.0. In some embodiments, the third mobile phase material has a sodium chloride concentration less than about 200 mM, about 150 mM, about 100 mM, or about 50 mM. In some embodiments, the method further comprises collecting one or more third fractions comprising hGH from an outflow of the third column. In some embodiments, the one or more third fractions are lean in a third type of impurity relative to the second fractions. In some embodiments, the one or more third fractions have a concentration of the third type of impurity that is at least about 50%, at least about 75%, at least about 90%, at least about 95%, or at least about 99% less than the concentration of the third type of impurity in the second fractions. In some embodiments, the third fractions are collected as a biologically-produced product stream.

In some embodiments, the method further comprises flowing the third fractions to a formulation module to produce a formulated product stream. In some embodiments, flowing the purified filtrate through the formulation module comprises flowing the purified filtrate through a tangential flow filtration device. In some embodiments, flowing the purified filtrate through the formulation module comprises flowing the purified filtrate through a viral filtration unit. In some embodiments, the formulated product stream is lean in one or more viruses relative to the purified filtrate stream. In some embodiments, flowing the purified filtrate through the formulation module comprises depositing one or more portions of the purified filtrate stream into one or more containers (e.g., bags, vials, syringes, bottles). In some embodiments, the one or more containers are aseptic and/or sterile containers.

### IFN-Specific Systems and Methods

Some embodiments described herein relate to systems for producing interferon α-2b (IFN-α2b). In some embodiments, the system comprises a bioreactor (e.g., a perfusion bioreactor). In some embodiments, the bioreactor comprises a reaction chamber containing suspension comprising at least one cell culture medium and at least a first type of biological cells configured to express IFN-α2b. In some embodiments, the biological cells are yeast cells. In some embodiments, the yeast cells are Pichia pastoris cells. In some embodiments, for example during a cell growth phase, the at least one cell culture medium comprises chemically defined media comprising a carbon source or buffered glycerol-complex medium (BMGY). In some embodiments, for example during an IFN-α2b production phase, the at least one cell culture medium comprises chemically defined media comprising a carbon source and/or other additive for induction of protein expression or buffered methanol-complex media (BMMY).

In some embodiments, the system further comprises a filter. In some embodiments, the at least one filter of the system comprises a filter probe, a filtration membrane, and/or a ceramic filter. The at least one filter may, in some embodiments, be fluidically connected (e.g., directly fluidically connected) to the bioreactor. For example, the at least one filter may comprise a filter probe at least partially submerged in the suspension in the bioreactor. In some embodiments, the at least one filter is configured to receive an output of the bioreactor and produce at least one filtrate lean in the first type of biological cells relative to the suspension. In some embodiments, the at least one filtrate comprises IFN-α2b.

In some embodiments, the system further comprises an adjustment module configured to adjust one or more properties (e.g., pH, conductivity, product stability) of an input stream received by the adjustment module (e.g., the at least one filtrate) to produce an adjusted output stream (e.g., an adjusted filtrate). In some embodiments, the adjustment module is fluidically connected (e.g., directly fluidically connected) to the bioreactor, the at least one filter, and/or the purification module. In some embodiments, the adjustment module comprises a surge tank. In certain cases, the surge tank has a volume of about 50 mL to about 2 L, about 2 L to about 10 L, or about 10 L to about 100 L. In some cases, the surge tank may advantageously facilitate connection of an upstream process associated with a first flow rate and/or pressure and a downstream process associated with a second, different flow rate and/or pressure.

In some embodiments, the adjustment module is configured to increase or decrease the pH of the at least one filtrate to produce a pH-adjusted filtrate. In some embodiments, the pH of the adjusted filtrate is compatible with a first column of the purification module. In certain cases, adjusting the pH of the at least one filtrate may facilitate effective capture of IFN-α2b on the first column of the purification module. In addition, adjusting the pH of the at least one filtrate may advantageously reduce the amount of product aggregates, increase product yield, increase IFN-α2b stability, and/or increase product quality. In some embodiments, the pH-adjusted filtrate has a pH in the range of about 4.0 to about 9.0 (e.g., about 4.0 to about 8.0, about 4.0 to about 7.0, about 6.0 to about 8.0). In some embodiments, the pH-adjusted filtrate has a pH of about 4.0, about 5.0, about 5.5, about 6.0, about 6.5, about 7.0, about 7.5, about 8.0, or about 9.0.

In some embodiments, the adjustment module is configured to minimize hold time after adjustment. Minimizing hold time after adjustment may, in some cases, advantageously maximize product quality. In certain embodiments, the hold time of the adjusted filtrate is about 12 hours or less, about 6 hours or less, about 1 hour or less, about 30 minutes or less, or about 10 minutes or less. In some embodiments, the hold time of the adjusted filtrate is in the range of about 10 minutes to about 30 minutes, about 10 minutes to about 1 hour, about 10 minutes to about 6 hours, about 10 minutes to about 12 hours, about 10 minutes to about 18 hours, about 10 minutes to about 24 hours, about 30 minutes to about 1 hour, about 30 minutes to about 6 hours, about 30 minutes to about 12 hours, about 30 minutes to about 18 hour, about 30 minutes to about 24 hours, about 1 hour to about 6 hours, about 1 hour to about 12 hours, about 1 hour to about 18 hours, about 1 hour to about 24 hours, about 6 hour to about 12 hours, about 6 hour to about 18 hours, or about 6 hours to about 24 hours.

In some embodiments, the system further comprises a purification module configured to remove at least a first type of impurity, a second type of impurity, and a third type of impurity from an input stream received by the purification module (e.g., the at least one filtrate, the adjusted filtrate) to produce a purified filtrate. In some embodiments, the purification module is fluidically connected (e.g., directly fluidically connected) to the at least one filter and/or the adjustment module.

In some embodiments, the purification module comprises a first partitioning unit configured to remove at least the first type of impurity, a second partitioning unit configured to remove at least the second type of impurity, and a third partitioning unit configured to remove at least the third type of impurity. In certain embodiments, the design framework described above may be used to generate and evaluate candidate sequences of partitioning steps. Using the design framework, for example, a sequence of chromatography columns and associated conditions suitable for capturing and purifying IFN-α2b may be identified.

In some embodiments, the purification module comprises a first column comprising a multimodal cation exchange resin. In some embodiments, the first column is configured to remove at least the first type of impurity to produce a first partitioned filtrate lean in the first type of impurity relative to the at least one filtrate or the adjusted filtrate. In some embodiments, the first partitioned filtrate comprises IFN-α2b. According to certain embodiments, the multimodal cation exchange resin comprises Capto MMC ImpRes, Capto MMC, Nuvia cPrime, Toyopearl MX-Trp-650M, CMM HyperCel, and/or Eshmuno HCX. In some embodiments, the multimodal cation exchange resin comprises a Capto MMC ImpRes resin.

In some embodiments, the purification module further comprises a second column comprising an HCIC resin. In some embodiments, the second column is fluidically connected (e.g., directly fluidically connected) to the first column. In some embodiments, the second column is configured to remove at least the second type of impurity to produce a second partitioned filtrate lean in the second type of impurity relative to the first partitioned filtrate. In some embodiments, the second partitioned filtrate comprises IFN-α2b. In certain cases, the HCIC resin comprises MEP HyperCel, PPA HyperCel, and/or HEA HyperCel. In some embodiments, the HCIC resin comprises an MEP HyperCel resin or an HEA HyperCel resin.

In some embodiments, the purification module further comprises a third column comprising a cation exchange resin. In some embodiments, the third column is fluidically connected (e.g., directly fluidically connected) to the second column. In some embodiments, the third column is configured to remove at least the third type of impurity to produce a third partitioned filtrate lean in the third type of impurity relative to the second partitioned filtrate. In some embodiments, the third partitioned filtrate comprises IFN-α2b. In some embodiments, the cation exchange resin comprises an SP Sepharose HP resin or a Toyopearl MX-Trp-650M resin.

As another example, in some embodiments, the purification module comprises a first column comprising a multimodal cation exchange resin. In some embodiments, the first column is configured to remove at least the first type of impurity to produce a first partitioned filtrate lean in the first type of impurity relative to the at least one filtrate or the adjusted filtrate. In some embodiments, the first partitioned filtrate comprises IFN-α2b. According to certain embodiments, the multimodal cation exchange resin comprises Capto MMC ImpRes, Capto MMC, CMM HyperCel, and/or Eshmuno HCX. In some embodiments, the multimodal cation exchange resin comprises a Capto MMC ImpRes resin.

The purification module may further comprise a second column comprising a flow-through resin. In some embodiments, the second column is fluidically connected (e.g., directly fluidically connected) to the first column. In some embodiments, the second column is configured to remove at least the second type of impurity to produce a second partitioned filtrate lean in the second type of impurity relative to the first partitioned filtrate. In some embodiments, the second partitioned filtrate comprises IFN-α2b. In certain cases, the flow-through resin comprises Q Sepharose HP resin, HyperCel STAR AX resin, and/or Toyopearl NH2-750F resin. In some embodiments, the flow-through resin comprises a Q Sepharose HP resin.

The purification module may further comprise a third column comprising an anion exchange resin. In some embodiments, the third column is fluidically connected (e.g., directly fluidically connected) to the second column. In some embodiments, the third column is configured to remove at least the third type of impurity to produce a third partitioned filtrate lean in the third type of impurity relative to the second partitioned filtrate. In some embodiments, the third partitioned filtrate comprises IFN-α2b. In some embodiments, the anion exchange resin comprises a Capto Adhere resin.

In other embodiments, the purification module comprises a first partitioning unit configured to remove at least the first type of impurity and a second partitioning unit configured to remove at least the second type of impurity. In some such cases, the first column may comprise a multimodal cation exchange resin, as described herein, and the second column may comprise an anion exchange resin, as described herein.

Regardless of the purification module used, in some embodiments, the system further comprises a formulation module configured to produce a formulated product stream. In some embodiments, the formulation module is fluidically connected (e.g., directly fluidically connected) to the purification module. In some embodiments, the formulation module comprises a filtration unit, a viral filtration unit, and/or a product packaging unit. In some embodiments, the filtration unit comprises a tangential flow filtration device.

In some embodiments, the product stream (e.g., the purified filtrate stream from the purification module, the formulated product stream from the formulation module) has a relatively high concentration of IFN. In certain embodiments, the product stream has a product concentration of at least about 0.01 mg/mL, at least about 0.02 mg/mL, at least about 0.03 mg/mL, at least about 0.04 mg/mL, at least about 0.05 mg/mL, at least about 0.1 mg/mL, at least about 0.5 mg/mL, at least about 1 mg/mL, at least about 5 mg/mL, at least about 10 mg/mL, at least about 25 mg/mL, at least about 50 mg/mL, at least about 75 mg/mL, or at least about at least about 90 mg/mL. In some embodiments, the product stream has a IFN concentration in the range of about 0.05 mg/mL to about 100 mg/mL, about 0.5 mg/mL to about 100 mg/mL, or about 1 mg/mL to about 100 mg/mL. In certain embodiments, the product stream has a product concentration in the range of about 0.01 mg/mL to about 0.1 mg/mL, about 0.02 mg/mL to about 0.1 mg/mL, about 0.03 mg/mL to about 0.1 mg/mL, about 0.04 mg/mL to about 0.1 mg/mL, or about 0.05 mg/mL to about 0.1 mg/mL.

In some embodiments, the product stream has a relatively high product yield. In certain embodiments, the product stream has a product yield of at least about 50%, at least about 60%, at least about 70%, at least about 80%, at least about 90%, at least about 95%, or at least about 99%. In certain embodiments, the product stream has a product yield in the range of about 50% to about 70%, about 50% to about 80%, about 50% to about 90%, about 50% to about 95%, about 50% to about 99%, about 60% to about 80%, about 60% to about 90%, about 60% to about 95%, about 60% to about 99%, about 70% to about 90%, about 70% to about 95%, about 70% to about 99%, about 80% to about 95%, about 80% to about 99%, or about 90% to about 99%.

In some embodiments, the product stream has a purity of at least about 50%, at least about 60%, at least about 70%, at least about 77%, at least about 80%, at least about 90%, at least about 95%, or at least about 99%. In some embodiments, the product stream has a purity in the range of about 50% to about 99%, about 60% to about 99%, about 70% to about 99%, about 77% to about 99%, about 80% to about 99%, or about 90% to about 99%. For example, in some embodiments in which the purification module comprises a flow-through resin, the product stream has a product yield of at least about 65% (e.g., in the range of about 65% to about 99%, at least about 70%, in the range of about 70% to about 99%).

In some embodiments, the product stream has a DNA concentration of about 1 ng/(mg IFN-α2b) or less, about 0.8 ng/(mg IFN-α2b) or less, about 0.6 ng/(mg IFN-α2b) or less, about 0.51 ng/(mg IFN-α2b) or less, or about 0.2 ng/(mg IFN-α2b) or less. In some embodiments, the product stream has a DNA concentration in the range of about 0.0 ng/(mg IFN-α2b) to about 0.2 ng/(mg IFN-α2b), 0.0 ng/(mg IFN-α2b) to about 0.51 ng/(mg IFN-α2b), about 0.0 ng/(mg IFN-α2b) to about 0.6 ng/(mg IFN-α2b), about 0.0 ng/(mg IFN-α2b) to about 0.8 ng/(mg IFN-α2b), about 0.0 ng/(mg IFN-α2b) to about 1 ng/(mg IFN-α2b), about 0.2 ng/(mg IFN-α2b) to about 0.6 ng/(mg IFN-α2b), about 0.2 ng/(mg IFN-α2b) to about 0.8 ng/(mg IFN-α2b), or about 0.2 ng/(mg IFN-α2b) to about 1 ng/(mg IFN-α2b)

In some embodiments, the product stream has an aggregate content of about 2% or less, about 1% or less, about 0.5% or less, about 0.1% or less, about 0.07% or less, or about 0.05% or less. In some embodiments, the product stream has an aggregate content in the range of about 0.05% to about 0.1%, about 0.05% to about 0.5%, about 0.05% to about 1%, or about 0.05% to about 2%.

In some embodiments, the system is configured to be continuously operated. In certain embodiments, for example, the bioreactor is configured to receive at least one feed stream comprising the at least one cell culture medium. In some embodiments, the at least one filtrate is an at least one filtrate stream. In some embodiments, the purified filtrate is a purified filtrate stream.

In some embodiments, the reactor chamber of the bioreactor has a volume of about 1 L or less. In certain of these embodiments, an input stream to the system (e.g., the at least one feed stream) and an output stream of the system (e.g., the purified filtrate stream, the formulated product stream) each have a flow rate of at least about 0.1 mL/min, at least about 0.5 mL/min, at least about 1.0 mL/min, at least about 1.5 mL/min, or at least about 2 mL/min over a period of at least about 1 day. In some embodiments, the input stream and the output stream each have a flow rate in the range of 0.1 mL/min to about 0.5 mL/min, about 0.1 mL/min to about 1.0 mL/min, about 0.1 mL/min to about 1.5 mL/min, about 0.1 mL/min to about 2 mL/min, about 0.5 mL/min to about 1.0 mL/min, about 0.5 mL/min to about 1.5 mL/min, about 0.5 mL/min to about 2 mL/min, or about 1 mL/min to about 2 mL/min over a period of at least about 1 day. In some embodiments, the system is configured to produce at least about 1 mg, at least about 5 mg, at least about 10 mg, at least about 20 mg, at least about 100 mg, at least about 500 mg, at least about 1 g, at least about 2 g, at least about 3 g, at least about 4 g, or at least about 50 mg of IFN-α2b per day. In some embodiments, the system is configured to produce an amount of IFN-α2b in the range of about 1 mg to about 5 mg, about 1 mg to about 10 mg, about 1 mg to about 20 mg, about 1 mg to about 50 mg, about 1 mg to about 100 mg, about 1 mg to about 500 mg, about 1 mg to about 1 g, about 1 mg to about 5 g, about 5 mg to about 10 mg, about 5 mg to about 20 mg, about 5 mg to about 50 mg, about 5 mg to about 100 mg, about 5 mg to about 500 mg, about 5 mg to about 1 g, about 5 mg to about 5 g, about 10 mg to about 20 mg, about 10 mg to about 50 mg, about 10 mg to about 100 mg, about 10 mg to about 500 mg, about 10 mg to about 1 g, about 10 mg to about 5 g, about 20 mg to about 100 mg, about 20 mg to about 500 mg, about 20 mg to about 1 g, or about 20 mg to about 50 mg, about 50 mg to about 100 mg, about 50 mg to about 500 mg, about 50 mg to about 1 g, about 50 mg to about 5 g, about 100 mg to about 500 mg, about 100 mg to about 1 g, about 100 mg to about 5 g, about 500 mg to about 1 g, about 500 mg to about 5 g, or about 1 g to about 5 g per day.

In some embodiments, the reactor chamber of the bioreactor has a volume of about 1 L to about 10 L. In certain of these embodiments, an input stream to the system (e.g., the at least one feed stream) and an output stream of the system (e.g., the purified filtrate stream, the formulated product stream) each have a flow rate of at least about 0.5 mL/min, at least about 1 mL/min, at least about 5 mL/min, at least about 10 mL/min, or at least about 20 mL/min over a period of at least about 1 day. In certain embodiments, the input stream and the output stream each have a flow rate in the range of about 0.5 mL/min to about 1 mL/min, about 0.5 mL/min to about 5 mL/min, about 0.5 mL/min to about 10 mL/min, about 0.5 mL/min to about 20 mL/min, about 1 mL/min to about 5 mL/min, about 1 mL/min to about 10 mL/min, about 1 mL/min to about 20 mL/min, about 5 mL/min to 10 mL/min, about 5 mL/min to about 20 mL/min, or about 10 mL/min to about 20 mL/min over a period of at least about 1 day. In some of these embodiments, the system is configured to produce at least about 50 mg, at least about 100 mg, at least about 200 mg, at least about 500 mg, at least about 1 g, at least about 5 g, at least about 10 g, or at least about 50 g of IFN-α2b per day In some of these embodiments, the system is configured to produce at least about 50 mg, at least about 100 mg, at least about 200 mg, at least about 500 mg, at least about 1 g, at least about 5 g, at least about 10 g, or at least about 500 mg of IFN-α2b per day. In some embodiments, the system is configured to produce an amount of IFN-α2b in the range of about 50 mg to about 100 mg, about 50 mg to about 200 mg, about 50 mg to about 500 mg, about 50 mg to about 1 g, about 50 mg to about 5 g, about 50 mg to about 10 g, about 50 mg to about 50 g, about 100 mg to about 200 mg, about 100 mg to about 500 mg, about 100 mg to about 1 g, about 100 mg to about 5 g, about 100 mg to about 10 g, about 100 mg to about 50 g, about 200 mg to about 1 g, about 200 mg to about 5 g, about 200 mg to about 10 g, or about 200 mg to about 500 mg, about 500 mg to about 1 g, about 500 mg to about 5 g, about 500 mg to about 10 g, about 500 mg to about 50 g, about 1 g to about 5 g, about 1 g to about 10 g, about 1 g to about 50 g, about 5 g to about 10 g, about 5 g to about 50 g, or about 10 g to about 50 g per day.

In some embodiments, the reactor chamber of the bioreactor has a volume of about 10 L to about 50 L. In certain of these embodiments, an input stream to the system (e.g., the at least one feed stream) and an output stream of the system (e.g., the purified filtrate stream, the formulated product stream) each have a flow rate of at least about 5 mL/min, at least about 10 mL/min, at least about 20 mL/min, at least about 50 mL/min, at least about 100 mL/min, at least about 150 mL/min, or at least about 200 mL/min over a period of at least about 1 day. In some embodiments, the input stream and the output stream each have a flow rate in the range of about 5 mL/min to about 10 mL/min, about 5 mL/min to about 20 mL/min, about 5 mL/min to about 50 mL/min, about 5 mL/min to about 100 mL/min, about 5 mL/min to about 150 mL/min, about 5 mL/min to about 200 mL/min, about 10 mL/min to about 20 mL/min, about 10 mL/min to about 50 mL/min, about 10 mL/min to about 100 mL/min, about 10 mL/min to about 150 mL/min, about 10 mL/min to about 200 mL/min, about 50 mL/min to about 100 mL/min, about 50 mL/min to about 150 mL/min, about 50 mL/min to about 200 mL/min, or about 100 mL/min to about 200 mL/min over a period of at least about 1 day. In some of these embodiments, the system is configured to produce at least about 500 mg, at least about 1 g, at least about 2 g, at least about 5 g, at least about 10 g, at least about 50 g, at least about 100 g, or at least about 10 250 g of IFN-α2b per day. In some embodiments, the system is configured to produce an amount of IFN-α2b in the range of about 500 mg to about 1 g, about 500 mg to about 2 g, about 500 mg to about 5 g, about 500 mg to about 10 g, about 500 mg to about 50 g, about 500 mg to about 100 g, about 500 mg to about 250 g, about 1 g to about 5 g, about 1 g to about 10 g, about 1 g to about 50 g, about 1 g to about 100 g, about 5 g to about 250 g, about 5 g to about 10 g, about 5 g to about 50 g, about 5 g to about 100 g, or about 52 g to about 250 g, about 10 g to about 100 g, about 10 g to about 250 g, about 50 g to about 100 g, about 50 g to about 250 g, or about 100 g to about 250 g per day.

Some embodiments described herein relate to methods for producing IFN-α2b. In certain embodiments, the method comprises supplying a growth cell medium to a bioreactor (e.g., a perfusion bioreactor). In some embodiments, the method further comprises incubating a first type of biological cells in the growth cell culture medium for a period of at least one day. In some embodiments, the method further comprises at least partially removing the growth cell culture medium from the bioreactor. In some embodiments, the method comprises supplying at least one cell culture medium (e.g., a production cell culture medium) to the bioreactor. In some embodiments, the method comprises producing, within the bioreactor, a suspension comprising the at least one cell culture medium and at least a first type of biological cells comprising IFN-α2b. In some embodiments, the method further comprises causing at least a portion of the suspension to flow through at least one filter to produce at least one filtrate lean in the first type of biological cells. In some embodiments, the at least one filtrate comprises IFN-α2b.

In some embodiments, the method comprises flowing the at least one filtrate to an adjustment module (e.g., from the at least one filter to the adjustment module). In some embodiments, the method further comprises adjusting, within the adjustment module, one or more properties (e.g., pH, conductivity, product stability) of the at least one filtrate. In certain embodiments, the pH of the at least one filtrate is increased or decreased (e.g., to be compatible with the first column of the purification module). In some embodiments, increasing the pH of the at least one filtrate comprises adding a base to the at least one filtrate. In some embodiments, decreasing the pH of the at least one filtrate comprises adding an acid to the at least one filtrate.

In some embodiments, the method further comprises flowing the at least one filtrate and/or the adjusted filtrate through a first column comprising a multimodal cation exchange resin. In certain embodiments, the multimodal cation exchange resin is a Capto MMC ImpRes resin. In some embodiments, the method further comprises collecting one or more first fractions comprising IFN-α2b from an outflow of the first column. In some embodiments, the one or more first fractions are lean in the first type of impurity relative to the at least one filtrate or the adjusted filtrate. In some embodiments, the one or more first fractions have a concentration of the first type of impurity that is at least about 50%, at least about 75%, at least about 90%, at least about 95%, or at least about 99% less than the concentration of the first type of impurity in the at least one filtrate or the adjusted filtrate.

In some embodiments, the method further comprises flowing the one or more first fractions through a second column. The method may further comprise collecting one or more second fractions comprising IFN-α2b from an outflow of the second column. In some such cases, the method may further comprise flowing the one or more second fractions through a third column. In some instances, the method may also comprise collecting one or more third fractions comprising IFN-α2b from an outflow of the third column. For example, in some embodiments, the method further comprises flowing the one or more first fractions through a second column comprising an HCIC resin. In some embodiments, the HCIC comprises an MEP HyperCel resin or an HEA HyperCel resin. In some embodiments, the method further comprises collecting one or more second fractions comprising IFN-α2b from an outflow of the second column. In some embodiments, the one or more second fractions are lean in the second type of impurity relative to the first fractions. In some embodiments, the one or more second fractions have a concentration of the second type of impurity that is at least about 50%, at least about 75%, at least about 90%, at least about 95%, or at least about 99% less than the concentration of the second type of impurity in the first fractions.

In some embodiments, the method further comprises flowing the one or more second fractions through a third column comprising a cation exchange resin. In some embodiments, the cation exchange resin comprises an SP Sepharose HP resin or a Toyopearl MX-Trp-650M resin. In some embodiments, the method further comprises collecting one or more third fractions comprising IFN-α2b from an outflow of the third column. In some embodiments, the one or more third fractions are lean in a third type of impurity relative to the second fractions. In some embodiments, the one or more third fractions have a concentration of the third type of impurity that is at least about 50%, at least about 75%, at least about 90%, at least about 95%, or at least about 99% less than the concentration of the third type of impurity in the second fractions. In some embodiments, the third fractions are collected as a biologically-produced product stream.

As another example, in some embodiments, the method further comprises flowing the one or more first fractions through a second column comprising a flow-through resin. In some embodiments, the flow-through resin comprises a Q Sepharose HP resin. In some embodiments, the method further comprises collecting one or more second fractions comprising IFN-α2b from an outflow of the second column. In some embodiments, the one or more second fractions are lean in the second type of impurity relative to the first fractions. In some embodiments, the one or more second fractions have a concentration of the second type of impurity that is at least about 50%, at least about 75%, at least about 90%, at least about 95%, or at least about 99% less than the concentration of the second type of impurity in the first fractions. In some embodiments, the method further comprises flowing the one or more second fractions through a third column comprising an anion exchange resin. In some embodiments, the anion exchange resin comprises a Capto Adhere resin. In some embodiments, the method further comprises collecting one or more third fractions comprising IFN-α2b from an outflow of the third column. In some embodiments, the one or more third fractions are lean in a third type of impurity relative to the second fractions. In some embodiments, the one or more third fractions have a concentration of the third type of impurity that is at least about 50%, at least about 75%, at least about 90%, at least about 95%, or at least about 99% less than the concentration of the third type of impurity in the second fractions. In some embodiments, the third fractions are collected as a biologically-produced product stream.

In some embodiments, the method further comprises flowing the third fractions to a formulation module to produce a formulated product stream. In some embodiments, flowing the purified filtrate through the formulation module comprises flowing the purified filtrate through a tangential flow filtration device. In some embodiments, flowing the purified filtrate through the formulation module comprises flowing the purified filtrate through a viral filtration unit. In some embodiments, the formulated product stream is lean in one or more viruses relative to the purified filtrate stream. In some embodiments, flowing the purified filtrate through the formulation module comprises depositing one or more portions of the purified filtrate stream into one or more containers (e.g., bags, vials, syringes, bottles). In some embodiments, the one or more containers are aseptic and/or sterile containers.

### Single-Domain Antibody-Specific Systems and Methods

Some embodiments described herein relate to systems for producing a single-domain antibody. According to some embodiments, the system comprises a bioreactor (e.g., a perfusion bioreactor), at least one filter, and a purification module. The bioreactor may comprise a reaction chamber, and, as described above, may have any suitable shape and be formed of any suitable material. In some embodiments, the reaction chamber contains a suspension comprising at least one cell culture medium and at least a first type of biological cells configured to express the single-domain antibody. In some embodiments, the biological cells are yeast cells. In certain cases, the yeast cells are Pichia pastoris cells. In some embodiments, for example during a cell growth phase, the at least one cell culture medium comprises chemically defined media comprising a carbon source or buffered glycerol-complex medium (BMGY). In some embodiments, for example during the single-domain antibody production phase, the at least one cell culture medium comprises chemically defined media comprising a carbon source and/or other additive for induction of protein expression or buffered methanol-complex media (BMMY).

In some embodiments, the at least one filter of the system comprises a filter probe, a filtration membrane, and/or a ceramic filter. The at least one filter may, in some embodiments, be fluidically connected (e.g., directly fluidically connected) to the bioreactor. For example, in certain embodiments, the at least one filter comprises at least one filter probe at least partially submerged in the suspension in the bioreactor. In some embodiments, the at least one filter is configured to receive an output of the bioreactor and produce at least one filtrate lean in the first type of biological cells relative to the suspension. In some embodiments, the at least one filtrate comprises a single-domain antibody.

In some embodiments, the system further comprises an adjustment module configured to adjust one or more properties (e.g., pH, conductivity, product stability) of an input stream received by the adjustment module (e.g., the at least one filtrate) to produce an adjusted output stream (e.g., an adjusted filtrate). According to some embodiments, the adjustment module is fluidically connected (e.g., directly fluidically connected) to the bioreactor, the at least one filter, and/or the purification module. In some embodiments, the adjustment module comprises a surge tank. In certain cases, the surge tank has a volume of about 50 mL to about 2 L, about 2 L to about 10 L, or about 10 L to about 100 L. In some cases, the surge tank may advantageously facilitate connection of an upstream process associated with a first flow rate and/or pressure and a downstream process associated with a second, different flow rate and/or pressure.

In certain illustrative embodiments, the adjustment module is configured to increase or decrease the pH of the at least one filtrate to produce a pH-adjusted filtrate. In some embodiments, the pH of the adjusted filtrate is compatible with a first column of the purification module. In certain cases, adjusting the pH of the at least one filtrate may facilitate effective capture of a single-domain antibody on the first column of the purification module. In addition, adjusting the pH of the at least one filtrate may advantageously reduce the amount of product aggregates, increase product yield, increase single-domain antibody stability, and/or maximize product quality. In some embodiments, the pH-adjusted filtrate has a pH of about 4.5, about 5.0, about 5.5, about 6.0, about 6.5, about 7.0, about 7.5, about 8.0, or about 9.0. In certain embodiments, the pH-adjusted filtrate has a pH in the range of about 4.5 to about 7.0 (e.g., about 4.5 and to about 6.0, about 4.5 and to about 5.5).

In some embodiments, the adjustment module is configured to minimize hold time after adjustment. Minimizing hold time after adjustment may, in some cases, advantageously maximize product quality. In certain embodiments, the hold time of the adjusted filtrate is about 12 hours or less, about 6 hours or less, about 1 hour or less, about 30 minutes or less, or about 10 minutes or less. In some embodiments, the hold time of the adjusted filtrate is in the range of about 10 minutes to about 30 minutes, about 10 minutes to about 1 hour, about 10 minutes to about 6 hours, about 10 minutes to about 12 hours, about 10 minutes to about 18 hours, about 10 minutes to about 18 hours, about 30 minutes to about 1 hour, about 30 minutes to about 6 hours, about 30 minutes to about 12 hours, about 30 minutes to about 18 hour, about 30 minutes to about 24 hours, about 1 hour to about 6 hours, about 1 hour to about 12 hours, about 1 hour to about 18 hours, about 1 hour to about 24 hours, about 6 hour to about 12 hours, about 6 hour to about 18 hours, or about 6 hours to about 24 hours.

In some embodiments, the purification module is configured to remove at least a first type of impurity, a second type of impurity, and/or a third type of impurity from an input stream received by the purification module (e.g., the at least one filtrate, the adjusted filtrate) to produce a purified filtrate. In some embodiments, the purification module is fluidically connected (e.g., directly fluidically connected) to the at least one filter and/or the adjustment module.

In some embodiments, the purification module comprises a first partitioning unit configured to remove at least the first type of impurity, a second partitioning unit configured to remove at least the second type of impurity, and optionally a third partitioning unit configured to remove at least the third type of impurity. In certain embodiments, the design framework described above may be used to generate and evaluate candidate sequences of partitioning steps. Using the design framework, for example, a sequence of chromatography columns and associated conditions suitable for capturing and purifying a single-domain antibody may be identified.

In some embodiments, the purification module comprises a first column comprising a multimodal cation exchange resin. In some embodiments, the first column is configured to remove at least the first type of impurity to produce a first partitioned filtrate that comprises a single-domain antibody and is lean in the first type of impurity relative to the first filtrate. According to certain embodiments, the multimodal cation exchange resin comprises Capto MMC, CMM HyperCel, Nuvia cPrime, Toyopearl MX-Trp-650M, Eshmuno HCX, and/or Capto MMC ImpRes. In some embodiments, the multimodal cation exchange resin comprises CMM HyperCel.

In some embodiments, the purification module further comprises a second column comprising an anion exchange resin (e.g., a salt-tolerant anion exchange resin). The second column may be fluidically connected (e.g., directly fluidically connected) to the first column, according to certain embodiments. In some embodiments, the second column is configured to remove at least the second type of impurity to produce a second partitioned filtrate that comprises a single-domain antibody and is lean in the second type of impurity relative to the first partitioned filtrate. In certain cases, the anion exchange resin comprises HyperCel STAR AX, Capto Adhere, and/or PPA HyperCel. In some embodiments, the anion exchange resin comprises HyperCel STAR AX.

In some embodiments, the system further comprises a formulation module configured to produce a formulated product stream. In some embodiments, the formulation module is fluidically connected (e.g., directly fluidically connected) to the purification module. In some embodiments, the formulation module comprises a filtration unit, a viral filtration unit, a dilution adjustment unit, and/or a product packaging unit. According to some embodiments, the filtration unit comprises a tangential flow filtration device.

In some embodiments, the product stream (e.g., the purified filtrate stream from the purification module, the formulated product stream from the formulation module) has a relatively high concentration of the single-domain antibody. In certain embodiments, the product stream has a single-domain antibody concentration of at least about 0.05 mg/mL, at least about 0.1 mg/mL, at least about 0.2 mg/mL, at least about 0.3 mg/mL, at least about 0.4 mg/mL, at least about 0.5 mg/mL, at least about 1 mg/mL, at least about 5 mg/mL, at least about 10 mg/mL, at least about 25 mg/mL, at least about 50 mg/mL, at least about 75 mg/mL, or at least about at least about 90 mg/mL. In some embodiments, the product stream has a single-domain antibody concentration in the range of about 0.05 mg/mL to about 100 mg/mL, about 0.5 mg/mL to about 100 mg/mL, or about 1 mg/mL to about 100 mg/mL. In certain embodiments, the product stream has a single-domain antibody concentration in the range of about 0.05 mg/mL to about 0.2 mg/mL, about 0.05 mg/mL to about 0.3 mg/mL, about 0.05 mg/mL to about 0.4 mg/mL, about 0.05 mg/mL to about 0.5 mg/mL, about 0.1 mg/mL to about 0.3 mg/mL, about 0.1 mg/mL to about 0.4 mg/mL, about 0.1 mg/mL to about 0.5 mg/mL, about 0.2 mg/mL to about 0.3 mg/mL, about 0.2 mg/mL to about 0.4 mg/mL, about 0.2 mg/mL to about 0.5 mg/mL, or about 0.3 mg/mL to about 0.5 mg/mL.

In some embodiments, the product stream has a relatively high product yield. In certain embodiments, the product stream has a product yield of at least about 50%, at least about 60%, at least about 70%, at least about 80%, at least about 90%, at least about 95%, or at least about 99%. In certain embodiments, the product stream has a product yield in the range of about 50% to about 70%, about 50% to about 80%, about 50% to about 90%, about 50% to about 95%, about 50% to about 99%, about 60% to about 80%, about 60% to about 90%, about 60% to about 95%, about 60% to about 99%, about 70% to about 90%, about 70% to about 95%, about 70% to about 99%, about 80% to about 95%, about 80% to about 99%, or about 90% to about 99%.

In some embodiments, the product stream has a relatively low concentration of impurities. In certain embodiments, for example, the product stream has a relatively low concentration of host cell proteins. In some embodiments, the product stream has a host cell protein concentration of about 100 ng/(mg single-domain antibody) or less, about 50 ng/(mg single-domain antibody) or less, about 20 ng/(mg single-domain antibody) or less, about 10 ng/(mg single-domain antibody) or less, about 8 ng/(mg single-domain antibody) or less, or about 5 ng/(mg single-domain antibody) or less. In some embodiments, the product stream has a host cell protein concentration in the range of about 0 ng/(mg single-domain antibody) to about 5 ng/(mg single-domain antibody), about 0 ng/(mg single-domain antibody) to about 8 ng/(mg single-domain antibody), about 0 ng/(mg single-domain antibody) to about 10 ng/(mg single-domain antibody), about 0 ng/(mg single-domain antibody) to about 20 ng/(mg single-domain antibody), about 0 ng/(mg single-domain antibody) to about 50 ng/(mg single-domain antibody), about 0 ng/(mg single-domain antibody) to about 100 ng/(mg single-domain antibody), about 5 ng/(mg single-domain antibody) to about 8 ng/(mg single-domain antibody), about 5 ng/(mg single-domain antibody) to about 10 ng/(mg single-domain antibody), about 5 ng/(mg single-domain antibody) to about 20 ng/(mg single-domain antibody), about 5 ng/(mg single-domain antibody) to about 50 ng/(mg single-domain antibody), about 5 ng/(mg single-domain antibody) to about 100 ng/(mg single-domain antibody), about 10 ng/(mg single-domain antibody) to about 20 ng/(mg single-domain antibody), about 10 ng/(mg single-domain antibody) to about 50 ng/(mg single-domain antibody), or about 10 ng/(mg single-domain antibody) to about 100 ng/(mg single-domain antibody).

In certain embodiments, the product stream has a relatively low concentration of DNA. For example, in some embodiments, the product stream has a DNA concentration of about 100 ng/(mg single-domain antibody) or less, about 50 ng/(mg single-domain antibody) or less, about 20 ng/(mg single-domain antibody) or less, about 10 ng/(mg single-domain antibody) or less, about 5 ng/(mg single-domain antibody) or less, about 1 ng/(mg single-domain antibody) or less, about 0.5 ng/(mg single-domain antibody) or less, about 0.1 ng/(mg single-domain antibody) or less, about 0.05 ng/(mg single-domain antibody) or less, about 0.01 ng/(mg single-domain antibody) or less, or about 0 ng/(mg single-domain antibody) (e.g., not detectable). In some embodiments, the product stream has a DNA concentration in the range of about 0 ng/(mg single-domain antibody) to about 0.01 ng/(mg single-domain antibody), about 0 ng/(mg single-domain antibody) to about 0.05 ng/(mg single-domain antibody), about 0 ng/(mg single-domain antibody) to about 0.1 ng/(mg single-domain antibody), about 0 ng/(mg single-domain antibody) to about 0.5 ng/(mg single-domain antibody), about 0 ng/(mg single-domain antibody) to about 1 ng/(mg single-domain antibody), about 0 ng/(mg single-domain antibody) to about 5 ng/(mg single-domain antibody), about 0 ng/(mg single-domain antibody) to about 10 ng/(mg single-domain antibody), about 0 ng/(mg single-domain antibody) to about 20 ng/(mg single-domain antibody), about 0 ng/(mg single-domain antibody) to about 50 ng/(mg single-domain antibody), about 0 ng/(mg single-domain antibody) to about 100 ng/(mg single-domain antibody).

In some embodiments, the product stream contains a relatively low amount of aggregates (e.g., aggregates of the product). In some embodiments, the product stream has an aggregate content of about 2% or less, about 1% or less, about 0.90% or less, about 0.80% or less, about 0.70% or less, about 0.60% or less, or about 0.50% or less, about 0.40% or less, about 0.30% or less, about 0.20% or less, or about 0.10% or less. In some embodiments, the product stream has an aggregate content in the range of about 0.10% to about 0.20%, about 0.10% to about 0.30%, about 0.10% to about 0.40%, about 0.10% to about 0.50%, about 0.10% to about 0.60%, about 0.10% to about 0.70%, about 0.10% to about 0.80%, about 0.10% to about 0.90%, about 0.10% to about 1%, or about 0.10% to about 2%.

In some embodiments, the system is configured to be continuously operated. In certain embodiments, for example, the bioreactor is configured to receive at least one feed stream comprising the at least one cell culture medium. In some embodiments, the at least one filtrate is an at least one filtrate stream. In some embodiments, the purified filtrate is a purified filtrate stream.

In some embodiments, the reactor chamber of the bioreactor has a volume of about 1 L or less. In certain of these embodiments, an input stream to the system (e.g., the at least one feed stream) and an output stream of the system (e.g., the purified filtrate stream, the formulated product stream) each have a flow rate of at least about 0.1 mL/min, at least about 0.5 mL/min, at least about 1.0 mL/min, at least about 1.5 mL/min, or at least about 2 mL/min over a period of at least about 1 day. In some embodiments, the input stream and the output stream each have a flow rate in the range of 0.1 mL/min to about 0.5 mL/min, about 0.1 mL/min to about 1.0 mL/min, about 0.1 mL/min to about 1.5 mL/min, about 0.1 mL/min to about 2 mL/min, about 0.5 mL/min to about 1.0 mL/min, about 0.5 mL/min to about 1.5 mL/min, about 0.5 mL/min to about 2 mL/min, or about 1 mL/min to about 2 mL/min over a period of at least about 1 day. In some embodiments, the system is configured to produce at least about 1 mg, at least about 5 mg, at least about 10 mg, at least about 20 mg, at least about 50 mg, at least about 100 mg, at least about 200 mg, at least about 500 mg, at least about 1 g, at least about 2 g, at least about 5 g, at least about 10 g, at least about 15 g, or at least about 18 g of a single-domain antibody per day. In some embodiments, the system is configured to produce an amount of a single-domain antibody in the range of about 1 mg to about 5 mg, about 1 mg to about 10 mg, about 1 mg to about 20 mg, about 1 mg to about 50 mg, about 1 mg to about 100 mg, about 1 mg to about 500 mg, about 1 mg to about 1 g, about 1 mg to about 2 g, about 1 mg to about 5 g, about 1 mg to about 10 g, about 1 mg to about 15 g, about 1 mg to about 20 g, about 10 mg to about 20 mg, about 10 mg to about 50 mg, about 10 mg to about 100 mg, about 10 mg to about 500 mg, about 10 mg to about 1 g, about 10 mg to about 2 g, about 10 mg to about 5 g, about 10 mg to about 10 g, about 10 mg to about 15 g, about 10 mg to about 20 g, about 50 mg to about 100 mg, about 50 mg to about 500 mg, about 50 mg to about 1 g, about 50 mg to about 2 g, about 50 mg to about 5 g, about 50 mg to about 10 g, about 50 mg to about 15 g, about 50 mg to about 20 g, about 100 mg to about 500 mg, about 100 mg to about 1 g, about 100 mg to about 2 g, about 100 mg to about 5 g, about 500 mg to about 1 g, about 500 mg to about 2 g, about 500 mg to about 5 g, about 1 g to about 5 g, about 1 g to about 10 g, about 1 g to about 15 g, or about 1 g to about 20 g per day.

In some embodiments, the reactor chamber of the bioreactor has a volume of about 1 L to about 10 L. In certain of these embodiments, an input stream to the system (e.g., the at least one feed stream) and an output stream of the system (e.g., the purified filtrate stream, the formulated product stream) each have a flow rate of at least about 0.5 mL/min, at least about 1 mL/min, at least about 5 mL/min, at least about 10 mL/min, or at least about 20 mL/min over a period of at least about 1 day. In certain embodiments, the input stream and the output stream each have a flow rate in the range of about 0.5 mL/min to about 1 mL/min, about 0.5 mL/min to about 5 mL/min, about 0.5 mL/min to about 10 mL/min, about 0.5 mL/min to about 20 mL/min, about 1 mL/min to about 5 mL/min, about 1 mL/min to about 10 mL/min, about 1 mL/min to about 20 mL/min, about 5 mL/min to 10 mL/min, about 5 mL/min to about 20 mL/min, or about 10 mL/min to about 20 mL/min over a period of at least about 1 day. In some of these embodiments, the system is configured to produce at least about 50 mg, at least about 100 mg, at least about 200 mg, at least about 500 mg, at least about 1 g, at least about 5 g, at least about 10 g, at least about 50 g, at least about 100 g, at least about 150 g, or at least about 175 g of a single-domain antibody per day. In some embodiments, the system is configured to produce an amount of a single-domain antibody in the range of about 50 mg to about 100 mg, about 50 mg to about 200 mg, about 50 mg to about 500 mg, about 50 mg to about 1 g, about 50 mg to about 5 g, about 50 mg to about 10 g, about 50 mg to about 50 g, about 50 mg to about 100 g, about 50 mg to about 150 g, about 50 mg to about 200 g, about 100 mg to about 500 mg, about 100 mg to about 1 g, about 100 mg to about 5 g, about 100 mg to about 10 g, about 100 mg to about 50 g, about 100 mg to about 100 g, about 100 mg to about 150 g, about 100 mg to about 200 g, about 500 mg to about 1 g, about 500 mg to about 5 g, about 500 mg to about 10 g, about 500 mg to about 50 g, about 1 g to about 10 g, about 1 g to about 50 g, about 10 g to about 50 g per day, about 1 g to about 100 g, about 1 g to about 150 g,or about 1 g to about 200 g.

In some embodiments, the reactor chamber of the bioreactor has a volume of about 10 L to about 50 L. In certain of these embodiments, an input stream to the system (e.g., the at least one feed stream) and an output stream of the system (e.g., the purified filtrate stream, the formulated product stream) each have a flow rate of at least about 5 mL/min, at least about 10 mL/min, at least about 20 mL/min, at least about 50 mL/min, at least about 100 mL/min, at least about 150 mL/min, or at least about 200 mL/min over a period of at least about 1 day. In some embodiments, the input stream and the output stream each have a flow rate in the range of about 5 mL/min to about 10 mL/min, about 5 mL/min to about 20 mL/min, about 5 mL/min to about 50 mL/min, about 5 mL/min to about 100 mL/min, about 5 mL/min to about 150 mL/min, about 5 mL/min to about 200 mL/min, about 10 mL/min to about 20 mL/min, about 10 mL/min to about 50 mL/min, about 10 mL/min to about 100 mL/min, about 10 mL/min to about 150 mL/min, about 10 mL/min to about 200 mL/min, about 50 mL/min to about 100 mL/min, about 50 mL/min to about 150 mL/min, about 50 mL/min to about 200 mL/min, or about 100 mL/min to about 200 mL/min over a period of at least about 1 day. In some of these embodiments, the system is configured to produce at least about 500 mg, at least about 1 g, at least about 2 g, at least about 5 g, at least about 10 g, at least about 50 g, at least about 100 g, at least about 200 g, at least about 500 g, at least about 750 g, or at least about 900 g of a single-domain antibody per day. In some embodiments, the system is configured to produce an amount of a single-domain antibody in the range of about 500 mg to about 1 g, about 500 mg to about 2 g, about 500 mg to about 5 g, about 500 mg to about 10 g, about 500 mg to about 50 g, about 500 mg to about 100 g, about 500 mg to about 200 g, about 500 mg to about 500 g, about 500 mg to about 750 g, about 500 mg to about 1,000 g, about 1 g to about 5 g, about 1 g to about 10 g, about 1 g to about 50 g, about 1 g to about 100 g, about 1 g to about 500 g, about 1 g to about 750 g, about 1 g to about 1,000 g, about 10 g to about 50 g, about 10 g to about 100 g, about 10 g to about 500 g, about 10 g to about 750 g, about 10 g to about 1,000 g, about 100 g to about 500 g, about 100 g to about 750 g, or about 100 g to about 1,000 g per day.

Some embodiments described herein relate to methods for producing a single-domain antibody. In certain embodiments, the method comprises supplying a growth cell medium to a bioreactor (e.g., a perfusion bioreactor). In some embodiments, the method further comprises incubating a first type of biological cells in the growth cell culture medium for a period of at least one day. In some embodiments, the method further comprises at least partially removing the growth cell culture medium from the bioreactor. In some embodiments, the method further comprises supplying at least one cell culture medium (e.g., a production cell culture medium) to the bioreactor. In some embodiments, the method comprises producing, within the bioreactor, a suspension comprising the at least one cell culture medium and at least a first type of biological cells expressing a single-domain antibody.

In some embodiments, the method further comprises causing at least a portion of the suspension to flow through at least one filter to produce at least one filtrate lean in the first type of biological cells. In some embodiments, the at least one filtrate comprises a single-domain antibody.

In some embodiments, the method comprises flowing the at least one filtrate to an adjustment module (e.g., from the at least one filter to the adjustment module). In some embodiments, the method further comprises adjusting, within the adjustment module, one or more properties (e.g., pH, conductivity, product stability) of the at least one filtrate. In certain embodiments, the pH of the at least one filtrate is increased or decreased (e.g., to be compatible with the first column of the purification module). In some embodiments, increasing the pH of the at least one filtrate comprises adding a base to the at least one filtrate. In some embodiments, decreasing the pH of the at least one filtrate comprises adding an acid to the at least one filtrate. In some embodiments, the method further comprises flowing the at least one filtrate and/or the adjusted filtrate through a first column comprising a multimodal cation exchange resin. In certain embodiments, the multimodal cation exchange resin comprises Capto MMC ImpRes, Capto MMC, Nuvia cPrime, Toyopearl MX-Trp-650M, Eshmuno HCX, and/or CMM HyperCel. In certain non-limiting embodiments, the multimodal cation exchange resin comprises a CMM HyperCel resin.

In certain embodiments, the first column is operated in bind-elute mode. In some embodiments, the method further comprises flowing a first mobile phase material through the first column. In some embodiments, the first mobile phase material is configured to promote binding of the single-domain antibody to the multimodal cation exchange resin. In some embodiments, the first mobile phase material comprises sodium citrate, sodium phosphate, sodium chloride, sodium acetate, Tris-HC1, glycine, and/or histidine. According to certain embodiments, the first mobile phase material comprises 20 mM sodium citrate. In some embodiments, the first mobile phase material has a pH in the range of about 4.0 to about 6.0 (e.g., about 4.5 to about 5.5, about 4.0, about 4.5, about 5.0, about 5.5, about 6.0). In some embodiments, the method further comprises flowing the at least one filtrate and/or the adjusted filtrate through the first column and, subsequently, flowing a second mobile phase material through the first column. In some embodiments, the second mobile phase material is configured to wash one or more impurities from the multimodal cation exchange resin. In some embodiments, the second mobile phase material comprises sodium citrate, sodium phosphate, sodium chloride, sodium acetate, Tris-HC1, glycine, and/or histidine. According to certain embodiments, the second mobile phase material comprises 20 mM sodium phosphate. In some embodiments, the second mobile phase material has a pH in the range of about 5.0 to about 7.0 (e.g., about 5.5 to about 6.5, about 5.0, about 5.5, about 5.8, about 6.0, about 6.2, about 6.5, about 7.0). In some embodiments, the method further comprises subsequently flowing a third mobile phase material through the first column. In some embodiments, the third mobile phase material is configured to elute single-domain antibody from the multimodal cation exchange resin. In some embodiments, the third mobile phase material comprises sodium citrate, sodium phosphate, sodium chloride, sodium acetate, Tris-HC1, glycine, and/or histidine. According to certain embodiments, the third mobile phase material comprises 20 mM sodium phosphate. In some embodiments, the third mobile phase material has a pH in the range of about 6.0 to about 8.0 (e.g., about 6.5 to about 7.5, about 6.0, about 6.5, about 7.0, about 7.5, about 8.0). In some embodiments, the third mobile phase material has a salt (e.g., sodium chloride) concentration in the range of about 0 mM to about 150 mM (e.g., 50 mM to about 150 mM, about 75 mM to about 125 mM, about 75 mM, about 100 mM, about 125 mM, about 150 mM). In some embodiments, the method further comprises collecting one or more first fractions comprising the single-domain antibody from an outflow of the first column. In some embodiments, the one or more first fractions are lean in the first type of impurity relative to the at least one filtrate or the adjusted filtrate. In some embodiments, the one or more first fractions have a concentration of the first type of impurity that is at least about 50%, at least about 75%, at least about 90%, at least about 95%, or at least about 99% less than the concentration of the first type of impurity in the at least one filtrate or the adjusted filtrate.

In some embodiments, the method further comprises flowing the one or more first fractions through a second column comprising an anion exchange resin. In some embodiments, the anion exchange resin comprises a HyperCel STAR AX resin. In certain embodiments, the second column is operated in flow-through mode. In some embodiments, the method comprises flowing through a first mobile phase material through the second column. In some embodiments, the first mobile phase material comprises sodium citrate, sodium phosphate, sodium chloride, sodium acetate, Tris-HC1, glycine, and/or histidine. According to certain embodiments, the first mobile phase material comprises 20 mM sodium phosphate. In some embodiments, the first mobile phase material has a pH in a range of about 6.0 to about 8.0 (e.g., about 6.5 to about 7.5, about 6.0, about 6.5, about 7.0, about 7.5, about 8.0). In some embodiments, the first mobile phase material has a salt (e.g., sodium chloride) concentration in a range of about 0 mM to about 150 mM (e.g., about 50 mM to about 150 mM, about 75 mM to about 125 mM, about 100 mM, about 125 mM, about 150 mM). In some embodiments, the method further comprises collecting one or more second fractions comprising the single-domain antibody from an outflow of the second column. In some embodiments, the one or more second fractions are lean in the second type of impurity relative to the first fractions. In some embodiments, the one or more second fractions have a concentration of the second type of impurity that is at least about 50%, at least about 75%, at least about 90%, at least about 95%, or at least about 99% less than the concentration of the second type of impurity in the first fractions.

In some embodiments, the method further comprises flowing the second fractions to a formulation module to produce a formulated product stream. In some embodiments, flowing the purified filtrate through the formulation module comprises flowing the purified filtrate through a tangential flow filtration device. In some embodiments, flowing the purified filtrate through the formulation module comprises flowing the purified filtrate through a viral filtration unit. In some embodiments, the formulated product stream is lean in one or more viruses relative to the purified filtrate stream. In some embodiments, flowing the purified filtrate through the formulation module comprises flowing the purified filtrate through a dilution adjustment unit. In some embodiments, flowing the purified filtrate through the dilution adjustment unit comprises adding a diluent to the purified filtrate. In some embodiments, flowing the purified filtrate through the formulation module comprises depositing one or more portions of the purified filtrate stream into one or more containers (e.g., bags, vials, syringes, bottles). In some embodiments, the one or more containers are aseptic and/or sterile containers.

The term “single-domain antibody” has its ordinary meaning in the art and may refer to a single domain polypeptide comprising (e.g., consisting of) a variable domain (e.g., variable heavy chain domain) that is capable of binding an antigen. In certain embodiments, a single-domain antibody may be a single domain polypeptide comprising (e.g., consisting of) a single variable domain (e.g., variable heavy chain single domain). In other embodiments, a single-domain antibody may be a single domain polypeptide comprising (e.g., consisting of) two or more (e.g., two, three or more, four or more) variable domains (e.g., variable heavy chain single domain) covalently attached. In some embodiments, a single-domain antibody may be derived from an antibody, a heavy chain antibody, an antibody naturally devoid of light chains, an engineered antibody, and/or a single domain scaffold other than those derived from antibodies. In some embodiments, a single-domain antibody may be a nanobody. The term “nanobody” has its ordinary meaning in the art and may include a single-domain antibody derived from a heavy chain antibody of a species such as Camelidae (e.g., camel, llama, dromedary, alpaca, guanaco) and a nurse shark, and humanized variants thereof. For instance, a nanobody may be derived from a heavy chain camelid antibody (e.g., camel antibody, llama antibody, dromedary antibody, alpaca antibody, guanaco antibody).

In general, the systems and methods described herein may be used to manufacture any suitable single-domain antibody. Non-limiting examples of single-domain antibodies that may be manufactured using the systems and methods described herein include camelid antibodies (e.g., 3B2, 2KD1), shark antibodies, and those described in Steeland, S.; Vandenbroucke, R. E.; Libert, C. Nanobodies as Therapeutics: Big Opportunities for Small Antibodies, Drug Discov. Today 2016, 21 (7), 1076-1113, which is incorporated by reference in its entirety. The single-domain antibodies described herein may have a range of indications including, but not limited to, anti-toxin, anti-venom, anti-viral, anti-bacterial, anti-fungal, anti-parasitic, anti-cytokine, anti-hormone, anti-blood factor, anti-tumor, and anti-cancer.

In some embodiments, a single-domain antibody may have an isoelectric point in the range of about 4.0 to about 12.0 (e.g., about 4.0 to about 11.0, about 5.0 to about 10.0, about 5.0 to about 5.5, about 6.0, about 6.5, about 7.0, about 7.5, about 8.0, about 8.5, about 9,0, about 9.5, about 10.0, about 10.5, or about 11.0). For instance, a single-domain antibody may have an isoelectric point in the range of about 5.5 to about 8.0, of about 6.0 to about 8.0, or of about 6.5 to about 8.0. In some embodiments, a single-domain antibody may have a relatively low molecular weight. For instance, in some embodiments, a single-domain antibody may have a molecular weight in the range of about 12 kDa to about 30 kDa (e.g., of about 12 kDa to about 25 kDa, of about 12 kDa to about 20 kDa, of about 12 kDa to about 18 kDa, of about 12 kDa to about 15 kDa).

As used herein, the term “antibody” refers to an immunoglobulin molecule or an immunologically active portion thereof (e.g., antigen-binding portion). The antibody may be naturally produced or wholly or partially synthetically produced. Examples of immunologically active portions of immunoglobulin molecules include F(ab), Fv, and F(ab′) fragments which can be generated by cleaving the antibody with an enzyme such as pepsin. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. An immunoglobulin molecule may be a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three subdomains, C_{H1}, C_{H2} and C_{H3}. Each light chain is comprised of a light chain variable region (abbreviated herein as V_{L}) and a light chain constant region. The light chain constant region is comprised of one subdomain, C_{L}. The V_{H} and V_{L} regions can be further subdivided into regions of hypervariability, termed complementarity determining regions (CDR), interspersed with regions that are more conserved, termed framework regions (FR). The variable regions of the heavy and light chains contain a binding domain that interacts with an antigen.

Exemplary embodiments of the present disclosure are provided below. One exemplary embodiment is generally directed to the following:
1. A biomanufacturing system, comprising a perfusion bioreactor, wherein the perfusion bioreactor comprises:

a reaction chamber configured to receive at least one feed stream comprising at least one cell culture medium;

a suspension comprising the at least one cell culture medium and at least a first type of biological cells configured to express at least one biologically-produced product; at least one filter probe at least partially submerged in the suspension, wherein the at least one filter probe is configured to produce at least one filtrate stream lean in the first type of biological cells relative to the suspension, wherein the at least one filtrate stream comprises the at least one biologically-produced product;

an adjustment module fluidically connected to the perfusion bioreactor, wherein the adjustment module is configured to adjust one or more properties of the at least one filtrate stream to produce an adjusted filtrate stream; and

a purification module fluidically connected to the adjustment module, wherein the purification module is configured to remove at least a first type of impurity and a second type of impurity from the adjusted filtrate stream to produce a purified filtrate stream, wherein the purification module comprises:

a first partitioning unit configured to remove at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and

a second partitioning unit configured to remove at least a second type of impurity from the first partitioned filtrate stream to produce a second partitioned filtrate stream lean in the second type of impurity relative to the first partitioned filtrate stream, wherein the second partitioned filtrate stream comprises the at least one biologically-produced product, wherein the system is configured to be continuously operated.
2. The biomanufacturing system of sentence 1, wherein the reactor chamber has a volume of about 1 L or less.
3. The biomanufacturing system of sentence 2, wherein the at least one feed stream and the purified filtrate stream each have a flow rate of at least about 0.1 mL/min over a period of at least about 1 day.
4. The biomanufacturing system of any one of sentences 2-3, wherein the system is configured to produce at least about 10 μg of the at least one biologically-produced product per day.
5. The biomanufacturing system of any one of sentences 2-4, wherein the system is configured to produce at least about 50 mg of the at least one biologically-produced product per day.
6. The biomanufacturing system of any one of sentences 2-5, wherein the system is configured to produce at least about 1 g of the at least one biologically-produced product per day.
7. The biomanufacturing system of sentence 1, wherein the reactor chamber has a volume of about 1 L to about 10 L.
8. The biomanufacturing system of sentence 7, wherein the at least one feed stream and the purified filtrate stream each have a flow rate of at least about 0.5 mL/min over a period of at least about 1 day.
9. The biomanufacturing system of any one of sentences 7-8, wherein the system is configured to produce at least about 100 ᵢ.t.g of the at least one biologically-produced product per day.
10. The biomanufacturing system of any one of sentences 7-9, wherein the system is configured to produce at least about 500 mg of the at least one biologically-produced product per day.
11. The biomanufacturing system of any one of sentences 7-10, wherein the system is configured to produce at least about 10 g of the at least one biologically-produced product per day.
12. The biomanufacturing system of sentence 1, wherein the reactor chamber has a volume of about 10 L to about 50 L.
13. The biomanufacturing system of sentence 12, wherein the at least one feed stream and the purified filtrate stream each have a flow rate of at least about 5 mL/min over a period of at least about 1 day.
14. The biomanufacturing system of any one of sentences 12-13, wherein the system is configured to produce at least about 500 ᵢ.t.g of the at least one biologically-produced product per day.
15. The biomanufacturing system of any one of sentences 12-14, wherein the system is configured to produce at least about 2.5 g of the at least one biologically-produced product per day.
16. The biomanufacturing system of any one of sentences 12-15, wherein the system is configured to produce at least about 50 g of the at least one biologically-produced product per day.
17. The biomanufacturing system of any one of sentences 1-16, wherein the first type of biological cells are microbial cells, yeast cells, filamentous fungal cells, microalgal cells, or diatom cells.
18. The biomanufacturing system of sentence 17, wherein the yeast cells are Pichia pastoris cells.
19. The biomanufacturing system of any one of sentences 1-18, wherein the at least one biologically-produced product comprises a cytokine, an antibody, an antibody fragment, a nanobody, a hormone, an enzyme, a growth factor, a blood factor, a recombinant immunogen, and/or a fusion protein.
20. The biomanufacturing of sentence 19, wherein the antibody is a single-chain antibody, a bispecific antibody, and/or a monoclonal antibody.
21. The biomanufacturing system of any one of sentences 1-19, wherein the at least one biologically-produced product comprises human growth hormone (hGH).
22. The biomanufacturing system of any one of sentences 1-19, wherein the at least one biologically-produced product comprises granulocyte colony-stimulating factor (G-CSF).
23. The biomanufacturing system of any one of sentences 1-19, wherein the at least one biologically-produced product comprises an interferon.
24. The biomanufacturing system of sentence 23, wherein the interferon is interferon a-2b.
25. The biomanufacturing system of any one of sentences 1-24, wherein the one or more properties comprise pH.
26. The biomanufacturing system of sentence 25, wherein the adjustment module is configured to reduce the pH of the at least one filtrate stream.
27. The biomanufacturing system of sentence 25, wherein the adjustment module is configured to increase the pH of the at least one filtrate stream.
28. The biomanufacturing system of any one of sentences 1-27, wherein the one or more properties comprise conductivity.
29. The biomanufacturing system of sentence 28, wherein the adjustment module is configured to increase or decrease the conductivity of the at least one filtrate stream.
30. The biomanufacturing system of any one of sentences 1-29, wherein the one or more properties comprise biologically-produced product stability.
31. The biomanufacturing system of sentence 30, wherein the adjustment module is configured to increase the stability of the at least one biologically-produced product in the at least one filtrate stream.
32. The biomanufacturing system of any one of sentences 1-31, wherein the first partitioning unit is directly fluidically connected to the second partitioning unit.
33. The biomanufacturing system of any one of sentences 1-32, wherein the first partitioning unit applies a partitioning technique and the second partitioning unit applies the same partitioning technique.
34. The biomanufacturing system of any one of sentences 1-32, wherein the first partitioning unit applies a partitioning technique and the second partitioning unit applies a different partitioning technique.
35. The biomanufacturing system of any one of sentences 1-34, wherein the first partitioning unit comprises a first column comprising at least a first stationary phase material.
36. The biomanufacturing system of sentence 35, wherein the first stationary phase material comprises a cation exchange resin, a multimodal cation exchange resin, an anion exchange resin, a multimodal anion exchange resin, a hydrophobic charge induction chromatography (HCIC) resin, or an affinity chromatography resin.
37. The biomanufacturing system of any one of sentences 1-36, wherein the first partitioning unit comprises a filter.
38. The biomanufacturing system of sentence 37, wherein the filter comprises a filtration membrane and/or a monolith.
39. The biomanufacturing system of any one of sentences 1-38, wherein the first partitioning unit comprises a precipitation apparatus.
40. The biomanufacturing system of any one of sentences 1-39, wherein the first partitioning unit comprises a crystallization apparatus.
41. The biomanufacturing system of any one of sentences 1-40, wherein the second partitioning unit comprises a second column comprising at least a first stationary phase material.
42. The biomanufacturing system of sentence 41, wherein the first stationary phase material comprises a cation exchange resin, a multimodal cation exchange resin, an anion exchange resin, a multimodal anion exchange resin, a hydrophobic charge induction chromatography (HCIC) resin, or an affinity chromatography resin.
43. The biomanufacturing system of any one of sentences 1-42, wherein the second partitioning unit comprises a filter.
44. The biomanufacturing system of sentence 43, wherein the filter comprises a filtration membrane and/or a monolith.
45. The biomanufacturing system of any one of sentences 1-44, wherein the second partitioning unit comprises a precipitation apparatus.
46. The biomanufacturing system of any one of sentences 1-45, wherein the second partitioning unit comprises a crystallization apparatus.
47. The biomanufacturing system of any one of sentences 1-46, wherein the first type of impurity is different from the second type of impurity.
48. The biomanufacturing system of any one of sentences 1-47, wherein the purification module further comprises a third partitioning unit configured to remove at least a third type of impurity from the second partitioned filtrate stream to produce a third partitioned filtrate stream lean in the third type of impurity relative to the second partitioned filtrate stream, wherein the third partitioned filtrate stream comprises the at least one biologically-produced product.
49. The biomanufacturing system of sentence 48, wherein the third partitioning unit comprises a third column comprising at least a first stationary phase material.
50. The biomanufacturing system of sentence 49, wherein the first stationary phase comprises a cation exchange resin, a multimodal cation exchange resin, an anion exchange resin, a multimodal anion exchange resin,a hydrophobic charge induction chromatography (HCIC) resin, or an affinity chromatography resin.
51. The biomanufacturing system of any one of sentences 48-50, wherein the third partitioning unit comprises a filter.
52. The biomanufacturing system of sentence 51, wherein the filter comprises a filtration membrane and/or a monolith.
53. The biomanufacturing system of any one of sentences 48-52, wherein the third partitioning unit comprises a precipitation apparatus.
54. The biomanufacturing system of any one of sentences 48-53, wherein the third partitioning unit comprises a crystallization apparatus.
55. The biomanufacturing system of any one of sentences 48-54, wherein the third type of impurity is different from the first type of impurity and/or the second type of impurity.
56. The biomanufacturing system of any one of sentences 1-55, further comprising a formulation module fluidically connected to the purification module, wherein the formulation module is configured to produce a formulated product stream.
57. The biomanufacturing system of sentence 56, wherein the formulation module comprises a filtration unit.
58. The biomanufacturing system of sentence 57, wherein the filtration unit comprises a tangential flow filtration device.
59. The biomanufacturing system of any one of sentences 56-58, wherein the formulation module comprises a viral filtration unit.
60. The biomanufacturing system of any one of sentences 56-59, wherein the formulation module comprises a packaging unit.
61. The biomanufacturing system of sentence 60, wherein the packaging unit is configured to package one or more doses of the at least one biologically-produced product into one or more bags, one or more vials, one or more syringes, and/or one or more bottles.
62. The biomanufacturing system of any one of sentences 56-61, wherein the formulation module comprises a dilution adjustment unit.
63. The biomanufacturing system of any one of sentences 1-62, further comprising a buffer delivery module fluidically connected to the purification module, wherein the buffer delivery module is configured to deliver at least one buffer to at least one partitioning unit.
64. The biomanufacturing system of any one of sentences 1-63, further comprising a gas concentration device fluidically connected to the bioreactor.
65. The biomanufacturing system of sentence 64, wherein the gas concentration device is an oxygen concentrator.
66. The biomanufacturing system of any one of sentences 1-65, wherein at least one module of the system is disposable.
67. The biomanufacturing system of sentence 66, wherein each module of the system is disposable.
68. The biomanufacturing system of any one of sentences 1-67, wherein at least one module of the system is configured to be cleaned in place.
69. The biomanufacturing system of sentence 68, wherein each module of the system is configured to be cleaned in place.
70. The biomanufacturing system of any one of sentences 1-69, wherein the purified filtrate stream and/or the formulated product stream has a concentration of the pharmaceutical product of at least about 10 μg/mL.
71. The biomanufacturing system of any one of sentences 1-70, wherein the purified filtrate stream and/or the formulated product stream has a concentration of host cell proteins of about 20 ng/(mg product) or less.
72. The biomanufacturing system of any one of sentences 1-71, wherein the purified filtrate stream and/or the formulated product stream has a concentration of DNA of about 100 ng/(mg product) or less.
73. A method of producing at least one biologically-produced product, comprising:

supplying at least one feed stream comprising at least one cell culture medium to a perfusion bioreactor at a first flow rate;

producing, within the perfusion bioreactor, a suspension comprising the at least one cell culture medium and at least a first type of biological cells expressing the at least one biologically-produced product;

causing at least a portion of the suspension to flow through at least one filter probe to produce at least one filtrate stream lean in the first type of biological cells, wherein the at least one filtrate stream comprises the at least one biologically-produced product, wherein the at least one filter probe is at least partially submerged in the suspension;

adjusting one or more properties of the at least one filtrate stream to produce an adjusted filtrate stream; removing, within a purification module, at least a first type of impurity and a second type of impurity from the adjusted filtrate stream to produce a purified filtrate stream flowing at a second flow rate, wherein the purified filtrate stream comprises the at least one biologically-produced product and is lean in the first type of impurity and the second type of impurity relative to the adjusted filtrate stream, wherein producing the purified filtrate stream comprises:
removing, within a first partitioning unit, at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and

removing, within a second partitioning unit, at least the second type of impurity from the first partitioned filtrate stream to produce a second partitioned filtrate stream lean in the second type of impurity relative to the first partitioned filtrate stream, wherein the second partitioned filtrate stream comprises the at least one biologically-produced product.
74. The method of sentence 73, further comprising, prior to supplying the at least one feed stream comprising the at least one cell culture medium to the perfusion bioreactor: supplying a growth cell culture medium to the perfusion bioreactor;
incubating the first type of biological cells in the growth cell culture medium for a period of at least about 1 day; and
at least partially removing the growth cell culture medium from the perfusion bioreactor.
75. The method of any one of sentences 73-74, wherein the at least one feed stream is continuously supplied to the perfusion bioreactor at the first flow rate over a period of at least about 1 day.
76. The method of any one of sentences 73-75, wherein the first flow rate and the second flow rate are substantially the same.
77. The method of any one of sentences 73-76, wherein the perfusion bioreactor comprises a reactor chamber having a volume of about 1 L or less.
78. The method of sentence 77, wherein the first flow rate and/or second flow rate are maintained at about 0.1 mL/min or more over a period of about 1 day or more.
79. The method of any one of sentences 77-78, wherein at least about 10 ᵢ.t.g of the at least one biologically-produced product is produced in about 1 day or less.
80. The method of any one of sentences 77-79, wherein at least about 50 mg of the at least one biologically-produced product is produced in about 1 day or less.
81. The method of any one of sentences 77-80, wherein at least about 1 g of the at least one biologically-produced product is produced in about 1 day or less.
82. The method of any one of sentences 73-76, wherein the perfusion bioreactor comprises a reactor chamber having a volume of about 1 L to about 10 L.
83. The method of sentence 82, wherein the first flow rate and/or second flow rate are maintained at about 0.5 mL/min or more over a period of about 1 day or more.
84. The method of any one of sentences 82-83, wherein at least about 100 μg of the at least one biologically-produced product is produced in about 1 day or less.
85. The method of any one of sentences 82-84, wherein at least about 500 mg of the at least one biologically-produced product is produced in about 1 day or less.
86. The method of any one of sentences 82-85, wherein at least about 10 g of the at least one biologically-produced product is produced in about 1 day or less.
87. The method of any one of sentences 73-76, wherein the perfusion bioreactor comprises a reactor chamber having a volume of about 10 L to about 50 L.
88. The method of sentence 87, wherein the first flow rate and/or second flow rate are maintained at about 5 mL/min or more over a period of about 1 day or more.
89. The method of any one of sentences 87-88, wherein at least about 500 ᵢ.t.g of the at least one biologically-produced product is produced in about 1 day or less.
90. The method of any one of sentences 87-89, wherein at least about 2.5 g of the at least one biologically-produced product is produced in about 1 day or less.
91. The method of any one of sentences 87-90, wherein at least about 50 g of the at least one biologically-produced product is produced in about 1 day or less.
92. The method of any one of sentences 73-91, wherein the first type of biological cells are yeast cells, filamentous fungal cells, microalgal cells, or diatom cells.
93. The method of sentence 92, wherein the yeast cells are Pichia pastoris cells.
94. The method of any one of sentences 73-93, wherein the at least one biologically-produced product comprises a cytokine, an antibody, an antibody fragment, a nanobody, a hormone, an enzyme, a growth factor, a blood factor, a recombinant immunogen, and/or a fusion protein.
95. The method of sentence 94, wherein the antibody is a single-chain antibody, a bispecific antibody, and/or a monoclonal antibody.
96. The method of any one of sentences 73-95, wherein the at least one biologically-produced product comprises human growth hormone (hGH).
97. The method of any one of sentences 73-96, wherein the at least one biologically-produced product comprises granulocyte colony-stimulating factor (G-CSF).
98. The method of any one of sentences 73-97, wherein the at least one biologically-produced product comprises an interferon.
99. The method of sentence 98, wherein the interferon is interferon α-2b.
100. The method of any one of sentences 73-99, wherein adjusting one or more properties of the at least one filtrate stream comprises decreasing the pH of the at least one filtrate stream.
101. The method of any one of sentences 73-99, wherein adjusting one or more properties of the at least one filtrate stream comprises increasing the pH of the at least one filtrate stream.
102. The method of any one of sentences 73-101, wherein adjusting one or more properties of the at least one filtrate stream comprises increasing the conductivity of the at least one filtrate stream.
103. The method of any one of sentences 73-101, wherein adjusting one or more properties of the at least one filtrate stream comprises decreasing the conductivity of the at least one filtrate stream.
104. The method of any one of sentences 73-103, wherein adjusting one or more properties of the at least one filtrate stream comprises reducing the pH of the at least one filtrate stream.
105. The method of any one of sentences 73-104, wherein adjusting one or more properties of the at least one filtrate stream comprises increasing the stability of the at least one biologically-produced product in the at least one filtrate stream.
106. The method of any one of sentences 73-105, wherein the first partitioning unit and the second partitioning unit are directly fluidically connected.
107. The method of any one of sentences 73-106, wherein the first partitioning unit and the second partitioning unit apply the same partitioning technique.
108. The method of any one of sentences 73-107, wherein the first partitioning unit and the second partitioning unit apply different partitioning techniques.
109. The method of any one of sentences 73-108, wherein removing at least the first type of impurity comprises causing the adjusted filtrate stream to flow through a first column comprising at least a first stationary phase material.
110. The method of any one of sentences 73-109, wherein removing at least the first type of impurity comprises causing the adjusted filtrate stream to flow through a filter.
111. The method of sentence 110, wherein the filter comprises a filtration membrane and/or a monolith.
112. The method of any one of sentences 73-111, wherein removing at least the first type of impurity comprises causing the first type of impurity to precipitate and/or crystallize.
113. The method of any one of sentences 73-112, wherein removing at least the second type of impurity comprises causing the first partitioned filtrate stream to flow through a second column comprising at least a first stationary phase material.
114. The method of any one of sentences 73-113, wherein removing at least the second type of impurity comprises causing the first partitioned filtrate stream to flow through a filter.
115. The method of sentence 114, wherein the filter comprises a filtration membrane and/or a monolith.
116. The method of any one of sentences 73-115, wherein removing at least the second type of impurity comprises causing the second type of impurity to precipitate and/or crystallize.
117. The method of any one of sentences 73-116, wherein the first type of impurity is different from the second type of impurity.
118. The method of any one of sentences 73-117, further comprising removing, within a third partitioning unit, at least a third type of impurity from the second partitioned filtrate stream to produce a third partitioned filtrate stream lean in the third type of impurity relative to the second partitioned filtrate stream, wherein the third partitioned filtrate stream comprises the at least one biologically-produced product.
119. The method of sentence 118, wherein removing at least the third type of impurity comprises causing the second partitioned filtrate stream to flow through a third column comprising at least a first stationary phase material.
120. The method of any one of sentences 118-119, wherein removing at least the third type of impurity comprises causing the second partitioned filtrate stream to flow through a filter.
121. The method of sentence 120, wherein the filter comprises a filtration membrane and/or a monolith.
122. The method of any one of sentences 118-121, wherein removing at least the third type of impurity comprises causing the third type of impurity to precipitate and/or crystallize.
123. The method of any one of sentences 118-122, wherein the third type of impurity is different from the first type of impurity and/or the second type of impurity.
124. The method of any one of sentences 73-123, further comprising flowing the purified filtrate stream through a formulation module configured to produce a formulated product stream.
125. The method of sentence 124, wherein flowing the purified filtrate stream through the formulation module comprises flowing the purified filtrate stream through a tangential flow filtration device.
126. The method of any one of sentences 124-125, wherein flowing the purified filtrate stream through the formulation module comprises flowing the purified filtrate stream through a dilution adjustment unit.
127. The method of sentence 126, further comprising adding a diluent to the purified filtrate stream.
128. The method of any one of sentences 124-127, wherein flowing the purified filtrate stream through the formulation module comprises flowing the purified filtrate stream through a viral filtration unit, wherein the formulated product stream is lean in one or more viruses relative to the purified filtrate stream.
129. The method of any one of sentences 124-128, wherein flowing the purified filtrate stream through the formulation module comprises depositing one or more portions of the purified filtrate stream into one or more containers.
130. The method of sentence 129, wherein the one or more containers are aseptic and/or sterile containers.
131. The method of any one of sentences 129-130, wherein the one or more containers comprise one or more bags, vials, syringes, and/or bottles.
132. The biomanufacturing system of any preceding sentence, further comprising a process and monitoring control system, optionally wherein the process and monitoring control system comprises one or more optical sensors, optionally wherein the one or more optical sensors comprises one or more cameras.
133. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
134. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A second exemplary embodiment is generally directed to the following:
1. A biomanufacturing system, comprising a perfusion bioreactor, wherein the perfusion bioreactor comprises:

a reaction chamber configured to receive at least one feed stream comprising at least one cell culture medium;

a suspension comprising the at least one cell culture medium and at least a first type of biological cells configured to express at least one biologically-produced product;

a level sensing system configured to measure a level of the suspension in the reactor chamber of the perfusion bioreactor;

at least one filter probe at least partially submerged in the suspension, wherein the at least one filter probe is configured to produce at least one filtrate stream lean in the first type of biological cells relative to the suspension, wherein the at least one filtrate stream comprises the at least one biologically-produced product;

an adjustment module fluidically connected to the perfusion bioreactor, wherein the adjustment module is configured to adjust one or more properties of the at least one filtrate stream to produce an adjusted filtrate stream; and

a purification module fluidically connected to the adjustment module, wherein the purification module is configured to remove at least a first type of impurity and a second type of impurity from the adjusted filtrate stream to produce a purified filtrate stream, wherein the purification module comprises:

a first partitioning unit configured to remove at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and

a second partitioning unit configured to remove at least a second type of impurity from the first partitioned filtrate stream to produce a second partitioned filtrate stream lean in the second type of impurity relative to the first partitioned filtrate stream, wherein the second partitioned filtrate stream comprises the at least one biologically-produced product, wherein the system is configured to be continuously operated.
2. The biomanufacturing system of sentence 1, wherein the level sensing system is a magnetic level sensing system.
3. The biomanufacturing system of sentence 2, wherein the magnetic level sensing system comprises a magnetic float, a non-magnetic shaft, and one or more magnetically-activated switches.
4. The biomanufacturing system of sentence 1, wherein the level sensing system is an optical level sensing system.
5. The biomanufacturing system of sentence 4, wherein the optical level sensing system comprises a colored float and/or a colored probe.
6. The biomanufacturing system of sentence 4, wherein the optical level sensing system comprises a probe comprising a plurality of colored bands.
7. The biomanufacturing system of any one of sentences 1-6, wherein the level sensing system comprises a capacitance-based probe.
8. The biomanufacturing system of any one of sentences 1-7, wherein the reactor chamber has a volume of about 1 L or less.
9. The biomanufacturing system of sentence 8, wherein the at least one feed stream and the purified filtrate stream each have a flow rate of at least about 0.1 mL/min over a period of at least about 1 day.
10. The biomanufacturing system of any one of sentences 8-9, wherein the system is configured to produce at least about 10 ᵢig of the at least one biologically-produced product per day.
11. The biomanufacturing system of any one of sentences 8-10, wherein the system is configured to produce at least about 50 mg of the at least one biologically-produced product per day.
12. The biomanufacturing system of any one of sentences 8-11, wherein the system is configured to produce at least about 1 g of the at least one biologically-produced product per day.
13. The biomanufacturing system of any one of sentences 1-7, wherein the reactor chamber has a volume of about 1 L to about 10 L.
14. The biomanufacturing system of sentence 13, wherein the at least one feed stream and the purified filtrate stream each have a flow rate of at least about 0.5 mL/min over a period of at least about 1 day.
15. The biomanufacturing system of any one of sentences 13-14, wherein the system is configured to produce at least about 100 μg of the at least one biologically-produced product per day.
16. The biomanufacturing system of any one of sentences 13-15, wherein the system is configured to produce at least about 500 mg of the at least one biologically-produced product per day.
17. The biomanufacturing system of any one of sentences 13-16, wherein the system is configured to produce at least about 10 g of the at least one biologically-produced product per day.
18. The biomanufacturing system of any one of sentences 1-7, wherein the reactor chamber has a volume of about 10 L to about 50 L.
19. The biomanufacturing system of sentence 18, wherein the at least one feed stream and the purified filtrate stream each have a flow rate of at least about 5 mL/min over a period of at least about 1 day.
20. The biomanufacturing system of any one of sentences 18-19, wherein the system is configured to produce at least about 500 μg of the at least one biologically-produced product per day.
21. The biomanufacturing system of any one of sentences 18-20, wherein the system is configured to produce at least about 2.5 g of the at least one biologically-produced product per day.
22. The biomanufacturing system of any one of sentences 18-21, wherein the system is configured to produce at least about 50 g of the at least one biologically-produced product per day.
23. The biomanufacturing system of any one of sentences 1-22, wherein the first type of biological cells are yeast cells, filamentous fungal cells, microalgal cells, or diatom cells.
24. The biomanufacturing system of sentence 23, wherein the yeast cells are Pichia pastoris cells.
25. The biomanufacturing system of any one of sentences 1-24, wherein the at least one biologically-produced product comprises a cytokine, an antibody, an antibody fragment, a nanobody, a hormone, an enzyme, a growth factor, a blood factor, a recombinant immunogen, and/or a fusion protein.
26. The biomanufacturing of sentence 25, wherein the antibody is a single-chain antibody, a bispecific antibody, and/or a monoclonal antibody.
27. The biomanufacturing system of any one of sentences 1-26, wherein the at least one biologically-produced product comprises human growth hormone (hGH).
28. The biomanufacturing system of any one of sentences 1-27, wherein the at least one biologically-produced product comprises granulocyte colony-stimulating factor (G-CSF).
29. The biomanufacturing system of any one of sentences 1-28, wherein the at least one biologically-produced product comprises an interferon.
30. The biomanufacturing system of sentence 29, wherein the interferon is interferon α-2b.
31. The biomanufacturing system of any one of sentences 1-40, wherein the one or more properties comprise pH.
32. The biomanufacturing system of sentence 31, wherein the adjustment module is configured to reduce the pH of the at least one filtrate stream.
33. The biomanufacturing system of sentence 31, wherein the adjustment module is configured to increase the pH of the at least one filtrate stream.
34. The biomanufacturing system of any one of sentences 1-33, wherein the one or more properties comprise conductivity.
35. The biomanufacturing system of sentence 34, wherein the adjustment module is configured to increase or decrease the conductivity of the at least one filtrate stream.
36. The biomanufacturing system of any one of sentences 1-35, wherein the one or more properties comprise biologically-produced product stability.
37. The biomanufacturing system of sentence 36, wherein the adjustment module is configured to increase the stability of the at least one biologically-produced product in the at least one filtrate stream.
38. The biomanufacturing system of any one of sentences 1-37, wherein the first partitioning unit is directly fluidically connected to the second partitioning unit.
39. The biomanufacturing system of any one of sentences 1-38, wherein the first partitioning unit applies a partitioning technique and the second partitioning unit applies the same partitioning technique.
40. The biomanufacturing system of any one of sentences 1-39, wherein the first partitioning unit applies a partitioning technique and the second partitioning unit applies a different partitioning technique.
41. The biomanufacturing system of any one of sentences 1-40, wherein the first partitioning unit comprises a first column comprising at least a first stationary phase material.
42. The biomanufacturing system of sentence 41, wherein the first stationary phase material comprises a cation exchange resin, a multimodal cation exchange resin, an anion exchange resin, a multimodal anion exchange resin, a hydrophobic charge induction chromatography (HCIC) resin, or an affinity chromatography resin.
43. The biomanufacturing system of any one of sentences 1-42, wherein the first partitioning unit comprises a filter.
44. The biomanufacturing system of sentence 43, wherein the filter comprises a filtration membrane and/or a monolith.
45. The biomanufacturing system of any one of sentences 1-44, wherein the first partitioning unit comprises a precipitation apparatus.
46. The biomanufacturing system of any one of sentences 1-45, wherein the first partitioning unit comprises a crystallization apparatus.
47. The biomanufacturing system of any one of sentences 1-46, wherein the second partitioning unit comprises a second column comprising at least a first stationary phase material.
48. The biomanufacturing system of sentence 47, wherein the first stationary phase material comprises a cation exchange resin, a multimodal cation exchange resin, an anion exchange resin, a multimodal anion exchange resin, a hydrophobic charge induction chromatography (HCIC) resin, or an affinity chromatography resin.
49. The biomanufacturing system of any one of sentences 1-48, wherein the second partitioning unit comprises a filter.
50. The biomanufacturing system of sentence 49, wherein the filter comprises a filtration membrane and/or a monolith.
51. The biomanufacturing system of any one of sentences 1-50, wherein the second partitioning unit comprises a precipitation apparatus.
52. The biomanufacturing system of any one of sentences 1-51, wherein the second partitioning unit comprises a crystallization apparatus.
53. The biomanufacturing system of any one of sentences 1-52, wherein the first type of impurity is different from the second type of impurity.
54. The biomanufacturing system of any one of sentences 1-53, wherein the purification module further comprises a third partitioning unit configured to remove at least a third type of impurity from the second partitioned filtrate stream to produce a third partitioned filtrate stream lean in the third type of impurity relative to the second partitioned filtrate stream, wherein the third partitioned filtrate stream comprises the at least one biologically-produced product.
55. The biomanufacturing system of sentence 54, wherein the third partitioning unit comprises a third column comprising at least a first stationary phase material.
56. The biomanufacturing system of sentence 55, wherein the first stationary phase comprises a cation exchange resin, a multimodal cation exchange resin, an anion exchange resin, a multimodal anion exchange resin, a hydrophobic charge induction chromatography (HCIC) resin, or an affinity chromatography resin.
57. The biomanufacturing system of any one of sentences 54-56, wherein the third partitioning unit comprises a filter.
58. The biomanufacturing system of sentence 57, wherein the filter comprises a filtration membrane and/or a monolith.
59. The biomanufacturing system of any one of sentences 54-58, wherein the third partitioning unit comprises a precipitation apparatus.
60. The biomanufacturing system of any one of sentences 54-59, wherein the third partitioning unit comprises a crystallization apparatus.
61. The biomanufacturing system of any one of sentences 54-60, wherein the third type of impurity is different from the first type of impurity and/or the second type of impurity.
62. The biomanufacturing system of any one of sentences 1-61, further comprising a formulation module fluidically connected to the purification module, wherein the formulation module is configured to produce a formulated product stream.
63. The biomanufacturing system of sentence 62, wherein the formulation module comprises a filtration unit.
64. The biomanufacturing system of sentence 63, wherein the filtration unit comprises a tangential flow filtration device.
65. The biomanufacturing system of any one of sentences 62-64, wherein the formulation module comprises a viral filtration unit.
66. The biomanufacturing system of any one of sentences 62-65, wherein the formulation module comprises a packaging unit.
67. The biomanufacturing system of sentence 66, wherein the packaging unit is configured to package one or more doses of the at least one biologically-produced product into one or more bags, one or more vials, one or more syringes, and/or one or more bottles.
68. The biomanufacturing system of any one of sentences 62-67, wherein the formulation module comprises a dilution adjustment unit.
69. The biomanufacturing system of any one of sentences 1-68, further comprising a buffer delivery module fluidically connected to the purification module, wherein the buffer delivery module is configured to deliver at least one buffer to at least one partitioning unit.
70. The biomanufacturing system of any one of sentences 1-69, further comprising a gas concentration device fluidically connected to the bioreactor.
71. The biomanufacturing system of sentence 70, wherein the gas concentration device is an oxygen concentrator.
72. The biomanufacturing system of any one of sentences 1-71, wherein at least one module of the system is disposable.
73. The biomanufacturing system of sentence 72, wherein each module of the system is disposable.
74. The biomanufacturing system of any one of sentences 1-73, wherein at least one module of the system is configured to be cleaned in place.
75. The biomanufacturing system of sentence 74, wherein each module of the system is configured to be cleaned in place.
76. The biomanufacturing system of any one of sentences 1-75, wherein the purified filtrate stream and/or the formulated product stream has a concentration of the pharmaceutical product of at least about 10 μg/mL.
77. The biomanufacturing system of any one of sentences 1-76, wherein the purified filtrate stream and/or the formulated product stream has a concentration of host cell proteins of about 20 ng/(mg product) or less.

78. The biomanufacturing system of any one of sentences 1-77, wherein the purified filtrate stream and/or the formulated product stream has a concentration of DNA of about 100 ng/(mg product) or less.
79. A method of producing at least one biologically-produced product, comprising:

supplying at least one feed stream comprising at least one cell culture medium to a perfusion bioreactor at a first flow rate;

producing, within the perfusion bioreactor, a suspension comprising the at least one cell culture medium and at least a first type of biological cells expressing the at least one biologically-produced product;

measuring a level of the suspension in a reaction chamber of the perfusion bioreactor using a level sensing system;

causing at least a portion of the suspension to flow through at least one filter probe to produce at least one filtrate stream lean in the first type of biological cells, wherein the at least one filtrate stream comprises the at least one biologically-produced product, wherein the at least one filter probe is at least partially submerged in the suspension;

adjusting one or more properties of the at least one filtrate stream to produce an adjusted filtrate stream;

removing, within a purification module, at least a first type of impurity and a second type of impurity from the adjusted filtrate stream to produce a purified filtrate stream flowing at a second flow rate, wherein the purified filtrate stream comprises the at least one biologically-produced product and is lean in the first type of impurity and the second type of impurity relative to the adjusted filtrate stream, wherein producing the purified filtrate stream comprises: removing, within a first partitioning unit, at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and

removing, within a second partitioning unit, at least the second type of impurity from the first partitioned filtrate stream to produce a second partitioned filtrate stream lean in the second type of impurity relative to the first partitioned filtrate stream, wherein the second partitioned filtrate stream comprises the at least one biologically-produced product.
80. The method of sentence 79, wherein the level sensing system is a magnetic level sensing system.
81. The method of sentence 80, wherein the magnetic level sensing system comprises a magnetic float, a non-magnetic shaft, and one or more magnetically-activated switches.
82. The method of sentence 79, wherein the level sensing system is an optical level sensing system.
83. The method of sentence 82, wherein the optical level sensing system comprises a colored float and/or a colored probe.
84. The method of sentence 82, wherein the optical level sensing system comprises a probe comprising a plurality of colored bands.
85. The method of any one of sentences 79-84, wherein the level sensing system comprises a capacitance-based probe.
86. The method of any one of sentences 79-85, further comprising, prior to supplying the at least one feed stream comprising the at least one cell culture medium to the perfusion bioreactor, supplying a growth cell culture medium to the perfusion bioreactor; incubating the first type of biological cells in the growth cell culture medium for a period of at least about 1 day; and at least partially removing the growth cell culture medium from the perfusion bioreactor.
87. The method of any one of sentences 79-86, wherein the at least one feed stream is continuously supplied to the perfusion bioreactor at the first flow rate over a period of at least about 1 day.
88. The method of any one of sentences 79-87, wherein the first flow rate and the second flow rate are substantially the same.
89. The method of any one of sentences 79-88, wherein the perfusion bioreactor comprises a reactor chamber having a volume of about 1 L or less.
90. The method of sentence 89, wherein the first flow rate and/or second flow rate are maintained at about 0.1 mL/min or more over a period of about 1 day or more.
91. The method of any one of sentences 89-90, wherein at least about 10 ᵢ.t.g of the at least one biologically-produced product is produced in about 1 day or less.
92. The method of any one of sentences 89-91, wherein at least about 50 mg of the at least one biologically-produced product is produced in about 1 day or less.
93. The method of any one of sentences 89-92, wherein at least about 1 g of the at least one biologically-produced product is produced in about 1 day or less.
94. The method of any one of sentences 79-88, wherein the perfusion bioreactor comprises a reactor chamber having a volume of about 1 L to about 10 L.
95. The method of sentence 94, wherein the first flow rate and/or second flow rate are maintained at about 0.5 mL/min or more over a period of about 1 day or more.
96. The method of any one of sentences 94-95, wherein at least about 100 μg of the at least one biologically-produced product is produced in about 1 day or less.
97. The method of any one of sentences 94-96, wherein at least about 500 mg of the at least one biologically-produced product is produced in about 1 day or less.
98. The method of any one of sentences 94-97, wherein at least about 10 g of the at least one biologically-produced product is produced in about 1 day or less.
99. The method of any one of sentences 79-88, wherein the perfusion bioreactor comprises a reactor chamber having a volume of about 10 L to about 50 L.
100. The method of sentence 99, wherein the first flow rate and/or second flow rate are maintained at about 5 mL/min or more over a period of about 1 day or more.
101. The method of any one of sentences 99-100, wherein at least about 500 ᵢ.t.g of the at least one biologically-produced product is produced in about 1 day or less.
102. The method of any one of sentences 99-101, wherein at least about 2.5 g of the at least one biologically-produced product is produced in about 1 day or less.
103. The method of any one of sentences 99-102, wherein at least about 50 g of the at least one biologically-produced product is produced in about 1 day or less.
104. The method of any one of sentences 79-103, wherein the first type of biological cells are yeast cells, filamentous fungal cells, microalgal cells, or diatom cells.
105. The method of sentence 104, wherein the yeast cells are Pichia pastoris cells.
106. The method of any one of sentences 79-105, wherein the at least one biologically-produced product comprises a cytokine, an antibody, an antibody fragment, a nanobody, a hormone, an enzyme, a growth factor, a blood factor, a recombinant immunogen, and/or a fusion protein.
107. The method of sentence 106, wherein the antibody is a single-chain antibody, a bispecific antibody, and/or a monoclonal antibody.
108. The method of any one of sentences 79-107, wherein the at least one biologically-produced product comprises human growth hormone (hGH).
109. The method of any one of sentences 79-108, wherein the at least one biologically-produced product comprises granulocyte colony-stimulating factor (G-CSF).
110. The method of any one of sentences 79-109, wherein the at least one biologically-produced product comprises an interferon.
111. The method of sentence 110, wherein the interferon is interferon α-2b.
112. The method of any one of sentences 79-111, wherein adjusting one or more properties of the at least one filtrate stream comprises decreasing the pH of the at least one filtrate stream.
113. The method of any one of sentences 79-112, wherein adjusting one or more properties of the at least one filtrate stream comprises increasing the pH of the at least one filtrate stream.
114. The method of any one of sentences 79-113, wherein adjusting one or more properties of the at least one filtrate stream comprises increasing the conductivity of the at least one filtrate stream.
115. The method of any one of sentences 79-114, wherein adjusting one or more properties of the at least one filtrate stream comprises decreasing the conductivity of the at least one filtrate stream.
116. The method of any one of sentences 79-115, wherein adjusting one or more properties of the at least one filtrate stream comprises reducing the pH of the at least one filtrate stream.
117. The method of any one of sentences 79-116, wherein adjusting one or more properties of the at least one filtrate stream comprises increasing the stability of the at least one biologically-produced product in the at least one filtrate stream.
118. The method of any one of sentences 79-117, wherein the first partitioning unit and the second partitioning unit are directly fluidically connected.
119. The method of any one of sentences 79-118, wherein the first partitioning unit and the second partitioning unit apply the same partitioning technique.
120. The method of any one of sentences 79-119, wherein the first partitioning unit and the second partitioning unit apply different partitioning techniques.
121. The method of any one of sentences 79-120, wherein removing at least the first type of impurity comprises causing the adjusted filtrate stream to flow through a first column comprising at least a first stationary phase material.
122. The method of any one of sentences 79-121, wherein removing at least the first type of impurity comprises causing the adjusted filtrate stream to flow through a filter.
123. The method of sentence 122, wherein the filter comprises a filtration membrane and/or a monolith.
124. The method of any one of sentences 79-123, wherein removing at least the first type of impurity comprises causing the first type of impurity to precipitate and/or crystallize.
125. The method of any one of sentences 79-124, wherein removing at least the second type of impurity comprises causing the first partitioned filtrate stream to flow through a second column comprising at least a first stationary phase material.
126. The method of any one of sentences 79-125, wherein removing at least the second type of impurity comprises causing the first partitioned filtrate stream to flow through a filter.
127. The method of sentence 126, wherein the filter comprises a filtration membrane and/or a monolith.
128. The method of any one of sentences 79-127, wherein removing at least the second type of impurity comprises causing the second type of impurity to precipitate and/or crystallize.
129. The method of any one of sentences 79-128, wherein the first type of impurity is different from the second type of impurity.
130. The method of any one of sentences 79-129, further comprising removing, within a third partitioning unit, at least a third type of impurity from the second partitioned filtrate stream to produce a third partitioned filtrate stream lean in the third type of impurity relative to the second partitioned filtrate stream, wherein the third partitioned filtrate stream comprises the at least one biologically-produced product.
131. The method of sentence 130, wherein removing at least the third type of impurity comprises causing the second partitioned filtrate stream to flow through a third column comprising at least a first stationary phase material.
132. The method of any one of sentences 130-131, wherein removing at least the third type of impurity comprises causing the second partitioned filtrate stream to flow through a filter.
133. The method of sentence 132, wherein the filter comprises a filtration membrane and/or a monolith.
134. The method of any one of sentences 130-133, wherein removing at least the third type of impurity comprises causing the third type of impurity to precipitate and/or crystallize.
135. The method of any one of sentences 130-134, wherein the third type of impurity is different from the first type of impurity and/or the second type of impurity.
136. The method of any one of sentences 79-135, further comprising flowing the purified filtrate stream through a formulation module configured to produce a formulated product stream.
137. The method of sentence 136, wherein flowing the purified filtrate stream through the formulation module comprises flowing the purified filtrate stream through a tangential flow filtration device.
138. The method of any one of sentences 136-137, wherein flowing the purified filtrate stream through the formulation module comprises flowing the purified filtrate stream through a dilution adjustment unit.
139. The method of sentence 138, further comprising adding a diluent to the purified filtrate stream.
140. The method of any one of sentences 136-139, wherein flowing the purified filtrate stream through the formulation module comprises flowing the purified filtrate stream through a viral filtration unit, wherein the formulated product stream is lean in one or more viruses relative to the purified filtrate stream.
141. The method of any one of sentences 136-140, wherein flowing the purified filtrate stream through the formulation module comprises depositing one or more portions of the purified filtrate stream into one or more containers.
142. The method of sentence 141, wherein the one or more containers are aseptic and/or sterile containers.
143. The method of any one of sentences 141-142, wherein the one or more containers comprise one or more bags, vials, syringes, and/or bottles.
144. The biomanufacturing system of any preceding sentence, further comprising a process and monitoring control system, optionally wherein the process and monitoring control system comprises one or more optical sensors, optionally wherein the one or more optical sensors comprises one or more cameras.
145. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
146. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A third exemplary embodiment is generally directed to the following:
1. A system for producing G-CSF, comprising a bioreactor, wherein the bioreactor comprises a reaction chamber containing a suspension comprising at least one cell culture medium and at least a first type of biological cells configured to express G-CSF;

at least one filter, wherein the at least one filter is configured to receive an output of the bioreactor and produce at least one filtrate lean in the first type of biological cells relative to the suspension, wherein the at least one filtrate comprises G-CSF; and

a purification module, wherein the purification module is configured to remove at least a first type of impurity, a second type of impurity, and a third type of impurity from the first filtrate to produce a purified filtrate, wherein the purification module comprises:

a first column comprising a multimodal cation exchange resin;

a second column comprising an anion exchange resin; and

a third column comprising an HCIC resin.
2. The system of sentence 1, wherein the bioreactor is a perfusion bioreactor.
3. The system of sentence 2, wherein the bioreactor is configured to receive at least one feed stream comprising the at least one cell culture medium; the at least one filter is fluidically connected to the bioreactor and the first filtrate is an at least one filtrate stream; the purification module is fluidically connected to the at least one filter and the purified filtrate is a purified filtrate stream; and the system is configured to be continuously operated.
4. The system of any one of sentences 1-3, wherein the at least one filter comprises at least one filter probe at least partially submerged in the suspension in the bioreactor.
5. The system of any one of sentences 1-4, wherein the system further comprises a pH adjustment module configured to increase or decrease the pH of the at least one filtrate to produce a pH-adjusted filtrate.
6. The system of sentence 5, wherein the pH adjustment module is fluidically connected to the bioreactor, the at least one filter, and/or the purification module.
7. The system of any one of sentences 5-6, wherein the pH-adjusted filtrate has a pH of about 5.0.
8. The system of any one of sentences 1-7, wherein the first column is configured to remove at least the first type of impurity from the at least one filtrate to produce a first partitioned filtrate lean in the first type of impurity relative to the at least one filtrate, wherein the first partitioned filtrate comprises G-CSF.
9. The system of any one of sentences 1-8, wherein the multimodal cation exchange resin comprises a Capto MMC ImpRes resin.
10. The system of any one of sentences 1-9, wherein the second column is configured to remove at least the second type of impurity from the first partitioned filtrate to produce a second partitioned filtrate lean in the second type of impurity relative to the first partitioned filtrate, wherein the second partitioned filtrate comprises G-CSF.
11. The system of any one of sentences 1-10, wherein the anion exchange resin comprises a HyperCel STAR AX resin.
12. The system of any one of sentences 1-11, wherein the third column is configured to remove at least the third type of impurity from the second partitioned filtrate to produce a third partitioned filtrate lean in the third type of impurity relative to the second partitioned filtrate, wherein the third partitioned filtrate comprises G-CSF.
13. The system of any one of sentences 1-12, wherein the HCIC resin comprises an MEP HyperCel resin.
14. The system of any one of sentences 1-13, wherein the first type of biological cells are yeast cells, filamentous fungal cells, microalgal cells, or diatom cells.
15. The system of sentence 14, wherein the yeast cells are Pichia pastoris cells.
16. The system of any one of sentences 1-15, wherein the cell culture medium comprises chemically defined media comprising a carbon source, chemically defined media comprising an additive, or buffered methanol-complex media (BMMY).
17. The system of any one of sentences 1-16, further comprising a formulation module fluidically connected to the purification module, wherein the formulation module is configured to produce a formulated biologically-produced product stream.
18. The system of sentence 17, wherein the formulation module comprises a filtration unit.
19. The system of sentence 18, wherein the filtration unit comprises a tangential flow filtration device.
20. The system of any one of sentences 17-19, wherein the formulation module comprises a viral filtration unit.
21. The system of any one of sentences 17-20, wherein the formulation module comprises a product packaging unit.
22. The system of sentence 21, wherein the packaging unit is configured to package one or more doses of the at least one biologically-produced product into one or more bags, one or more vials, one or more syringes, and/or one or more bottles.
23. The system of any one of sentences 1-22, wherein the purified filtrate and/or formulated biologically-produced product stream has a host cell protein concentration of about 20 ng/(mg G-CSF) or less.
24. The system of any one of sentences 1-23, wherein the purified filtrate and/or formulated biologically-produced product stream does not have a detectable level of DNA.
25. The system of any one of sentences 1-24, wherein the purified filtrate and/or formulated biologically-produced product stream has an aggregate content of about 1% or less.
26. The system of any of sentences 1-25, wherein the reactor chamber has a volume of about 1 L or less.
27. The system of sentence 26, wherein the at least one feed stream and the purified filtrate stream each have a flow rate of at least about 0.1 mL/min over a period of at least about 1 day.
28. The system of any one of sentences 26-27, wherein the system is configured to produce at least about 10 mg of G-CSF per day.
29. The system of any one of sentences 1-26, wherein the reactor chamber has a volume of about 1 L to about 10 L.
30. The system of sentence 29, wherein the at least one feed stream and the purified filtrate stream each have a flow rate of at least about 0.5 mL/min over a period of at least about 1 day. 31. The system of any one of sentences 29-30, wherein the system is configured to produce at least about 50 mg of G-CSF per day.
32. The system of any one of sentences 1-31, wherein the reactor chamber has a volume of about 10 L to about 50 L.
33. The system of sentence 32, wherein the at least one feed stream and the purified filtrate stream each have a flow rate of at least about 5 mL/min over a period of at least about 1 day.
34. The system of any one of sentences 32-33, wherein the system is configured to produce at least about 500 mg of G-CSF per day.
35. A method of producing G-CSF, comprising:

applying at least one cell culture medium to a bioreactor; producing, within the bioreactor, a suspension comprising the at least one cell culture medium and at least a first type of biological cells expressing G-CSF;

causing at least a portion of the suspension to flow through at least one filter to produce at least one filtrate lean in the first type of biological cells, wherein the at least one filtrate comprises G-CSF;

flowing the at least one filtrate through a purification module to produce a purified filtrate, wherein producing the purified filtrate comprises flowing the at least one filtrate through a first column comprising a multimodal cation exchange resin;

collecting one or more first fractions comprising G-CSF from an outflow of the first column; flowing the one or more first fractions through a second column comprising an anion exchange resin;

collecting one or more second fractions comprising G-CSF from an outflow of the second column;

flowing the one or more second fractions through a third column comprising an HCIC resin; and

collecting one or more third fractions comprising G-CSF from an outflow of the third column.
36. The method of sentence 35, wherein the bioreactor is a perfusion bioreactor.
37. The method of sentence 36, wherein at least one feed stream comprising the at least one cell culture medium is continuously supplied to the perfusion bioreactor at a first flow rate over a period of at least about 1 day; the at least one filter is fluidically connected to the bioreactor and the at least one filtrate is an at least one filtrate stream; and the purified filtrate is a purified filtrate stream flowing at a second flow rate, wherein the purified filtrate stream comprises the one or more third fractions.
38. The method of any one of sentences 35-37, further comprising, prior to supplying the at least one cell culture medium to the bioreactor, supplying a growth cell culture medium to the bioreactor; incubating the first type of biological cells in the growth cell culture medium for a period of at least about 1 day; and at least partially removing the growth cell culture medium from the bioreactor.
39. The method of any one of sentences 35-38, wherein the at least one filter comprises at least one filter probe at least partially submerged in the suspension in the bioreactor.
40. The method of any one of sentences 35-39, further comprising adjusting the pH of the at least one filtrate to produce a pH-adjusted filtrate.
41. The method of sentence 40, wherein the pH-adjusted filtrate has a pH of about 5.0.
42. The method of any one of sentences 40-41, wherein the pH is adjusted in a pH adjustment module that is fluidically connected to the at least one filter and the first column of the purification module.
43. The method of any one of sentences 35-42, wherein the one or more first fractions are lean in a first type of impurity relative to the at least one filtrate.
44. The method of any one of sentences 35-43, wherein the multimodal cation exchange resin comprises a Capto MMC ImpRes resin.
45. The method of any one of sentences 35-44, wherein the first column is operated in bind-elute mode.
46. The method of sentence 45, further comprising flowing a first mobile phase material through the first column prior to flowing the at least one filtrate through the first column, wherein the first mobile phase material is configured to promote binding of G-CSF to the multimodal cation exchange resin.
47. The method of sentence 46, wherein the first mobile phase material has a pH of about 5.0.
48. The method of any one of sentences 46-47, further comprising flowing a second mobile phase material through the first column after flowing the at least one filtrate through the first column.
49. The method of sentence 48, wherein the second mobile phase material has a pH of about 5.8 and a salt concentration of about 150 mM.
50. The method of any one of sentences 46-49, further comprising flowing a third mobile phase material through the first column after flowing the second mobile phase material through the first column, wherein the third mobile phase material is configured to elute G-CSF from the first column.
51. The method of sentence 50, wherein the third mobile phase material has a pH of about 7.0 and a salt concentration of about 150 mM.
52. The method of any one of sentences 35-51, wherein the one or more second fractions are lean in a second type of impurity relative to the first fractions.
53. The method of any one of sentences 35-52, wherein the anion exchange resin comprises a HyperCel STAR AX resin.
54. The method of any one of sentences 35-53, wherein the second column is operated in flow-through mode.
55. The method of any one of sentences 35-54, wherein the one or more third fractions are lean in a third type of impurity relative to the second fractions.
56. The method of any one of sentences 35-55, wherein the HCIC resin comprises an MEP HyperCel resin.
57. The method of any one of sentences 35-56, wherein the third column is operated in bind-elute mode.
58. The method of sentence 57, further comprising flowing a first mobile phase material through the third column prior to flowing the second fractions through the third column, wherein the first mobile phase material is configured to promote binding of G-CSF to the HCIC resin.
59. The method of sentence 58, wherein the first mobile phase material has a pH of about 7.0 and a salt concentration of about 150 mM.
60. The method of any one of sentences 57-59, further comprising flowing a second mobile phase material through the third column after flowing the second fractions through the third column.
61. The method of sentence 60, wherein the second mobile phase material has a pH of about 5.5 and a salt concentration less than about 150 mM.
62. The method of any one of sentences 57-61, further comprising flowing a third mobile phase material through the third column after flowing the second mobile phase material through the third column, wherein the third mobile phase material is configured to elute G-CSF from the third column.
63. The method of sentence 62, wherein the third mobile phase material has a pH of about 3.0 and a salt concentration less than about 150 mM.
64. The method of any one of sentences 35-63, wherein the first type of biological cells are yeast cells, filamentous fungal cells, microalgal cells, or diatom cells.
65. The method of sentence 64, wherein the yeast cells are Pichia pastoris cells.
66. The method of any one of sentences 35-65, wherein the at least one cell culture medium comprises chemically defined media comprising a carbon source, chemically defined media comprising an additive, or buffered methanol-complex media (BMMY).
67. The method of any one of sentences 35-66, further comprising flowing the purified filtrate through a formulation module configured to produce a formulated product stream.
68. The method of sentence 67, wherein flowing the purified filtrate through the formulation module comprises flowing the purified filtrate through a tangential flow filtration device.
69. The method of any one of sentences 67-68, wherein flowing the purified filtrate through the formulation module comprises flowing the purified filtrate stream through a viral filtration unit, wherein the formulated product stream is lean in one or more viruses relative to the purified filtrate stream.
70. The method of any one of sentences 67-69, wherein flowing the purified filtrate through the formulation module comprises depositing one or more portions of the purified filtrate stream into one or more containers.
71. The method of sentence 70, wherein the one or more containers are aseptic and/or sterile containers.
72. The method of any one of sentences 70-71, wherein the one or more containers comprise one or more bags, vials, syringes, and/or bottles.
73. The method of any one of sentences 35-72, wherein the purified filtrate and/or the formulated stream have a host cell protein concentration of about 20 ng/(mg G-CSF) or less.
74. The method of any one of sentences 35-73, wherein the purified filtrate and/or the formulated stream do not have a detectable level of DNA.
75. The method of any one of sentences 35-74, wherein the purified filtrate and/or the formulated stream have an aggregate content of about 1% or less.
76. The method of any one of sentences 36-75, wherein the bioreactor comprises a reactor chamber having a volume of about 1 L or less.
77. The method of sentence 76, wherein the first flow rate and/or the second flow rate are maintained at about 0.1 mL/min or more over a period of about 1 day or more.
78. The method of any one of sentences 76-77, wherein at least about 10 mg of G-CSF is produced in about 1 day or less.
79. The method of any one of sentences 36-75, wherein the bioreactor comprises a reactor chamber having a volume of about 1 L to about 10 L.
80. The method of sentence 79, wherein the first flow rate and/or the second flow rate are maintained at about 0.5 mL/min or more over a period of about 1 day or more.
81. The method of any one of sentences 79-80, wherein at least about 50 mg of G-CSF is produced in about 1 day or less.
82. The method of any one of sentences 35-75, wherein the bioreactor comprises a reactor chamber having a volume of about 10 L to about 50 L.
83. The method of sentence 82, wherein the first flow rate and/or the second flow rate are maintained at about 5 mL/min or more over a period of about 1 day or more.
84. The method of any one of sentences 82-83, wherein at least about 500 mg of G-CSF is produced in about 1 day or less.
85. The method of any one of sentences 67-84, wherein flowing the purified filtrate through the formulation module comprises flowing the purified filtrate through a dilution adjustment unit.
86. The method of sentence 85, wherein flowing the purified filtrate through the dilution adjustment unit comprises adding a diluent to the purified filtrate.
87. The system of any one of sentences 17-34, wherein the formulation module comprises a dilution adjustment unit.
88. The biomanufacturing system of any preceding sentence, further comprising a process and monitoring control system, optionally wherein the process and monitoring control system comprises one or more optical sensors, optionally wherein the one or more optical sensors comprises one or more cameras.
89. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
90. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A fourth exemplary embodiment is generally directed to the following:
1. A system for producing interferon-α2b (IFN), comprising:

a bioreactor, wherein the bioreactor comprises a reaction chamber containing a suspension comprising at least one cell culture medium and at least a first type of biological cells configured to express interferon-α2b;

at least one filter, wherein the at least one filter is configured to receive an output of the bioreactor and produce at least one filtrate lean in the first type of biological cells relative to the suspension, wherein the at least one filtrate comprises interferon-α2b; and

a purification module, wherein the purification module is configured to remove at least a first type of impurity, a second type of impurity, and a third type of impurity from the first filtrate to produce a purified filtrate, wherein the purification module comprises:

a first column comprising a multimodal cation exchange resin;

a second column comprising an HCIC resin; and

a third column comprising a cation exchange resin.
2. The system of sentence 1, wherein the bioreactor is a perfusion bioreactor.
3. The system of sentence 2, wherein the bioreactor is configured to receive at least one feed stream comprising the at least one cell culture medium; the at least one filter is fluidically connected to the bioreactor and the first filtrate is an at least one filtrate stream; the purification module is fluidically connected to the at least one filter and the purified filtrate is a purified filtrate stream; and the system is configured to be continuously operated.
4. The system of any one of sentences 1-3, wherein the at least one filter comprises at least one filter probe at least partially submerged in the suspension in the bioreactor.
5. The system of any one of sentences 1-4, wherein the system further comprises a pH adjustment module configured to increase or decrease the pH of the at least one filtrate to produce a pH-adjusted filtrate.
6. The system of sentence 5, wherein the pH adjustment module is fluidically connected to the bioreactor, the at least one filter, and/or the purification module.
7. The system of any one of sentences 5-6, wherein the pH-adjusted filtrate has a pH of about 4.0.
8. The system of any one of sentences 1-7, wherein the first column is configured to remove at least the first type of impurity from the at least one filtrate to produce a first partitioned filtrate lean in the first type of impurity relative to the at least one filtrate, wherein the first partitioned filtrate comprises interferon-α2b.
9. The system of any one of sentences 1-8, wherein the multimodal cation exchange resin comprises a Capto MMC ImpRes resin.
10. The system of any one of sentences 1-9, wherein the second column is configured to remove at least the second type of impurity from the first partitioned filtrate to produce a second partitioned filtrate lean in the second type of impurity relative to the first partitioned filtrate, wherein the second partitioned filtrate comprises interferon-α2b.
11. The system of any one of sentences 1-10, wherein the HCIC resin comprises an MEP HyperCel resin and/or a HEA HyperCel resin.
12. The system of any one of sentences 1-11, wherein the third column is configured to remove at least the third type of impurity from the second partitioned filtrate to produce a third partitioned filtrate lean in the third type of impurity relative to the second partitioned filtrate, wherein the third partitioned filtrate comprises interferon-α2b.
13. The system of any one of sentences 1-12, wherein the cation exchange resin comprises an SP Sepharose HP resin and/or Toyopearl MX-Trp-650M resin.
14. The system of any one of sentences 1-13, wherein the first type of biological cells are yeast cells, filamentous fungal cells, microalgal cells, or diatom cells.
15. The system of sentence 14, wherein the yeast cells are Pichia pastoris cells.
16. The system of any one of sentences 1-15, wherein the cell culture medium comprises chemically defined media comprising a carbon source, chemically defined media comprising an additive, or buffered methanol-complex media (BMMY).
17. The system of any one of sentences 1-16, further comprising a formulation module fluidically connected to the purification module, wherein the formulation module is configured to produce a formulated product stream.
18. The system of sentence 17, wherein the formulation module comprises a filtration unit.
19. The system of sentence 18, wherein the filtration unit comprises a tangential flow filtration device.
20. The system of any one of sentences 17-19, wherein the formulation module comprises a viral filtration unit.
21. The system of any one of sentences 17-20, wherein the formulation module comprises a packaging unit.
22. The system of sentence 21, wherein the packaging unit is configured to package one or more doses of the at least one biologically-produced product into a bag, one or more vials, one or more syringes, and/or one or more bottles.
23. The system of any one of sentences 1-22, wherein the purified filtrate and/or formulated product stream comprises interferon-α2b having a purity of at least about 77%.
24. The system of any one of sentences 1-23, wherein the purified filtrate and/or formulated product stream has a DNA concentration of about 0.51 ng/(mg IFN) or less.
25. The system of any one of sentences 1-24, wherein the purified filtrate and/or formulated product stream has an aggregate content of about 0.5% or less.
26. The system of any of sentences 1-25, wherein the reactor chamber has a volume of about 1 L or less.
27. The system of sentence 26, wherein the at least one feed stream and the purified filtrate stream each have a flow rate of at least about 0.1 mL/min over a period of at least about 1 day.
28. The system of any one of sentences 26-27, wherein the system is configured to produce at least about 10 mg of IFN per day.
29. The system of any one of sentences 1-26, wherein the reactor chamber has a volume of about 1 L to about 10 L.
30. The system of sentence 29, wherein the at least one feed stream and the purified filtrate stream each have a flow rate of at least about 0.5 mL/min over a period of at least about 1 day.
31. The system of any one of sentences 29-30, wherein the system is configured to produce at least about 50 mg of IFN per day.
32. The system of any one of sentences 1-31, wherein the reactor chamber has a volume of about 10 L to about 50 L.
33. The system of sentence 32, wherein the at least one feed stream and the purified filtrate stream each have a flow rate of at least about 5 mL/min over a period of at least about 1 day.
34. The system of any one of sentences 32-33, wherein the system is configured to produce at least about 500 mg of IFN per day.
35. A method of producing interferon-α2b (IFN), comprising:

supplying at least one cell culture medium to a bioreactor; producing, within the bioreactor, a suspension comprising the at least one cell culture medium and at least a first type of biological cells expressing interferon-α2b;

causing at least a portion of the suspension to flow through at least one filter to produce at least one filtrate lean in the first type of biological cells, wherein the at least one filtrate comprises interferon-α2b; and

flowing the at least one filtrate through a purification module to produce a purified filtrate, wherein producing the purified filtrate comprises flowing the at least one filtrate through a first column comprising a multimodal cation exchange resin;

collecting one or more first fractions comprising interferon-α2b from an outflow of the first column;

flowing the one or more first fractions through a second column comprising an HCIC resin;

collecting one or more second fractions comprising interferon-α2b from an outflow of the second column;

flowing the one or more second fractions through a third column comprising a cation exchange resin; and

collecting one or more third fractions comprising interferon-α2b from an outflow of the third column.
36. The method of sentence 35, wherein the bioreactor is a perfusion bioreactor.
37. The method of sentence 36, wherein at least one feed stream comprising the at least one cell culture medium is continuously supplied to the perfusion bioreactor at a first flow rate over a period of at least about 1 day; the at least one filter is fluidically connected to the bioreactor and the at least one filtrate is an at least one first filtrate stream; and the purified filtrate is a purified filtrate stream flowing at a second flow rate, wherein the purified filtrate stream comprises the one or more third fractions.
38. The method of any one of sentences 35-37, further comprising, prior to supplying the at least one cell culture medium to the bioreactor, supplying a growth cell culture medium to the bioreactor; incubating the first type of biological cells in the growth cell culture medium for a period of at least about 1 day; and at least partially removing the growth cell culture medium from the bioreactor.
39. The method of any one of sentences 35-38, wherein the at least one filter comprises at least one filter probe at least partially submerged in the suspension in the bioreactor.
40. The method of any one of sentences 35-39, further comprising adjusting the pH of the at least one filtrate to produce a pH-adjusted filtrate.
41. The method of sentence 40, wherein the pH-adjusted filtrate has a pH of about 4.0.
42. The method of any one of sentences 40-41, wherein the pH is adjusted in a pH adjustment module that is fluidically connected to the at least one filter.
43. The method of any one of sentences 35-42, wherein the one or more first fractions are lean in a first type of impurity relative to the at least one filtrate.
44. The method of any one of sentences 35-43, wherein the multimodal cation exchange resin comprises a Capto MMC ImpRes resin.
45. The method of any one of sentences 35-44, wherein the one or more second fractions are lean in a second type of impurity relative to the first fractions.
46. The method of any one of sentences 35-45, wherein the HCIC resin comprises an MEP HyperCel resin and/or a HEA HyperCel resin.
47. The method of any one of sentences 35-46, wherein the one or more third fractions are lean in a third type of impurity relative to the second fractions.
48. The method of any one of sentences 35-47, wherein the cation exchange resin comprises an SP Sepharose HP resin and/or Toyopearl MX-Trp-650M resin.
49. The method of any one of sentences 35-48, wherein the first type of biological cells are yeast cells, filamentous fungal cells, microalgal cells, or diatom cells.
50. The method of sentence 49, wherein the yeast cells are Pichia pastoris cells.
51. The method of any one of sentences 35-50, wherein the at least one cell culture medium comprises chemically defined media comprising a carbon source, chemically defined media comprising an additive, or buffered methanol-complex media (BMMY).
52. The method of any one of sentences 35-51, further comprising flowing the purified filtrate through a formulation module configured to produce a formulated product stream.
53. The method of sentence 52, wherein flowing the purified filtrate through the formulation module comprises flowing the purified filtrate through a tangential flow filtration device.
54. The method of any one of sentences 52-53, wherein flowing the purified filtrate through the formulation module comprises flowing the purified filtrate stream through a viral filtration unit, wherein the formulated product stream is lean in one or more viruses relative to the purified filtrate stream.
55. The method of any one of sentences 52-54, wherein flowing the purified filtrate through the formulation module comprises depositing one or more portions of the purified filtrate stream into one or more containers.
56. The method of sentence 55, wherein the one or more containers are aseptic and/or sterile containers.
57. The method of any one of sentences 55-56, wherein the one or more containers comprise one or more bags, vials, syringes, and/or bottles.
58. The method of any one of sentences 35-57, wherein the purified filtrate and/or the formulated product stream comprise interferon-α2b having a purity of at least about 77%.
59. The method of any one of sentences 35-58, wherein the purified filtrate and/or the formulated product stream have a DNA concentration of about 0.51 ng/(mg IFN) or less.
60. The method of any one of sentences 35-59, wherein the purified filtrate and/or the formulated product stream have an aggregate content of about 0.5% or less.
61. The method of any one of sentences 35-60, wherein the bioreactor comprises a reactor chamber having a volume of about 1 L or less.
62. The method of sentence 61, wherein the first flow rate and/or the second flow rate are maintained at about 0.1 mL/min or more over a period of about 1 day or more.
63. The method of any one of sentences 61-62, wherein at least about 10 mg of IFN is produced in about 1 day or less.
64. The method of any one of sentences 35-60, wherein the bioreactor comprises a reactor chamber having a volume of about 1 L to about 10 L.
65. The method of sentence 64, wherein the first flow rate and/or the second flow rate are maintained at about 0.5 mL/min or more over a period of about 1 day or more.
66. The method of any one of sentences 64-65, wherein at least about 50 mg of IFN is produced in about 1 day or less.
67. The method of any one of sentences 35-60, wherein the bioreactor comprises a reactor chamber having a volume of about 10 L to about 50 L.
68. The method of sentence 67, wherein the first flow rate and/or the second flow rate are maintained at about 5 mL/min or more over a period of about 1 day or more.
69. The method of any one of sentences 67-68, wherein at least about 500 mg of IFN is produced in about 1 day or less.
70. The method of any one of sentences 52-69, wherein flowing the purified filtrate through the formulation module comprises flowing the purified filtrate stream through a dilution adjustment unit.
71. The method of sentence 70, wherein flowing the purified filtrate stream through the dilution adjustment unit comprises adding a diluent to the purified filtrate stream.
72. The system of any one of sentences 17-34, wherein the formulation module comprises a dilution adjustment unit.
73. The biomanufacturing system of any preceding sentence, further comprising a process and monitoring control system, optionally wherein the process and monitoring control system comprises one or more optical sensors, optionally wherein the one or more optical sensors comprises one or more cameras.
74. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
75. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A fifth exemplary embodiment is generally directed to the following:
1. A system for producing interferon-α2b (IFN), comprising:

a bioreactor, wherein the bioreactor comprises a reaction chamber containing a suspension comprising at least one cell culture medium and at least a first type of biological cells configured to express interferon-α2b;

at least one filter, wherein the at least one filter is configured to receive an output of the bioreactor and produce at least one filtrate lean in the first type of biological cells relative to the suspension, wherein the at least one filtrate comprises interferon-α2b; and

a purification module, wherein the purification module is configured to remove at least a first type of impurity, a second type of impurity, and a third type of impurity from the first filtrate to produce a purified filtrate, wherein the purification module comprises:

a first column comprising a multimodal cation exchange resin;

a second column comprising a flow-through resin; and

a third column comprising an anion exchange resin.
2. The system of sentence 1, wherein the bioreactor is a perfusion bioreactor.
3. The system of sentence 2, wherein the bioreactor is configured to receive at least one feed stream comprising the at least one cell culture medium; the at least one filter is fluidically connected to the bioreactor and the first filtrate is an at least one filtrate stream; the purification module is fluidically connected to the at least one filter and the purified filtrate is a purified filtrate stream; and the system is configured to be continuously operated.
4. The system of any one of sentences 1-3, wherein the at least one filter comprises at least one filter probe at least partially submerged in the suspension in the bioreactor.
5. The system of any one of sentences 1-4, wherein the system further comprises a pH adjustment module configured to increase or decrease the pH of the at least one filtrate to produce a pH-adjusted filtrate.
6. The system of sentence 5, wherein the pH adjustment module is fluidically connected to the bioreactor, the at least one filter, and/or the purification module.
7. The system of any one of sentences 5-6, wherein the pH-adjusted filtrate has a pH of about 5.0.
8. The system of any one of sentences 1-7, wherein the first column is configured to remove at least the first type of impurity from the at least one filtrate to produce a first partitioned filtrate lean in the first type of impurity relative to the at least one filtrate, wherein the first partitioned filtrate comprises interferon-α2b.
9. The system of any one of sentences 1-8, wherein the multimodal cation exchange resin comprises a Capto MMC ImpRes resin.
10. The system of any one of sentences 1-9, wherein the second column is configured to remove at least the second type of impurity from the first partitioned filtrate to produce a second partitioned filtrate lean in the second type of impurity relative to the first partitioned filtrate, wherein the second partitioned filtrate comprises interferon-α2b.
11. The system of any one of sentences 1-10, wherein the flow-through resin comprises a Q Sepharose HP resin.
12. The system of any one of sentences 1-11, wherein the third column is configured to remove at least the third type of impurity from the second partitioned filtrate to produce a third partitioned filtrate lean in the third type of impurity relative to the second partitioned filtrate, wherein the third partitioned filtrate comprises interferon-α2b.
13. The system of any one of sentences 1-12, wherein the anion exchange resin comprises a Capto Adhere resin.
14. The system of any one of sentences 1-13, wherein the first type of biological cells are yeast cells, filamentous fungal cells, microalgal cells, or diatom cells.
15. The system of sentence 14, wherein the yeast cells are Pichia pastoris cells.
16. The system of any one of sentences 1-15, wherein the cell culture medium comprises chemically defined media comprising a carbon source, chemically defined media comprising an additive, or buffered methanol-complex media (BMMY).
17. The system of any one of sentences 1-16, further comprising a formulation module fluidically connected to the purification module, wherein the formulation module is configured to produce a formulated product stream.
18. The system of sentence 17, wherein the formulation module comprises a filtration unit.
19. The system of sentence 18, wherein the filtration unit comprises a tangential flow filtration device.
20. The system of any one of sentences 17-19, wherein the formulation module comprises a viral filtration unit.
21. The system of any one of sentences 17-20, wherein the formulation module comprises a packaging unit.
22. The system of sentence 21, wherein the packaging unit is configured to package one or more doses of the at least one biologically-produced product into a bag, one or more vials, one or more syringes, and/or one or more bottles.
23. The system of any one of sentences 1-22, wherein the purified filtrate and/or formulated product stream comprises interferon-α2b having a purity of at least about 77%.
24. The system of any one of sentences 1-23, wherein the purified filtrate and/or formulated product stream has a DNA concentration of about 0.51 ng/(mg IFN) or less.
25. The system of any one of sentences 1-24, wherein the purified filtrate and/or formulated product stream has an aggregate content of about 0.5% or less.
26. The system of any of sentences 1-25, wherein the reactor chamber has a volume of about 1 L or less.
27. The system of sentence 26, wherein the at least one feed stream and the purified filtrate stream each have a flow rate of at least about 0.1 mL/min over a period of at least about 1 day.
28. The system of any one of sentences 26-27, wherein the system is configured to produce at least about 10 mg of IFN per day.
29. The system of any one of sentences 1-26, wherein the reactor chamber has a volume of about 1 L to about 10 L.
30. The system of sentence 29, wherein the at least one feed stream and the purified filtrate stream each have a flow rate of at least about 0.5 mL/min over a period of at least about 1 day.
31. The system of any one of sentences 29-30, wherein the system is configured to produce at least about 50 mg of IFN per day.
32. The system of any one of sentences 1-31, wherein the reactor chamber has a volume of about 10 L to about 50 L.
33. The system of sentence 32, wherein the at least one feed stream and the purified filtrate stream each have a flow rate of at least about 5 mL/min over a period of at least about 1 day.
34. The system of any one of sentences 32-33, wherein the system is configured to produce at least about 500 mg of IFN per day.
35. A method of producing interferon-α2b (IFN), comprising:

supplying at least one cell culture medium to a bioreactor; producing, within the bioreactor, a suspension comprising the at least one cell culture medium and at least a first type of biological cells expressing interferon-α2b;

causing at least a portion of the suspension to flow through at least one filter to produce at least one filtrate lean in the first type of biological cells, wherein the at least one filtrate comprises interferon-α2b;

flowing the at least one filtrate through a purification module to produce a purified filtrate, wherein producing the purified filtrate comprises, flowing the at least one filtrate through a first column comprising a multimodal cation exchange resin;

collecting one or more first fractions comprising interferon-α2b from an outflow of the first column;

flowing the one or more first fractions through a second column comprising a flow-through resin;

collecting one or more second fractions comprising interferon-α2b from an outflow of the second column;

flowing the one or more second fractions through a third column comprising an anion exchange resin; and

collecting one or more third fractions comprising interferon-α2b from an outflow of the third column.
36. The method of sentence 35, wherein the bioreactor is a perfusion bioreactor.
37. The method of sentence 36, wherein at least one feed stream comprising the at least one cell culture medium is continuously supplied to the perfusion bioreactor at a first flow rate over a period of at least about 1 day; the at least one filter is fluidically connected to the bioreactor and the at least one filtrate is an at least one first filtrate stream; and the purified filtrate is a purified filtrate stream flowing at a second flow rate, wherein the purified filtrate stream comprises the one or more third fractions.
38. The method of any one of sentences 35-37, further comprising, prior to supplying the at least one cell culture medium to the bioreactor, supplying a growth cell culture medium to the bioreactor; incubating the first type of biological cells in the growth cell culture medium for a period of at least about 1 day; and at least partially removing the growth cell culture medium from the bioreactor.
39. The method of any one of sentences 35-38, wherein the at least one filter comprises at least one filter probe at least partially submerged in the suspension in the bioreactor.
40. The method of any one of sentences 35-39, further comprising adjusting the pH of the at least one filtrate to produce a pH-adjusted filtrate.
41. The method of sentence 40, wherein the pH-adjusted filtrate has a pH of about 5.0.
42. The method of any one of sentences 40-41, wherein the pH is adjusted in a pH adjustment module that is fluidically connected to the at least one filter.
43. The method of any one of sentences 35-42, wherein the one or more first fractions are lean in a first type of impurity relative to the at least one filtrate.
44. The method of any one of sentences 35-43, wherein the multimodal cation exchange resin comprises a Capto MMC ImpRes resin.
45. The method of any one of sentences 35-44, wherein the one or more second fractions are lean in a second type of impurity relative to the first fractions.
46. The method of any one of sentences 35-45, wherein the flow-through resin comprises a Q Sepharose HP resin.
47. The method of any one of sentences 35-46, wherein the one or more third fractions are lean in a third type of impurity relative to the second fractions.
48. The method of any one of sentences 35-47, wherein the anion exchange resin comprises a Capto Adhere resin.
49. The method of any one of sentences 35-48, wherein the first type of biological cells are yeast cells, filamentous fungal cells, microalgal cells, or diatom cells.
50. The method of sentence 49, wherein the yeast cells are Pichia pastoris cells.
51. The method of any one of sentences 35-50, wherein the at least one cell culture medium comprises chemically defined media comprising a carbon source, chemically defined media comprising an additive, or buffered methanol-complex media (BMMY).
52. The method of any one of sentences 35-51, further comprising flowing the purified filtrate through a formulation module configured to produce a formulated product stream.
53. The method of sentence 52, wherein flowing the purified filtrate through the formulation module comprises flowing the purified filtrate through a tangential flow filtration device.
54. The method of any one of sentences 52-53, wherein flowing the purified filtrate through the formulation module comprises flowing the purified filtrate stream through a viral filtration unit, wherein the formulated product stream is lean in one or more viruses relative to the purified filtrate stream.
55. The method of any one of sentences 52-54, wherein flowing the purified filtrate through the formulation module comprises depositing one or more portions of the purified filtrate stream into one or more containers.
56. The method of sentence 55, wherein the one or more containers are aseptic and/or sterile containers.
57. The method of any one of sentences 55-56, wherein the one or more containers comprise one or more bags, vials, syringes, and/or bottles.
58. The method of any one of sentences 35-57, wherein the purified filtrate and/or the formulated product stream comprise interferon-α2b having a purity of at least about 77%.
59. The method of any one of sentences 35-58, wherein the purified filtrate and/or the formulated product stream have a DNA concentration of about 0.51 ng/(mg IFN) or less.
60. The method of any one of sentences 35-59, wherein the purified filtrate and/or the formulated product stream have an aggregate content of about 0.5% or less.
61. The method of any one of sentences 35-60, wherein the bioreactor comprises a reactor chamber having a volume of about 1 L or less.
62. The method of sentence 61, wherein the first flow rate and/or the second flow rate are maintained at about 0.1 mL/min or more over a period of about 1 day or more.
63. The method of any one of sentences 61-62, wherein at least about 10 mg of IFN is produced in about 1 day or less.
64. The method of any one of sentences 35-60, wherein the bioreactor comprises a reactor chamber having a volume of about 1 L to about 10 L.
65. The method of sentence 64, wherein the first flow rate and/or the second flow rate are maintained at about 0.5 mL/min or more over a period of about 1 day or more.
66. The method of any one of sentences 64-65, wherein at least about 50 mg of IFN is produced in about 1 day or less.
67. The method of any one of sentences 35-60, wherein the bioreactor comprises a reactor chamber having a volume of about 10 L to about 50 L.
68. The method of sentence 67, wherein the first flow rate and/or the second flow rate are maintained at about 5 mL/min or more over a period of about 1 day or more.
69. The method of any one of sentences 67-68, wherein at least about 500 mg of IFN is produced in about 1 day or less.
70. The method of any one of sentences 52-69, wherein flowing the purified filtrate through the formulation module comprises flowing the purified filtrate stream through a dilution adjustment unit.
71. The method of sentence 70, wherein flowing the purified filtrate stream through the dilution adjustment unit comprises adding a diluent to the purified filtrate stream.
72. The system of any one of sentences 17-34, wherein the formulation module comprises a dilution adjustment unit.
73. The biomanufacturing system of any preceding sentence, further comprising a process and monitoring control system, optionally wherein the process and monitoring control system comprises one or more optical sensors, optionally wherein the one or more optical sensors comprises one or more cameras.
74. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
75. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A sixth exemplary embodiment is generally directed to the following:
1. A system for producing human growth hormone, comprising:

a bioreactor, wherein the bioreactor comprises a reaction chamber containing a suspension comprising at least one cell culture medium and at least a first type of biological cells configured to express human growth hormone;

at least one filter, wherein the at least one filter is configured to receive an output of the bioreactor and produce at least one filtrate lean in the first type of biological cells relative to the suspension, wherein the at least one filtrate comprises human growth hormone; and

a purification module, wherein the purification module is configured to remove at least a first type of impurity and a second type of impurity from the at least one filtrate to produce a purified filtrate, wherein the purification module comprises:

a first column comprising a multimodal cation exchange resin; and a second column comprising an anion exchange resin.
2. The system of sentence 1, wherein the bioreactor is a perfusion bioreactor.
3. The system of sentence 2, wherein the bioreactor is configured to receive at least one feed stream comprising the at least one cell culture medium the at least one filter is fluidically connected to the bioreactor and the at least one filtrate is an at least one filtrate stream; the purification module is fluidically connected to the at least one filter and the purified filtrate is a purified filtrate stream; and the system is configured to be continuously operated.
4. The system of any one of sentences 1-3, wherein the at least one filter comprises at least one filter probe at least partially submerged in the suspension in the bioreactor.
5. The system of any one of sentences 1-4, wherein the system further comprises a pH adjustment module configured to increase or decrease the pH of the at least one filtrate to produce a pH-adjusted filtrate.
6. The system of sentence 5, wherein the pH adjustment module is fluidically connected to the bioreactor, the at least one filter, and/or the purification module.
7. The system of any one of sentences 5-6, wherein the pH-adjusted filtrate has a pH of about 5.0.
8. The system of any one of sentences 1-7, wherein the first column is configured to remove at least the first type of impurity from the at least one filtrate to produce a first partitioned filtrate lean in the first type of impurity relative to the at least one filtrate, wherein the first partitioned filtrate comprises human growth hormone.
9. The system of any one of sentences 1-8, wherein the multimodal cation exchange resin comprises a Capto MMC resin.
10. The system of any one of sentences 1-9, wherein the second column is configured to remove at least the second type of impurity from the first partitioned filtrate to produce a second partitioned filtrate lean in the second type of impurity relative to the first partitioned filtrate, wherein the second partitioned filtrate comprises human growth hormone.
11. The system of any one of sentences 1-10, wherein the anion exchange resin comprises a HyperCel STAR AX resin.
12. The system of any one of sentences 1-11, further comprising a third column comprising a hydrophobic charge induction chromatography (HCIC) resin.
13. The system of sentence 12, wherein the third column is configured to remove at least a third type of impurity from the second partitioned filtrate to produce a third partitioned filtrate lean in the third type of impurity relative to the second partitioned filtrate, wherein the third partitioned filtrate comprises human growth hormone.
14. The system of any one of sentences 12-13, wherein the HCIC resin comprises an MEP HyperCel resin.
15. The system of any one of sentences 1-14, wherein the first type of biological cells are yeast cells, filamentous fungal cells, microalgal cells, or diatom cells.
16. The system of sentence 15, wherein the yeast cells are Pichia pastoris cells.
17. The system of any one of sentences 1-16, wherein the at least one cell culture medium comprises chemically defined media comprising a carbon source, chemically defined media comprising an additive, or buffered methanol-complex media (BMMY).
18. The system of any one of sentences 1-17, further comprising a formulation module fluidically connected to the purification module, wherein the formulation module is configured to produce a formulated pharmaceutical product stream.
19. The system of sentence 18, wherein the formulation module comprises a filtration unit.
20. The system of sentence 19, wherein the filtration unit comprises a tangential flow filtration device.
21. The system of any one of sentences 18-20, wherein the formulation module comprises a viral filtration unit.
22. The system of any one of sentences 18-21, wherein the formulation module comprises a packaging unit.
23. The biomanufacturing system of sentence 22, wherein the packaging unit is configured to package one or more doses of the at least one pharmaceutical product into one or more bags, one or more vials, one or more syringes, and/or one or more bottles.
24. The system of any one of sentences 1-23, wherein the purified filtrate and/or formulated pharmaceutical product stream has a host cell protein concentration of about 50 ng/(mg hGH) or less.
25. The system of any one of sentences 1-24, wherein the purified filtrate and/or formulated pharmaceutical product stream has a DNA concentration of about 100 ng/(mg hGH) or less.
26. The system of any one of sentences 1-25, wherein the purified filtrate and/or formulated pharmaceutical product stream has an aggregate content of about 1% or less.
27. The system of any of sentences 1-26, wherein the reactor chamber has a volume of about 1 L or less.
28. The method of sentence 27, wherein the at least one feed stream and the purified filtrate stream each have a flow rate of at least about 0.1 mL/min over a period of at least about 1 day.
29. The method of any one of sentences 27-28, wherein the system is configured to produce at least about 10 mg of hGH per day.
30. The system of any one of sentences 1-26, wherein the reactor chamber has a volume of about 1 L to about 10 L.
31. The system of sentence 30, wherein the at least one feed stream and the purified filtrate stream each have a flow rate of at least about 0.5 mL/min over a period of at least about 1 day.
32. The system of any one of sentences 30-31, wherein the system is configured to produce at least about 50 mg of hGH per day.
33. The system of any one of sentences 1-26, wherein the reactor chamber has a volume of about 10 L to about 50 L.
34. The system of sentence 33, wherein the at least one feed stream and the purified filtrate stream each have a flow rate of at least about 5 mL/min over a period of at least about 1 day.
35. The system of any one of sentences 33-34, wherein the system is configured to produce at least about 500 mg of hGH per day.
36. A method of producing human growth hormone, comprising:

supplying at least one cell culture medium to a bioreactor;

producing, within the bioreactor, a suspension comprising the at least one cell culture medium and at least a first type of biological cells expressing human growth hormone;

causing at least a portion of the suspension to flow through at least one filter to produce at least one filtrate lean in the first type of biological cells, wherein the at least one filtrate comprises human growth hormone;

flowing the at least one filtrate through a purification module to produce a purified filtrate, wherein producing the purified filtrate comprises flowing the at least one filtrate through a first column comprising a multimodal cation exchange resin;

collecting one or more first fractions comprising human growth hormone from an outflow of the first column;

flowing the one or more first fractions through a second column comprising an anion exchange resin; and

collecting one or more second fractions comprising human growth hormone from an outflow of the second column.
37. The method of sentence 36, wherein the bioreactor is a perfusion bioreactor.
38. The method of sentence 37, wherein at least one feed stream comprising the at least one cell culture medium is continuously supplied to the perfusion bioreactor at a first flow rate over a period of at least about 1 day; the at least one filter is fluidically connected to the bioreactor and the at least one filtrate is an at least one filtrate stream; and the purified filtrate is a purified filtrate stream flowing at a second flow rate.
39. The method of any one of sentences 36-38, further comprising, prior to supplying the at least one cell culture medium to the bioreactor, supplying a growth cell culture medium to the bioreactor; incubating the first type of biological cells in the growth cell culture medium for a period of at least about 1 day; and at least partially removing the growth cell culture medium from the bioreactor.
40. The method of any one of sentences 36-39, wherein the at least one filter comprises at least one filter probe at least partially submerged in the suspension in the bioreactor.
41. The method of any one of sentences 36-40, further comprising adjusting the pH of the at least one filtrate to produce a pH-adjusted filtrate.
42. The method of sentence 41, wherein the pH-adjusted filtrate has a pH of about 5.0.
43. The method of any one of sentences 41-42, wherein the pH is adjusted in a pH adjustment module that is fluidically connected to the at least one filter.
44. The method of any one of sentences 36-43, wherein the one or more first fractions are lean in a first type of impurity relative to the at least one filtrate.
45. The method of any one of sentences 36-44, wherein the multimodal cation exchange resin comprises a Capto MMC resin.
46. The method of any one of sentences 36-45, The method of sentence 46, further comprising flowing a first mobile phase material through the first column prior to flowing the at least one filtrate through the first column, wherein the first mobile phase material is configured to promote binding of human growth hormone to the multimodal cation exchange resin.
48. The method of sentence 47, wherein the first mobile phase material has a pH of about 5.0.
49. The method of any one of sentences 46-48, further comprising flowing a second mobile phase material through the first column after flowing the at least one filtrate through the first column.
50. The method of sentence 49, wherein the second mobile phase material has a pH of about 5.0 and a sodium chloride concentration of about 500 mM.
51. The method of any one of sentences 46-50, further comprising flowing a third mobile phase material through the first column after flowing the second mobile phase material through the first column, wherein the third mobile phase material is configured to elute human growth hormone from the first column.
52. The method of sentence 51, wherein the third mobile phase material has a pH of about 6.0 and a sodium chloride concentration of about 100 mM.
53. The method of any one of sentences 36-52, wherein the one or more second fractions are lean in a second type of impurity relative to the first fractions.
54. The method of any one of sentences 36-53, wherein the anion exchange resin comprises a HyperCel STAR AX resin.
55. The method of any one of sentences 36-54, wherein the second column is operated in flow-through mode.
56. The method of any one of sentences 36-55, further comprising flowing the one or more second fractions through a third column comprising an HCIC resin.
57. The method of sentence 56, further comprising collecting one or more third fractions comprising human growth hormone from an outflow of the third column.
58. The method of sentence 57, wherein the one or more third fractions are lean in a third type of impurity relative to the second fractions.
59. The method of any one of sentences 56-58, wherein the HCIC resin comprises an MEP HyperCel resin.
60. The method of any one of sentences 56-59, wherein the third column is operated in bind-elute mode.
61. The method of sentence 60, further comprising flowing a first mobile phase material through the third column prior to flowing the second fractions through the first column, wherein the first mobile phase material is configured to promote binding of human growth hormone to the HCIC resin.
62. The method of sentence 61, wherein the first mobile phase material has a pH of about 6.0 and a sodium chloride concentration of about 100 mM.
63. The method of any one of sentences 60-62, further comprising flowing a second mobile phase material through the third column after flowing the second fractions through the third column.
64. The method of sentence 63, wherein the second mobile phase material has a pH of about 5.1 and a sodium chloride concentration less than about 100 mM.
65. The method of any one of sentences 60-64, further comprising flowing a third mobile phase material through the third column after flowing the second mobile phase material through the third column, wherein the third mobile phase material is configured to elute human growth hormone from the third column.
66. The method of sentence 65, wherein the third mobile phase material has a pH of about 3.0 and a sodium chloride concentration less than about 100 mM.
67. The method of any one of sentences 36-66, wherein the first type of biological cells are yeast cells, filamentous fungal cells, microalgal cells, or diatom cells.
68. The method of sentence 67, wherein the yeast cells are Pichia pastoris cells.
69. The method of any one of sentences 36-68, wherein the at least one cell culture medium comprises chemically defined media comprising a carbon source, chemically defined media comprising an additive, or buffered methanol-complex media (BMMY).
70. The method of any one of sentences 36-69, further comprising flowing the purified filtrate through a formulation module configured to produce a formulated pharmaceutical product stream.
71. The method of sentence 70, wherein flowing the purified filtrate through the formulation module comprises flowing the purified filtrate through a tangential flow filtration device.
72. The method of any one of sentences 70-71, wherein flowing the purified filtrate through the formulation module comprises flowing the purified filtrate stream through a viral filtration unit, wherein the formulated pharmaceutical product stream is lean in one or more viruses relative to the purified filtrate stream.
73. The method of any one of sentences 70-72, wherein flowing the purified filtrate through the formulation module comprises depositing one or more portions of the purified filtrate stream into one or more containers.
74. The method of sentence 73, wherein the one or more containers are aseptic and/or sterile containers.
75. The method of any one of sentences 73-74, wherein the one or more containers comprise one or more bags, vials, syringes, and/or bottles.
76. The method of any one of sentences 36-75, wherein the purified filtrate and/or the formulated pharmaceutical stream have a host cell protein concentration of about 50 ng/(mg hGH) or less.
77. The method of any one of sentences 36-76, wherein the purified filtrate and/or the formulated pharmaceutical stream have a DNA concentration of about 100 ng/(mg hGH) or less.
78. The method of any one of sentences 36-77, wherein the purified filtrate and/or the formulated pharmaceutical stream have an aggregate content of about 1% or less.
79. The method of any one of sentences 36-78, wherein the bioreactor comprises a reactor chamber having a volume of about 1 L or less.
80. The method of sentence 79, wherein the first flow rate and/or the second flow rate are maintained at about 0.1 mL/min or more over a period of about 1 day or more.
81. The method of any one of sentences 79-80, wherein at least about 10 mg of hGH is produced in about 1 day or less.
82. The method of any one of sentences 36-78, wherein the bioreactor comprises a reactor chamber having a volume of about 1 L to about 10 L.
83. The method of sentence 82, wherein the first flow rate and/or the second flow rate are maintained at about 0.5 mL/min or more over a period of about 1 day or more.
84. The method of any one of sentences 82-83, wherein at least about 50 mg of hGH is produced in about 1 day or less.
85. The method of any one of sentences 36-78, wherein the bioreactor comprises a reactor chamber having a volume of about 10 L to about 50 L.
86. The method of sentence 85, wherein the first flow rate and/or the second flow rate are maintained at about 5 mL/min or more over a period of about 1 day or more.
87. The method of any one of sentences 85-86, wherein at least about 500 mg of hGH is produced in about 1 day or less.
88. The biomanufacturing system of any preceding sentence, further comprising a process and monitoring control system, optionally wherein the process and monitoring control system comprises one or more optical sensors, optionally wherein the one or more optical sensors comprises one or more cameras.
89. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
90. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

A seventh exemplary embodiment is generally directed to the following:
1. A system for producing a single-domain antibody, comprising, a bioreactor, wherein the bioreactor comprises a reaction chamber containing a suspension comprising at least one cell culture medium and at least a first type of biological cells configured to express a single-domain antibody; at least one filter, wherein the at least one filter is configured to receive an output of the bioreactor and produce at least one filtrate lean in the first type of biological cells relative to the suspension, wherein the at least one filtrate comprises the single-domain antibody; and a purification module, wherein the purification module is configured to remove at least a first type of impurity and a second type of impurity from the at least one filtrate to produce a purified filtrate, wherein the purification module comprises, a first column comprising a multimodal cation exchange resin; and a second column comprising an anion exchange resin.
2. The system of sentence 1, wherein the bioreactor is a perfusion bioreactor.
3. The system of sentence 2, wherein: the bioreactor is configured to receive at least one feed stream comprising the at least one cell culture medium; the at least one filter is fluidically connected to the bioreactor and the at least one filtrate is an at least one filtrate stream; the purification module is fluidically connected to the at least one filter and the purified filtrate is a purified filtrate stream; and the system is configured to be continuously operated.
4. The system of any one of sentences 1-3, wherein the at least one filter comprises at least one filter probe at least partially submerged in the suspension in the bioreactor.
5. The system of any one of sentences 1-4, wherein the system further comprises a pH adjustment module configured to increase or decrease the pH of the at least one filtrate to produce a pH-adjusted filtrate.
6. The system of sentence 5, wherein the pH adjustment module is fluidically connected to the bioreactor, the at least one filter, and/or the purification module.
7. The system of any one of sentences 5-6, wherein the pH-adjusted filtrate has a pH of about 5.0.
8. The system of any one of sentences 1-7, wherein the first column is configured to remove at least the first type of impurity from the at least one filtrate to produce a first partitioned filtrate lean in the first type of impurity relative to the at least one filtrate, wherein the first partitioned filtrate comprises human growth hormone.
9. The system of any one of sentences 1-8, wherein the multimodal cation exchange resin comprises a CMM HyperCel resin.
10. The system of any one of sentences 1-8, wherein the single-domain antibody comprises a single variable domain.
11. The system of any one of sentences 1-10, wherein the second column is configured to remove at least the second type of impurity from the first partitioned filtrate to produce a second partitioned filtrate lean in the second type of impurity relative to the first partitioned filtrate, wherein the second partitioned filtrate comprises the single-domain antibody.
12. The system of any one of sentences 1-11, wherein the anion exchange resin comprises a HyperCel STAR AX resin.
13. The system of any one of sentences 1-11, wherein the anion exchange resin comprises a Capto Adhere resin.
14. The system of any one of sentences 1-11, wherein the single-domain antibody is a camelid single-domain antibody.
15. The system of any one of sentences 1-14, wherein the first type of biological cells are yeast cells, filamentous fungal cells, microalgal cells, or diatom cells.
16. The system of sentence 15, wherein the yeast cells are Pichia pastoris cells.
17. The system of any one of sentences 1-16, wherein the at least one cell culture medium comprises chemically defined media comprising a carbon source, chemically defined media comprising an additive, or buffered methanol-complex media (BMMY).
18. The system of any one of sentences 1-17, further comprising a formulation module fluidically connected to the purification module, wherein the formulation module is configured to produce a formulated pharmaceutical product stream.
19. The system of sentence 18, wherein the formulation module comprises a filtration unit.
20. The system of sentence 19, wherein the filtration unit comprises a tangential flow filtration device.
21. The system of any one of sentences 18-20, wherein the formulation module comprises a viral filtration unit.
22. The system of any one of sentences 18-21, wherein the formulation module comprises a packaging unit.
23. The biomanufacturing system of sentence 22, wherein the packaging unit is configured to package one or more doses of the at least one pharmaceutical product into one or more bags, one or more vials, one or more syringes, and/or one or more bottles.
24. The system of any one of sentences 1-23, wherein the purified filtrate and/or formulated pharmaceutical product stream has a host cell protein concentration of about 50 ng/(mg single-domain antibody) or less.
25. The system of any one of sentences 1-24, wherein the purified filtrate and/or formulated pharmaceutical product stream has a DNA concentration of about 100 ng/(mg single-domain antibody) or less.
26. The system of any one of sentences 1-25, wherein the purified filtrate and/or formulated pharmaceutical product stream has an aggregate content of about 1% or less.
27. The system of any of sentences 1-26, wherein the reactor chamber has a volume of about 1 L or less.
28. The method of sentence 27, wherein the at least one feed stream and the purified filtrate stream each have a flow rate of at least about 0.1 mL/min over a period of at least about 1 day.
29. The method of any one of sentences 27-28, wherein the system is configured to produce at least about 10 mg of single-domain antibody per day.
30. The system of any one of sentences 1-26, wherein the reactor chamber has a volume of about 1 L to about 10 L.
31. The system of sentence 30, wherein the at least one feed stream and the purified filtrate stream each have a flow rate of at least about 0.5 mL/min over a period of at least about 1 day.
32. The system of any one of sentences 30-31, wherein the system is configured to produce at least about 50 mg of single-domain antibody per day.
33. The system of any one of sentences 1-26, wherein the reactor chamber has a volume of about 10 L to about 50 L.
34. The system of sentence 33, wherein the at least one feed stream and the purified filtrate stream each have a flow rate of at least about 5 mL/min over a period of at least about 1 day.
35. The system of any one of sentences 33-34, wherein the system is configured to produce at least about 500 mg of single-domain antibody per day.
36. A method of producing a single-domain antibody, comprising :

supplying at least one cell culture medium to a bioreactor;

producing, within the bioreactor, a suspension comprising the at least one cell culture medium and at least a first type of biological cells expressing a single-domain antibody;

causing at least a portion of the suspension to flow through at least one filter to produce at least one filtrate lean in the first type of biological cells, wherein the at least one filtrate comprises the single-domain antibody; and

flowing the at least one filtrate through a purification module to produce a purified filtrate, wherein producing the purified filtrate comprises:

flowing the at least one filtrate through a first column comprising a multimodal cation exchange resin;

collecting one or more first fractions comprising the single-domain antibody from an outflow of the first column;

flowing the one or more first fractions through a second column comprising an anion exchange resin; and

collecting one or more second fractions comprising the single-domain antibody from an outflow of the second column.
37. The method of sentence 36, wherein the bioreactor is a perfusion bioreactor.
38. The method of sentence 37, wherein at least one feed stream comprising the at least one cell culture medium is continuously supplied to the perfusion bioreactor at a first flow rate over a period of at least about 1 day;

the at least one filter is fluidically connected to the bioreactor and the at least one filtrate is an at least one filtrate stream; and the purified filtrate is a purified filtrate stream flowing at a second flow rate.
39. The method of any one of sentences 36-38, further comprising, prior to supplying the at least one cell culture medium to the bioreactor, supplying a growth cell culture medium to the bioreactor; incubating the first type of biological cells in the growth cell culture medium for a period of at least about 1 day; and at least partially removing the growth cell culture medium from the bioreactor.
40. The method of any one of sentences 36-39, wherein the at least one filter comprises at least one filter probe at least partially submerged in the suspension in the bioreactor.
41. The method of any one of sentences 36-40, further comprising adjusting the pH of the at least one filtrate to produce a pH-adjusted filtrate.
42. The method of sentence 41, wherein the pH-adjusted filtrate has a pH of about 5.0.
43. The method of any one of sentences 41-42, wherein the pH is adjusted in a pH adjustment module that is fluidically connected to the at least one filter.
44. The method of any one of sentences 36-43, wherein the one or more first fractions are lean in a first type of impurity relative to the at least one filtrate.
45. The method of any one of sentences 36-44, wherein the multimodal cation exchange resin comprises a CMM HyperCel resin.
46. The method of any one of sentences 36-44, wherein the single-domain antibody comprises a single variable domain.
47. The method of any one of sentences 36-46, wherein the first column is operated in bind-elute mode.
48. The method of sentence 47, further comprising flowing a first mobile phase material through the first column prior to flowing the at least one filtrate through the first column, wherein the first mobile phase material is configured to promote binding of the single-domain antibody to the multimodal cation exchange resin.
49. The method of sentence 48, wherein the first mobile phase material has a pH of about 5.0.
50. The method of any one of sentences 47-49, further comprising flowing a second mobile phase material through the first column after flowing the at least one filtrate through the first column.
51. The method of sentence 50, wherein the second mobile phase material has a pH of about 6.0.
52. The method of any one of sentences 47-51, further comprising flowing a third mobile phase material through the first column after flowing the second mobile phase material through the first column, wherein the third mobile phase material is configured to elute the single-domain antibody from the first column.
53. The method of sentence 52, wherein the third mobile phase material has a pH of about 7.0 and a sodium chloride concentration of about 100 mM.
54. The method of any one of sentences 36-53, wherein the one or more second fractions are lean in a second type of impurity relative to the first fractions.
55. The method of any one of sentences 36-54, wherein the anion exchange resin comprises a HyperCel STAR AX resin.
56. The method of any one of sentences 36-54, wherein the anion exchange resin comprises a Capto Adhere resin.
57. The method of any one of sentences 36-56, wherein the second column is operated in flow-through mode.
58. The system of any one of sentences 36-57, wherein the single-domain antibody is a camelid single-domain antibody.
59. The method of any one of sentences 36-58, wherein the first type of biological cells are yeast cells, filamentous fungal cells, microalgal cells, or diatom cells.
60. The method of sentence 59, wherein the yeast cells are Pichia pastoris cells.
61. The method of any one of sentences 36-60, wherein the at least one cell culture medium comprises chemically defined media comprising a carbon source, chemically defined media comprising an additive, or buffered methanol-complex media (BMMY).
62. The method of any one of sentences 36-61, further comprising flowing the purified filtrate through a formulation module configured to produce a formulated pharmaceutical product stream.
63. The method of sentence 62, wherein flowing the purified filtrate through the formulation module comprises flowing the purified filtrate through a tangential flow filtration device.
64. The method of any one of sentences 62-63, wherein flowing the purified filtrate through the formulation module comprises flowing the purified filtrate stream through a viral filtration unit, wherein the formulated pharmaceutical product stream is lean in one or more viruses relative to the purified filtrate stream.
65. The method of any one of sentences 62-64, wherein flowing the purified filtrate through the formulation module comprises depositing one or more portions of the purified filtrate stream into one or more containers.
66. The method of sentence 65, wherein the one or more containers are aseptic and/or sterile containers.
67. The method of any one of sentences 65-66, wherein the one or more containers comprise one or more bags, vials, syringes, and/or bottles.
68. The method of any one of sentences 36-67, wherein the purified filtrate and/or the formulated pharmaceutical stream have a host cell protein concentration of about 50 ng/(mg single-domain antibody) or less.
69. The method of any one of sentences 36-68, wherein the purified filtrate and/or the formulated pharmaceutical stream have a DNA concentration of about 100 ng/(mg single-domain antibody) or less.
70. The method of any one of sentences 36-69, wherein the purified filtrate and/or the formulated pharmaceutical stream have an aggregate content of about 1% or less.
71. The method of any one of sentences 36-70, wherein the bioreactor comprises a reactor chamber having a volume of about 1 L or less.
72. The method of sentence 71, wherein the first flow rate and/or the second flow rate are maintained at about 0.1 mL/min or more over a period of about 1 day or more.
73. The method of any one of sentences 71-72, wherein at least about 10 mg of single-domain antibody is produced in about 1 day or less.
74. The method of any one of sentences 36-70, wherein the bioreactor comprises a reactor chamber having a volume of about 1 L to about 10 L.
75. The method of sentence 74, wherein the first flow rate and/or the second flow rate are maintained at about 0.5 mL/min or more over a period of about 1 day or more.
76. The method of any one of sentences 74-75, wherein at least about 50 mg of single-domain antibody is produced in about 1 day or less.
77. The method of any one of sentences 36-70, wherein the bioreactor comprises a reactor chamber having a volume of about 10 L to about 50 L.
78. The method of sentence 77, wherein the first flow rate and/or the second flow rate are maintained at about 5 mL/min or more over a period of about 1 day or more.
79. The method of any one of sentences 77-78, wherein at least about 500 mg of single-domain antibody is produced in about 1 day or less.
80. The biomanufacturing system of any preceding sentence, further comprising a process and monitoring control system, optionally wherein the process and monitoring control system comprises one or more optical sensors, optionally wherein the one or more optical sensors comprises one or more cameras.
81. The method of any preceding sentence, further comprising monitoring one or more steps of the method using a process and monitoring control system, optionally wherein the process and monitoring control system comprises one or more optical sensors, optionally wherein the one or more optical sensors comprises one or more cameras.
82. The method of any preceding sentence, further comprising implementing one or more corrective action based on information derived from a process and monitoring control system, optionally wherein the process and monitoring control system comprises one or more optical sensors, optionally wherein the one or more optical sensors comprises one or more cameras.

### EXAMPLES

Precision medicine holds the promise of improved treatments for small, well-defined populations of patients or even individuals. This model, however, presents a significant challenge for manufacturing protein biopharmaceuticals, which currently relies on centralized, large-scale facilities to supply global markets. One potential solution for agile and timely delivery of precision biologics to patients is small-scale, on-demand manufacturing within local pharmacies, hospitals, and healthcare clinics. An automated bench-top manufacturing system for end-to-end production of 100's to 1,000's of doses of high-quality, formulated biologic drugs in about 3 days without human intervention was produced. This system is paired with a complementary, integrated approach for accelerating process development of new biologics from sequence to purified drug in as few as 12 weeks. The use of this system with different well-characterized biologic drugs and demonstrate that the biologics have similar identity, purity, and potency as reference products is described below. Process simplification and rapid manufacturing of small volumes of formulated high-quality protein drugs using functionally closed, fully-integrated production systems could provide unique capabilities to supply medicines to patients when and where they need them.

Biologic medicines are an important class of drugs that include recombinant proteins such as cytokines, hormones, replacement enzymes, blood factors, and antibodies. Their therapeutic use in oncology and rare diseases increasingly relies on precise molecular profiles that define diseases of certain, often small, cohorts of patients. This precision in turn reduces the total supply of drug required, but expands the future plurality of medicines needed. The current manufacturing strategy for biologics, however, relies on large-volume, centralized facilities to provide economical production of a few products that support the overall costs of drug development.

Intensification of manufacturing processes with mammalian cells through continuous operations has established alternative cost-effective approaches that can reduce the size of facilities and equipment while retaining large volumetric production of a single drug substance. New technologies to manufacture many different high-quality biopharmaceuticals in small quantities with efficiency and agility are needed to make precision biologic medicines both available and economically feasible. A bench-scale, integrated, and automated manufacturing system that produces, purifies, and formulates high-quality recombinant protein drugs in less than 80 hours without human intervention is described. Additionally, complementary, integrated approach for accelerated process development to make and purify new products on the system in as few as 12 weeks is described.

To accelerate the timing for development and production, these examples utilized as a host the yeast Pichia pastoris, which can grow quickly to high cell densities and secrete recombinant proteins. Other advantages of P. pastoris include low levels of secreted host-cell proteins, little to no risk of viral contamination, documented expression of many classes of proteins including FDA-approved therapeutics, and the capability of human-like post-translational modifications. This host allowed rapid cycles of process development and simplified the architecture of the production system.

The benchtop system described in the certain examples included fluidically connected elements for fermentation, multi-stage chromatography, and ultrafiltration/diafiltration as well as integrated sensors and system controllers for programmed operations.

### Example 1

### Chromatographic Process Design for Purification of Granulocyte-Colony Stimulating Factor (G-CSF)

Fractionation Experiments for Cell Culture Fluid Spiked with G-CSF

Formulated drug substance Filgrastim, similar to naturally occurring granulocyte-colony stimulating factor (G-CSF) and considered for the purposes of these experiments to be equivalent to G-CSF and hereafter referred to interchangeably, was directly spiked into null strain Pichia pastoris cell culture fluid (CCF) at a concentration of 1.18 mg/mL and the resulting solution was titrated to pH 3.0, 5.0, 6.0, or 7.0 as needed from a starting pH of approximately 6.5. Titrated CCF was diluted with deionized (DI) water to a total CCF dilution of three times (3×) and filtered using a filter having a pore size of 0.2 μm. CCF was titrated to lower pH using 100 mM citric acid. CCF was titrated to higher pH using 100 mM Tris base because, by contrast, the addition of strong bases such as sodium hydroxide induced the irreversible local precipitation of some CCF components. CCF was prepared no greater than 24 hours in advance of its use. Fractionation experiments on the null Pichia CCF spiked with G-CSF were carried out using an AKTA Explorer 10 system (GE Healthcare) equipped with a Frac-950 fraction collector and a P-960 sample pump and controlled by Unicorn 5.1 software. The resins listed in Table 3 were packed in GE Tricorn 5/20 columns at approximately 0.5 mL column volumes (CVs), and gradient elution experiments were performed using the gradient conditions listed in Table 3.

Table 3 lists the chromatographic resins used in product and impurity characterization screens and the corresponding gradients performed on them. In Table 3, “salt” denotes salt gradients (0-1.5 M NaCl) at pH 5.0, 6.0, and 7.0, while pH gradients were run from pH 3.0 to 7.0 or 7.0 to 3.0 using a 20 mM citrate buffer. This set of resins was selected for the ability to bind material at elevated conductivities and for maximum operational flexibility across a small set of resins.

TABLE 3

| | | | Gradients | |
|---|---|---|---|---|
| Resin | Manufacturer | Type | Used | Functional Group |
| Capto MMC | GE | Weak MMC | Salt, increasing pH | 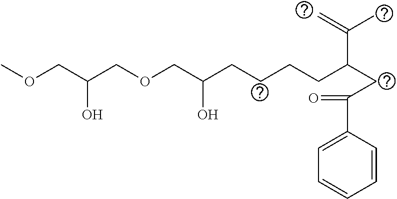 [SMILES: C=C(C)C(CCCCC(O)COCC(O)COC)CC(=O)c1ccccc1] |
| Capto MMC ImpRes | GE | Weak MMC | Salt, increasing pH | 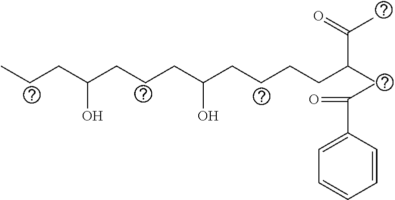 [SMILES: CCCC(O)CCCC(O)CCCCC(CC(=O)c1ccccc1)C(C)=O] |
| Nuvia cPrime | Bio-Rad | Weak MMC | Salt, increasing pH | 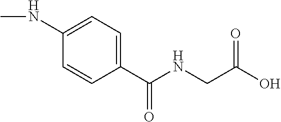 |
| Toyopearl MX-Trp- 650M | Tosoh | Weak MMC | Salt, increasing pH | 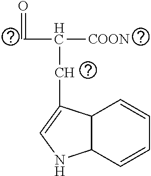 |
| CMM HyperCel | Pall | Weak MMC | Salt, increasing pH | 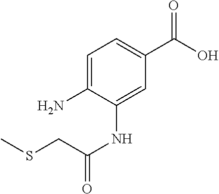 [SMILES: CSCC(=O)Nc1cc(C(=O)O)ccc1N] |
| Eshmuno HCX | Millipore | Weak MMC | Salt, increasing pH | 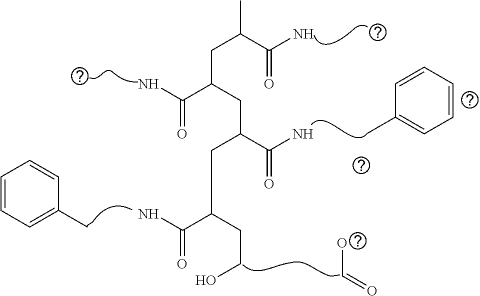 |
| Capto Adhere | GE | Strong MMA | Salt, increasing pH | 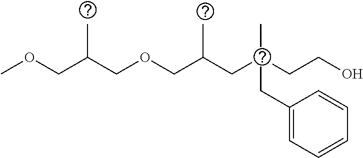 [SMILES: COCC(C)COCC(C)CC(C)(CCO)Cc1ccccc1] |
| PPA HyperCel | Pall | MMA/HCIC (aromatic) | Salt, decreasing pH | 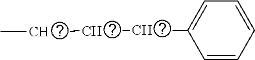 |
| HEA | Pall | MMA/HCIC | Salt, | —CH [symbol] —(CH [symbol] ) [symbol] —CH [symbol] |
| HyperCel | | (aliphatic) | decreasing pH | |
| MEP | Pall | HCIC | Decreasing | Primary Amine |
| HyperCel | | | pH | |
| HyperCel | Pall | Salt-tolerant | Salt, | Polyamine |
| STAR AX | | AEX | decreasing pH | |
| Toyopearl NH2—750F | Tosoh | Salt-tolerant AEX | Salt, decreasing pH | 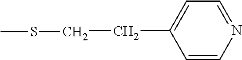 |

[symbol]

indicates data missing or illegible when filed

Columns were equilibrated with 10 CV of buffer A, loaded with 120 CV of conditioned null Pichia CCF spiked with G-CSF, washed with 20 CV of buffer A, eluted with a 40 CV gradient to buffer B, and washed with 10 CV of buffer B. Table 4 lists the compositions of the buffers used, each of which contained 0.02% azide.

TABLE 4

| Gradient | Buffer A | Buffer B |
|---|---|---|
| Salt gradient at pH 5.0 | 20 mM sodium citrate, pH 5.0 | 1.5M sodium chloride, 20 mM |
| | | sodium citrate, pH 5.0 |
| Salt gradient at pH 6.0 | 20 mM sodium phosphate, pH 6.0 | 1.5M sodium chloride, 20 mM |
| | | sodium phosphate, pH 6.0 |
| Salt gradient at pH 7.0 | 20 mM sodium phosphate, pH 7.0 | 1.5M sodium chloride, 20 mM |
| | | sodium phosphate, pH 7.0 |
| Increasing pH gradient | 20 mM sodium citrate, pH 3.0 | 20 mM sodium citrate, pH 7.0 |
| Decreasing pH gradient | 20 mM sodium citrate, pH 7.0 | 20 mM sodium citrate, pH 3.0 |

All solvents used 20 mM sodium citrate as a buffer species. Multimodal cation exchange resins (MMC) were stripped with 20 CV of 0.1 M sodium hydroxide. Multimodal anion exchange resins (MMA), hydrophobic charge-induction chromatography resins (HCIC), and salt-tolerant anion exchange resins (AEX) were stripped with 20 CV of 0.1 M citric acid.

For null Pichia CCF spiked with G-CSF, the flow rate was 0.5 CV/min during the load stage and 1 CV/min for all other stages. CCF flow-through was collected in two 60 CV fractions. Gradient elutions were collected in 2 CV fractions, which were combined into 4 CV fractions for analysis. The first 10 CVs of the strip were collected for analysis.

### RP-UPLC Analysis of CCF Partitioning Fractionation Experiments

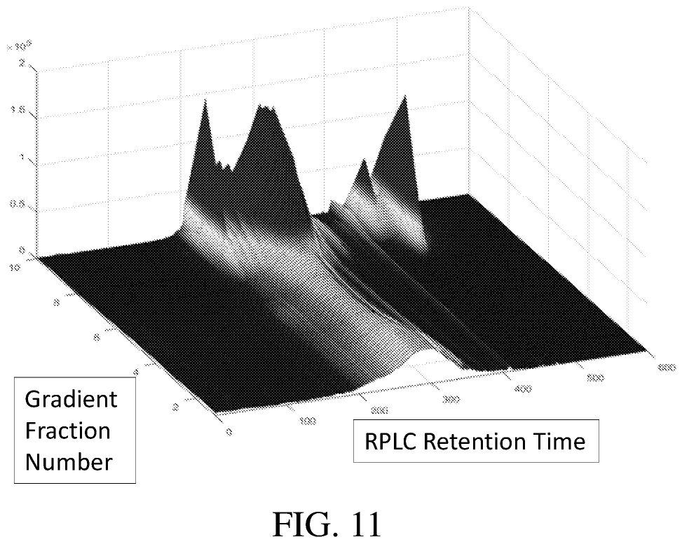

Reversed phase-ultra high pressure liquid chromatography (RP-UPLC) analysis of samples from the Pichia CCF partitioning fractionations was performed on a Waters Acquity ultra high pressure liquid chromatography (UPLC) H-class system equipped with a photodiode array (PDA) detector and controlled by Empower 3 software. Samples were run on an Acquity UPLC Protein bridged ethylsiloxane/silica hybrid (BEH) C₄ column (300 angstrom, 1.7 μm, 2.1 mm×100 mm) with an Acquity UPLC Protein BEH VanGuard Pre-Column (300 angstrom, 1.7 μm, 2.1 mm×5 mm). Column temperature was set to 60° C. Sample temperature was set to 8° C. Buffer A was 0.1% formic acid in water, and buffer B was 0.1% formic acid in acetonitrile (ACN) (v/v basis). System flow rate was held constant at 0.5 mL/min. The gradient method used was a 1 minute hold at 0% B, followed by a 7 minute linear gradient to 100% with a 2 minute hold prior to re-equilibration. Total method time was 12.5 min. Sample injection volumes were 50 μL. UV absorbance was collected as a wavelength scan from 200 to 400 nm at 2.4 nm resolution and 40 Hz frequency. FIG. 11 shows 2-dimensional chromatographic fingerprints that were generated for each resin and gradient type to characterize retention behavior of host cell proteins. G-CSF data was co-obtained from these experiments for each resin and gradient type. Peak integration was performed using Waters Empower 3 Software, and the results were exported for processing in MATLAB.

### Construction of Process-Related Impurity Retention Data Sets

RP-UPLC chromatograms were taken as raw data .ARW files at A210, A260, and A280 with 40 Hz resolution. A MATLAB script was written to convert all raw RP-UPLC data from .ARW files to a single .MAT file. Chromatograms corresponding to blank deionized water (DI) injections were used to baseline subtract from RP-UPLC chromatograms. These DI injections were performed each day, and fractionated Pichia CCF RP-UPLC chromatograms were baseline-subtracted using the appropriate DI injection run.

Data reduction was performed by integrating each chromatogram over 0.5 second time intervals. Only data from 1.5 to 10.0 minutes were considered in order to eliminate the effects of t₀ baseline disturbances. The process-related impurity retention data set was then stored as a 5-dimensional array with the first dimension corresponding to the wavelength of the data collected, the second dimension corresponding to the resin, the third dimension corresponding to the gradient type, the fourth dimension corresponding to the AKTA fraction number, and the fifth dimension corresponding to the integration window from RP-UPLC data.

### Downstream Process (DSP) Generation Tool

The downstream process (DSP) generation tool consisted of subroutines for total process generation and characterization, process constraint implementation, process ranking, table generation and graphical process output generation.

Downstream Process Generation: Process Generation with Implementation of Process Constraints

Data from the RP-UPLC regarding host cell proteins (HCP) and G-CSF was loaded into the program. Each resin or step was categorized as bind-elute, explicit flow-through, or implicit flow-through. Explicit flow-through steps were identified as such when the product was experimentally observed to flow through at the selected solution conditions of the load solution for the column. Implicit flow-through steps were identified as such when the load solution for the column was at a condition later in the salt or pH gradient than the condition at which the product eluted. Steps in which the product eluted in the strip were not considered.

The data was used as inputs to generate a list of all candidate 3-step process sequences, of both resin types and operating conditions, which recover the product, wherein each step corresponded to running the Pichia CCF spiked with G-CSF through a column with a resin from the screening process (Table 3) using a pH or salt gradient (Table 4). A number of constraints were implemented to reject undesirable processes. Capture steps were required to operate in bind-elute mode. The elution condition from one column was required to be the load condition for the subsequent column without adjustment to the salt and pH of the elution pool, so as to allow for integrated manufacturing. For sequences containing MEP HyperCel, only pH transitions were considered since salt gradient elution experiments were not performed. HCIC resins were not permitted to be used as a capture step. Each process was only permitted to use a given resin once. Since the first step of each candidate process was bind-elute, the second and third steps were classified as bind-elute, flow-through (explicit or implicit), or non-allowable transitions. The list of processes was further trimmed by imposing the constraint of no more than one (implicit or explicit) flow-through step.

### Downstream Process Generation Tool: Process Ranking, Table Generation, and Graphical Visualization

Process ranking for predicted process-related impurity clearance was performed using the following equation, for which lower scores indicated better (more orthogonal) processes:

Score=Σᵢ₌₁^{K}(Πⱼ₌₁^{p}A_{i,j}) (1)

### Where:

$$\{ \begin{matrix}j ∈ F , & {A}_{i , j} = {( {\sum }_{n = {R}_{Elute} ( j ) + 1}^{N} {a}_{i , j} ( n ) )}^{- 1} \\ j ∈ B , & {A}_{i , j} = {a}_{i , j} ( {R}_{Elute} ( j ) )\end{matrix} \qquad ( 2 )$$

In Equation 1, K is the number of UPLC integration fractions; P is the number of purification steps in a given process; F is a set of steps which are flow-through steps; B is a set of steps which are bind-elute steps; a_{i,j} is the area under the RP-UPLC chromatogram for a given wavelength, resin, gradient type, AKTA fraction number, and RP-UPLC integration window; R_{Elute} is the elution fraction number of the current column (corresponding to a specific pH and salt content); and N is the number of fractions collected in the ÄKTA gradient. Assumptions were made of optimized washing and relatively sharp elution peaks, so that in bind-elute operations, process-related impurities were considered to co-elute with the product in only a single gradient elution fraction. For flow-through steps, impurity clearance was determined using the sum of the RPLC chromatograms of all gradient elution fractions subsequent to the column inlet condition.

By use of the equation, the resins and conditions that offered the highest degree of orthogonal selectivity for process-related impurities were determined. By this method, scores were assigned and each set was rank-ordered (Table 5). Scores were assigned for data collected at a wavelength of 210 nm. This wavelength was chosen because it offered high sensitivity of detection and non-specificity so as to measure as many impurities as possible at a single wavelength. Profiles of process-related impurity removal were generated to visualize step orthogonality for each sequence.

Table 5 shows the top 20 process sequences selected by the process selection tool for the purification of G-CSF. Sequences are presented with their scores calculated by Equation 1 along with a score normalized to that of the top-ranked process. Individual steps are presented in the format of “resin, operating mode/pH, product elution condition”, where an operating pH is given when using a salt gradient elution and “flow-through” is used to indicate that the product elution condition is the same as the load condition.

TABLE 5

| | | | | Process | Normalized |
|---|---|---|---|---|---|
| Rank | Step 1 | Step 2 | Step 3 | Score | Score |
| 1 | Toyopearl MX- | Capto Adhere, pH | Capto MMC | 43869 | 1 |
| | Trp-650M, pH 5.0, | 5.0, flow-through | ImpRes, pH grad, | | |
| | 420 mM NaCl | | pH 7.0 | | |
| 2 | Toyopearl MX- | Capto Adhere, pH | HyperCel STAR | 79824 | 2 |
| | Trp-650M, pH 5.0, | 5.0, flow-through | AX, pH grad, pH | | |
| | 420 mM NaCl | | 3.8 | | |
| 3 | Toyopearl MX- | Capto Adhere, pH | Capto MMC, pH | 108575 | 2 |
| | Trp-650M, pH 5.0, | 5.0, flow-through | grad, pH 6.1 | | |
| | 420 mM NaCl | | | | |
| 4 | Toyopearl MX- | Capto MMC | HyperCel STAR | 117127 | 3 |
| | Trp-650M, pH 5.0, | ImpRes, pH grad, | AX, pH 7.0, flow- | | |
| | 420 mM NaCl | pH 7.0 | through | | |
| 5 | Capto MMC, pH | HyperCel STAR | CMM HyperCel, | 130359 | 3 |
| | grad, pH 6.1 | AX, pH 6.0, flow- | pH 6.0, 1170 mM | | |
| | | through | NaCl | | |
| 6 | Toyopearl MX- | Capto Adhere, pH | Nuvia cPrime, pH | 134433 | 3 |
| | Trp-650M, pH 5.0, | 5.0, flow-through | grad, pH 6.7 | | |
| | 420 mM NaCl | | | | |
| 7 | Capto MMC | MEP HyperCel, | Capto Adhere, pH | 148276 | 3 |
| | ImpRes, pH grad, | pH grad, pH 4.6 | 5.0, flow-through | | |
| | pH 7.0 | | | | |
| 8 | Toyopearl MX- | Capto MMC, pH | HyperCel STAR | 178594 | 4 |
| | Trp-650M, pH 5.0, | grad, pH 6.1 | AX, pH 6.0, flow- | | |
| | 420 mM NaCl | | through | | |
| 9 | Nuvia cPrime, pH | Capto MMC, pH | HyperCel STAR | 184011 | 4 |
| | 6.0, 410 mM NaCl | grad, pH 6.1 | AX, pH 6.0, flow- | | |
| | | | through | | |
| 10 | Capto MMC, pH | MEP HyperCel, | Capto Adhere, pH | 199204 | 5 |
| | grad, pH 6.1 | pH grad, pH 4.6 | 5.0, flow-through | | |
| 11 | Nuvia cPrime, pH | HyperCel STAR | Capto MMC, pH | 289979 | 7 |
| | 6.0, 410 mM NaCl | AX, pH 6.0, flow- | grad, pH 6.1 | | |
| | | through | | | |
| 12 | Toyopearl MX- | Capto MMC | Toyopearl NH2- | 299964 | 7 |
| | Trp-650M, pH 5.0, | ImpRes, pH grad, | 750F, pH 7.0, flow- | | |
| | 420 mM NaCl | pH 7.0 | through | | |
| 13 | Capto MMC | CMM HyperCel, | HyperCel STAR | 310653 | 7 |
| | ImpRes, pH 5.0, | pH 6.0, 1170 mM | AX, pH 6.0, flow- | | |
| | 1490 mM NaCl | NaCl | through | | |
| 14 | CMM HyperCel, | HyperCel STAR | MEP HyperCel, pH | 318628 | 7 |
| | pH 6.0, 1170 mM | AX, pH 6.0, flow- | grad, pH 4.6 | | |
| | NaCl | through | | | |
| 15 | Toyopearl MX- | CMM HyperCel, | HyperCel STAR | 346620 | 8 |
| | Trp-650M, pH | pH 6.0, 1170 mM | AX, pH 6.0, flow- | | |
| | grad, pH 5.9 | NaCl | through | | |
| 16 | CMM HyperCel, | Capto MMC, pH | HyperCel STAR | 346728 | 8 |
| | pH 6.0, 1170 mM | 6.0, flow-through | AX, pH grad, pH | | |
| | NaCl | | 3.8 | | |
| 17 | Toyopearl MX- | Toyopearl NH2- | CMM HyperCel, | 403873 | 9 |
| | Trp-650M, pH | 750F, pH 6.0, | pH 6.0, 1170 mM | | |
| | grad, pH 5.9 | flow-through | NaCl | | |
| 18 | Nuvia cPrime, pH | MEP HyperCel, | Capto Adhere, pH | 403965 | 9 |
| | grad, pH 6.7 | pH grad, pH 4.6 | 5.0, flow-through | | |
| 19 | Capto MMC | HyperCel STAR | MEP HyperCel, pH | 432654 | 10 |
| | ImpRes, pH grad, | AX, pH 7.0, flow- | grad, pH 4.6 | | |
| | pH 7.0 | through | | | |
| 20 | Toyopearl MX- | HyperCel STAR | CMM HyperCel, | 445098 | 10 |
| | Trp-650M, pH | AX, pH 6.0, flow- | pH 6.0, 1170 mM | | |
| | grad, pH 5.9 | through | NaCl | | |

### Decision Process to Narrow the Downstream Process Candidate List

Once potential sequences were identified using the process generation tool, a decision process was implemented to select the specific potential sequence for process development. This was important since the sequences were generated based on orthogonal selectivity while not including additional important considerations such as binding capacity and yield constraints. One constraint that was used to eliminate potential sequences was that for bind/elute steps, the loading and elution condition should be sufficiently different, because this generally resulted in higher binding capacities. For example, in sequence number 4 (Table 5), for the first step using Toyopearl MX-Trp-650M, the elution salt concentration was 300 mM, which was close to the conductivity of the Pichia CCF load. Thus, this sequence was not selected. This heuristic was employed to select “straw man” processes which were then used to initiate process development as described below.

### Downstream Purification Process Development for G-CSF

The development of a process to purify G-CSF from Pichia CCF was initiated using the output from the process selection tool shown in Table 5. The specified purification targets were host cell proteins (HCP) less than 100 ppm and DNA less than limit of detection (LOD) of a Quant-iT™ PicoGreen® dsDNA Assay Kit (ThermoFisher) kit. Sequences 1-4 and 6 were discounted due to the use of Toyopearl MX-Trp-650M as the capture step since the elution salt concentration was 300 mM, which was close to the conductivity of the Pichia CCF load. For G-CSF sequence 5, this particular combination of Capto MMC and CMM HyperCel was highly sensitive to the pH of the Capto MMC elution buffer/CMM HyperCel binding buffer. In order to circumvent this problem, sequence 7 was selected as the starting point for G-CSF process development.

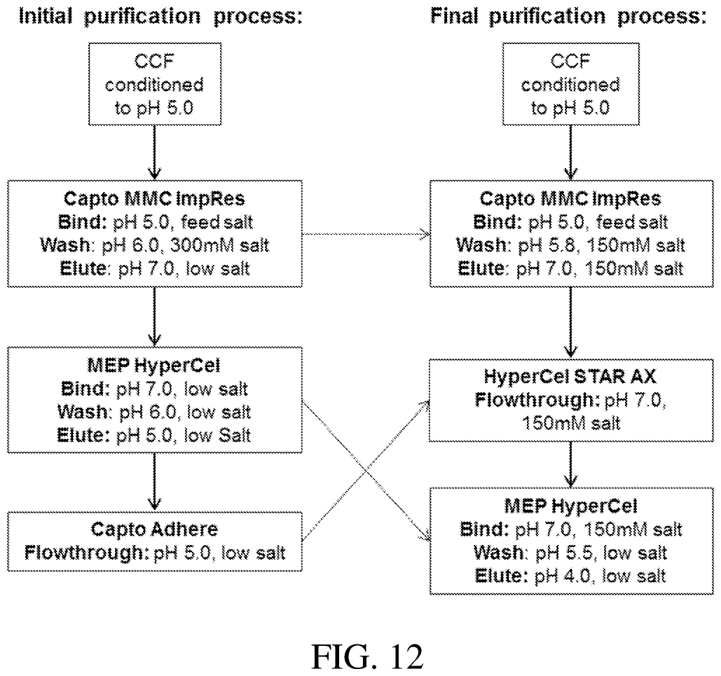

Upon experimental process optimization, the Capto MMC ImpRes and MEP HyperCel steps were developed, and it was found that recovery for Capto Adhere was poor. The next highly ranked process, which contained Capto MMC ImpRes and MEP HyperCel, was selected, only requiring development of the HyperCel STAR AX step. FIG. 12 shows the original process selected from software output and the final optimized process.

The final process (shown in FIG. 12) was used to purify G-CSF from Pichia CCF, and the resulting solution was characterized for product recovery and clearance of HCP, DNA, and product aggregates. The load challenge for capture step was approximately 2.5 mg G-CSF/mL resin. An SDS-PAGE gel was run to test product quality. Enzyme-linked immunosorbent assay (ELISA) (Cygnus Technologies) was carried out on Pichia to determine information on process contaminants including host cell proteins (HCPs). A Quant-iT™ PicoGreen( )dsDNA Assay Kit (ThermoFisher) was carried out to determine information on DNA process contaminants. Size-exclusion chromatography (SEC) was used to quantify aggregates. Reverse phase liquid chromatography (RPLC) was used to determine charge variants and product titer. Capillary electrophoresis (Perkin Elmer GXII) was also used to determine product titer. A cell-based proliferation assay was conducted to determine product activity.

PicoGreen DNA analysis was performed using the Quant-iT™ PicoGreen® dsDNA Assay Kit from ThermoFisher Scientific. To generate a dsDNA calibration curve, samples were prepared from lambda DNA standard included with the kit diluted to concentrations of 500, 100, 50, 25, 10, 5, and 1 pg/μL in TE buffer included with the kit. Pure TE buffer was also included in the calibration as a null concentration point. Samples for analysis were diluted with TE buffer if needed. 20 μL of each sample to be analyzed were added to a Costar black 96-well plate in triplicate. In darkness, PicoGreen reagent was prepared by mixing with TE buffer, and then 200 μL was added to each well for analysis and mixed by pipetting. The plate was then analyzed on a plate reader, which included a 2 minute agitation period to further mix the wells followed by excitation at 480 nm and measurement of emission at 520 nm.

The results presented in Table 6 show that overall product recovery was approximately 80% with total HCP clearance (5.4 logs) and total DNA clearance (greater than 4.1 logs) exceeding the purification targets. The final product concentration was 0.232 mg/mL, and the final aggregate content was 0.60%. For DNA, error bars represent one standard deviation of triplicate measurements. For HCP ELISA, error bars denote the 95% confidence interval.

TABLE 6

| | Prod. Conc. | | HCP | DNA | |
|---|---|---|---|---|---|
| Sample | (mg/ml) | Recovery | (PPM) | (PPM) | Aggregate |
| G-CSF CCF | 0.023 | — | 1,976,522 ± 6,715,217 | 278,261 ± 29,074 | — |
| Capto MMC | 0.612 | 100%* | 6,989 ± 3.023 | 28 ± 11 | 1.10% |
| ImpRes eluate | | | | | |
| HyperCel STAR | 0.217 | 88% | 66 ± 17.9 | <LOD | 1.11% |
| AX eluate | | | | | |
| MEP HyperCel | 0.232 | 91% | 8.0 ± 0.8 | <LOD | 0.60% |
| eluate | | | | | |

*Value was found to be greater than 100%

### Example 2

### Chromatographic Process Design for Purification of Human Growth Hormone (hGH)

### Cell Culture Fluid Partitioning Fractionation Experiments and hGH Fractionation Experiments

Null strain Pichia pastoris cell culture fluid (CCF) fractionation experiments were carried out as described in Example 1.

Pure human growth hormone (hGH) was dissolved from lyophilized powder form into pH 3.0 citrate buffer, pH 4.0 citrate buffer, pH 5.0 citrate buffer, pH 6.0 citrate buffer, or pH 7.0 Tris buffer as needed at approximately 0.5 mg/mL and 0.2 μm filtered. The final concentration of hGH in the solution was 640 μg/mL. The hGH solution was prepared less than 24 hours in advance of its use. hGH fractionation experiments were carried out according to nearly the same protocols and using the same chromatographic resins and buffers as the CCF G-CSF fractionation experiments described in Example 1. The difference was that in this case system flow rate was kept constant at 1 CV/min throughout the method. Column load challenge was kept constant at 0.3 mg/mL, and product elution pH or salt concentration was determined by peak maximum at 280 nm. Pure component hGH retention data was obtained directly from the

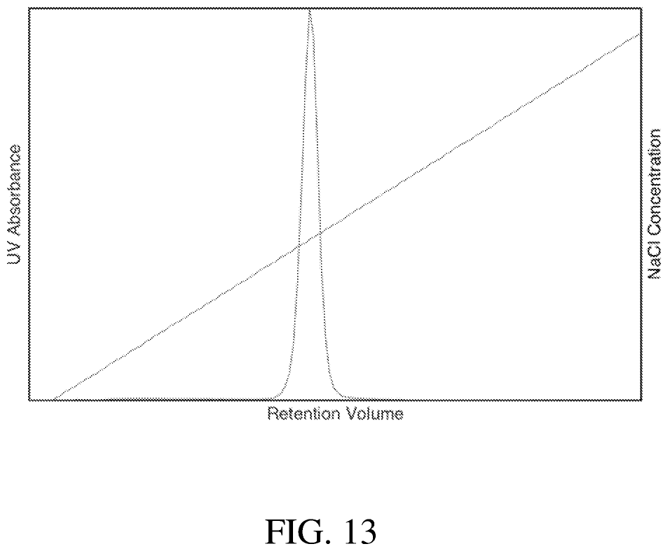

AKTA chromatogram, shown in FIG. 13.

### RP-UPLC Analysis of CCF Fractionation Experiments

RP-UPLC analysis of samples from the CCF partitioning fractionations was performed according to the protocol described in Example 1. The process-relatedimpurity retention data set was constructed according to the protocols described in Example 1, and the downstream process (DSP) generation tool described in Example 1 was used to design a downstream process appropriate for hGH.

Initially, hGH product retention data was loaded into the program. As described in Example 1, each resin or step was categorized as bind-elute, explicit flow-through, or implicit flow-through. The hGH product retention data was used as inputs to generate a list of all candidate 3-step process sequences, of both resin types and operating conditions, which recover the product, wherein each step corresponded to running the hGH product solution through a column with a resin from the screening process using a pH or salt gradient. The constraints described above in Example 1 were implemented to reject undesirable processes.

Then, using both the hGH product retention data and host cell protein (HCP) data from the null cell culture fluid (CCF) fractionation experiments, the processes were ranked using Equation 1 as described in Example 1. Using this equation, scores were assigned and each set was rank-ordered. The top 20 process sequences selected by the process selection tool for the purification of hGH are shown in Table 7.

TABLE 7

| | | | | Process | Normalized |
|---|---|---|---|---|---|
| Rank | Step 1 | Step 2 | Step 3 | score | score |
| 1 | Capto MMC, pH | HyperCel STAR | CMM HyperCel, | 90555 | 1 |
| | grad, pH 6.1 | AX, pH 6.0, flow- | pH 6.0. 770 mM | | |
| | | through | NaCl | | |
| 2 | Capto MMC | HyperCel STAR | CMM HyperCel, | 99254 | 1 |
| | ImpRes, pH grad, | AX, pH 6.0, flow- | pH 6.0, 770 mM | | |
| | pH 5.9 | through | NaCl | | |
| 3 | Capto MMC | HyperCel STAR | MEP HyperCel, pH | 119087 | 1 |
| | ImpRes, pH grad, | AX, pH 6.0, flow- | grad, pH 4.3 | | |
| | pH 5.9 | through | | | |
| 4 | Toyopearl MX- | Capto MMC, pH | HyperCel STAR | 151025 | 2 |
| | Trp-650M, pH 5.0, | grad, pH 6.1 | AX, pH 6.0, flow- | | |
| | 300 mM NaCl | | through | | |
| 5 | Capto MMC, pH | HyperCel STAR | Nuvia cPrime, pH | 184011 | 2 |
| | grad, pH 6.1 | AX, pH 6.0, flow- | 6.0, 460 mM NaCl | | |
| | | through | | | |
| 6 | Toyopearl MX- | Capto MMC | HyperCel STAR | 199617 | 2 |
| | Trp-650M, pH 5.0, | ImpRes, pH grad, | AX, pH 6.0, flow- | | |
| | 300 mM NaCl | pH 5.9 | through | | |
| 7 | Capto MMC | Toyopearl NH2- | CMM HyperCel, | 204148 | 2 |
| | ImpRes, pH grad, | 750F, pH 6.0, | pH 6.0, 770 mM | | |
| | pH 5.9 | flow-through | NaCl | | |
| 8 | Toyopearl MX- | Capto MMC | Toyopearl NH2- | 248579 | 3 |
| | Trp-650M, pH 5.0, | ImpRes, pH grad, | 750F, pH 6.0, flow- | | |
| | 300 mM NaCl | pH 5.9 | through | | |
| 9 | Capto MMC, pH | Toyopearl NH2- | CMM HyperCel, | 251969 | 3 |
| | grad, pH 6.1 | 750F, pH 6.0, | pH 6.0, 770 mM | | |
| | | flow-through | NaCl | | |
| 10 | Capto MMC | HyperCel STAR | Nuvia cPrime, pH | 254799 | 3 |
| | ImpRes, pH grad, | AX, pH 6.0, flow- | 6.0. 460 mM NaCl | | |
| | pH 5.9 | through | | | |
| 11 | Capto Capto | Nuvia cPrime, pH | HyperCel STAR | 289979 | 3 |
| | MMC, pH grad, | 6.0, 460 mM NaCl | AX, pH 6.0, flow- | | |
| | pH 6.1 | | through | | |
| 12 | Capto Adhere, pH | Capto MMC | HyperCel STAR | 351568 | 4 |
| | grad, pH 4.1 | ImpRes, pH grad, | AX, pH 6.0, flow- | | |
| | | pH 5.9 | through | | |
| 13 | CMM HyperCel, | HyperCel STAR | MEP HyperCel, pH | 356769 | 4 |
| | pH grad, pH 5.8 | AX, pH 6.0, flow- | grad, pH 4.3 | | |
| | | through | | | |
| 14 | Capto MMC | HyperCel STAR | Capto MMC, pH | 365771 | 4 |
| | ImpRes, pH grad, | AX, pH 6.0, flow- | 6.0, 420 mM NaCl | | |
| | pH 5.9 | through | | | |
| 15 | Toyopearl MX- | HyperCel STAR | Capto MMC, pH | 384298 | 4 |
| | Trp-650M, pH 5.0, | AX, pH 5.0, flow- | grad, pH 6.1 | | |
| | 300 mM NaCl | through | | | |
| 16 | CMM HyperCel, | Toyopearl NH2- | MEP HyperCel, pH | 392500 | 4 |
| | pH 6.0, 770 mM | 750F, pH 6.0, | grad, pH 4.3 | | |
| | NaCl | flow-through | | | |
| 17 | Capto MMC | Toyopearl NH2- | MEP HyperCel, pH | 393897 | 4 |
| | ImpRes, pH grad, | 750F, pH 6.0, | grad, pH 4.3 | | |
| | pH 5.9 | flow-through | | | |
| 18 | Toyopearl MX- | CMM HyperCel, | Toyopearl NH2- | 397645 | 4 |
| | Trp-650M, pH 5.0, | pH grad, pH 5.8 | 750F, pH 6.0, flow- | | |
| | 300 mM NaCl | | through | | |
| 19 | Capto MMC, pH | HyperCel STAR | MEP HyperCel, pH | 408842 | 5 |
| | grad, pH 6.1 | AX, pH 6.0, flow- | grad, pH 4.3 | | |
| | | through | | | |
| 20 | Capto MMC | Nuvia cPrime, pH | HyperCel STAR | 413913 | 5 |
| | ImpRes, pH grad, | 6.0, 460 mM | AX, pH 6.0, flow- | | |
| | pH 5.9 | NaCl | through | | |

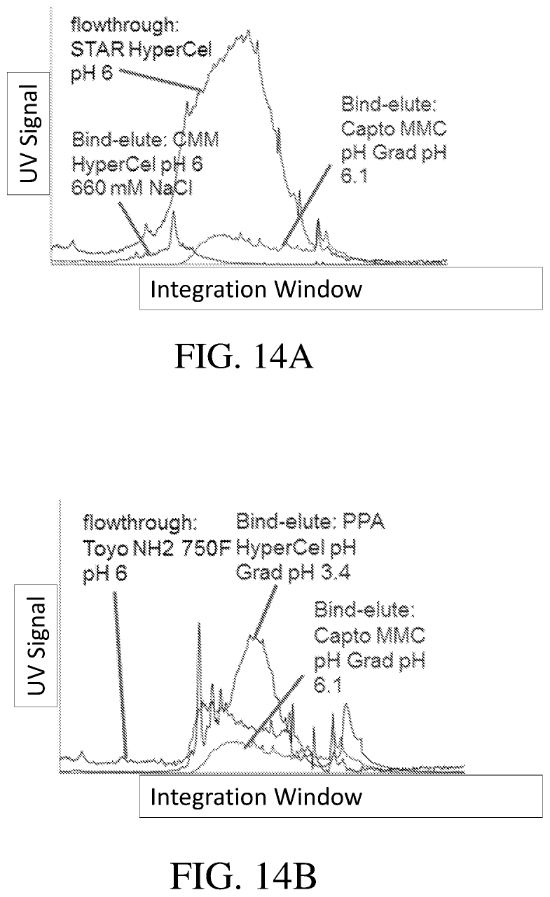

Profiles of process-related impurity removal were generated to visualize step orthogonality for each sequence. FIG. 14A is an example of a highly orthogonal 3-step process, and FIG. 14B is an example of a non-orthogonal 3-step process.

### Decision Process to Narrow the Downstream Process Candidate List

Once potential sequences were identified using the process generation tool, a decision process was implemented to select the specific potential sequence for process development according to the protocols described in Example 1.

### Downstream Purification Process Development for hGH

The development of a process to purify hGH from Pichia CCF was initiated using the output from the process selection tool shown in Table 7. The specified purification targets were host cell proteins (HCP) less than 100 ppm and DNA less than the limit of detection (LOD) of a Quant-iT™ PicoGreen® dsDNA Assay Kit (ThermoFisher) kit. Sequence 1 was selected as the starting point for hGH process development.

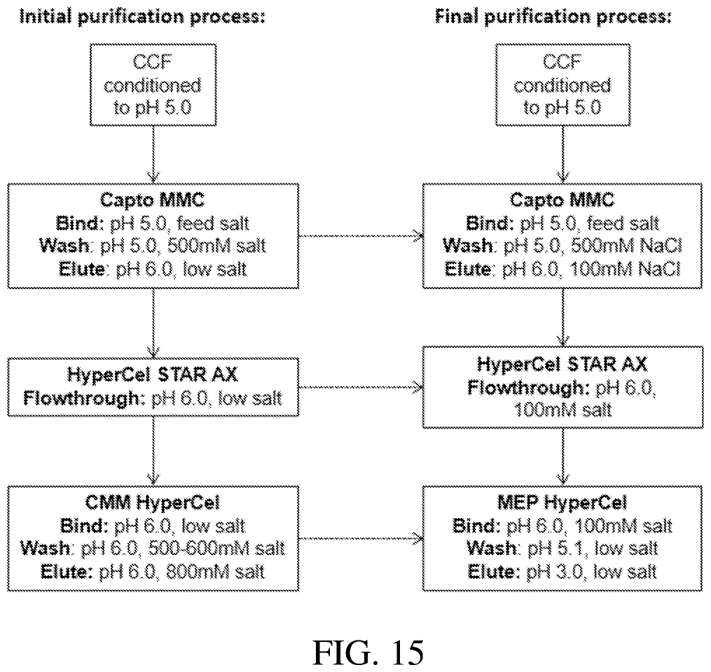

The initial top-scoring process (FIG. 15, left) utilized CMM HyperCel as a second polish step. Upon experimental validation, capacity and process robustness were improved by replacing this step with MEP HyperCel (FIG. 15, right). This revised process corresponds to process 19, another top-scoring process. FIG. 15 shows the overall original process selected from software output (left) and the final optimized process (right).

The final process shown in FIG. 15 (right) was used to purify hGH from Pichia CCF, and the resulting solution was characterized for product recovery and clearance of HCP, DNA, and product aggregates. The load challenge for capture step was approximately 0.3 mg hGH/mL resin. An SDS-PAGE gel was run to test product quality. Enzyme-linked immunosorbent assay (ELISA) (Cygnus Technologies) was carried out on Pichia to determine information on process contaminants including host cell proteins (HCPs). A Quant-iT™ PicoGreen® dsDNA Assay Kit (ThermoFisher) was carried out to determine information on DNA process contaminants. Size-exclusion chromatography (SEC) was used to quantify aggregates. Reverse phase liquid chromatography (RPLC) was used to determine charge variants and product titer. Capillary electrophoresis (Perkin Elmer GXII) was also used to determine product titer. A cell-based proliferation assay was conducted to determine product activity.

The results presented in Table 8 show that overall product recovery was approximately 86%, with total HCP clearance (4.5 logs) exceeding the purification target and total DNA clearance (2.7 logs) falling short of the purification target. The final product concentration was 0.631 mg/mL, and the final aggregate content was 0.48%. In Table 8, for DNA, error bars represent one standard deviation of triplicate measurements, and for HCP ELISA, error bars denote the 95% confidence interval.

TABLE 8

| | Prod. conc. | | HCP | DNA | |
|---|---|---|---|---|---|
| Sample | (mg/ml) | Recovery | (PPM) | (PPM) | Aggregate |
| hGH CCF | 0.114 | — | 387,895 ± 260,263 | 37,878 ± 5,196 | — |
| Capto MMC | 0.654 | 98% | 33.49 ± 18.77 | 109 ± 7 | 1.65% |
| eluate | | | | | |
| HyperCel STAR | 0.283 | 92% | 4.87 ± 2.51 | 161 ± 10 | 0.51% |
| AX flow-through | | | | | |
| MEP HyperCel | 0.631 | 96% | 13.72 ± 13.08 | 77 ± 22 | 0.48% |
| eluate | | | | | |

### Example 3

### Chromatographic Process Design for Purification of Interferon α-2β (IFN)

### IFN Variant Identification

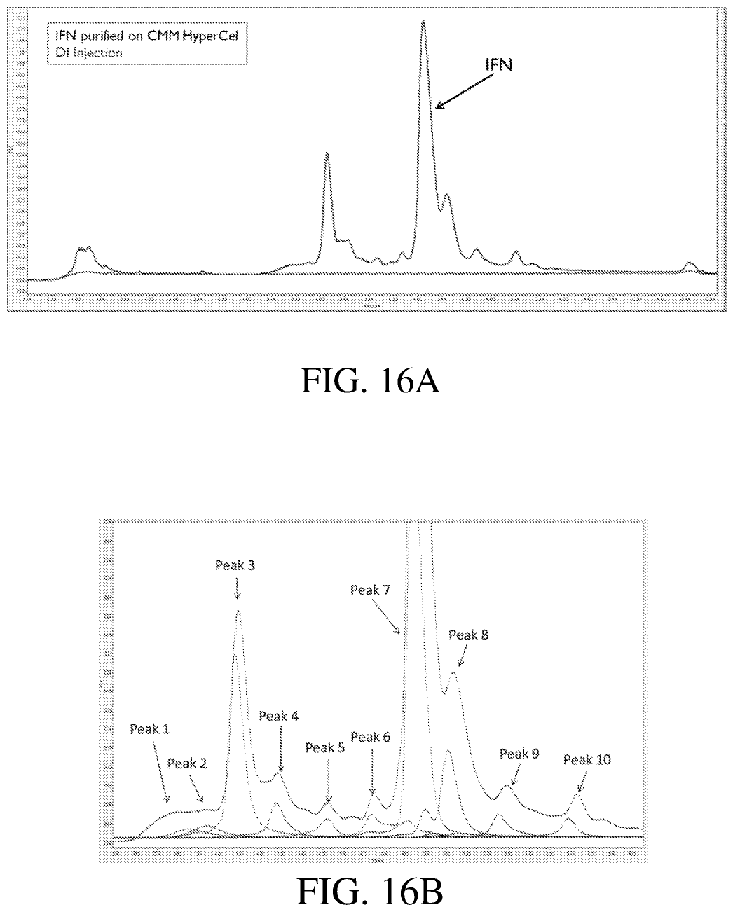

Interferon α-2β (IFN) was partially purified from a cell culture fluid from a Pichia pastoris culture expressing IFN in order to concentrate the problematic product variants being produced. A multimodal cation exchange (MMC) CMM HyperCel column was used to capture the IFN and related products and separate them from many of the other host impurities. Partially purified IFN was fractionated by reversed phase-ultra high pressure liquid chromatography (RP-UPLC) using a linear gradient elution and analyzed by direct injection electrospray ionization mass spectrometry (ESI-MS). FIG. 16A shows an RP-UPLC chromatogram of partially purified interferon α-2β.

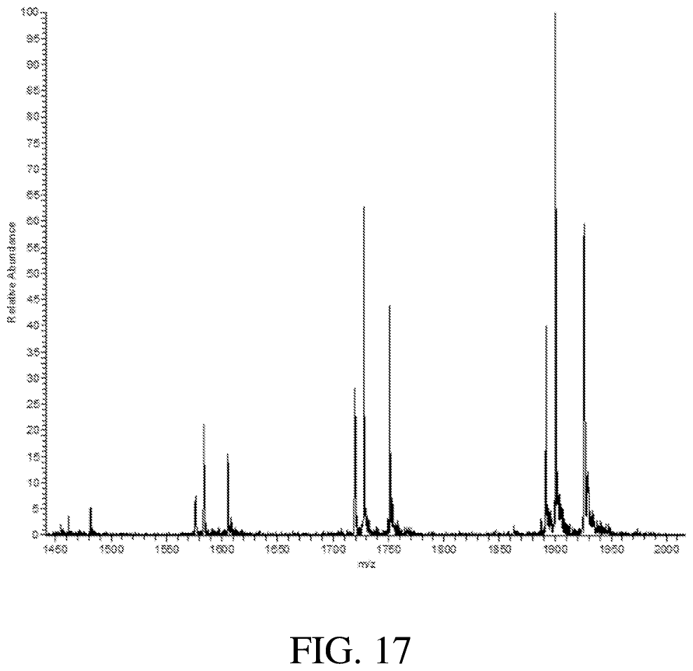

To identify variants, ten fractions were collected from the RP-UPLC fractionation of IFN, each enriched with one of the dominant peaks observed by RP-UPLC. FIG. 16B shows chromatograms for these ten fractions, with impurity peaks numbered with arrow indicators. Fractions were confirmed by RPLC, and assessed by direct injection on ESI-MS. Intact mass analysis of mass spectrum data was conducted to determine the molecular weight associated with each peak from RP-UPLC. FIG. 17 shows a representative mass spectrum from ESI-MS carried out on a fraction of IFN from RP-UPLC.

Charge state isoforms of the proteins in each injection allowed for identification of charge state and molecular weight. Molecular weights were matched with a pool of possible identities based upon protein and signal peptide sequence, cleavages and excisions therein, and common post-translational modifications (PTM). Species identified consisted primarily of incomplete signal peptide cleavage or C-terminal cleavage variants (Table 9). A singularly oxidized variant was also observed (Table 9). There was no evidence of glycosylated variants from MS. This was confirmed with an enzymatic deglycosylation assay, using Jack Bean mannosidase specific to Pichia glycosylation patterns. Problematic product variants were identified and assigned corresponding RPLC peaks for tracking through a downstream purification process selection screening process that will be described next.

TABLE 9
Product variant identities for IFN determined by ESI-MS

| Peak No. | |
|---|---|
| (Fraction No.) | Molecular weight and identity |
| Peak 1 | 19.251 kDa - - - IFN |
| Peak 2 | No detectable protein |
| Peak 3 | 20.748 kDa - - - 14 residues of |
| | signal peptide attached |
| | 20.478 kDa - - - 11 residues of |
| | signal peptide attached |
| | 20.663 kDa - - - 13 residues of |
| | signal peptide attached |
| Peak 4 | 20.278 kDa - - - 9 residues of |
| | signal peptide attached |
| | 20.748 kDa - - - 14 residues of |
| | signal peptide attached |
| Peak 5 | No detectable protein |
| Peak 6 | 19.267 kDa - - - Oxidized IFN (+16 Da) |
| | 19.235 kDa - - - Misincorporation of |
| | methoxine (−16 Da) |
| Peak 7 | 19.250 kDa - - - IFN |
| Peak 8 | 18.992 kDa - - - C-terminal KE deletion |
| | 19.250 kDa - - - IFN |
| | 18.905 kDa - - - C-terminal SKE deletion |
| Peak 9 | 19.251 kDa - - - IFN |
| | 18.750 kDa - - - C-terminal RSKE deletion |
| | 18.993 kDa - - - C-terminal KE deletion |
| | 18.905 kDa - - - C-terminal SKE deletion |
| Peak 10 | 19.250 kDa - - - IFN |
| | 18.992 kDa - - - C-terminal KE deletion |

### Downstream Purification Process Generation for IFN

### Cell Culture Fluid Partitioning Fractionation Experiments and IFN Fractionation Experiments

Null strain Pichia pastoris cell culture fluid (CCF) fractionation experiments were performed as described in Example 1.

Partially purified IFN was diluted to approximately 0.5 mg/mL in an equilibration buffer 20 mM sodium citrate at pH 4.0 and 0.2 μm filtered, and the solution was titrated to pH 3.0, 5.0, 6.0, or 7.0 as needed. The solution was titrated to lower pH using 100 mM citric acid. The solution was titrated to higher pH using 100 mM Tris base. The final concentration of IFN in the solution ranged from 100 to 500 μg/mL. The IFN solution was prepared less than 24 hours in advance of its use. IFN fractionation experiments were carried out according to the protocol described for G-CSF fractionation experiments in Example 1.

Reversed phase-ultra high pressure liquid chromatography (RP-UPLC) analysis of samples from the CCF partitioning fractionations and IFN solution fractionations was performed according to the protocol described in Example 1.

### Construction of Process-Related Impurity and Product-Related Impurity Retention Data Set

RP-UPLC chromatograms were taken as raw data .ARW files at A210, A260, and A280 with 40 Hz resolution. A MATLAB script was written to convert all raw RP-UPLC data from .ARW files to a single .MAT file. Chromatograms corresponding to blank deionized water (DI) injections were used to baseline subtract from RP-UPLC chromatograms. These DI injections were performed each day, and fractionated CCF and IFN RP-UPLC chromatograms were baseline-subtracted using the appropriate DI injection run.

Data reduction was performed by integrating each chromatogram over 0.5 second time intervals. Only data from 1.5 to 10.0 minutes were considered in order to eliminate the effects of t₀ baseline disturbances. The process-related impurity and product-related impurity retention data set was then stored as a 5-dimensional array with the first dimension corresponding to the wavelength of the data collected, the second dimension corresponding to the resin, the third dimension corresponding to the gradient type, the fourth dimension corresponding to the AKTA fraction number, and the fifth dimension corresponding to the integration window from RP-UPLC data.

A partial selection of results from IFN purification using different resins with characterizing language is presented in Table 10.

TABLE 10

| | Gradient | | Selectivity for N- | |
|---|---|---|---|---|
| Resin Name | Type | Selectivity for Aggregate | Terminal Variant | Notes |
| MEP | pH | Aggregate not present | None | Gives best resolution |
| HyperCel | | in elution | | of aggregate |
| MMC | Salt, pH 7 | Aggregate in [symbol] | None | Very promising as |
| [symbol] | | [symbol] binds | | capture step |
| [symbol] | Salt, pH [symbol] | [symbol] | [symbol] | [symbol] |
| Toyo TRP | All Salt & | Offers promising | Very little | |
| | pH Grad [symbol] | selectivity | [symbol] | |
| [symbol] | Salt, pH [symbol] | [symbol] | None | |
| NEA | pH | Offers good | [symbol] selectivity | Very promising |
| | | selectivity | for N-terminal | for N-terminal |
| | | | variant | variant removal |
| Toyo NH2 | Salt, pH 7 | None | Gives some | |
| 750F | | | selectivity | |

[symbol]

indicates data missing or illegible when filed

### Downstream Purification Process Development for IFN

The development of a process to purify IFN from Pichia CCF and product variants was initiated using the product-related impurity retention data set. The specified purification targets were host cell proteins (HCP) less than 100 ppm and DNA less than limit of detection (LOD) of a Quant-iT™ PicoGreen® dsDNA Assay Kit (ThermoFisher) kit.

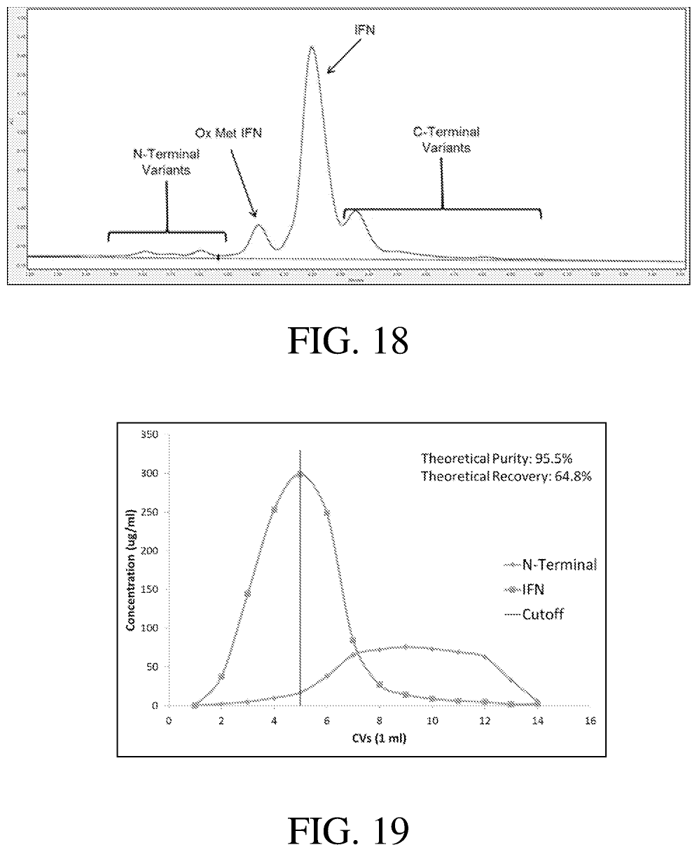

An example of a chromatogram analyzing the purity of IFN after a downstream process purification is shown in FIG. 18.

Only one resin—HEA HyperCel, an MMA/HCIC (aliphatic)—showed a high degree of selectivity for the N-terminal variant (HEA, Table 10). Process development of the purification step demonstrated that the step was highly sensitive to pH. Exploration of additional resins for N-terminal selectivity identified SP Sepharose HP as being capable of resolving the variant using a salt elution.

The final process, which employed MMC ImpRes as a capture step, MEP HyperCel as a first polishing step, and SP Sepharose HP as a second polishing step, was used to purify IFN from Pichia CCF and problematic IFN variants, and the resulting solution was characterized for product recovery and clearance of HCP, DNA, product variants, and product aggregates. For MMC ImpRes, the bind condition was 20 mM sodium citrate, pH 5.0; the wash condition was 20 mM sodium phosphate, pH 6.8; and the elute condition was 200 mM sodium citrate, 100 mM sodium chloride, 20 mM sodium phosphate, pH 7.6. For MEP HyperCel, the bind condition was 200 mM sodium citrate, 100 mM sodium chloride, 20 mM sodium phosphate, pH 7.6; the wash condition was 20 mM sodium citrate, pH 5.; and the elute condition was 20 mM sodium citrate, pH 4.0. For SP Sepharose HP, the bind condition was 10 mM sodium citrate, pH 4.3; the wash condition was 180 mM sodium chloride, 10 mM sodium citrate, pH 4.3; and the elute condition was 250 mM sodium chloride, 10 mM sodium citrate, pH 4.3. The load challenge for capture step was approximately 10 mg IFN/mL resin. An SDS-PAGE gel was run to test product quality. Enzyme-linked immunosorbent assay (ELISA) (Cygnus Technologies) was carried out on Pichia to determine information on process contaminants including host cell proteins (HCPs). A Quant-iT™ PicoGreen® dsDNA Assay Kit (ThermoFisher) was carried out to determine information on DNA process contaminants. Size-exclusion chromatography (SEC) was used to quantify aggregates. Reverse phase liquid chromatography (RPLC) was used to determine charge variants and product titer. Capillary electrophoresis (Perkin Elmer GXII) was also used to determine product titer. A cell-based proliferation assay was conducted to determine product activity.

The results presented in Table 11 show that overall product purity was 94.80%. The final product concentration (conc.) was 0.058 mg/mL, and the final aggregate content was 0.63%.

TABLE 11
IFN Purification Process Performance

| | | Percent | | | | DNA | HCP |
|---|---|---|---|---|---|---|---|
| | Total | N- | Percent | Overall | IFN | Conc. | Conc. |
| Sample | Conc. | Terminal | Aggregate | Purity* | Recovery | (ng/mg | (ng/mg |
| Name | (mg/mL) | (%) | (%) | (%) | (%) | IFN) | IFN) |
| CCF | 0.215 | 33.39 | — | — | — | 12154.66 | 1299164.7 |
| MMC | 0.467 | 35.67 | 1.68 | 65.75 | 65.79 | 122.33 | 59.2 |
| ImpRes | | | | | | | |
| Eluate | | | | | | | |
| MEP | 0.511 | 30.53 | 0.66 | 69.88 | 78.85 | 159.36 | 143.32 |
| Eluate | | | | | | | |
| SP | 0.058 | 4.79 | 0.63 | 94.80 | 58.01 | 163.84 | <LOD |
| Sepharose | | | | | | | |
| HP Eluate | | | | | | | |

*Overall Purity considers all impurities as determined by A215 (or A280 when A215 is saturated)

In the three-column process, the most problematic product variants were greatly reduced. By using the selected process, N-terminal variant was reduced to 4.79%. A composition profile was determined by RP-HPLC for IFN and N-terminal variants during the isocratic elution condition identified for the final process. FIG. 19 shows a composition profile using data from RP-HPLC, where each fraction is expressed as a number of column volumes (CVs). By changing the cutting conditions, lower N-terminal content was obtained, directly resulting in higher product purity.

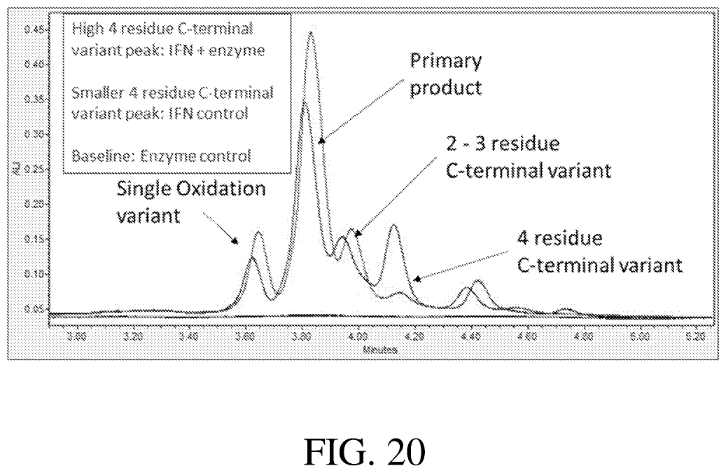

To ensure that glycosylation was not a lingering issue in the final product pool, enzymatic deglycosylation was conducted. The C-terminal deletion peak was the only one exhibiting an increase over the incubation period, which did not suggest glycosylation. This can be seen from FIG. 20, which shows chromatograms analyzing purified IFN sample before (smaller 4 residue C-terminal variant peak) and after (high 4 residue C-terminal variant peak) deglycosylation.

### Example 4

### Chromatographic Process Performance for G-CSF

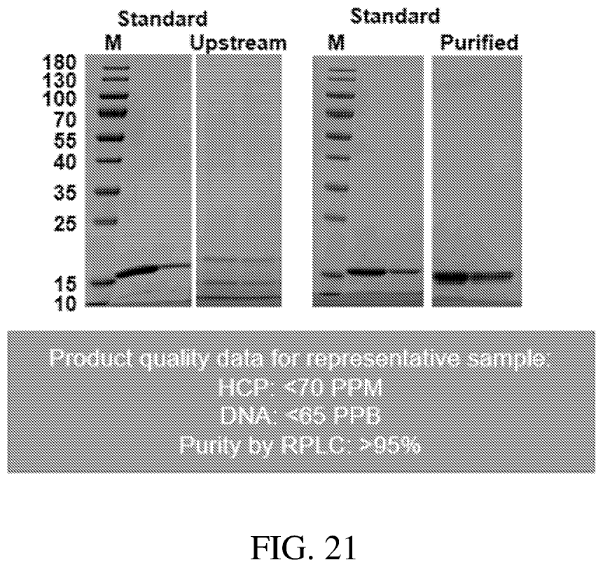

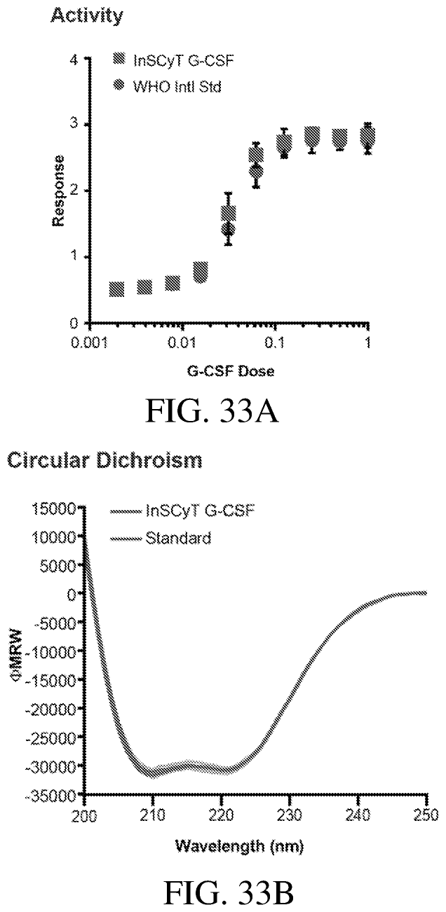

The downstream purification process determined as in Example 1 was used to purify granulocyte-colony stimulating factor under similar operating conditions to those used in Example 1. The product quality obtained contained host cell proteins (HCP) in an amount less than 70 PPM as determined by ELISA (Cygnus Technologies), DNA in an amount less than 65 PPB as determined by qPCR (resDNASEQ Quantitative Pichia DNA Kit—Life Technologies), and an overall purity of greater than 95% as determined by RPLC, as in FIG. 21 which includes an SDS-PAGE gel. The product had substantially the same activity and circular dichroism as the standard as shown in FIGS. 33A and 33B. FIG. 33A shows the bioactivity of experimentally purified G-CSF (InSCyT) as compared with the WHO International Standard (WHO Intl Std, NIBSC 98/574). Bioactivity data was determined by a cell-based proliferation assay conducted by a contract research organization. FIG. 33B shows the circular dichroism of experimentally purified G-CSF (InSCyT) as compared with a reference standard. The product as achieved by the process was produced in an amount greater than 150 doses in a total time from initiation of the bioreactor to completion of purification of 6 days.

The pharmacokinetic profile as well as pharmacodynamic effect (neutrophil stimulation) of the experimentally purified G-CSF was tested in an animal model (Sprague Dawley rats) compared to Neupogen (predicate control) following a single subcutaneous administration. Briefly, thirty-nine male Sprague Dawley rats were assigned to four groups: 12 animals in each of Groups 2-4 and three animals in untreated control Group 1. Animals in Groups 2 and 3 received experimentally purified G-CSF at concentrations of 115 μg/kg and 575 μg/kg respectively and animals in Group 4 received predicate control (Neupogen) at a concentration of 115 μg/kg. Blood samples were collected for pharmacokinetic assessment (four time points from three rats per group) at pre-dose, 0.5, 1, 2, 4, 8, 12, 24, 48, 72, 96, and 120 hours post-dose.

Blood samples were also collected from three rats per group for neutrophil analysis 24 hours post dose. A statistically significant increase in relative neutrophil counts was observed in the peripheral blood 24-hours post dose in both experimentally purified G-CSF and Neupogen® treated groups compared to vehicle control group. Increased relative neutrophil counts observed were considered to be treatment related and an expected pharmacological or pharmacodynamic effect of both experimentally purified G-CSF and Neupogen®. The pharmacokinetic profile and the pharmacodynamic effect (neutrophil stimulation) of the experimentally purified G-CSF and Neupogen were similar.

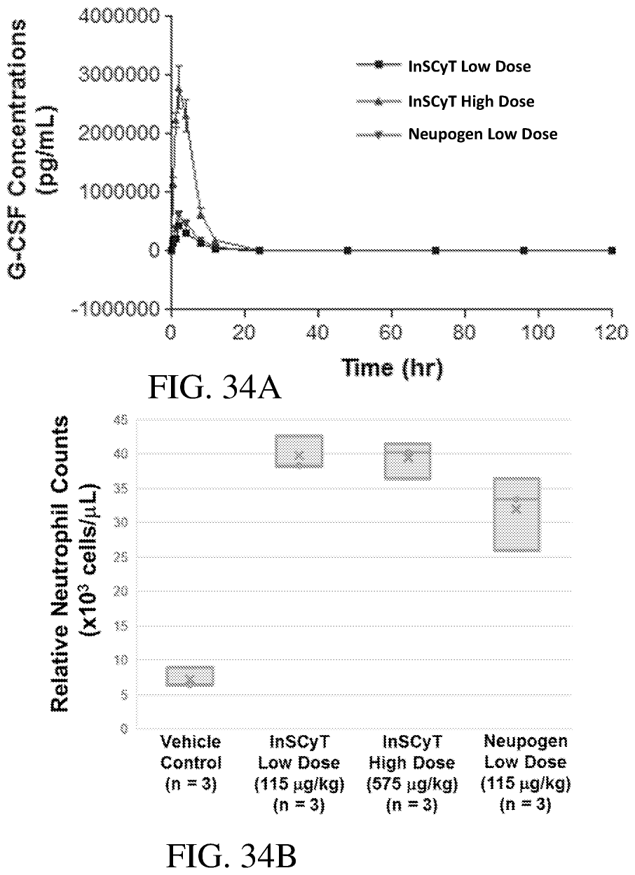

As shown in FIG. 34A, the experimentally purified G-CSF had substantially the same pharmokinetics as the standard (i.e., neupogen) when administered to animals at 115 μg/kg (shown by InSCyT low dose). The higher dose of experimentally purified G-CSF (575 μg/kg) resulted in a higher plasma concentration of G-CSF in treated animals over time (as shown by InSCyT high dose) than the standard. Experimentally purified G-CSF also had a greater pharmacodynamic effect than the standard. As shown in FIG. 34B, animals treated with either a 115 μg/kg or a 575 μg/kg dose of experimentally purified G-CSF had a higher neutrophil counts than those animals treated with the Neupogen standard at 115 μg/kg. In addition, experimentally purified G-CSF exhibited good safety: all animals survived to the last blood collection time point. No abnormal clinical signs or injection site reactions were observed in any animals treated with the experimentally purified G-CSF.

### Example 5

### Chromatographic Process Performance for Human Growth Hormone (hGH)

Another downstream purification process was used to purify human growth hormone (hGH). The downstream purification process used in this example included a first chromatography column with Pall CMM HyperCel (multimodal cation exchange) resin operated in bind-elute mode. The bind conditions were pH 5.5 (from 5.0 to 6.8 possible), conductivity in the range 0 to 50 mS/cm. The wash conditions were pH 6.0 (from 5.8 to 6.5 possible), conductivity 4 mS/cm (from 0 to 10 mS/cm possible). The elution conditions were pH 8.0 (from 7.5 to 8.5 possible), conductivity 4 mS/cm (from 0 to 10 mS/cm possible). The next step in the downstream purification process was a second chromatography column with Pall HyperCel STAR AX (anion exchange) resin operated in flow-through mode. In this example, there were no further purification modules.

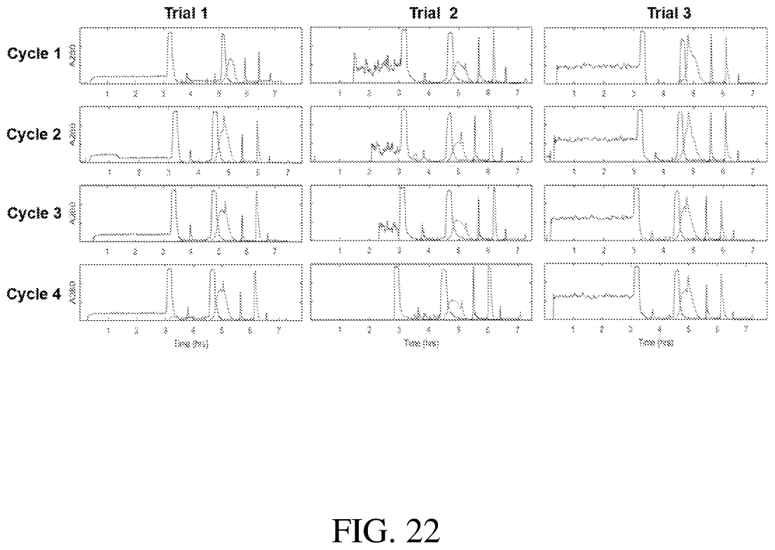

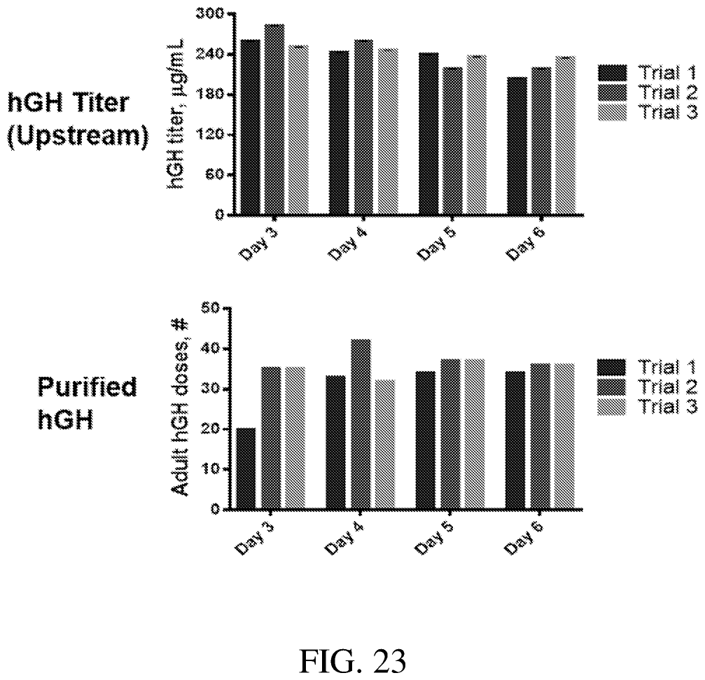

The purification of hGH was found to be consistent across multiple cycles and multiple runs, as shown by the chromatograms in FIG. 22. This consistent purification was determined to lead to consistent hGH production quantities, as shown in FIG. 23. In FIG. 23, hGH concentration before purification was measured using RPLC, and hGH concentration after purification was measured by NanoDrop (Thermo Scientific; A280 signal), and then converted to number of doses using the maximum adult dosage of hGH of 1.75 mg.

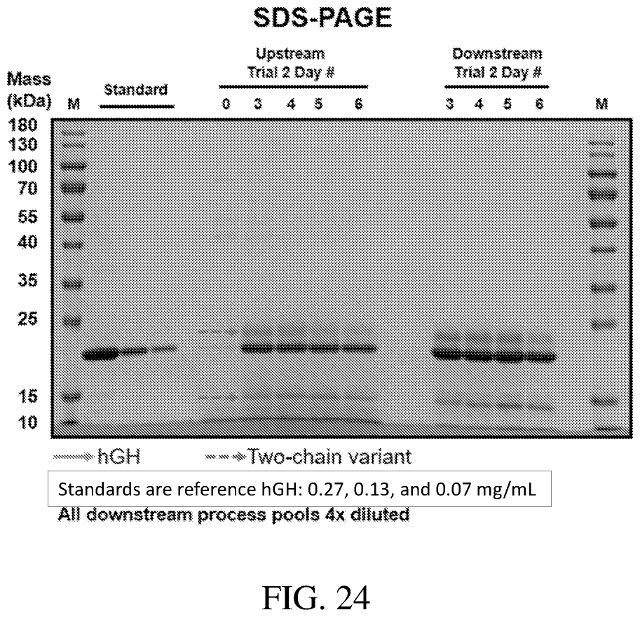

SDS-PAGE was used to analyze the purified product, as shown in FIG. 24, and it was determined that host cell proteins were reduced by 2-3 logs to less than 100 PPM as determined by ELISA (Cygnus Technologies); DNA was reduced by greater than 4 logs to less than 45 PPB as determined by qPCR (resDNASEQ Quantitative Pichia DNA Kit—Life Technologies). Therefore, process-related variants were cleared during purification.

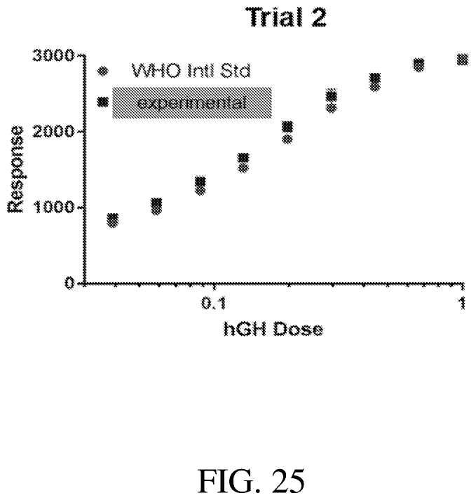

FIG. 25 shows the bioactivity of experimentally purified hGH (experimental) as compared with the WHO International Standard (WHO Intl Std). Bioactivity data was determined by a cell-based proliferation assay conducted by a contract research organization. Table 12 shows liquid chromatography mass spectrometry (LCMS) results, which revealed product variants detected but potency not affected. Data exists in the scientific literature to support that two-chain variants are not clinically relevant.

TABLE 12
hGH Purification Process Performance LCMS

| | Range | Average | Standard |
|---|---|---|---|
| Sequence Coverage | 100% | 100% | |
| Oxidation (M14) | 2.9-15.5% | 8.8% | 3.6% |
| Oxidation (M125) | 3.1-8.3% | 4.7% | 2.4% |
| *Oxidation (M170) | 0.7-1.5% | 1.0% | n/a |
| Deamidation (N149) | 7.8-16.5% | 10.8% | 0.4% |
| Two chain (Q141) | 0.4-17% | 5.9% | n/a |

*For the above product variants only oxidation at M170 has been shown to be detrimental to activity

### Example 6

### Chromatographic Process Performance for IFN

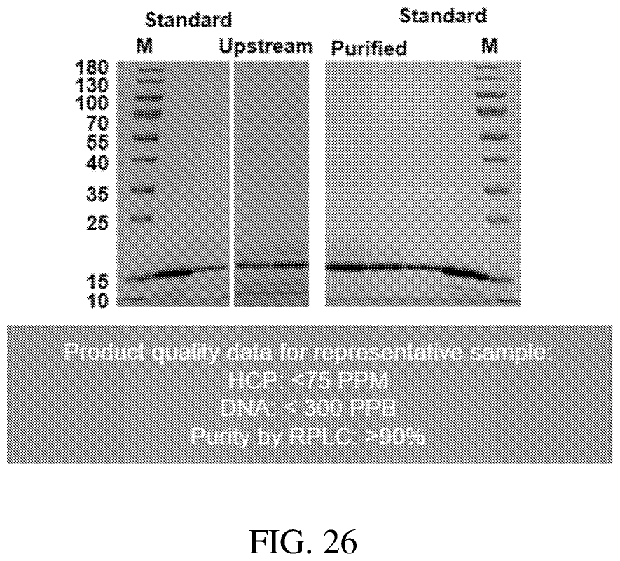

The downstream purification process determined as in Example 3 was used to purify interferon α-2β (IFN) under similar operating conditions to those used in Example 3. The product quality obtained contained host cell proteins (HCP) in an amount less than 75 PPM, DNA in an amount less than 300 PPB, and an overall purity of greater than 90%, as in FIG. 26 which includes an SDS-PAGE gel. The purification process resulted in the production of greater than 4000 doses of IFN in 6 days. The final dosage form of IFN was 0.024 mg/mL in aqueous solution with 7.5 mg/mL NaCl, 1.8 mg/mL sodium phosphate dibasic, 1.3 mg/mL sodium phosphate monobasic, and pH 6.85.

### Example 7

This Example describes a system comprising a magnetic level sensor and a perfusion bioreactor.

Sensing the level of liquids in fluid-holding systems such as bioreactors, hold tanks, surge tanks, etc. can provide one measure for enabling process control since the rate of perfusion and feeding can be regulated as required by the operator to create the optimized production conditions in the vessel. Current approaches for perfusion bioreactors use in-vessel level probes for this purpose. Non-invasive sensing can offer multiple benefits, including reduced risk of contamination, reduced geometric complexity, and potential cost savings. For disposable bioreactors, non-invasive sensors could allow for reuse of the sensor, facilitating rapid turnaround of process equipment without necessitating sterilization.

The few non-invasive technologies for sensing liquid level that exist in other industries do not typically lend themselves to applications in bioreactor vessels. For example, external capacitive level sensors work by measuring the change in dielectric constant through the vessel wall. The ionic conductivity of the fluid in fermentation typically changes during the course of a run, however, and can interfere with these measurements.

In this Example, a non-invasive liquid level sensing technique using a magnetic float and externally-mounted reed switches is described. This technique does not depend on fluid properties, but instead leverages the fluid level itself to allow direct measures of the fluid level in the reactor.

### Materials and Methods

A magnetic liquid level sensor using reed switches and a magnetic float was designed and prototyped. The magnetic liquid level sensor was a discrete sensor suitable for use in applications where the process control is based upon point-level control. With high-resolution, multi-level modifications, it can also be used in a pseudo-continuous manner if the required liquid level resolution is sufficiently high.

A magnetic float was suspended around a non-magnetic shaft placed in a vessel. The proximity to the edge of the vessel ensured that the magnetic field penetrated the outer surface of the vessel. To achieve a specific gravity less than that of the reactor fluid (approximately that of water), a foam polystyrene floatation ring was used as the float. Four nickel-coated neodymium [NdFeB] magnets were equidistantly spaced and embedded in the float. The magnets were of a ½″ disc diameter and ¼″ thickness, with an individual pull force of 6.1 pounds. An array of reed switches were placed external to the vessel. Reed switches generally refer to electric switches that turn on in the presence of a magnetic field. Depending on the specific reed switch and magnet strength, the distance and orientation required to turn them on may vary. In this Example, the reed switches used were SPST-NO, with a magnetic sensitivity of 12-18 Ampere-turns. The probe holding the magnetic float was a plastic shaft. The vessel was a glass beaker with a diameter of 4.25 inches (10.80 cm) and a wall thickness of 0.15 inches (0.38 cm). The vessel geometry can affect sensor performance, since level sensing resolution is based on height, and a smaller diameter vessel can lead to increased resolution for the same volume change.

One configuration tested included an array of 5 reed switches. To provide visual feedback on the system for the active switch, each reed switch was placed in series with a different colored light-emitting diode (LED). In a setup for process control, other means of communications such as current sensors or a direct voltage or current signal can be sent to the processor, instead or in addition to LEDs. In this Example, the spacing of the reed switches was intended to turn on either one or two LEDs.

### Results and Discussion

The liquid level was varied between 0 mL and 1000 mL. To minimize the effect of LEDs flickering on and off due to the magnetic field only temporarily reaching the reed switches, the reading was allowed to stabilize for 10 seconds before the LED was said to be ON. This mimicked a process control step that verifies a positive signal for 10 seconds before processing the level. The results corresponded very closely to the designed logic. The discrepancies occurred due to lack of robustness of the experimental setup and the fluctuations and instabilities introduced due to testing.

In a stable bioreactor environment, with a commercially prototyped array of reed switches, and knowing the exact magnetic field environment, as well as the interference from surrounding objects, a precise sensor can easily be developed to follow its assigned logic perfectly. Additionally, the discrete level sensor can transform into a pseudo-continuous sensor by adding reed switches and increasing the amount of steps (n). As n goes to infinity, the sensor will overlap the actual level exactly, and the sensor will become continuous. Depending on the resolution of liquid level sensing required, n will need not go to infinity, but instead be high enough to achieve sensing to the accuracy desired. If the logic dictates that each step is evenly spaced (which is not the case in the current setup due to leveraging combinations of ON states to achieve more steps), then n equals the number of reed switches, and the number of different level states that can be predicted. As n increases, the resolution becomes fine enough to resemble a continuous sensor. For example, at n=100, the measured liquid level overlaps with the true level.

An improved version of the Magnetic Float Liquid Level Sensor would comprise a custom-made adhesive strip of reed switches that attaches externally to the vessel. All the necessary information regarding which switch is sensing current would be parsed through to one end of the strip and output to the processor. The strip would be carefully manufactured so that the switches are equidistant but also with the knowledge of the rest of the system (such as the magnetic float, vessel wall properties, etc.) so as to ensure perfect relay of logic as the magnetic float changes with level. The magnetic float itself would have a uniform distribution of magnetic material so that rotation of the float does not change the magnetic field. The binary nature of reed switches makes them reliable and robust, but Hall Effect sensors are an alternative to reed switches that can also be used. Hall Effect sensors are similar to reed switches in that they react to a magnetic field, but they can act as a continuous sensor due to their ability to linearly increase output voltage (until saturation) with increasing magnetic field density.

### Conclusion

A liquid level sensor using a magnetic float and an array of reed switches was successfully tested. This non-invasive option to liquid level sensing may have a wide range of applications in biotechnology as well as other industries in which non-invasive fluid sensing would be beneficial. While the magnetic level sensor is primarily a discrete sensor that lends itself to point-level alarm control, with increased resolution (or implementation of Hall Effect sensors), it can also be used for process control when continuous level sensing is required. Using an external magnetic level sensor allows for reduced reactor complexity, reduced risk of contamination, increased cost savings from reuse, and other benefits when used for process control in a single-use, continuous perfusion bioreactor.

### Example 8

This Example describes a system comprising an optical level sensor and a perfusion bioreactor.

To enable perfusion in small-scale bioreactors, hold tanks, surge tanks or other fluid-holding tanks, there is an opportunity for advancing probes for real-time, online monitoring of various process parameters such as temperature, dissolved oxygen, pH, and cell growth, for process control. Liquid level in the reactor is another parameter that can be monitored for controlling the rate of perfusion, the rate of nutrient feed, or in any step that requires volume control. Depending on the nature and requirements for process control, the liquid level sensor could sense a single point, multiple discrete points, or continuous levels.

Stirred single-use bioreactors configured for perfusion currently use invasive liquid level sensors such as conductive level probes. Having non-invasive disposable alternatives to sense liquid level could offer several advantages, including reduced vessel and headplate complexity, reduced risk of contamination, and increased cost savings.

A few non-invasive level sensing methods exist, but are typically unsuitable for use in bioreactors. Externally-mounted capacitive level sensors that sense the changing dielectric constant of the fluid are generally sensitive to changes in ionic conductivity of the reactor fluid during a bioreactor run. Methods such as load cells and pressure transducers are often subject to variations in the density of the reactor fluid, as well as altered by vibrational noise. Time of flight methods such as ultrasonic or lasers typically require very high resolution due to the small geometries, and are susceptible to change due to a wide range of parameters, thus needing to be fairly expensive in order to work in this scenario, negating the benefits of the non-invasive method.

In this Example, three different optical-based level sensors that provide low-cost and accurate means of discrete as well as continuous level monitoring were designed and demonstrated.

### Discrete Optical Level Sensor

A clear plastic probe was constructed with an outer diameter of 8 mm, a wall thickness of 1 mm, and a height of 13 inches. At a distance of 3.5″ from the bottom of the probe, bright blue tape (VWR) with a width of ½″ was wrapped around the length of the plastic probe once, to cover exactly ½″ of height. After a spacing of ⅛″ below the end of the blue tape, orange tape (VWR) with a width of ½″ was wrapped the length of the plastic probe once, to cover exactly ½″ of height. Similarly, keeping the spacing and length consistent, green tape (VWR), bright red tape (VWR), and lavender tape (VWR) were respectively wrapped around the probe. The plastic probe was tape-free for the last half-inch at the bottom.

Continuous Co-ordinate-based Optical Level Sensor

A clear plastic probe was constructed with an outer diameter of 8 mm, a wall thickness of 1 mm, and a height of 13 inches. A float was constructed by using an annular, semi-hollow polypropylene piece having an outer diameter 27.5 mm, an inner diameter of 11 mm (thickness of 8.25 mm), a height of 12 mm, and a weight of 4.19g (density of 175 kg/m³). Bright red tape (VWR) with a width of ¹/₂″ was wrapped around the float so as to cover it completely. After the addition of the tape, the float weighed 4.52 g. The float was then placed around the plastic probe.

### Continuous Area-based Optical Level Sensor

A clear plastic probe was constructed with an outer diameter of 8 mm, a wall thickness of 1 mm, and a height of 13 inches. Bright red tape (VWR) with a width of ½″ was wrapped around the length of the plastic probe to cover 10 inches of height. The tape was overlapped such that the plastic was not visible along the 10 inches. Above 10 inches, the clear plastic was visible.

### Image Processing Algorithms

A laptop running MATLAB R2015b (Windows 8.1), with an Intel® Core™ i7-3630QM CPU @ 2.40 GHz processor with 8.00 GB of installed memory RAM, was used for its built-in webcam for image acquisition, and to run the MATLAB image processing algorithms online, in real-time. The liquid level was then varied as required, while the process computer's webcam acquired images. For all three methods described here, the algorithms developed as described were then run continuously using the acquired images, and the level was predicted. Chrominance-based Binarization (CBB) Algorithm

A CBB algorithm was developed to create a binary (black and white) image from the original image by selecting for the colors of interest. The acquired image was first converted from the RGB (red, green, blue) space to the HSV (hue, saturation, value) space. The H-value generally provides a truer representation of the color of the object and is less sensitive to environmental lighting conditions, unlike the RGB values in which color and luminance information are coupled. The HSV image was then filtered using thresholding of the different hue, saturation, and value data against the known ranges of the colors of interest. To remove optical and physical noise, the holes in the binary image (for example, a couple of black pixels amongst many white ones) were then filled by using the “imfill” function in order to ensure objects remain together and are not compromised.

### Colored Object Detection (COD) Algorithm

The binary image generated from the CBB algorithm was then converted into discrete objects. The COD algorithm applied a Gaussian blur and filter to the binary image to smooth erroneous pixels, physical, and optical imperfections and to reduce vibrational noise. This transformation was done by clustering binary data and creating “blobs” that represented objects of the specific color in the original image. To avoid flecks of the specific color in the image, and other minor objects of the same color that may interfere with the process, an area-based filter was then applied to only retain objects within a certain pixel area range. For the geometric setup and camera resolution (640x800 pixels) implemented, the allowed range of area was set to filter objects smaller than 40 pixels when searching for the colored float or the painted bands, and 500 pixels when searching for the colored shaft. This range was determined by knowing the positioning of the camera in relation to the setup, as well as the camera resolution, which allowed correlations to the range of sizes of the binary objects expected.

The retained objects of the specific color were then counted. Within the specified range, only one object should remain for each color of interest, since the choice of apparatus and geometry were designed to leave one remaining object per color. These visualized objects were leveraged in different ways for each sensor method described.

### Painted Bands (PB) Algorithm

The PB algorithm used the COD algorithm to extract information from the binary images it created. The objects for each band color were counted in the COD algorithm and fed to the PB algorithm. The PB algorithm used these counts to detect the presence or absence of the specific painted band in the image and correlated that to the point-level being above or below certain values associated with the bands at those levels.

### Red-colored Float (RCF) Algorithm

The RCF algorithm used the COD algorithm to extract information from the binary image it created. The algorithm detected the location of the red object and determined its centroid. By pre-determined geometrical calculations and knowledge of camera-acquired image specifications, the centroid of the red float that was detected was then used to correlate to liquid level. An average of five level readings were conducted at each point, and the calculated mean value was the resulting predicted level value. In case of an erroneous zero reading, the code disregarded this value in calculating the mean.

### Red-colored Shaft (RCS) Algorithm

The RCS algorithm used the COD algorithm to extract information from the binary image it created. This algorithm detected the residual size of the red probe. By pre-determined geometrical calculations and knowledge of camera-acquired image specifications, the area was then used to determine the liquid level. An average of five level readings were conducted at each point, and the calculated mean value was the resulting predicted level value. In there was an erroneous zero reading, the code disregarded this value in calculating the mean.

### Experimental Setup

A 1000 mL glass beaker with wall thickness of 0.15 inches (0.38 cm) was used to model the bioreactor. The beaker was filled with a spent cell culture of Pichia pastoris post-run that was stirred at 600 rpm using a VWR mini-magnetic stirrer and plate, thus simulating an agitated bioreactor. Along with the image-capturing camera and process control computer, the elements constructed for each level sensing method were then introduced into the assembly for each experiment.

### Results and Discussion

Process control in bioreactors generally requires continuous monitoring of liquid level. Due to the drawbacks of in-vessel level sensors, three non-invasive optical methods were developed.

Discrete Optical Level Sensor In lieu of an in-vessel point level sensor, an optical method comprising painted bands along the impeller shaft was designed. Taking advantage of the typical opacity of the reactor fluid, the camera could only see the colored bands above the surface. To sense certain pre-defined measurement points, the system read the visible colors to determine the current level. To ensure the image processing algorithms worked robustly, the colors were chosen to be different enough from each other and from the reactor fluid. In this Example, five colors were used—blue, orange, green, red, lavender. To test the Discrete Optical Level Sensor, the vessel volume was varied from 0 mL to 1000 mL. The camera and computer sensed the presence or absence of the five colors and predicted the point level. As a safety margin, the process control set the level above the associated level when a color was detected. This setting could be set at the lower bound as well.

The number of cells generally varies during the course of fermentation, and the opacity of the reactor fluid also generally changes. To determine if the level sensor could accurately report the fluid level throughout the process, the optical densities (OD) of reactor fluid were varied from 0 to 0.600, equivalent to a cell concentration of 0 to 3.6×10⁷ cells/mL. To do this, the OD₆₀₀ measurement of BMGY media without cells was used as a blank reference in the UV spectrophotometer. The cell optical density was then varied from an OD₆₀₀ value of 0 to 0.600, while the level sensor was tested.

The lighting conditions where the reactor is housed could also fluctuate during long fermentations. To test the robustness of the optical level sensors with changing light surroundings, the experiment was repeated by changing the light intensity incident on the setup, in a room with only one source of light, from 1,000 to 100,000 lux, while the percentage of failed readings (no detection of desired objects, or a zero reading) was measured. The optical density was set to a constant OD₆₀₀ of 0.300 (cell concentration of 1.8×10⁷ cells/mL) for this experiment.

In the experiment, the sensor triggered the level above “X” to which it was assigned accurately for each level, with the highest absolute mean error being 4 mL and the highest percentage mean error being 0.67%. Each individual calculation took an average of 1.4 seconds to complete. The results show that the sensor worked at optical densities above 0.171 OD600 (1.03×10⁷ cells/mL) and had no failed readings once the illuminance was above 29100 lux on the apparatus, at an optical density of 0.300 OD₆₀₀.

Continuous Co-ordinate-based Optical Level Sensor

While a point-level sensor may be appropriate in some applications, a continuous level sensor that tracked liquid level directly by using a float was developed to provide more control in the process while maintaining the benefits of a non-invasive optical level sensor. As the float varied directly with the level of liquid in the vessel, capturing the co-ordinates of the float enabled prediction of the liquid level. To ensure the float was easily detectable by the algorithms, its color was different from that of the liquid. In this Example, a red float was used, and the experiment was set up and built as detailed above. To test the Continuous Coordinate-based Optical Level Sensor, the vessel volume was varied from 400 mL to 1000 mL while the camera and computer sensed the position of the red float and used it to predict the liquid level. Similar to the case of the Discrete Optical Level Sensor, the OD and light intensity experiments were also conducted to determine the range in which the sensor works.

The results show that there were no erroneous zero readings in the primary experiment conducted due to good lighting conditions. The average time taken for each individual level calculation using the current setup was 1.6 seconds, with 7.9 seconds required to predict a level using five averaged readings. To assess the accuracy of the predicted values, a range of liquid level values (between 400 mL and 1000 mL) was measured. The highest absolute error was 10.8 mL, with a highest percentage error of 2.38%. The standard deviation for the individual level readings had a highest value of 8.7 mL and a highest standard error value of 2.6 mL. The sensor worked at all tested optical densities between 0 to 0.600 OD₆₀₀ and had no failed readings once the illuminance was above 1800 lux on the apparatus, at an optical density of 0.300 OD₆₀₀.

### Continuous Area-based Optical Level Sensor

The benefits of a non-invasive, continuous liquid level monitoring system were achieved using the previous setup. However, the use of an additional part (the float) was necessary to achieve the desired functionality. In order to try and achieve the same functionality without the addition of added parts, a different continuous level sensor was created using the agitator shaft that was already in the vessel. By detecting the amount of visible shaft area using a camera, the computer predicted the level by assuming the liquid in the vessel blocked the rest of the agitator shaft. To ensure the shaft was easily distinguishable so the algorithms worked appropriately, the agitator shaft of the bioreactor was a contrasting color to the reactor fluid—bright red was used in this Example.

In order to test the Continuous Area-based Optical Level Sensor, the experiment was built and set up as detailed above. The vessel volume was varied from 400 mL to 1000 mL, while the camera and computer sensed the amount of visible shaft area and correlated it to liquid level. Similar to the previous optical level sensors, the OD and light intensity experiments were also conducted to determine the range in which the sensor works.

The results show that there were no erroneous zero readings in the primary experiment conducted due to good lighting conditions. The average time taken for each individual level calculation using the current setup was 1.5 seconds, with 7.7 seconds required to predict a level using five averaged readings. To assess the accuracy of the predicted values, a range of liquid level values (between 400 mL and 1000 mL) was measured. The highest absolute error was 15.0 mL, with a highest percentage error of 2.34%. The standard deviation had a highest standard deviation value of 5.0 mL and a highest standard error value of 1.5 mL. The results also show that the sensor worked at optical densities above 0.133 OD600 (7.98×10⁶ cells/mL) and had no failed readings once the illuminance was above 2900 lux on the apparatus, at an optical density of 0.300 OD₆₀₀.

### Discussion

The successful testing of the optical level sensors validated a novel solution to process control using level sensing in bioreactors. There is no longer a requirement for an invasive sensor that inconveniences the setup as well as introduces a potential sterility concern. An external sensor may allow for reusability even in the case of disposable bioreactors and may be cost effective in the long run. Two of methods created were continuous to offer complete process control, whereas one method was discrete, lending itself to point-level applications in biotechnology.

As seen in the results, the sensors worked on the order of seconds. The reactor fluid volume typically changes at a rate of 15-30 mL/hour, which means the sensors developed are adequate for process control. Typically, the rate of fluid flux incoming or outgoing in perfusion bioreactors is not fast enough to alter the liquid level faster than the sensor calculates the level. The results also demonstrate that the sensors work in a large range of lighting conditions and a large range of optical densities. The Continuous Coordinate-based Optical Level Sensor worked at all densities, whereas the other two designs began to work at very low cell concentrations as well. With regards to illuminance, the Continuous Coordinate-based Optical Level Sensor worked perfectly even at 1800 lux, whereas the Continuous Area-based Optical Level Sensor required marginally more illuminance at 2900 lux to ensure no zero readings. The Discrete Optical Level Sensor required the most light to work all the time, with a minimum illuminance of 29100 lux. The Continuous Coordinate-based Optical Level Sensor required the addition of the float as a piece of equipment, whereas the other two methods used an existing part of the setup (the shaft) to achieve functionality. Since the Discrete Optical Level Sensor only gauged the presence or absence of a specific color band, it was more robust for its application, as opposed to the continuous level sensors. However, the continuous sensors offered more control due to the ability to predict level to a closer degree.

Since all methods used optical sensing and chrominance filtering to achieve their respective results, after a certain point, the drastically changing light conditions made the optical level sensors vulnerable to stop working. This problem may be circumvented in future designs by using a more sophisticated setup with a controlled and consistent light source inside a closed environment. Alternatively, a slightly less robust but cheaper modification to the current setup would be to use a “gray card” approach, where the code uses the known gray card HSV values as a reference and corrects the image for changing lighting before implementing the algorithms. The formation of a vortex at certain stirrer speeds could potentially slightly skew the level sensors since the liquid surface becomes concave and has different heights at different points. If the formation of a vortex is a concern to proper process control, and very precise level sensing is required, the two continuous techniques can be used in tandem to correct for the vortex. With the float level at the wall, and the agitator shaft level at the center, the two methods will experience opposite effects of the vortex level change, and can be combined to correct to the true level (volume) of the vessel. Alternatively, knowledge of the vortex formed with stirrer speed can be used to correct and recalibrate the level sensor.

### Conclusions

Overall, the methods and algorithms developed worked effectively to gauge liquid level for the purpose of process control in running a single-use, continuous perfusion bioreactor, and offer plenty of advantages over traditional level sensors. The optical methods offer a non-invasive alternative that do not cause sterility concerns, avoid added reactor complexity and cost, and are reusable even in a single-use bioreactor setup.

### Example 9

This example describes an alternative process for the downstream purification of IFN. The chromatographic process design was the same as in Example 3, except an additional constraint was placed on the downstream process generation tool. The 3-step process was required to have a single flow-through step. The downstream purification process having a single flow-through step had a higher percent product recovery than the process in Example 3 and a similar host cell proteins (HCP) concentration and total variant content.

### Downstream Process Generation Tool: Process Sequences

Table 13 shows the top 20 process sequences selected by the process selection tool for the purification of IFN. Sequences are presented with their scores calculated by Equation 1 along with a host-related impurity rank, variant removal rank, and a summed rank. Individual steps are presented in the format of “resin, operating mode/pH, product elution condition”, where an operating pH is given when using a salt gradient elution and “flow-through” is used to indicate that the product elution condition is the same as the load condition.

TABLE 13

| | | | | Host-Related | | |
|---|---|---|---|---|---|---|
| | | | | Impurity | Variant | |
| | | | | Removal | Removal | Summed |
| Rank | Step 1 | Step 2 | Step 3 | Rank | Rank | Rank |
| 1 | Nuvia cPrime | Q Sepharose | Capto Adhere | 64 | 90 | 154 |
| | pH 7.0, 70 mM | HP | pH 7.0, Salt | | | |
| | NaCl | pH 7.0 | Step, 610 mM | | | |
| | | Flowthrough | NaCl | | | |
| 2 | Capto Adhere | Q Sepharose | SP Sepharose | 51 | 151 | 202 |
| | pH Step, pH | HP | HP | | | |
| | 5.0 | pH 5.0 | pH 4.0, | | | |
| | | Flowthrough | 420 mM NaCl | | | |
| 3 | Toyopearl | HyperCel | Nuvia cPrime | 6 | 208 | 214 |
| | MX-Trp- | STAR AX | pH 7.0, 70 mM | | | |
| | 650M | pH 6.0 | NaCl | | | |
| | pH Step, pH | Flowthrough | | | | |
| | 5.8 | | | | | |
| 4 | Toyopearl | Toyopearl | Nuvia cPrime | 19 | 208 | 227 |
| | MX-Trp- | NH2-750F | pH 7.0, 70 mM | | | |
| | 650M | pH 6.0 | NaCl | | | |
| | pH Step, pH | Flowthrough | | | | |
| | 5.8 | | | | | |
| 5 | Capto Adhere | SP Sepharose | Q Sepharose | 64 | 90 | 154 |
| | pH Step, pH | HP | HP | | | |
| | 5.0 | pH Step, pH | pH 6.0 | | | |
| | | 5.7 | Flowthrough | | | |
| 6 | Toyop earl | Nuvia cPrime | Q Sepharose | 41 | 208 | 249 |
| | MX-Trp- | pH 7.0, 70 mM | HP | | | |
| | 650M | NaCl | Flowthrough | | | |
| | pH Step, pH | | pH 7.0 | | | |
| | 5.8 | | | | | |
| 7 | Nuvia cPrime | Q Sepharose | PPA HyperCel, | 35 | 214 | 249 |
| | pH 7.0, 70 mM | HP | pH Step, pH | | | |
| | NaCl | pH 7.0 | 3.8 | | | |
| | | Flowthrough | | | | |
| 8 | Toyopearl | MEP | Capto Adhere, | 86 | 166 | 252 |
| | MX-Trp- | HyperCel, pH | pH 5.0, | | | |
| | 650M | Step, pH 5.0 | Flowthrough | | | |
| | pH Step, pH | | | | | |
| | 5.8 | | | | | |
| 9 | Capto Adhere, | Q Sepharose | SP Sepharose | 155 | 98 | 253 |
| | pH Step, pH | HP | HP, pH Step, | | | |
| | 5.0 | pH 5.0 | pH 5.7 | | | |
| | | Flowthrough | | | | |
| 10 | Nuvia cPrime | Toyopearl MX- | HEA | 55 | 201 | 256 |
| | pH 7.0, 70 mM | Trp-650M, pH | HyperCel, pH | | | |
| | NaCl | 7.0 | Step, pH 4.1 | | | |
| | | Flowthrough | | | | |
| 11 | Toyopearl | Q Sepharose | Nuvia cPrime, | 48 | 208 | 256 |
| | MX-Trp- | HP | pH 7.0, 70 mM | | | |
| | 650M | pH 6.0, | NaCl | | | |
| | pH Step, pH | Flowthrough | | | | |
| | 5.8 | | | | | |
| 12 | Capto Adhere, | SP Sepharose | Q Sepharose | 127 | 151 | 278 |
| | pH Step, pH | HP, pH 4.0, | HP, pH 4.0, | | | |
| | 5.0 | 420 mM NaCl | Flowthrough | | | |
| 13 | Nuvia cPrime | Q Sepharose | HEA | 81 | 201 | 282 |
| | pH 7.0, 70 mM | HP | HyperCel, pH | | | |
| | NaCl | pH 7.0, | Step, pH 4.1 | | | |
| | | Flowthrough | | | | |
| 14 | Nuvia cPrime | Capto MMC | Capto Adhere, | 15 | 267 | 282 |
| | pH 7.0, 70 mM | ImpRes, pH | pH 6.0, | | | |
| | NaCl | 6.0, 410 mM | Flowthrough | | | |
| | | NaCl | | | | |
| 15 | Nuvia cPrime | HyperCel | PPA HyperCel, | 82 | 214 | 296 |
| | pH 7.0, 70 mM | STAR AX, pH | pH Step, pH | | | |
| | NaCl | 7.0, | 3.8 | | | |
| | | Flowthrough | | | | |
| 16 | Nuvia cPrime | Toyopearl MX- | PPA HyperCel, | 84 | 214 | 298 |
| | pH 7.0, 70 mM | Trp-650M, pH | pH Step, pH | | | |
| | NaCl | 7.0 | 3.8 | | | |
| | | Flowthrough | | | | |
| 17 | Nuvia cPrime | Q Sepharose | Capto MMC | 33 | 282 | 315 |
| | pH 7.0, 70 mM | HP | ImpRes, pH | | | |
| | NaCl | pH 7.0, | 7.0, 210 mM | | | |
| | | Flowthrough | NaCl | | | |
| 18 | Nuvia cPrime | Q Sepharose | Capto Adhere, | 74 | 245 | 319 |
| | pH 7.0, 70 mM | HP | pH 6.0, 410 | | | |
| | NaCl | pH 7.0, | mM NaCl | | | |
| | | Flowthrough | | | | |
| 19 | Capto MMC | Q Sepharose | Capto Adhere | 243 | 79 | 322 |
| | ImpRes | HP | pH 7.0 Salt | | | |
| | pH 7.0 Salt | pH 7.0, | Step, 610 mM | | | |
| | Step, 210 mM | Flowthrough | NaCl | | | |
| | NaCl | | | | | |
| 20 | Capto Adhere, | Q Sepharose | SP Sepharose | 32 | 298 | 330 |
| | pH Step, pH | HP, pH 5.0, | HP, pH 5.0, | | | |
| | 5.0 | Flowthrough | 170 mM NaCl | | | |

### Downstream Purification Process Development for IFN

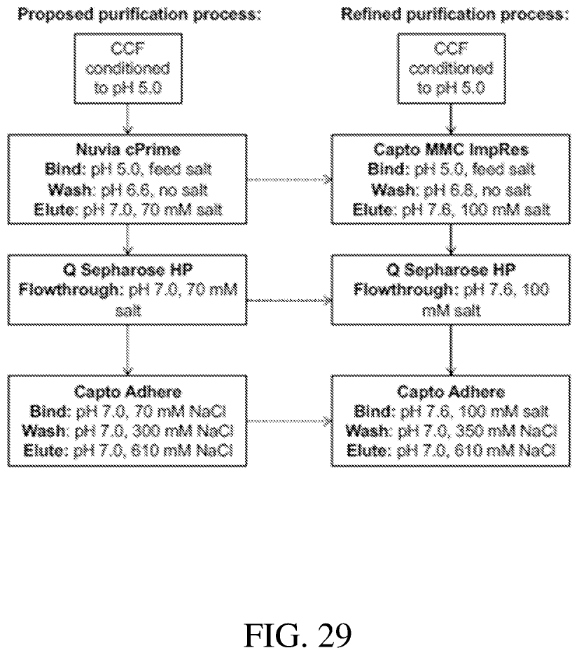

The development of a process to purify IFN from Pichia CCF was initiated using the output from the process selection tool shown in Table 13. The specified purification targets were host cell proteins (HCP) less than 100 ppm and DNA less than limit of detection (LOD) of a Quant-iT™ PicoGreen® dsDNA Assay Kit (ThermoFisher) kit. The initial top- ranked process (i.e., sequence 1) was discounted due to the use of Nuvia cPrime as the capture step, because the wash conditions for Nuvia cPrime resulted in a significant loss of the product. Sequences 3 and 4 were discounted due to the use of Toyopearl MX-Trp-650M as the capture step since the elution salt concentration was 300 mM, which was close to the conductivity of the Pichia CCF load. Sequence 19 was selected, because step 1 was the same as the process utilized in Example 3 and steps 2 and 3 were the same as sequence 1. FIG. 29 shows the original process (left) selected from software output and the final optimized process (right).

The final process, which employed Capto MMC ImpRes as a capture step, Q Sepharose HP as a flow-through step, and Capto Adhere as a polishing step, was used to purify IFN from Pichia CCF and problematic IFN variants, and the resulting solution was characterized for product recovery and clearance of HCP, DNA, product variants, and product aggregates. For Capto MMC ImpRes, the bind condition was 20 mM sodium citrate, pH 5.0; the wash condition was 20 mM sodium phosphate, pH 6.8; and the elute condition was 100 mM sodium chloride, 20 mM sodium phosphate, pH 7.6. For Q Sepharose HP, the flow-through condition was 100 mM sodium chloride, 20 mM sodium phosphate, pH 7.6. For Capto Adhere, the bind condition was 100 mM sodium chloride, 20mM sodium phosphate, pH 7.6; the wash condition was 350 mM sodium chloride, 20 mM sodium phosphate, pH 7.0; and the elute condition was 610 mM sodium chloride, 20 mM sodium phosphate, pH 7.0. The load challenge for capture step was approximately 9.2 mg IFN/mL resin. An SDS-PAGE gel was run to test product quality. Enzyme-linked immunosorbent assay (ELISA) (Cygnus Technologies) was carried out on Pichia to determine information on process contaminants including host cell proteins (HCPs). A Quant-iT™ PicoGreen® dsDNA Assay Kit (ThermoFisher) was carried out to determine information on DNA process contaminants. Size-exclusion chromatography (SEC) was used to quantify aggregates. Reverse phase liquid chromatography (RPLC) was used to determine charge variants and product titer. Capillary electrophoresis (Perkin Elmer GXII) was also used to determine product titer. A cell-based proliferation assay was conducted to determine product activity.

The results presented in Table 14 show that overall product yield was 71.1%. The final product concentration was 0.172 mg/mL and the final total variant content was 7.9%. FIG. 30 shows RP-UPLC chromatograms of the cell culture fluid containing IFN prior to purification and after each purification step.

TABLE 14
IFN Purification Process Performance

| | Product | | | | |
|---|---|---|---|---|---|
| | Concentration | Step | HCP conc. | DNA conc. | Total Variant |
| Sample | (mg/mL) | Recovery | (ng/mg IFN) | (ng/mg IFN) | Content |
| IFN CCF | 0.167 | — | 258,802.4 ± 132,934.1 | 45,893.6 ± 1,000.9 | 31.4% |
| Capto MMC | 1.182 | 77.7% | 62.6 ± 45.4 | 188.6 ± 22.2 | 15.3% |
| ImpRes Eluate | | | | | |
| Q Sepharose | 0.286 | 93.6% | 6.0 ± 1.4 | 173.1 ± 21.7 | 15.5% |
| HP | | | | | |
| Flowthrough | | | | | |
| Capto Adhere | 0.172 | 71.1% | <LOD* | 222.9 ± 16.8 | 7.9% |
| Eluate | | | | | |

*LOD is limit of detection

### Example 10

### Chromatographic Process Design for Purification of Camelid Single-domain Antibody 3B2

### Fractionation Experiments for Cell Culture Fluid Containing Single-domain Antibody 3B2

Null strain Pichia pastoris cell culture fluid (CCF) fractionation experiments were carried out as described in Example 1.

In order to generate the product retention database, single-domain antibody 3B2 (i.e., SEQ. ID. No. 1), which is specific for the VP6 protein of rotavirus A, was produced using an upstream component, as described herein, and partially purified on a multimodal cation exchange resin to concentrate the product and reduce host cell proteins.

The partially purified 3B2 at a concentration of about 4 mg/ml was diluted 20× into a load buffer for the various gradient screens (i.e., 20 mM citrate, pH 5 for the pH 5 salt gradient screen; 20 mM sodium phosphate, pH 6 for the pH 6 salt gradient screen; or 20 mM sodium phosphate, pH 7 for the pH 7 salt gradient screen). The 3B2 solution was prepared less than 24 hours in advance of its use. 3B2 fractionation experiments were carried out according to nearly the same protocols and using the same chromatographic resins and buffers as the CCF G-CSF fractionation experiments described in Example 1. The difference was that in this case system flow rate was kept constant at 1 CV/min throughout the method. Column load challenge was kept constant at 2.5 mg/mL, and product elution pH or salt concentration was determined by peak maximum at 280 nm. Pure component 3B2 retention data was obtained directly from the AKTA chromatogram.

### RP-UPLC Analysis of CCF Partitioning Fractionation Experiments

RP-UPLC analysis of samples from the CCF partitioning fractionations was performed according to the protocol described in Example 1. The process-related impurity retention data set was constructed according to the protocols described in Example 1. The downstream process (DSP) generation tool was used as described in Example 1, except an additional constraint was placed on the downstream process generation tool. The process was required to be two steps.

Initially, 3B2 product retention data was loaded into the program. As described in Example 1, each resin or step was categorized as bind-elute, explicit flow-through, or implicit flow-through. The 3B2 product retention data was used as inputs to generate a list of all candidate 2-step process sequences, of both resin types and operating conditions, which recover the product, wherein each step corresponded to running the 3B2 product solution through a column with a resin from the screening process using a pH or salt gradient. The constraints described above in Example 1 were implemented to reject undesirable processes.

Then, using both the 3B2 product retention data and host cell protein (HCP) data from the null cell culture fluid (CCF) fractionation experiments, the processes were ranked using Equation 1 as described in Example 1. Using this equation, scores were assigned and each set was rank-ordered. The top 20 process sequences selected by the process selection tool for the purification of 3B2 are shown in Table 15.

TABLE 15

| | | | Process |
|---|---|---|---|
| Rank | Step 1 | Step 2 | Score |
| 1 | Capto MMC, pH | Capto Adhere, pH | 36 |
| | Gradient, pH 7 | 7.0, Flowthrough | |
| 2 | Capto MMC, pH | HyperCel STAR | 78 |
| | Gradient, pH 7 | AX, pH 7.0, | |
| | | Flowthrough | |
| 3 | Capto MMC, pH | Capto MMC | 374 |
| | Gradient, pH 7 | ImpRes, pH 7.0, | |
| | | Flowthrough | |
| 4 | CMM HyperCel, | Capto Adhere, pH | 450 |
| | pH Gradient, pH | 7.0, Flowthrough | |
| | 6.8 | | |
| 5 | Capto MMC, pH | Capto MMC | 461 |
| | 6.0, 370 mM NaCl | ImpRes, pH 6.0, | |
| | | Flowthrough | |
| 6 | CMM HyperCel, | Capto Adhere, pH | 760 |
| | pH 6.0, 410 mM | 6.0, Flowthrough | |
| | NaCl | | |
| 7 | CMM HyperCel, | Capto MMC | 811 |
| | pH Gradient, pH | ImpRes, pH 7.0, | |
| | 6.8 | Flowthrough | |
| 8 | CMM HyperCel, | Capto MMC, pH | 871 |
| | pH 6.0, 410 mM | 6.0, Flowthrough | |
| | NaCl | | |
| 9 | Nuvia cPrime, pH | Capto Adhere, pH | 917 |
| | Gradient, pH 5.7 | 6.0, Flowthrough | |
| 10 | CMM HyperCel, | Capto MMC | 975 |
| | pH 6.0, 410 mM | ImpRes, pH 6.0, | |
| | NaCl | Flowthrough | |
| 11 | CMM HyperCel, | HyperCel STAR | 1102 |
| | pH 6.0, 410 mM | AX, pH 6.0, | |
| | NaCl | Flowthrough | |
| 12 | CMM HyperCel; | Toyopearl MX- | 1313 |
| | pH 6.0; 410 mM | Trp-650M, pH | |
| | NaCl | 6.0, Flowthrough | |
| 13 | CMM HyperCel, | Nuvia cPrime, pH | 1995 |
| | pH 6.0, 410 mM | 6.0, Flowthrough | |
| | NaCl | | |
| 14 | Nuvia cPrime, pH | HEA HyperCel, pH | 2930 |
| | 5.0, 410 mM NaCl | 5.0, Flowthrough | |
| 15 | Nuvia cPrime, pH | HyperCel STAR | 4160 |
| | Gradient, pH 5.7 | AX, pH 6.0, | |
| | | Flowthrough | |
| 16 | Capto MMC, pH | HyperCel STAR | 4253 |
| | 6.0, 370 mM NaCl | AX, pH 6.0, | |
| | | Flowthrough | |
| 17 | Nuvia cPrime, pH | Capto Adhere, pH | 4496 |
| | 5.0, 410 mM NaCl | 5.0, Flowthrough | |
| 18 | Capto MMC, pH | Capto Adhere, pH | 6868 |
| | 5.0, 690 mM NaCl | 5.0, Flowthrough | |
| 19 | Capto MMC, pH | HyperCel STAR | 7374 |
| | 5.0, 690 mM NaCl | AX, pH 5.0, | |
| | | Flowthrough | |
| 20 | Capto MMC, pH | HEA HyperCel, pH | 11295 |
| | 5.0, 690 mM NaCl | 5.0, Flowthrough | |

### Decision Process to Narrow the Downstream Process Candidate List

Once potential sequences were identified using the process generation tool, a decision process was implemented to select the specific potential sequence for process development according to the protocols described in Example 1.

### Downstream Purification Process Development for Camelid Single-domain Antibody 3B2

The development of a process to purify 3B2 from Pichia CCF was initiated using the output from the process selection tool shown in Table 15. The specified purification targets were host cell proteins (HCP) less than 100 ppm and DNA less than the limit of detection (LOD) of a Quant-iT™ PicoGreen® dsDNA Assay Kit (ThermoFisher) kit. Sequence 1 was selected as the starting point for 3B2 process development.

The initial top-scoring process utilized Capto MMC as the capture step and Capto Adhere as the polishing step. Upon experimental validation, capacity and process robustness were improved by replacing the capture step with CMM HyperCel and the polishing step with HyperCel STAR AX. Capto MMC was found to have poor product recovery and was replaced with CMM HyperCel, which appeared in other top ranking processes. Capto Adhere resulted in substantial dilution of the final product. HyperCel STAR AX, which appeared in other top ranking processes, was found to provide comparable performance with respect to impurity clearance and resulted in less dilution. This revised process corresponds to process 25.

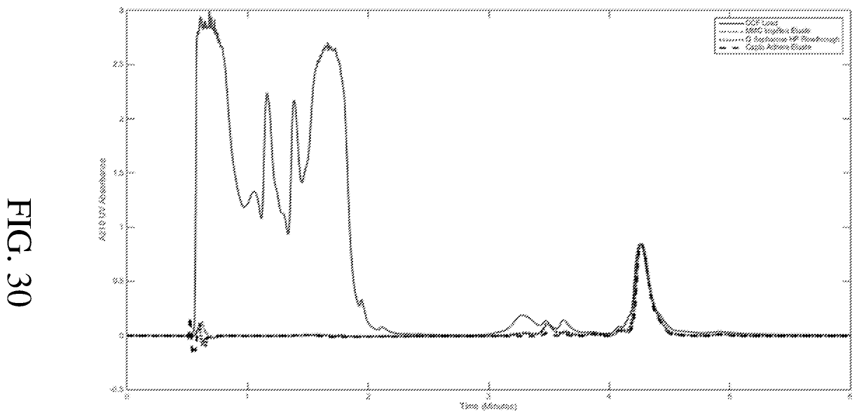

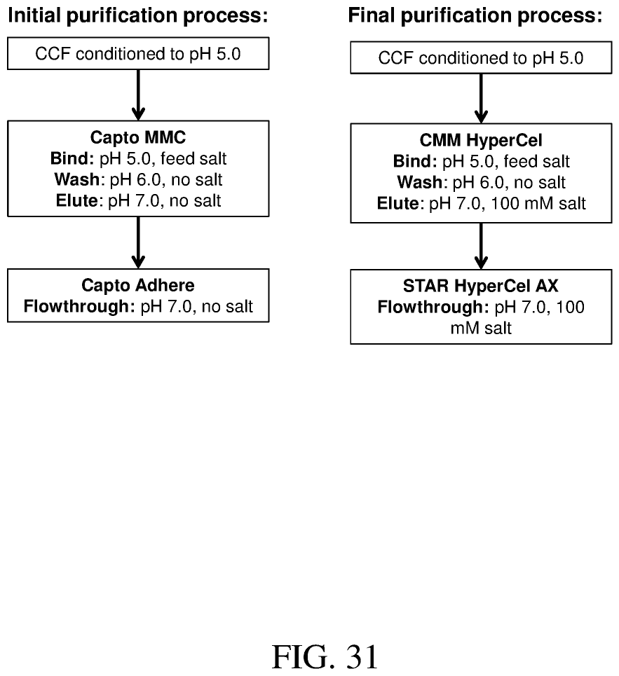

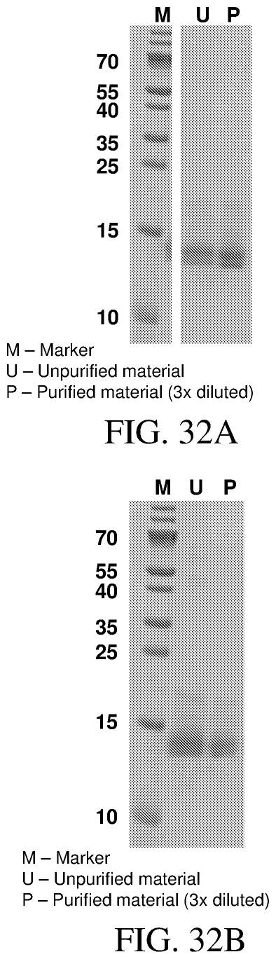

FIG. 31 shows the original process (left) selected from software output and the final optimized process (right).The final process was used to purify 3B2 from Pichia CCF, and the resulting solution was characterized for product recovery and clearance of HCP, DNA, and product aggregates. The load challenge for capture step was approximately 5 mg 3B2/mL resin. An SDS-PAGE gel was run to test product quality. FIG. 32A shows the results of the SDS-page of 3B2. Enzyme-linked immunosorbent assay (ELISA) (Cygnus Technologies) was carried out on Pichia to determine information on process contaminants including host cell proteins (HCPs). A Quant-iT™ PicoGreen® dsDNA Assay Kit (ThermoFisher) was carried out to determine information on DNA process contaminants. Reverse phase liquid chromatography (RPLC) was used to determine product titer. Absorbance spectroscopy was also used to determine product titer.

The results presented in Table 16 show that overall product recovery was approximately 94.8%. The final product concentration was 0.244 mg/mL.

TABLE 16

| | Prod. conc. | | DNA |
|---|---|---|---|
| Sample | (mg/ml) | Recovery | (PPM) |
| 3B2 CCF | 0.122 | — | 1.18 × 10⁶ |
| CMM HyperCel | 0.530 | 98.9% | 5.2 |
| HyperCel STAR | 0.244 | 95.9% | 15.9 |
| flow-through | | | |

### Example 11

### Purification of Camelid Single-domain Antibody 2KD1

This example describes the purification of camelid single-domain antibody 2KD1 using the downstream purification process for camelid single-domain antibody 3B2 described in Example 10. Though single-domain antibodies 3B2 and 2KD1 differ in both sequence and isoelectric point (i.e., 6.71 and 7.75, respectively), the process developed in Example 10 effectively purified the single domain antibody 2KD1 (i.e., protein SEQ. ID. No. 2).

The final process described in Example 10 was used to purify 2KD1 from Pichia CCF, and the resulting solution was characterized for product recovery and clearance of HCP, DNA, and product aggregates. The load challenge for capture step was approximately 10 mg 2KD1/mL resin. An SDS-PAGE gel was run to test product quality. FIG. 32B shows the results of the SDS-page of 3B2. Enzyme-linked immunosorbent assay (ELISA) (Cygnus Technologies) was carried out on Pichia to determine information on process contaminants including host cell proteins (HCPs). A Quant-iT™ PicoGreen® dsDNA Assay Kit (ThermoFisher) was carried out to determine information on DNA process contaminants. Size-exclusion chromatography (SEC) was used to quantify aggregates. Reverse phase liquid chromatography (RPLC) was used to determine charge variants and product titer. Absorbance spectroscopy was also used to determine product titer.

The results presented in Table 17 show that overall product recovery was approximately 60%. The final product concentration was 0.398 mg/mL.

TABLE 17

| | Prod. conc. | | HCP | DNA |
|---|---|---|---|---|
| Sample | (mg/ml) | Recovery | (PPM) | (PPM) |
| 2KD1 CCF | 0.258 | — | 115,711.9 | 3.17 × 10⁶ |
| CMM HyperCel | 0.475 | 59.8% | 420.7 | 65.6 |
| HyperCel STAR | 0.398 | 101.6% | 9.5 | 19.3 |
| flow-through | | | | |

### Example 12

### Chromatographic Process Design for Purification of Interferon a-213 (IFN)

IFN variant identification

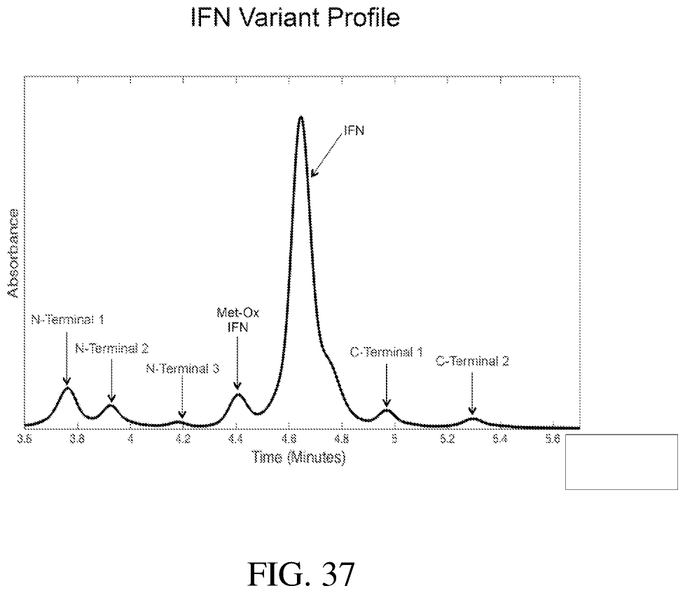

IFN was partially purified from cell culture fluid from a Pichia pastoris culture expressing IFN as described in Example 3. Partially purified IFN was fractionated by RP-UPLC. FIG. 37 shows a chromatogram of partially purified IFN. Product variants identified included N-terminal variants (labeled in FIG. 37 as N-Terminal 1, N-Terminal 2, and N-Terminal 3) and C-terminal variants (labeled in FIG. 37 as C-Terminal 1 and C-Terminal 2). A singularly oxidized variant was also observed (labeled in FIG. 37 as Met-Ox IFN). It was determined that the N-terminal variants and the C-terminal variants should be removed through a downstream purification process.

### Downstream Purification Process Development for IFN

Null strain Pichia pastoris cell culture fluid (CCF) fractionation experiments were performed as described in Example 1, and IFN fractionation experiments were carried out as described in Example 3. Reversed phase-ultra high pressure liquid chromatography (RP-UPLC) analysis of samples from the CCF partitioning fractionations and IFN solution fractionations was performed according to the protocol described in Example 1. Based on this analysis, a process-related impurity retention data set and a product-related impurity retention data set were constructed according to the protocol described in Example 3.

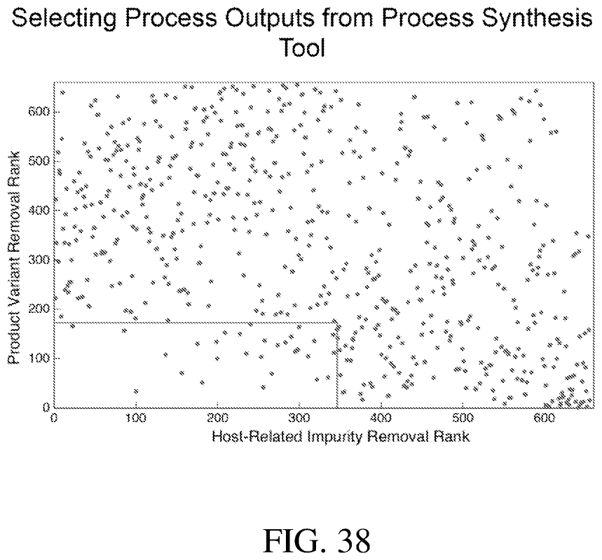

Using the process-related and product-related impurity retention data sets, each candidate sequence of partitioning steps was assigned a rank for expected removal of process-related impurities and, independently, a rank for expected removal of product-related impurities. To illustrate, FIG. 38 shows a plot of each candidate sequence according to process-related impurity removal rank (x-axis) and product-related impurity removal rank (y-axis). Certain criteria for process-related impurity removal rank and product-related impurity removal rank were established, and candidate sequences satisfying those criteria (e.g., those sequences falling within the box in the lower left hand corner of FIG. 38) were identified.

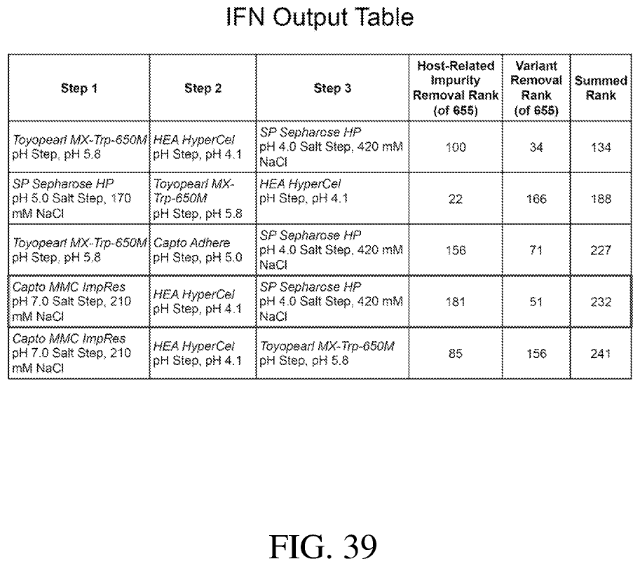

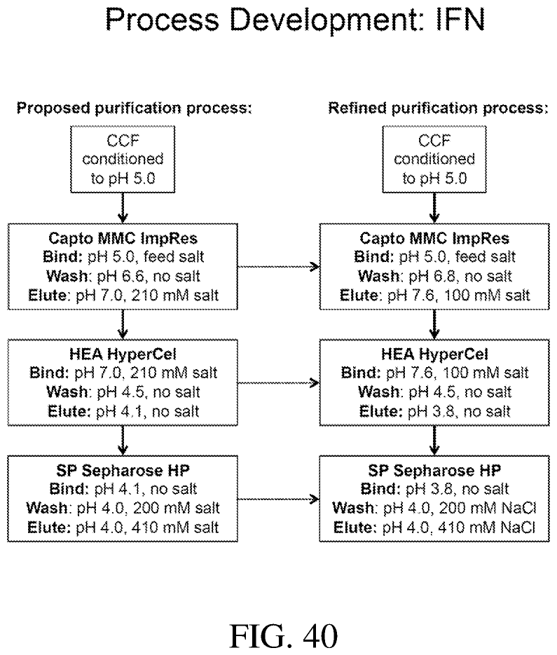

Based on a combination of the process-related impurity removal rank and the product-related impurity removal rank, the top five candidate sequences (out of 655 possible sequences) were identified, as shown in FIG. 39. In particular, each candidate sequence was assigned a process-related impurity removal rank (out of 655) and was independently assigned a product-related impurity removal rank (out of 655). The process-related and product-related impurity removal ranks were summed to obtain a summed rank, and the five sequences shown in FIG. 39 represent the five candidate sequences having the highest summed ranks out of the 655 possible sequences.A sequence employing Capto MMC ImpRes as a capture step, HEA HyperCel as a first polishing step, and SP Sepharose HP as a second polishing step, was selected for further optimization. A schematic representation of the refined purification process is shown in FIG. 40. For Capto MMC ImpRes, the bind condition was pH 5.0, feed salt; the wash condition was pH 6.8, no salt; and the elute condition was pH 7.6, 100 mM salt. For HEA HyperCel, the bind condition was pH 7.6, 100 mM salt; the wash condition was pH 4.5, no salt; and the elute condition was pH 3.8, no salt. For SP Sepharose HP, the bind condition was pH 3.8, no salt; the wash condition was pH 4.0, 200 mM NaCl; and the elute condition was pH 4.0, 410 mM NaCl.

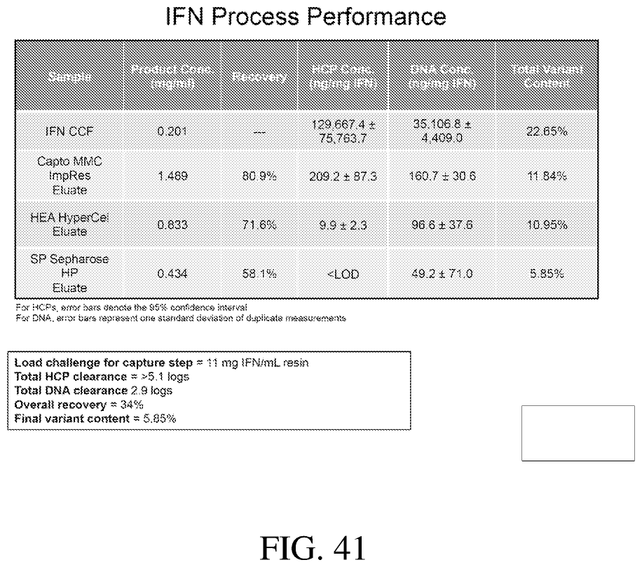

This refined purification process was used to purify IFN from Pichia CCF and IFN variants, and the resulting solution was characterized for product recovery, HCP concentration, DNA concentration, and total product variant content. FIG. 41 presents the characterization results after each step of the refined purification process. From FIG. 41, it can be seen that the overall recovery was 34% and the final product concentration was 0.434 mg/mL. By using the selected purification process, the total product variant content was greatly reduced from 22.65% to 5.85%. The HCP concentration was also lower than the detectable limit, and the DNA concentration was 49.2 ±71.0 ng/mg IFN.

### Example 13

### Chromatographic Process Design for Purification of IFN

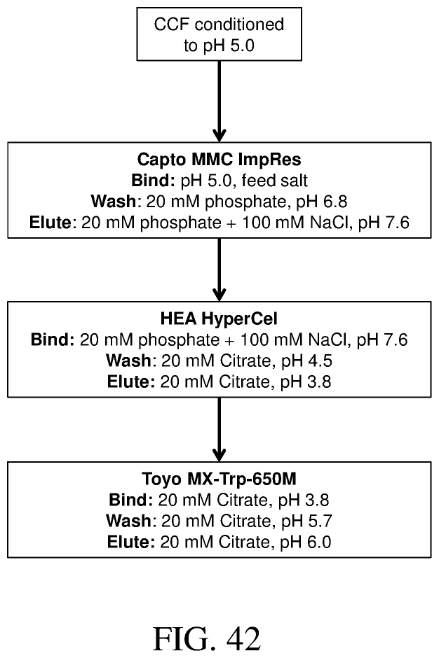

The process described in Example 12 was used to identify another IFN purification process. A schematic representation of this sequence is shown in FIG. 42. The sequence employed Capto MMC ImpRes as a capture step, HEA HyperCel as a first polishing step, and Toyo MX-Trp-650M as a second polishing step. For Capto MMC ImpRes, the bind condition was pH 5.0, feed salt; the wash condition was pH 6.8, 20 mM phosphate; and the elute condition was pH 7.6, 20 mM phosphate and 100 mM NaCl. For HEA HyperCel, the bind condition was pH 7.6, 20 mM phosphate and 100 mM NaCl; the wash condition was pH 4.5, 20 mM citrate; and the elute condition was pH 3.8, 20 mM citrate. For Toyo MX-Trp-650M, the bind condition was pH 3.8, 20 mM citrate; the wash condition was pH 5.7, 20 mM citrate; and the elute condition was pH 6.0, 20 mM citrate.

### Example 14

### Chromatographic Process Design for Purification of IFN

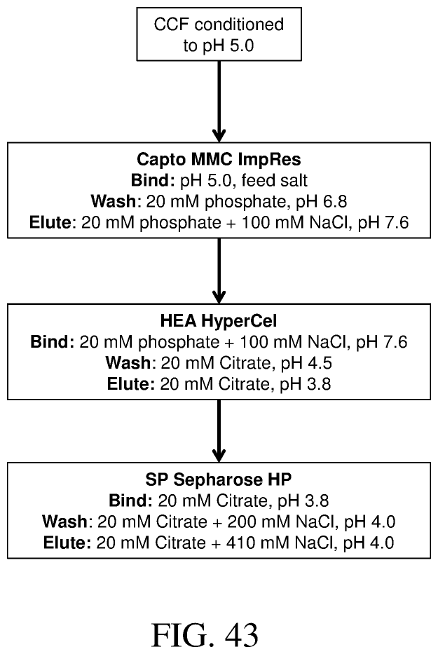

The process described in Example 12 was used to identify another IFN purification process. A schematic representation of this sequence is shown in FIG. 43. The sequence employed Capto MMC ImpRes as a capture step, HEA HyperCel as a first polishing step, and SP Sepharose HP as a second polishing step. For Capto MMC ImpRes, the bind condition was pH 5.0, feed salt; the wash condition was pH 6.8, 20 mM phosphate; and the elute condition was pH 7.6, 20 mM phosphate and 100 mM NaCl. For HEA HyperCel, the bind condition was pH 7.6, 20 mM phosphate and 100 mM NaCl; the wash condition was pH 4.5, 20 mM citrate; and the elute condition was pH 3.8, 20 mM citrate. For SP Sepharose HP, the bind condition was pH 3.8, 20 mM citrate; the wash condition was pH 4.0, 20 mM citrate and 200 mM NaCl; and the elute condition was pH 4.0, 20 mM citrate and 410 mM NaCl.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-14. (canceled)

15. A biomanufacturing system, comprising:
a perfusion bioreactor, wherein the perfusion bioreactor comprises:
a reaction chamber configured to receive at least one feed stream comprising at least one cell culture medium;
a suspension comprising the at least one cell culture medium and at least a first type of biological cells configured to express at least one biologically-produced product;
at least one filter probe at least partially submerged in the suspension, wherein the at least one filter probe is configured to produce at least one filtrate stream lean in the first type of biological cells relative to the suspension, wherein the at least one filtrate stream comprises the at least one biologically-produced product;
an adjustment module fluidically connected to the perfusion bioreactor, wherein the adjustment module is configured to adjust one or more properties of the at least one filtrate stream to produce an adjusted filtrate stream; and
a purification module fluidically connected to the adjustment module, wherein the purification module is configured to remove at least a first type of impurity and a second type of impurity from the adjusted filtrate stream to produce a purified filtrate stream, wherein the purification module comprises:
a first partitioning unit configured to remove at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and
a second partitioning unit configured to remove at least a second type of impurity from the first partitioned filtrate stream to produce a second partitioned filtrate stream lean in the second type of impurity relative to the first partitioned filtrate stream, wherein the second partitioned filtrate stream comprises the at least one biologically-produced product,
wherein the system is configured to be continuously operated.

16. The biomanufacturing system of claim 15, further comprising a level sensing system configured to measure a level of the suspension in the reaction chamber of the perfusion bioreactor.

17. The biomanufacturing system of claim 15, wherein the first type of biological cell is microbial.

18. The biomanufacturing system of claim 17, wherein the microbial cell is a yeast, optionally being Pichia pastoris.

19. The biomanufacturing system of claim 15, wherein the biologically-produced product is selected from granulocyte-colony stimulating factor (G-CSF), human growth hormone (hGH), interferon α-2β (IFN), and a single domain antibody.

20. The biomanufacturing system of claim 19, wherein the purification module comprises:
a first column comprising a multimodal cation exchange resin;
a second column comprising a resin selected from an anion exchange resin and a hydrophobic charge induction chromatography (HCIC) resin; and, optionally,
a third column comprising a resin selected from an HCIC resin and a cation exchange resin.

21. The biomanufacturing system of claim 20, wherein the purification module is configured to remove a third type of impurity.

22. The biomanufacturing system of claim 15, wherein the impurity is a host-related impurity, optionally being a host cell protein (HCP).

23. A method of producing at least one biologically-produced product, comprising:
supplying at least one feed stream comprising at least one cell culture medium to a perfusion bioreactor at a first flow rate;
producing, within the perfusion bioreactor, a suspension comprising the at least one cell culture medium and at least a first type of biological cells expressing the at least one biologically-produced product;
causing at least a portion of the suspension to flow through at least one filter probe to produce at least one filtrate stream lean in the first type of biological cells, wherein the at least one filtrate stream comprises the at least one biologically-produced product, wherein the at least one filter probe is at least partially submerged in the suspension;
adjusting one or more properties of the at least one filtrate stream to produce an adjusted filtrate stream;
removing, within a purification module, at least a first type of impurity and a second type of impurity from the adjusted filtrate stream to produce a purified filtrate stream flowing at a second flow rate, wherein the purified filtrate stream comprises the at least one biologically-produced product and is lean in the first type of impurity and the second type of impurity relative to the adjusted filtrate stream, wherein producing the purified filtrate stream comprises:
removing, within a first partitioning unit, at least the first type of impurity from the adjusted filtrate stream to produce a first partitioned filtrate stream lean in the first type of impurity relative to the adjusted filtrate stream, wherein the first partitioned filtrate stream comprises the at least one biologically-produced product; and
removing, within a second partitioning unit, at least the second type of impurity from the first partitioned filtrate stream to produce a second partitioned filtrate stream lean in the second type of impurity relative to the first partitioned filtrate stream, wherein the second partitioned filtrate stream comprises the at least one biologically-produced product.

24. The method of claim 23, further comprising measuring a level of the suspension in a reaction chamber of the perfusion bioreactor using a level sensing system.

25. The method of claim 23, wherein the first type of biological cell is microbial.

26. The method of claim 25, wherein the microbial cell is a yeast, optionally being Pichia pastoris.

27. The method of claim 23, wherein the biologically-produced product is selected from granulocyte-colony stimulating factor (G-CSF), human growth hormone (hGH), interferon α-2β (IFN), and a single domain antibody.

28. The method of claim 27, wherein producing the purified filtrate stream further comprises:
flowing the at least one filtrate through a first column comprising a multimodal cation exchange resin;
collecting one or more first fractions comprising the biologically-produced product from an outflow of the first column;
flowing the one or more first fractions through a second column comprising a resin selected from an anion exchange resin and a hydrophobic charge induction chromatography (HCIC) resin;
collecting one or more second fractions comprising the biologically-produced product from an outflow of the second column; and, optionally,
flowing the one or more second fractions through a third column comprising a resin selected from an HCIC resin and a cation exchange resin; and, optionally,
collecting one or more third fractions comprising the biologically-produced product from an outflow of the third column.

29. The method of claim 28, wherein producing the purified filtrate stream comprises removing a third type of impurity.

30. The method of claim 23, wherein the impurity is a host-related impurity, optionally being a host cell protein (HCP).


SEQUENCE LISTING (2 sequences)

SEQ ID NO: 1 - protein, 128 aa, Artificial Sequence
  Synthetic polypeptide
MADVQLQASGGGLAQAGDSLTLSCAASGRTFSGYVVGWFRQAPGAEREFVGAIRWSEDSTWYGDSMKGRILISRNNIKNTVNLQMFNLKPEDTAVYVCAAGAGDIVTTETSYNYWGRGTQVTVSSRGR

SEQ ID NO: 2 - protein, 126 aa, Artificial Sequence
  Synthetic polypeptide
MADVQLQASGGGFVQPGDSLSLSCAASGGTFSSYSIGWFRQGPGKEREFVATISSSDSPWYGEPAKGRFTVARVNAKNTAYLHLNRLKPEDTATYYCAAGSVQHMANENEYVYWGQGTQVTVSSGR